(19) United States
(12) Patent Application Publication
(10) Pub. No.: US 2005/0059657 A1
(43) Pub. Date: Mar. 17, 2005

(54) Aminoisoxazole derivatives active as kinase inhibitors

(76) Inventors: Marcello Cavicchioli, Lyon (FR); Paolo Pevarello, Pavia (IT); Barbara Salom, Vedano al Lambro (IT); Anna Vulpetti, Brugherio (IT)

Correspondence Address: AKIN, GUMP, STRAUSS, HAUER & FELD, 1111 LOUISIANA STREET, 44TH FLOOR, HOUSTON, TX, 77002, US

(21) Appl. No.: 10/485,871

(22) PCT Filed: Jul. 29, 2002
(86) PCT No.: PCT/EP02/08634

(30) Foreign Application Priority Data
Aug. 6, 2001 (US) 09921751

(51) Int. Cl. A61K 31/541; A61K 31/5377; A61K 31/496; A61K 31/454; A61K 31/42

(52) U.S. Cl. 514/227.8; 514/235.2; 514/254.04; 514/326; 514/378

(57) ABSTRACT

Compounds (I) which are aminoisoxazole derivatives or pharmaceutically acceptable salts thereof, together with pharmaceutical compositions comprising them are disclosed; these compounds or compositions are useful in the treatment of diseases caused by and/or associated with an altered protein kinase activity such as cancer, cell proliferative disorders, Alzheimer's disease, viral infections, auto-immune diseases and neurodegenerative disorders.

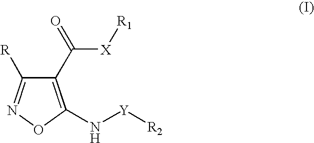

The present invention relates to aminoisoxazole derivatives active as kinase inhibitors and, more particularly, to 3-heteroaryl-aminoisoxazole derivatives, to a process for their preparation, to pharmaceutical compositions comprising them and to their use as therapeutic agents, particularly in the treatment of diseases linked to disregulated protein kinases.

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neurofibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

It is an object of the invention to provide compounds which are useful in therapy as agents against a host of diseases caused by and/or associated to a disregulated protein kinase activity.

It is another object to provide compounds which are endowed with multiple protein kinase inhibiting activity. The present inventors have now discovered that some 3-heteroaryl-aminoisoxazole derivatives, hereinafter shortly referred to as aminoisoxazole derivatives or aminoisoxazoles, are endowed with multiple protein kinase inhibiting activity and are thus useful in therapy in the treatment of diseases associated with disregulated protein kinases.

More specifically, the aminoisoxazoles of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Due to the key role of PKs in the regulation of cellular proliferation, these aminoisoxazoles are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of the invention are useful as cyclin dependent kinase (cdk) inhibitors and also as inhibitors of other protein kinases such as, for instance, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, VEGF-R, PI3K, weel kinase, Src, Abl, Akt, ILK, MK-2, IKK-2, Cdc7, Nek, and thus be effective in the treatment of diseases associated with other protein kinases.

Some 5-amino-isoxazole derivatives structurally related to the compounds of formula (I) are known in the art, either for pharmaceutical or non pharmaceutical use.

As an example, the international patent application WO 98/47880 discloses guanidino isoxazoles useful in the treatment of autoimmune and inflammatory diseases.

WO 98/28282 discloses a very broad class of widely substituted isoxazole derivatives, useful in therapy as factor Xa inhibitors.

WO 97/34881 discloses isoxazole and isoxazoline derivatives, further substituted in position 5 by penta-atomic heterocyclic rings, as antitumor agents.

Nitrofuryl-isoxazole derivatives are known in the art as antibacterial and antiprotozoal agents (see, for a reference, U.S. Pat. No. 3,631,169 and GB 1250219).

Other 5-aminoisoxazole derivatives further substituted in position 3 by given heteroaryl groups are known in the art. See, in particular, Chemical Abstracts 79(1973):61409 which discloses 5-amino-3-(2-aminofuryl-5-yl)-4-methoxycarbonyl-isoxazole in a study to assess the stability of 3-(nitrofuryl)isoxazoles; or Indian J. Chem., Sect. B (1984), 23B(10), 926-9 which discloses 5-amino-3-(indol-3-yl)-4-ethoxycarbonyl-isoxazoles optionally substituted in the indole moiety, as end products of synthetic schemes.

Accordingly, the present invention provides a method for treating diseases caused by and/or associated with an altered protein kinase activity, by administering to a mammal in need thereof an effective amount of an aminoisoxazole derivative represented by formula (I): 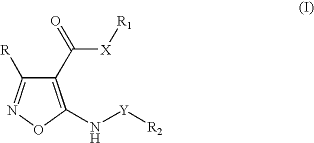
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆ cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;
R₂ and R₃ have, each independently, the meanings above reported for R₁ or represent an optionally substituted straight or branched C₂-C₆ alkenyl or alkynyl group; and the pharmaceutically acceptable salts thereof.

In a preferred embodiment of the method described above, the disease caused by and/or associated with an altered protein kinase activity is selected from the group consisting of cancer, cell proliferative disorders, Alzheimer's disease, viral infections, auto-immune diseases and neurodegenerative disorders.

Specific types of cancer that may be treated include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderoma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

In addition, the method object of the present invention, also provides tumor angiogenesis and metastasis inhibition.

The present invention further provides an aminoisoxazole derivative represented by formula (I): 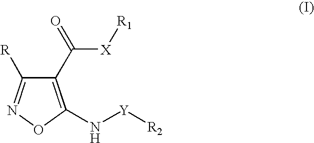
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆ cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;
R₂ and R₃ have, each independently, the meanings above reported for R₁ or represent an optionally substituted straight or branched C₂-C₆ alkenyl or alkynyl group; and the pharmaceutically acceptable salts thereof; provided that:
a) R is other than nitrofuryl; and
b) 5-amino-3-(2-aminofuryl-5-yl)-4-methoxycarbonyl-isoxazole and 5-amino-4-ethoxycarbonyl-3-(indol-3-yl)-isoxazole, optionally further substituted at the indole moiety, being excluded.

The compounds of formula (I), object of the present invention, may have asymmetric carbon atoms and may therefore exist either as racemic admixtures or as individual optical isomers.

Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

In the present description, unless otherwise specified, the meaning of aromatic or non-aromatic ring system is the one conventionally adopted in organic chemistry and clearly encompasses carbocyclic and heterocyclic ring systems.

With the term aryl we typically intend any aromatic either carbocyclic as well as heterocyclic hydrocarbon with 1 or 2 ring moieties, either fused or linked to each other by single bonds, wherein at least one of the carbocyclic or heterocyclic rings is aromatic.

Non limiting examples of aryl groups are, for instance, phenyl, indenyl, biphenyl, α- or β-naphthyl, pyridyl, pyrazinyl, pyrimidinyl, pyridazinyl, indolyl, imidazolyl, imidazopyridyl, 1,2-methylenedioxyphenyl, thiazolyl, isothiazolyl, pyrrolyl, pyrrolyl-phenyl, furyl, phenyl-furyl, benzotetrahydrofuranyl, oxazolyl, isoxazolyl, pyrazolyl, chromenyl, thienyl, benzothienyl, isoindolinyl, benzoimidazolyl, tetrazolylphenyl, pyrrolidinyl-tetrazolyl, quinolinyl, isoquinolinyl quinoxalinyl, benzofurazanyl, 1,2,3-triazolyl, 1-phenyl-1,2,3-triazolyl, and the like.

With the term 5 or 6 membered heteroaryl group with from 1 to 3 heteroatoms selected among nitrogen, oxygen or sulfur we specifically intend an aromatic heterocycle such as, for instance, furan, oxazole, isoxazole, pyrrole, imidazole, pyrazole, thiophene, thiazole, isothiazole, thiadiazole, triazole, pyridine, pyrazine, pyrimidine, pyridazine, and the like.

With the term 5 to 7 membered aromatic or non-aromatic carbocycle we intend, unless otherwise specified, aromatic or non aromatic cyclic hydrocarbons with from 5 to 7 carbon atoms which thus include saturated, partly unsaturated or fully unsaturated carbocyclic rings.

Examples of the above carbocycles according to the invention are, for instance, cyclopentane, cyclopentene, cyclopentadiene, cyclohexane, cyclohexene, 1,3- or 1,4-cyclohexadiene, benzene, cycloheptane, cycloheptene, and the like.

With the term 5 to 7 membered aromatic or non-aromatic heterocycle we intend, unless otherwise specified, any saturated, partly unsaturated or fully unsaturated heterocycle, hence comprising aromatic systems also referred to as heteroaryl groups, with from 1 to 3 heteroatoms selected among nitrogen, oxygen or sulfur.

Therefore, apart from any 5 or 6 membered heteroaryl group previously reported, examples of the above heterocyles are, for instance, 2H-pyrrole, pyran, pyrrolidine, pyrroline, imidazolidine, imidazoline, pyrazolidine, pyrazoline, piperidine, piperazine, morpholine, tetrahydrofuran, oxazolidine, oxazoline, azepine, diazepine, and the like.

From all of the above, it is clear to the skilled man that the term aryl refers to aromatic ring systems comprising heterocyclic and carbocyclic hydrocarbons; the term heteroaryl specifically refers to aromatic heterocyclic rings; the term aromatic or non-aromatic carbocycle encompasses carbocyclic aryl groups as well as saturated or partly unsaturated, hence non-aromatic, carbocyles; the term aromatic or non-aromatic heterocyle refers to heteroaryl groups and saturated or partly unsaturated, hence non-aromatic, heterocyclic rings.

In addition, any heteroaryl R group and any one of R₁, R₂ or R₃ when representing, each independently, a cycloalkyl, aryl or heterocyclyl group, may be optionally condensed through any one of their available bonds with an aromatic or non-aromatic, either carbocyclic as well as heterocyclic ring, substantially as set forth above.

Examples of R groups which are further condensed are, for instance, isobenzofuran, indole, isoindole, 1H-indazole, purine, quinoline, isoquinoline, phthalazine, quinoxaline, quinazoline, cinnoline, pteridine, benzofuran, and the like, which are all bonded through the heteroaryl moiety to the isoxazole in formula (I).

Preferably, when further condensed, the heteroaryl group is condensed with benzene or pyridine rings.

With the term straight or branched C₁-C₆ alkyl we intend a group such as, for instance, methyl, ethyl, n.propyl, isopropyl, n.butyl, isobutyl, sec-butyl, tert-butyl, n.pentyl, n.hexyl and the like.

With the term straight or branched C₂-C₆ alkenyl or alkynyl we intend an unsaturated hydrocarbon chain having a double or triple bond such as, for instance, vinyl, ethynyl, 1-propenyl, allyl, 1- or 2-propynyl, 1-, 2- or 3-butenyl, pentenyl, pentynyl, hexenyl, hexynyl and the like.

With the term C₃-C₆ cycloalkyl group we intend cyclopropyl, cyclobutyl, cyclopentyl, and cyclohexyl.

With the term heterocyclyl group we intend any aromatic or non-aromatic heterocyclic group, as formerly indicated.

According to the meanings provided to R, R₁, R₂ and R₃, any of the above groups may be further optionally substituted in any of the free positions by one or more groups, for instance 1 to 6 groups such as, for instance: halogen, nitro, oxo groups (═O), carboxy, cyano, straight or branched C₁-C₆ alkyl or perfluorinated alkyl, C₃-C₆ cycloalkyl, aryl, heterocyclyl, amino, alkylamino, dialkylamino, arylamino, diarylamino, ureido, alkylureido, arylureido, formylamino, alkylcarbonylamino, alkenylcarbonylamino, arylcarbonylamino, alkoxycarbonylamino, sulphonamido, alkylsulphonamido, arylsulphonamido, hydroxy, straight or branched C₁-C₆ alkoxy, aryloxy, alkylcarbonyloxy, arylcarbonyloxy, alkylcarbonyl, arylcarbonyl, alkoxycarbonyl, aryloxycarbonyl, cycloalkyloxycarbonyl, aminocarbonyl, alkylaminocarbonyl, dialkylaminocarbonyl; alkylthio, arylthio, alkylsulphonyl, arylsulphonyl, arylsulphonyloxy, aminosulphonyl, alkylaminosulphonyl or dialkylaminosulphonyl.

In their turn, whenever appropriate, each of the above groups may also be optionally further substituted with one or more of the aforementioned groups.

When referring, in particular, to the meanings of R, R₂ or R₃, these same groups may be optionally substituted, in any of the free positions, also by straight or branched C₂-C₆ alkenyl or alkynyl groups.

Unless otherwise specified, with the term perfluorinated alkyl we intend an alkyl group wherein two or more hydrogen atoms are replaced by fluorine atoms such as, for instance trifluoromethyl, 2,2,2-trifluoroethyl and the like.

With the term halogen atom we intend a fluorine, chlorine, bromine or iodine atom.

From all of the above, it is clear to the skilled man that any group, which name has been identified as a composite name, has to be intended as construed from the single moieties. As an example, the term alkenylcarbonylamino has to be intended as referring to a carbonylamino group which is further substituted by an alkenyl group; the term heterocyclyl C₁-C₆ alkyl has to be intended as an alkyl group which is substituted by a heterocyclyl group, and the like.

Pharmaceutically acceptable salts of the compounds of formula (I) are the acid addition salts with inorganic or organic, e.g. nitric, hydrochloric, hydrobromic, sulfuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

A first class of preferred compounds of the invention is represented by the compounds of formula (I) wherein Y is a single bond and R₂ is a hydrogen atom, and R, R₁ and X are as above defined.

Still more preferred, within this class, are the compounds wherein X is a —N(R₃)— group and R₃ is as above defined.

Another class of preferred compounds of the invention is represented by the compounds of formula (I) wherein Y is a —CO— group and R, R₁, R₂ and X are as above defined.

Still more preferred, within this class, are the compounds wherein X is —N(R₃)— and R₁ and R₃ are both hydrogen atoms.

Another class of preferred compounds of the invention is represented by the compounds of formula (I) wherein Y is a —CONH— group and R, R₁, R₂ and X are as above defined.

Still more preferred, within this class, are the compounds wherein X is —N(R₃)— and R₁ and R₃ are both hydrogen atoms.

Another class of preferred compounds of the invention is represented by the compounds of formula (I) wherein Y is a —SO₂— group and R, R₁, R₂ and X are as above defined.

Still more preferred, within this class, are the compounds wherein X is —N(R₃)— and R₁ and R₃ are both hydrogen atoms.

Preferably, in any one of the above classes, when the heteroaryl group R is further condensed, it is condensed with a benzene or pyridine ring.

Also preferred are the compounds of formula (I) wherein any heteroaryl or heterocyclyl group has 1 or 2 heteroatoms selected among nitrogen, oxygen or sulfur.

Specific examples of compounds of formula (I), optionally in the form of pharmaceutically acceptable salts, are conveniently listed in the experimental section.

As formerly indicated, the process for preparing the compounds of formula (I) represents a further object of the invention.

The amino isoxazole derivatives of formula (I), object of the invention, are thus obtainable through a synthetic process comprising well known reactions carried out according to conventional techniques, as well as through a new and extremely versatile solid-phase combinatorial process, being both comprised within the scope of the invention.

The compounds of formula (I) and the pharmaceutically acceptable salts thereof may be thus prepared according to a process comprising:
a) reacting a compound of formula (II) with a compound of formula (III) 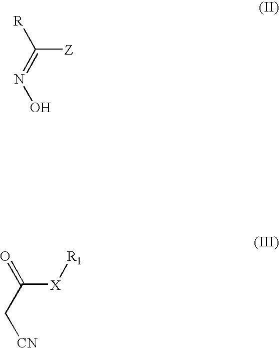
   wherein R, R₁ and X are as above defined and Z represents a halogen atom or a suitable leaving group, so as to obtain a compound of formula (I) 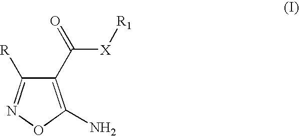
   and, optionally, reacting the compound of formula (I) according to any one of the alternative steps b) below
b.1) with a compound of formula (IV), (V) or (VI)
   R₂—COW (IV),
   R₂—SO₂W′ (V),
   R₂—NCO (VI)
   wherein R₂ is as above defined, W is hydroxy or a suitable leaving group and W′ is a suitable leaving group, so as to obtain the compounds of formula (I) wherein Y is —CO—, —SO₂— or —CONH—, respectively, and R, R₁, R₂ and X are as above defined;
b.2) with a suitable aldehyde or ketone derivative of formula (VII)
   R₂—CO—R₃ (VII)
   wherein R₂ and R₃ are as above defined, under reductive conditions, so as to obtain a compound of formula (I) wherein Y is a group —CH(R₃)— and R, R₁, R₂, R₃ and X are as above defined;
b.3) with a suitable acylating agent in the presence of ammonia, so as to obtain a compound of formula (I) wherein Y is —CONH—, R₂ is hydrogen and R, R₁ and X are as above defined; and, optionally
c) converting the thus obtained compound of formula (I) into another compound of formula (I) and/or into a pharmaceutically acceptable salt thereof.

The above process is an analogy process which can be carried out according to well known methods.

It is clear to the person skilled in the art that if a compound of formula (I), prepared according to the above process, is obtained as an admixture of isomers, their separation into the single isomers of formula (I), carried out according to conventional techniques, is still within the scope of the present invention.

Likewise, the conversion into the free compound (I) of a corresponding salt thereof, according to well-known procedures in the art, is still within the scope of the invention.

According to step a) of the process, the compound of formula (II) is reacted with a compound of formula (III). This reaction is carried out in the presence of a base such as triethylamine, N,N-diisopropylethylamine, 1,8-diazabicyclo[5.4.0]undec-7-ene, pyridine, lithium diisopropylamide or lithium bis(trimethylsilyl)amide, and in a suitable solvent such as, for instance, dichloromethane, chloroform, tetrahydrofuran, acetonitrile, diethyl ether, 1,4-dioxane or N,N-dimethylformamide, at a temperature ranging from about −40° C. to room temperature and for a suitable time, for instance from about 30 minutes to about 96 hours.

The 5-aminoisoxazoles of formula (I) thus obtained can be easily converted into a variety of derivatives of formula (I) and/or into salts thereof.

According to step b.1) of the process, the compound of formula (I) is reacted with any one of the compounds of formula (IV), (V) or (VI), so as to obtain the corresponding derivative of formula (I). In this respect, it is clear to the skilled man that a carboxamido derivative of formula (I) wherein Y is (>C═O) is obtained through reaction with a compound of formula (IV); a sulphonamido derivative of formula (I) wherein Y is (—SO₂—) is obtained through reaction with a compound of formula (V); and an ureido derivative of formula (I) wherein Y is (—NHCO—) is obtained though reaction with a compound of formula (VI).

As formerly indicated, within the compound of formula (IV) W is hydroxy or a suitable leaving group such as, for instance, a halogen atom. Preferably, W is a hydroxy group or a chlorine or bromine atom.

The reaction between a compound of formula (I) and a carboxylic acid derivative of formula (IV) wherein W is hydroxy can be carried out in the presence of a condensing agent such as, for instance, benzotriazol-1-yloxytris(pyrrolidino)phosphonium hexafluorophosphatecarbodiimide, 1,3-dicyclohexylcarbodiimide, bromo-tris-pyrrolidino-phosphonium hexafluorophosphate, 1,3-diisopropylcarbodiimide, o-benzotriazol-1-yl-n,n,n′,n′-tetra-methyluronium tetrafluoroborate, 1-(3-dimethylaminopropyl)-3-ethylcarbodiimide, N-cyclohexylcarbodiimide-N′-propyloxymethyl polystyrene or N-cyclohexylcarbodiimide-N′-methyl polystyrene, in a suitable solvent such as, for instance, dichloromethane, chloroform, tetrahydrofuran, diethyl ether, 1,4-dioxane, acetonitrile, toluene or N,N-dimethylformamide, at a temperature ranging from about −10° C. to reflux and for a suitable time ranging from about 30 minutes to about 96 hours.

The said reaction is optionally carried out in the presence of a suitable catalyst, for instance 4-dimethylaminopyridine, or in the presence of a further condensing agent such as N-hydroxybenzotriazole. The reaction between a compound of formula (I) and a compound of formula (IV) can be also carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, acetonitrile, diethyl ether, 1,4-dioxane or N,N-dimethylformamide, and at a temperature ranging from about −30° C. to room temperature.

The reaction between a compound of formula (I) and a compound of formula (IV) wherein W is a suitable leaving group, for instance chlorine or bromine, can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, and at a temperature ranging from about −10° C. to reflux.

The said reaction is optionally carried out in the presence of a suitable catalyst, for instance 4-dimethylaminopyridine, or in the presence of a further condensing agent such as N-hydroxybenzotriazole.

As formerly indicated, within the compound of formula (V) W′ is a suitable leaving group such as, for instance, a halogen atom; chlorine or bromine being preferred.

The reaction between a compound of formula (I) and a sulphonyl derivative of formula (V) can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, at a temperature ranging from about −10° C. to reflux.

The said reaction is optionally carried out in the presence of a suitable catalyst, for instance 4-dimethylaminopyridine, or in the presence of a further condensing agent such as N-hydroxybenzotriazole.

The reaction between a compound of formula (I) and an isocyanate derivative of formula (VI) can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile, or N,N-dimethylformamide, and at a temperature ranging from about −10° C. to reflux.

According to step b.2) of the process, the compound of formula (I) is reacted, under reductive conditions, with an aldehyde or ketone derivative of formula (VII) so as to obtain the corresponding compound of formula (I) wherein Y is as above defined.

From the above, it is clear to the skilled man that by reacting an aldehyde derivative of formula (VII), for instance having R₃ as a hydrogen atom, the corresponding derivative of formula (I) wherein Y is a —CH₂— group may be obtained. Likewise, by reacting a ketone derivative of formula (VII) wherein both R₂ and R₃ are other than hydrogen, the corresponding derivative of formula (I) wherein Y is a —CH(R₃)— group and R₃ is other than hydrogen, may be obtained.

By analogy, if the ketone derivative of formula (VII) is represented by a compound (R₂)═O wherein R₂ is a heterocyclyl or cyclolakyl moiety, e.g. cyclohexanone, the above reaction according to step b.2) gives rise to a corresponding derivative of formula (I) wherein Y is a single bond and R₂ is just the said cyclolalkyl or heterocyclyl group.

All of these reactions are widely known in the art as reductive alkylation of amines and occur in the presence of a reducing agent such as, for instance, sodium borohydride, sodium cyanoborohydride or sodium triacetoxyborohydride, in a suitable solvent such as N,N-dimethylformamide, N,N-dimethylacetamide, chloroform, dichloromethane, tetrahydrofuran or acetonitrile, optionally in the presence of acetic acid, methanol or ethanol as co-solvents, at a temperature ranging from about −10° C. to reflux and for a time varying from about 30 minutes to about 96 hours.

According to step b.3) of the process, ureido derivatives of formula (I) wherein Y is —NHCO— and R₂ is a hydrogen atom may be prepared by reacting the compound of formula (I) with a suitable acylating agent such as, for instance, triphosgene or trichloromethyl chloroformate, in the presence of aqueous or gaseous ammonia, according to conventional techniques for preparing ureido —NHCONH₂ derivatives starting from the corresponding amino —NH₂ derivatives.

Finally, according to step c) of the process and whenever desired, a given compound of formula (I) may be optionally converted into another compound of formula (I), by working according to well known methods.

Just as an example, a given 4-carboxy-isoxazole derivative of formula (I), wherein X is —O— and R₁ is hydrogen, may be easily converted into the corresponding ester of formula (I) wherein X is —O— and R₁ is other than hydrogen, for instance an alkyl group, or into the corresponding carboxamido derivative of formula (I) wherein X is —NH(R₃)— and, as an example, R₃ is a hydrogen atom and R₁ is an alkyl group.

In this specific case, the above reactions may be performed according to well known methods of esterification or amidation, by reacting the given carboxy derivative of formula (I) with a suitable alcohol R₁—OH (VIII) or amino derivative R₁—NH(R₃) (IX), both bearing the desired R₁ and R₃ groups.

Likewise, the optional salification of a compound of formula (I) or the conversion of its salt into the free compound, may be all carried out by conventional methods. The compounds of formula (II) and (III) according to the process object of the present invention are known compounds which are easily prepared according to known methods. As an example, the compounds of formula (II) wherein Z is a chlorine atom may be prepared from the corresponding oximes, wherein Z is hydrogen, by working as described in J. Org. Chem., (1980), 3916; or J. Org. Chem., (1992), 6649.

The oximes, in their turn, are commercially available or readily obtainable from the corresponding aldehyde derivatives, according to conventional methods [see, for a reference, Org. Synth. Coll., 2, 70, 313 (1955)].

Also the compounds of formula (III) are commercially available or they may be prepared according to conventional techniques for carboxylic ester or carboxamide syntheses, by reacting cyanoacetic acid or a suitable derivative thereof with a compound of formula (VIII) or (IX), as above defined.

Likewise, all of the compounds of formula from (IV) to (IX) are known or may be prepared according to conventional methods.

As it will be really appreciated by the man skilled in the art, when preparing the compounds of formula (I) object of the invention, optional functional groups within both the starting materials or the intermediates thereof, which could give rise to unwanted side reactions, need to be properly protected according to conventional techniques. Likewise, the conversion of these latter into the free deprotected compounds may be carried out according to known procedures.

According to a preferred aspect of the process of the invention, the compounds of formula (I) may be also prepared by working on solid phase synthesis (SPS).

Therefore, it is a further object of the invention a process for preparing the compounds of formula (I), and the pharmaceutically acceptable salts thereof, which process comprises:
a′) reacting cyanoacetic acid with a suitable polystyrenic resin of formula (X) or (XI)
   Resin-W (X),
   Resin-NHR₁ (XI)
   wherein R₁ is as above defined and W is hydroxy or a suitable leaving group, so as to obtain the compounds of formula (XII) or (XIII), respectively 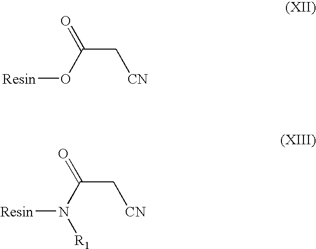
   wherein R₁ is as above reported;
b′) reacting any one of the compounds of formula (XII) or (XIII) with a compound of formula (II) 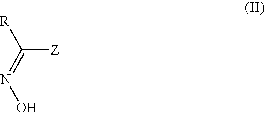
   wherein R and Z are as above defined, so as to obtain a compound of formula (XIV) or (XV) 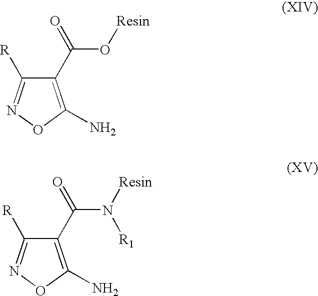
c′) cleaving the resin from the compounds of formula (XIV) or (XV) under acidic or basic conditions, so as to obtain a compound of formula (I) wherein X is a group —O— and R₁ is hydrogen, or X is a group —N(R₃)— wherein R₃ is hydrogen and R₁ is as above defined, respectively; or, alternatively, reacting the above compounds of formula (XIV) or (XV) according to any one of the alternative steps d′) below
d.1′) with a compound of formula (IV), (V) or (VI)
   R₂—COW (IV),
   R₂—SO₂W′ (V),
   R₂—NCO (VI)
   wherein R₂ is as above defined, W is hydroxy or a suitable leaving group and W′ is a suitable leaving group, so as to obtain the compounds of formula (XVI) or (XVII) 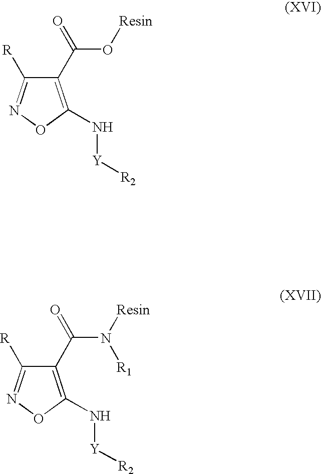
   wherein Y is —CO—, —SO₂— or —CONH—, respectively, and R, R₁ and R₂ are as above defined;
d.2′) with a suitable aldehyde or ketone derivative of formula (VII)
   R₂—CO—R₃ (VII)
   wherein R₂ and R₃ are as above defined, under reductive conditions, so as to obtain the compounds of the above formula (XVI) or (XVII) wherein Y is a group —CH(R₃)— and R, R₁, R₂ and R₃ are as above defined;
d.3′) with a suitable acylating agent in the presence of ammonia, so as to obtain a compound of the above formula (XVI) or (XVII) wherein Y is —CONH—, R₂ is hydrogen and R and R₁ are as above defined;
e′) cleaving the resin from the compounds of formula (XVI) or (XVII) under acidic or basic conditions, so as to obtain a compound of formula (I) wherein X is a group —O— and R₁ is hydrogen, or X is a group —N(R₃)— wherein R₃ is hydrogen and R₁ is as above defined, respectively; and, optionally,
f′) converting the thus obtained compound of formula (I) into another compound of formula (I) and/or into a pharmaceutically acceptable salt thereof.

According to step a′) of the process, cyanoacetic acid is reacted with a suitable polystyrenic resin of formula (X) or (XI) according to conventional operative conditions, in the presence of a base and of optional condensing agents or catalysts.

Likewise, according to step b′) of the process, the thus obtained compounds of formula (XII) or (XIII) are then reacted with a compound of formula (II), substantially as set forth above per step a) of the process under homogeneous conditions.

Analogous considerations apply to the alternative steps from d.1′) to d.3′) which are all carried out by working as described in corresponding steps from b.1) to b.3) of the process under homogeneous conditions.

According to any one of steps c′) or e′) of the process, the compounds of formula (I) can be obtained by cleaving the compounds of formula (XIV), (XV), (XVI) or (XVII) from the resin, under acidic or basic conditions.

The acidic cleavage can be performed in the presence of suitable acids such as, for instance, hydrochloric, trifluoroacetic, methanesulphonic or p-toluensulphonic acid, as well as by using conventional acid ion exchange resins. The reaction is carried out under conventional methods, for instance by using a solution of the acid, e.g. a 10% to 100% (v/v) solution of trifluoroacetic acid in dichloromethane, at a temperature ranging from about 0° C. to reflux, and for a suitable time, for instance from about 5 minutes to about 2 hours.

The basic cleavage can be performed under basic hydrolysis conditions, for instance in the presence of a conventional aqueous base such as sodium, potassium or lithium hydroxide. The reaction is carried out in a suitable solvent such as, for instance, N,N-dimethylformamide, ethanol, methanol isopropanol or tetrahydrofuran, at a temperature comprised from about 20° C. to reflux and for a suitable time, for instance from about 30 minutes to about 96 hours.

Finally, according to step f′), any obtained compound of formula (I) may be optionally converted into another compound of formula (I) by working according to conventional methods, for instance as reported in step c) of the process under homogeneous conditions.

From all of the above it is clear to the skilled man that the above optional conversion of a compound of formula (I) into another compound of formula (I) may be also carried out after step c′), that is after having carried out the resin cleavage form the compounds of formula (XIV) or (XV).

The polystyrenic resins of formula (X) or (XI) are commercially available and comprise, for instance, Wang resin, trityl resin, Cl-trityl resin, Rink amide resin Tentagel OH resin and derivatives thereof.

The resins of formula (XI), wherein R₁ is other than hydrogen may be also prepared according to conventional methods, for instance by reacting a commercially available formyl polystyrene resin with a suitable amine derivative R₁—NH₂ of formula (IX) under reductive conditions, as set forth above.

Cyanoacetic acid is also a known, commercially available, compound.

From what above reported, it is clear to the man skilled in the art that the compounds of formula (I) of the invention can be advantageously prepared by performing the above described reactions in a combinatorial fashion, for example according to the aforementioned solid-phase-synthesis (SPS) techniques, so as to get a combinatorial library of compounds.

As an example, the compounds of formula (XII) or (XIII) supported onto resin particles and prepared as above described, may be reacted with a variety of compounds of formula (II) so as to obtain a plurality of compounds of formula (XIV) or (XV), to be further reacted with a variety of compounds, for instance of formula (IV), (V), (VI) or (VII), so as to get thousands of different compounds of formula (XVI) or (XVII), according to combinatorial chemistry methods.

These latter derivatives, in their turn, are then conveniently converted into the derivatives of formula (I) of the invention, by cleaving the resin support as formerly reported.

It is therefore a further object of the invention a library of two or more compounds of formula (I) 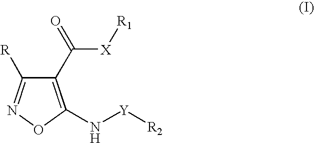
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆ cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;

R₂ and R₃ have, each independently, the meanings above reported for R₁ or represent an optionally substituted straight or branched C₂-C₆ alkenyl or alkynyl group; and the pharmaceutically acceptable salts thereof.

All of the compounds of formula (I) which are prepared according to combinatorial chemistry techniques, for instance as reported in the examples, whenever appropriate in the form of pharmaceutically acceptable salts, are herewith conveniently indicated and defined as “products by process”, that is as compounds of formula (I) which are obtainable through a given process.

As such, it is a further object of the present invention any specific compound of formula (I) which is obtainable, for instance through a combinatorial chemistry technique, by reacting each of the derivatives of formula (II), as set forth in table I, with any one of the derivatives of formula (XIII) which are obtainable as above indicated from the amines of formula (IX), as set forth in table II, and by subsequently operating as per the process of the invention by reacting any one of the obtained derivatives of formula (XV) with any one of the derivatives of formula (IV), as set forth in table III.

It is a further object of the present invention any specific compound of formula (I) which is obtainable, for instance through a combinatorial chemistry technique, by reacting each of the derivatives of formula (II), as set forth in table I, with any one of the derivatives of formula (XIII) which are obtainable as above indicated from the amines of formula (IX), as set forth in table II, and by subsequently operating as per the process of the invention by reacting any one of the obtained derivatives of formula (XV) with any one of the derivatives of formula (V), as set forth in table IV.

It is a further object of the present invention any specific compound of formula (I) which is obtainable, for instance through a combinatorial chemistry technique, by reacting each of the derivatives of formula (II), as set forth in table I, with any one of the derivatives of formula (XIII) which are obtainable as above indicated from the amines of formula (IX), as set forth in table II, and by subsequently operating as per the process of the invention by reacting any one of the obtained derivatives of formula (XV) with any one of the derivatives of formula (VI), as set forth in table V.

TABLE I
Compounds of formula (II)

| | |
|---|---|
| | (II) |
| 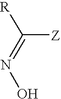 | |

| | |
|---|---|
| 1 | 3-Thiophenylhydroxyaminomethyl chloride |
| 2 | Pyridine-3-hydroxyaminomethyl chloride |
| 3 | Pyridine-2-hydroxyaminomethyl chloride |
| 4 | 2-Thiophenehydroxyaminomethyl chloride |
| 5 | Pyrrole-2-hydroxyaminomethyl chloride |
| 6 | 5-Methylfurfurhydroxyaminomethyl chloride |
| 7 | Indole-3-hydroxyaminomethyl chloride |
| 8 | 5-bromo-2-thiophenehydroxyaminomethyl chloride |
| 9 | 1-methylpyrrole-2-hydroxyaminomethyl chloride |
| 10 | 4-pyridinehydroxyaminomethyl chloride |
| 11 | 3-methylthiophene-2-hydroxyaminomethyl chloride |
| 12 | 5-methyl-2-thiophenehydroxyaminomethyl chloride |
| 13 | 6-methyl-2-pyridinehydroxyaminomethyl chloride |
| 14 | 5-methoxyindole-3-hydroxyaminomethyl chloride |
| 15 | 1-methylindole-3-hydroxyaminomethyl chloride |
| 16 | 4-bromothiophene-2-hydroxyaminomethyl chloride |
| 17 | N-ethyl-carbazole-3-hydroxyaminomethyl chloride |
| 18 | Furfurhydroxyaminomethyl chloride |
| 19 | 3-furhydroxyaminomethyl chloride |
| 20 | 5-fluoroindole-3-hydroxyaminomethyl chloride |
| 21 | 5-ethyl-2-thiophenehydroxyaminomethyl chloride |
| 22 | 3-phenyl-1H-pyrazole-4-hydroxyaminomethyl |
| | chloride |
| 23 | Imidazole-4-hydroxyaminomethyl chloride |
| 24 | Benzo[b]furan-2-hydroxyaminomethyl chloride |
| 25 | Indole-7-hydroxyaminomethyl chloride |
| 26 | 4-benzyloxyindole-3-hydroxyaminomethyl chloride |
| 27 | 3-methylbenzo[b]thiophene-2-hydroxyaminomethyl |
| | chloride |
| 28 | 3,5-dimethyl-1-phenylpyrazole-4- |
| | hydroxyaminomethyl chloride |
| 29 | Thiazole-2-hydroxyaminomethyl chloride |
| 30 | 7-methylindole-3-hydroxyaminomethyl chloride |
| 31 | 2,5-dimethyl-1-[3- |
| | (trifluoromethyl) phenyl]pyrrole-3- |
| | hydroxyaminomethyl chloride |
| 32 | 5-acetoxymethyl-2-furhydroxyaminomethyl |
| | chloride |
| 33 | 5-[2- |
| | (trifluoromethyl) phenyl]furfurhydroxyaminomethyl |
| | chloride |
| 34 | 5-(4-chlorophenyl) furfurhydroxyaminomethyl |
| | chloride |
| 35 | 5-(2-chlorophenyl) furfurhydroxyaminomethyl |
| | chloride |
| 36 | 5-(4-nitrophenyl)-2-furhydroxyaminomethyl |
| | chloride |
| 37 | 5-(2-nitrophenyl) furfurhydroxyaminomethyl |
| | chloride |
| 38 | 5-bromo-2-furhydroxyaminomethyl chloride |
| 39 | 5-(4-bromophenyl) furfurhydroxyaminomethyl |
| | chloride |
| 40 | 5-(3-chlorophenyl) furfurhydroxyaminomethyl |
| | chloride |
| 41 | 5-[2-chloro-5-(trifluoromethyl)phenyl]- |
| | furfurhydroxyaminomethyl chloride |
| 42 | 5-(3-trifluoromethylphenyl) furan-2- |
| | hydroxyaminomethyl chloride |
| 43 | 5-ethyl-2-furhydroxyaminomethyl chloride |
| 44 | 5-chloro-2-thiophenehydroxyaminomethyl chloride |
| 45 | 2-chloro-3-quinolinehydroxyaminomethyl chloride |
| 46 | 2,4-dimethoxy-5-pyrimidinehydroxyaminomethyl |
| | chloride |
| 47 | N-hydroxy-1-methyl-1H-indole-2-carboximidoyl |
| | chloride |
| 48 | Methyl 3-[chloro (hydroxyimino) methyl]-2-methyl- |
| | 1H-indole-6-carboxylate |
| 49 | N-hydroxy-6-methoxypyridine-3-carboximidoyl |
| | chloride |
| 50 | N-hydroxy-1-benzofuran-2-carboximidoyl chloride |
| 51 | 5-chloro-N-hydroxyfuran-2-carboximidoyl |
| | chloride |
| 52 | N-hydroxy-1,3-thiazole-5-carboximidoyl chloride |

TABLE II
Compounds of formula (IX)
R₁—NH₂ (IX)

| | |
|---|---|
| 1 | 2-aminofluorene |
| 2 | Cyclopropylamine |
| 3 | Cyclopentylamine |
| 4 | 2-(2-aminoethyl)-1-methylpyrrolidine |
| 5 | 2-(1-pyrrolidinyl)ethylamine |
| 6 | Furfurylamine |
| 7 | (+/−)-1-(1-naphthyl)ethylamine |
| 8 | 2-aminothiazole |
| 9 | 1-(2-aminophenyl)pyrrole |
| 10 | Tetrahydrofurfurylamine |
| 11 | 2-amino-5-chlorobenzoxazole |
| 12 | 4-aminobenzo-2,1,3-thiadiazole |
| 13 | 2-aminopyrimidine |
| 14 | 4-(4-aminophenyl)morpholine |
| 15 | 3-amino-2-chloropyridine |
| 16 | 2-aminopyridine |
| 17 | 2-amino-5-chloropyridine |
| 18 | 4-(aminomethyl)pyridine |
| 19 | Ethyl 4-amino-1-piperidinecarboxylate |
| 20 | 3,4-ethylenedioxyaniline |
| 21 | Aniline |
| 22 | 2-(methylthio)aniline |
| 23 | 3,4-difluoroaniline |
| 24 | 3-chloro-4-methylaniline |
| 25 | m-anisidine |
| 26 | 3-aminobenzotrifluoride |
| 27 | 3,4-dimethylaniline |
| 28 | 4-fluoro-2-methylaniline |
| 29 | N1-(4-aminophenyl)-N1-methylacetamide |
| 30 | N,N-diethyl-p-phenylenediamine |
| 31 | 4-butoxyaniline |
| 32 | 2-aminopentane |
| 33 | 2-methoxybenzylamine |
| 34 | 4-fluorobenzylamine |
| 35 | 4-chlorobenzylamine |
| 36 | N,N-dimethylethylenediamine |
| 37 | 2-(4-methoxyphenyl)ethylamine |
| 38 | 5-amino-2-methylisoindoline-1,3-dione |
| 39 | 3-methoxypropylamine |
| 40 | Sulfapyridine |
| 41 | 3-phenoxyaniline |
| 42 | 2-amino-4-methoxy-6-methylpyrimidine |
| 43 | 4-(difluoromethoxy)aniline |
| 44 | N-(4′-aminophenyl)-4-methyl-benzene-sulfonamide |
| 45 | 1,2,3,4-Tetrahydro-1-Naphthylamine |
| 46 | 1,2-Dimethylpropylamine |
| 47 | 1,2-Diphenylethylamine |
| 48 | 1,3-Dimethylbutylamine |
| 49 | 1-Aminoindan |
| 50 | 1-Methyl-3-Phenylpropylamine |
| 51 | 1-Naphthalenemethylamine |
| 52 | 2-(1-Cyclohexenyl)ethylamine |
| 53 | 2-(3-Chlorophenyl)ethylamine |
| 54 | 2-(Trifluoromethyl)Benzylamine |
| 55 | 2,4-Dimethoxybenzylamine |
| 56 | 2-Amino-6-Methylheptane |
| 57 | 2-Aminoheptane |
| 58 | 2-Aminooctane |
| 59 | 2-Bromobenzylamine |
| 60 | 2-Chlorobenzylamine |
| 61 | 2-Ethylhexylamine |
| 62 | 2-Fluorobenzylamine |
| 63 | 2-Methoxyisopropylamine |
| 64 | 2-Methylbenzylamine |
| 65 | 2-Methylcyclohexylamine |
| 66 | 3-(Di-N-Butylamino)Propylamine |
| 67 | 3-(Methylthio)Propylamine |
| 68 | 3-(Trifluoromethyl)Benzylamine |
| 69 | 3,3-Diphenylpropylamine |
| 70 | 3,4-Difluorobenzylamine |
| 71 | 3,4-Dimethoxybenzylamine |
| 72 | 3,4-Methylenedioxybenzylamine |
| 73 | 3,5-Bis(Trifluoromethyl)Benzylamine |
| 74 | 3-Aminopentane |
| 75 | 3-Chlorobenzylamine |
| 76 | 3-Fluorobenzylamine |
| 77 | 3-Methylbenzylamine |
| 78 | 3-Phenylpropylamine |
| 79 | 4-(Trifluoromethoxy)Benzylamine |
| 80 | 4-(Trifluoromethyl)Benzylamine |
| 81 | 4-Amino-1-Benzylpiperidine |
| 82 | 4-Methoxybenzylamine |
| 83 | 4-Methylbenzylamine |
| 84 | 4-Phenylbutylamine |
| 85 | 4-Tert-Butylcyclohexylamine |
| 86 | 5-Methylfurfurylamine |
| 87 | Allylamine |
| 88 | Aminomethylcyclohexane |
| 89 | Benzhydrylamine |
| 90 | Benzylamine |
| 91 | Cyclobutylamine |
| 92 | Cycloheptylamine |
| 93 | Cyclohexylamine |
| 94 | Cyclopropanemethylamine |
| 95 | DL-Alpha-Methylbenzylamine |
| 96 | Hexylamine |
| 97 | Isoamylamine |
| 98 | Isobutylamine |
| 99 | Isopropylamine |
| 100 | n-Amylamine |
| 101 | n-Butylamine |
| 102 | n-Heptylamine |
| 103 | n-Octylamine |
| 104 | n-Propylamine |
| 105 | Phenethylamine |
| 106 | Propargylamine |
| 107 | Sec-Butylamine |
| 108 | Tert-Butylamine |
| 109 | Tetrahydrofurfurylamine |
| 110 | Thiophene-2-Methylamine |

TABLE III
Compounds of formula (IV)
R₂—COW (IV)

| | |
|---|---|
| 1. | 9-fluorenecarboxylic acid; |
| 2. | 1-phenyl-1-cyclopropanecarboxylic acid; |
| 3. | 1-methylcyclopropane-1-carboxylic acid; |
| 4. | Cyclobutanecarboxylic acid; |
| 5. | cyclopentanecarboxylic acid; |
| 6. | (−)-menthoxyacetic acid; |
| 7. | 1,2,3,4-tetrahydro-2-naphthoic acid; |
| 8. | 2-fluorobenzoic acid; |
| 9. | 2,5-dimethoxybenzoic acid; |
| 10. | 2-biphenylcarboxylic acid; |
| 11. | 2-(4-chlorobenzoyl)benzoic acid; |
| 12. | 2,6-dimethylbenzoic acid; |
| 13. | 3-cyanobenzoic acid; |
| 14. | 3-bromobenzoic acid; |
| 15. | 3,4-dimethoxybenzoic acid; |
| 16. | 3,4,5-trimethoxybenzoic acid; |
| 17. | 3,4-diethoxybenzoic acid; |
| 18. | 4-cyanobenzoic acid; |
| 19. | 4-iodobenzoic acid; |
| 20. | 4-diethylaminobenzoic acid; |
| 21. | 4-biphenylcarboxylic acid; |
| 22. | 3-methyl-2-oxovaleric acid; |
| 23. | pyruvic acid; |
| 24. | 2-methylvaleric acid; |
| 25. | tert-butylacetic acid; |
| 26. | 3-(2-methoxyphenyl)propionic acid; |
| 27. | 5-nitro-2-furoic acid; |
| 28. | 1-naphthoic acid; |
| 29. | 2-naphthoic acid; |
| 30. | 2-ketobutyric acid; |
| 31. | pivalic acid; |
| 32. | 2,2-dimethylbutyric acid; |
| 33. | diphenylacetic acid; |
| 34. | N,N-dimethylglycine; |
| 35. | 2,3-dichlorophenoxyacetic acid; |
| 36. | phenylacetic acid; |
| 37. | 2,4-dichlorophenylacetic acid; |
| 38. | 3-fluorophenylacetic acid; |
| 39. | 4-ethoxyphenylacetic acid; |
| 40. | p-tolylacetic acid; |
| 41. | 4-pentynoic acid; |
| 42. | mono-methyl glutarate; |
| 43. | monomethyl adipate; |
| 44. | 6-acetamidohexanoic acid; |
| 45. | l-pyroglutamic acid; |
| 46. | 3-furoic acid; |
| 47. | thiophene-3-carboxylic acid; |
| 48. | thiophene-3-acetic acid; |
| 49. | nicotinic acid; |
| 50. | nalidixic acid; |
| 51. | 2-nitro-4-trifluoromethylbenzoic acid; |
| 52. | 4-methyl-3-nitrobenzoic acid; |
| 53. | 3-nitrobenzoic acid; |
| 54. | 3-nitrophenylacetic acid; |
| 55. | 4-carboxybenzenesulfonamide; |
| 56. | succinamic acid; |
| 57. | N-(4-nitrobenzoyl)-beta-alanine; |
| 58. | 3-(phenylsulfonyl)propionic acid; |
| 59. | 2,2,3,3-tetramethylcyclopropanecarboxylic acid; |
| 60. | 2-(4-nitrophenyl)propionic acid; |
| 61. | 2,2-dimethyl-4-pentenoic acid; |
| 62. | 3-(diethylamino)propionic acid hydrochloride; |
| 63. | 4-dimethylaminobutyric acid hydrochloride; |
| 64. | 4-isopropylphenoxyacetic acid; |
| 65. | 5-benzoylpentanoic acid; |
| 66. | 4-acetamido-3-nitrobenzoic acid; |
| 67. | d-campholic acid; |
| 68. | 2,5-dibromobenzoic acid; |
| 69. | 3-acetoxybenzoic acid; |
| 70. | 2,4,6-trimethoxyphenylacetic acid; |
| 71. | 2-benzyloxyphenylacetic acid; |
| 72. | (3,5-dimethoxyphenyl)acetic acid; |
| 73. | 2-nitrophenoxyacetic acid; |
| 74. | chromone-3-carboxylic acid; |
| 75. | N-acetyl-4-fluoro-dl-phenylalanine; |
| 76. | N-m-tolylphthalamic acid; |
| 77. | 4-acetamidobutyric acid; |
| 78. | 3-(2-thenoyl)-propionic acid; |
| 79. | 3,5-diacetamidobenzoic acid; |
| 80. | 5-acetamido-2-nitrobenzoic acid; |
| 81. | acetic acid; |
| 82. | 5-methylhexanoic acid; |
| 83. | N-benzoyl-b-alanine; |
| 84. | 4-bromo-3-methylbenzoic acid; |
| 85. | 4,5-dibromothiophene-2-carboxylic acid; |
| 86. | 2-acetamido-5-bromobenzoic acid; |
| 87. | 4-bromo-2-methylbenzoic acid; |
| 88. | 2-fluoro-6-iodobenzoic acid; |
| 89. | 2-furanglyoxylic acid; |
| 90. | N,N-dimethylsuccinamic acid; |
| 91. | 2-(2-methoxyethoxy)acetic acid; |
| 92. | 4-chloro-alpha-methylphenylacetic acid; |
| 93. | l-(p-tolyl)-1-cyclopentanecarboxylic acid; |
| 94. | picolinic acid hydrochloride; |
| 95. | 3,5-dibromobenzoic acid; |
| 96. | 5-chlorothianaphthene-3-acetic acid; |
| 97. | 2-nitrothiophene-4-carboxylic acid; |
| 98. | 3-chloro-2-methylbenzoic acid; |
| 99. | 2-bromo-4-fluorobenzoic acid; |
| 100 | 3-(2-chloro-6-fluorophenyl)-5-methylisoxazole- |
| | 4-carboxylic acid; |
| 101 | fenbufen; |
| 102 | indoprofen; |
| 103 | chrysanthemum monocarboxylic acid; |
| 104 | 6-acetoxy-2-naphthoic acid; |
| 105 | 3-methylthiopropionic acid; |
| 106 | (r)-(+)-N-(1-phenylethyl)phthalamic acid; |
| 107 | alpha-ketovaleric acid; |
| 108 | 5-methyl-1-phenylpyrazole-4-carboxylic acid; |
| 109 | 3-methyl-1-cyclohexanecarboxylic acid; |
| 110 | 3-methoxycyclohexanecarboxylic acid; |
| 111 | dicyclohexylacetic acid; |
| 112 | 5,6-dichloronicotinic acid; |
| 113 | 4-(dimethylamino)phenylacetic acid; |
| 114 | (r)-(+)-N-(1-phenylethyl)succinamic acid; |
| 115 | (s)-(−)-N-(1-phenylethyl)succinamic acid; |
| 116 | (+)-menthyloxyacetic acid; |
| 117 | suprofen; |
| 118 | N,N-dimethyl-l-phenylalanine; |
| 119 | 4-iodophenylacetic acid; |
| 120 | 4-(3,4-dimethoxyphenyl)butyric acid; |
| 121 | 2-fluoro-5-nitrobenzoic acid; |
| 122 | N,N-diethyl-3,6-difluorophthalamic acid; |
| 123 | 2-bromo-5-nitrobenzoic acid; |
| 124 | 4-bromo-2-fluorobenzoic acid; |
| 125 | 5-(2-thienyl)pentanoic acid; |
| 126 | isoxazole-5-carboxylic acid; |
| 127 | 5-nitrothiophene-2-carboxylic acid; |
| 128 | 2-(4-pyridyl)thiazole-4-carboxylic acid; |
| 129 | 2-methyl-4,4,4-trifluorobutyric acid; |
| 130 | 1-(aminocarbonyl)-1-cyclopropanecarboxylic |
| | acid; |
| 131 | 1-cyanocyclopropanecarboxylic acid; |
| 132 | (s)-(−)-2-acetoxypropionic acid; |
| 133 | 3-(methylsulfonyl)benzoic acid; |
| 134 | 2-chloro-4-methylsulfonylbenzoic acid; |
| 135 | 2,6-dichloropyridine-4-carboxylic acid; |
| 136 | 3-pyridinepropionic acid; |
| 137 | 5-(4-chloro-2-nitrophenyl)-2-furoic acid; |
| 138 | 7-chloro-1-ethyl-6-fluoro-4-oxohydroquinoline- |
| | 3-carboxylic acid; |
| 139 | cis-2-(2-thiophenecarbonyl)-1- |
| | cyclohexanecarboxylic acid; |
| 140 | 5-bromo-3-pyridylacetic acid; |
| 141 | 5-methylisoxazole-4-carboxylic acid; |
| 142 | 2,2-dimethylhexanoic acid; |
| 143 | 3-carboxypropanesulfonamide; |
| 144 | 6-cyanonicotinic acid; |
| 145 | (r)-(−)-2-methoxypropionic acid; |
| 146 | (s)-(+)-2-methoxypropionic acid; |
| 147 | 4-(tert-butoxymethyl)benzoic acid; |
| 148 | cis-2-(benzyloxycarbonylamino)- |
| | cyclohexanecarboxylic acid; |
| 149 | cis-2-(benzyloxycarbonylamino)-4-cyclohexene-1- |
| | carboxylic acid. |

TABLE IV
Compounds of formula (V)
R₂—SO₂W′ (V)

| | |
|---|---|
| 1. | 1-naphthalenesulfonyl chloride |
| 2. | 2-naphthalenesulfonyl chloride |
| 3. | 2-thiophenesulfonyl chloride |
| 4. | 8-quinolinesulfonyl chloride |
| 5. | benzenesulfonyl chloride |
| 6. | 2,4,5-trichlorobenzenesulfonyl chloride |
| 7. | 2,5-dichlorobenzenesulfonyl chloride |
| 8. | 3,5-dichloro-2-hydroxybenzenesulfonyl chloride |
| 9. | 2-mesitylenesulfonyl chloride |
| 10. | 4-bromobenzenesulfonyl chloride |
| 11. | 4-fluorobenzenesulfonyl chloride |
| 12. | 4-chlorobenzenesulfonyl chloride |
| 13. | pipsyl chloride |
| 14. | 4-methoxybenzenesulfonyl chloride |
| 15. | 4-tert-butylbenzenesulfonyl chloride |
| 16. | p-toluenesulfonyl chloride |
| 17. | isopropylsulfonyl chloride |
| 18. | methanesulfonyl chloride |
| 19. | alpha-toluenesulfonyl chloride |
| 20. | ethanesulfonyl chloride |
| 21. | 1-propanesulfonyl chloride |
| 22. | 1-butanesulfonyl chloride |
| 23. | Pentamethylbenzenesulfonyl chloride |
| 24. | 2,3,5,6-tetramethylbenzenesulfonyl chloride |
| 25. | 3-(trifluoromethyl)benzenesulphonyl chloride |
| 26. | 3,5-bis(trifluoromethyl)benzenesulfonyl chloride |
| 27. | 2,3,4-trichlorobenzenesulfonyl chloride |
| 28. | 2,5-dimethoxybenzenesulfonyl chloride |
| 29. | 4-methoxy-2,3,6-trimethylbenzenesulfonyl |
| | chloride |
| 30. | 3,4-dichlorobenzenesulfonyl chloride |
| 31. | 4,5-dibromothiophene-2-sulfonyl chloride |
| 32. | 3-chloro-4-fluorobenzenesulphonyl chloride |
| 33. | 4-ethylbenzenesulfonyl chloride |
| 34. | 4-N-propylbenzenesulfonyl chloride |
| 35. | 4-N-amylbenzenesulfonyl chloride |
| 36. | 4-isopropylbenzenesulphonyl chloride |
| 37. | 4-bromo-2,5-difluorobenzenesulphonyl chloride |
| 38. | 2-fluorobenzenesulphonyl chloride |
| 39. | 3-fluorobenzenesulphonyl chloride |
| 40. | 4-(trifluoromethoxy)benzenesulphonyl chloride |
| 41. | 4-(trifluoromethyl)benzenesulfonyl chloride |
| 42. | 2,4-difluorobenzenesulphonyl chloride |
| 43. | 2,4-dichloro-5-methylbenzenesulfonyl chloride |
| 44. | 4-chloro-2,5-dimethylbenzenesulphonyl chloride |
| 45. | 2-chlorobenzenesulfonyl chloride |
| 46. | 4-bromo-2,5-dichlorothiophene-3-sulfonyl |
| | chloride |
| 47. | 2,5-dichlorothiophene-3-sulphonyl chloride |
| 48. | 5-chlorothiophene-2-sulfonyl chloride |
| 49. | 2-(trifluoromethyl)benzenesulfonyl chloride |
| 50. | 3-chlorobenzenesulfonyl chloride |
| 51. | 3,5-dichlorobenzenesulfonyl chloride |
| 52. | m-toluenesulfonyl chloride |
| 53. | 2-chloro-6-methylbenzenesulfonyl chloride |
| 54. | 5-bromo-2-methoxybenzenesulfonyl chloride |
| 55. | 3,4-dimethoxybenzenesulfonyl chloride |
| 56. | 2,3-dichlorobenzenesulfonyl chloride |
| 57. | 2-bromobenzenesulfonyl chloride |
| 58. | 2,3-dichlorothiophene-5-sulphonyl chloride |
| 59. | 4-phenylthiophene-2,4-disulfonyl |
| 60. | 5-phenylthiophene-2,5-disulfonyl chloride |
| 61. | 3-chloro-2-methylbenzenesulfonyl chloride |
| 62. | 2-chloro-5-(trifluoromethyl)benzenesulfonyl |
| | chloride |
| 63. | 2,6-dichlorobenzenesulfonyl chloride |
| 64. | 3-bromobenzenesulfonyl chloride |
| 65. | 2-(trifluoromethoxy)benzenesulfonyl chloride |
| 66. | 4-cyanobenzenesulfonyl chloride |
| 67. | 2-cyanobenzenesulfonyl chloride |
| 68. | 4-(N-butoxy)benzenesulfonyl chloride |
| 69. | 4-acetamido-3-chlorobenzenesulfonyl chloride |
| 70. | 3,5-dimethylisoxazole-4-sulfonyl chloride |
| 71. | 2,4-dichlorobenzenesulfonyl chloride |
| 72. | 2-chloro-4-fluorobenzenesulphonyl chloride |
| 73. | 5-fluoro-2-methylbenzenesulphonyl chloride |
| 74. | 5-chloro-2-methoxybenzenesulfonyl chloride |
| 75. | 2,4,6-trichlorobenzenesulfonyl chloride |
| 76. | 4-biphenylsulfonyl chloride |
| 77. | 5-bromothiophene-2-sulfonyl chloride |
| 78. | 2,6-difluorobenzenesulfonyl chloride |
| 79. | 4-n-butylbenzenesulfonyl chloride |
| 80. | 4-methylsulfonylbenzenesulfonyl chloride |
| 81. | 2-methylsulfonylbenzenesulfonyl chloride |
| 82. | 4-acetylbenzenesulfonyl chloride |
| 83. | 3-methoxybenzenesulphonyl chloride |
| 84. | 2-methoxy-4-methylbenzenesulphonyl chloride |

TABLE V
Compounds of formula (VI)
R₂—NCO (VI)

| | |
|---|---|
| 1. | phenyl isocyanate |
| 2. | 2-bromophenyl isocyanate |
| 3. | 2-fluorophenyl isocyanate |
| 4. | 2,4-difluorophenyl isocyanate |
| 5. | 2,6-difluorophenyl isocyanate |
| 6. | 2-chlorophenyl isocyanate |
| 7. | 2,3-dichlorophenyl isocyanate |
| 8. | 2,4-dichlorophenyl isocyanate |
| 9. | 2,5-dichlorophenyl isocyanate |
| 10. | 2,6-dichlorophenyl isocyanate |
| 11. | 2-methoxyphenyl isocyanate |
| 12. | 2,4-dimethoxyphenyl isocyanate |
| 13. | 2,5-dimethoxyphenyl isocyanate |
| 14. | 2-ethoxyphenyl isocyanate |
| 15. | 2-(trifluoromethyl)phenyl isocyanate |
| 16. | o-tolyl isocyanate |
| 17. | 2,6-dimethylphenyl isocyanate |
| 18. | 2-ethylphenyl isocyanate |
| 19. | 3-bromophenyl isocyanate |
| 20. | 3-fluorophenyl isocyanate |
| 21. | 3-chlorophenyl isocyanate |
| 22. | 3,4-dichlorophenyl isocyanate |
| 23. | 3-methoxyphenyl isocyanate |
| 24. | 3-(trifluoromethyl)phenyl isocyanate |
| 25. | m-tolyl isocyanate |
| 26. | 4-bromophenyl isocyanate |
| 27. | 4-fluorophenyl isocyanate |
| 28. | 4-chlorophenyl isocyanate |
| 29. | 4-methoxyphenyl isocyanate |
| 30. | 4-(trifluoromethyl)phenyl isocyanate |
| 31. | p-tolyl isocyanate |
| 32. | benzoyl isocyanate |
| 33. | tert-butyl isocyanate |
| 34. | (S)-(−)-1-phenylethyl isocyanate |
| 35. | Isopropyl isocyanate |
| 36. | Ethyl isocyanate |
| 37. | Allyl isocyanate |
| 38. | N-propyl isocyanate |
| 39. | n-butyl isocyanate |
| 40. | Cyclohexyl isocyanate |
| 41. | 1-naphthyl isocyanate |
| 42. | (R)-(−)-1-(1-naphthyl)ethyl isocyanate |
| 43. | Benzyl isocyanate |
| 44. | 3,5-bis(trifluoromethyl)phenyl isocyanate |
| 45. | 2,5-difluorophenyl isocyanate |
| 46. | 2,4,5-trichlorophenyl isocyanate |
| 47. | 2,4,6-trichlorophenyl isocyanate |
| 48. | 2-isopropylphenyl isocyanate |
| 49. | 2,3-dimethylphenyl isocyanate |
| 50. | 4-methoxy-2-methylphenyl isocyanate |
| 51. | 2,4-dimethylphenyl isocyanate |
| 52. | 2,5-dimethylphenyl isocyanate |
| 53. | 2-ethyl-6-methylphenyl isocyanate |
| 54. | 3-cyanophenyl isocyanate |
| 55. | 5-chloro-2,4-dimethoxyphenyl isocyanate |
| 56. | 3-chloro-4-methylphenyl isocyanate |
| 57. | 3,5-dichlorophenyl isocyanate |
| 58. | 5-chloro-2-methoxyphenyl isocyanate |
| 59. | 3,4,5-trimethoxyphenyl isocyanate |
| 60. | 3,5-dimethoxyphenyl isocyanate |
| 61. | 3-(methylthio)phenyl isocyanate |
| 62. | 3-acetylphenyl isocyanate |
| 63. | 3,4-dimethylphenyl isocyanate |
| 64. | 3,5-dimethylphenyl isocyanate |
| 65. | 2-methoxy-5-methylphenyl isocyanate |
| 66. | 3-ethylphenyl isocyanate |
| 67. | 4-bromo-2-(trifluoromethyl)phenyl isocyanate |
| 68. | 4-chloro-2-(trifluoromethyl)phenyl isocyanate |
| 69. | 4-chloro-3-(trifluoromethyl)phenyl isocyanate |
| 70. | 4-iodophenyl isocyanate |
| 71. | 4-phenoxyphenyl isocyanate |
| 72. | 4-ethoxyphenyl isocyanate |
| 73. | 4-acetylphenyl isocyanate |
| 74. | 4-isopropylphenyl isocyanate |
| 75. | 4-ethylphenyl isocyanate |
| 76. | 4-n-butylphenyl isocyanate |
| 77. | 2,4,6-trimethylphenyl isocyanate |
| 78. | 2-isopropyl-6-methylphenyl isocyanate |
| 79. | 2,6-diethylphenyl isocyanate |
| 80. | 5-chloro-2-methylphenyl isocyanate |
| 81. | 4-chloro-2-methylphenyl isocyanate |
| 82. | 4-(trifluoromethoxy)phenyl isocyanate |
| 83. | 2-chloro-5-(trifluoromethyl)phenyl isocyanate |
| 84. | 2-chloro-6-methylphenyl isocyanate |
| 85. | 2,4,5-trimethylphenyl isocyanate |
| 86. | 3-chloro-2-methoxyphenyl isocyanate |
| 87. | 3-chloro-2-methylphenyl isocyanate |
| 88. | 3-chloro-4-fluorophenyl isocyanate |
| 89. | 4-bromo-2-methylphenyl isocyanate |
| 90. | 4-bromo-2,6-dimethylphenyl isocyanate |
| 91. | 2,6-dibromo-4-fluorophenyl isocyanate |
| 92. | 4-butoxyphenyl isocyanate |
| 93. | 3-fluoro-4-methylphenyl isocyanate |
| 94. | 5-fluoro-2-methylphenyl isocyanate |
| 95. | 2-biphenylyl isocyanate |
| 96. | 4-biphenylyl isocyanate |
| 97. | 2-bromo-4,6-difluorophenyl isocyanate |
| 98. | (r)-(+)-1-phenylethyl isocyanate |
| 99. | 1-(1-naphthyl)ethyl isocyanate |
| 100 | (s)-(+)-1-(1-naphthyl)ethyl isocyanate |
| 101 | 3,4-difluorophenyl isocyanate |
| 102 | 3-isopropenyl-alpha,alpha-dimethylbenzyl |
| | isocyanate |
| 103 | 2-(trifluoromethoxy)phenyl isocyanate |
| 104 | 4-benzyloxyphenyl isocyanate |
| 105 | 4-bromo-2-chlorophenyl isocyanate |
| 106 | 4-bromo-2-fluorophenyl isocyanate |
| 107 | 2-fluoro-5-methylphenyl isocyanate |
| 108 | 2,3,4-trifluorophenyl isocyanate |
| 109 | 2-(difluoromethoxy)phenyl isocyanate |
| 110 | 4-(difluoromethoxy)phenyl isocyanate |
| 111 | 2-methylbenzyl isocyanate |
| 112 | 2-chlorobenzyl isocyanate |
| 113 | 4-fluorobenzyl isocyanate |
| 114 | 4-methoxybenzyl isocyanate |
| 115 | 2,6-difluorobenzoyl isocyanate |
| 116 | 4-fluorobenzoyl isocyanate |
| 117 | 2-fluoro-3-(trifluoromethyl)phenyl isocyanate |
| 118 | 2-fluoro-5-(trifluoromethyl)phenyl isocyanate |
| 119 | 2-fluoro-6-(trifluoromethyl)phenyl isocyanate |
| 120 | 4-fluoro-2-(trifluoromethyl)phenyl isocyanate |
| 121 | 2-(tert-butyl)phenyl isocyanate |
| 122 | 3-pyridyl isocyanate |

From all of the above, it is clear to the skilled man that once a library of aminoisoxazoles derivatives is thus prepared, for instance consisting of a few thousands of compounds of formula (I), the said library can be very advantageously used for screening towards given target kinases, as formerly reported.

See, for a general reference to libraries of compounds and uses thereof as tools for screening biological activities, J. Med. Chem. 1999, 42, 2373-2382; and Bioorg. Med. Chem. Lett. 10 (2000), 223-226.

### Pharmacology

The compounds of formula (I) are active as protein kinase inhibitors and are therefore useful, for instance, to restrict the unregulated proliferation of tumor cells.

In therapy, they may be used in the treatment of various tumors such as, for instance, carcinomas, e.g. mammary carcinoma, lung carcinoma, bladder carcinoma, colon carcinoma, ovary and endometrial tumors, sarcomas, e.g. soft tissue and bone sarcomas, and the hematological malignancies such as, e.g., leukemias.

In addition, the compounds of formula (I) are also useful in the treatment of other cell proliferative disorders such as psoriasis, vascular smooth cell proliferation associated with atherosclerosis and post-surgical stenosis and restenosis and in the treatment of Alzheimer's disease. The inhibiting activity of putative protein kinase inhibitors and the potency of selected compounds was determined through a method of assay based on the use of the MultiScreen-PH 96 well plate (Millipore), in which a phosphocellulose filter paper was placed at each well bottom allowing binding of positive charged substrate after a washing/filtration step.

When a radioactivity labeled phosphate moiety was transferred by the ser/threo kinase to the filter-bound histone, light emitted was measured in a scintillation counter.

Or through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted was measured in a scintillation counter.

### Inhibition Assay of cdk2/Cyclin A Activity

Kinase reaction: 4 μM in house biotinylated histone H1 (Sigma # H-5505) substrate, 10 μM ATP (0.1 microCi P³³γ-ATP), 4.2 ng Cyclin A/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 ul PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the plate and let stand 4 hours before radioactivity counting in the Top-Count instrument

IC50 determination: inhibitors were tested at different concentrations ranging from 0.0015 to 10 μM. Experimental data were analyzed by the computer program GraphPad Prizm using the four parameter logistic equation:
y=bottom+(top-bottom)/(1+10\hat{ }((log IC50−x)*slope))
where x is the logarithm of the inhibitor concentration, y is the response; y starts at bottom and goes to top with a sigmoid shape.
Ki Calculation

Experimental method: Reaction was carried out in buffer (10 mM Tris, pH 7.5, 10 mM MgCl₂, 0.2 mg/ml BSA, 7.5 mM DTT) containing 3.7 nM enzyme, histone and ATP (constant ratio of cold/labeled ATP 1/3000). Reaction was stopped with EDTA and the substrate captured on phosphomembrane (Multiscreen 96 well plates from Millipore). After extensive washing, the multiscreen plates are read on a top counter. Control (time zero) for each ATP and histone concentrations was measured.

Experimental design: Reaction velocities are measured at different four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allow the selection of a single endpoint time (10 min) in the linear range of the reaction for the Ki determination experiment.

Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{\begin{matrix}α \cdot Ka \cdot Kb + α \cdot Ka \cdot B + a \cdot Kb \cdot \\ A + A \cdot B + α \cdot \frac{Ka}{Ki} \cdot I \cdot ( Kb + \frac{B}{β} )\end{matrix}} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively. □ and □ the cooperativity factor between substrate and ATP binding and substrate and inhibitor binding respectively.

In addition the selected compounds have been characterized on a panel of ser/threo kinases strictly related to cell cycle (cdk2/cyclin E, cdk1/cyclin B1, cdk5/p25, cdk4/cyclin D1), and also for specificity on MAPK, PKA, EGFR, IGF1-R, and Aurora-2.

Inhibition Assay of cdk2/Cyclin E Activity

Kinase reaction: 10 μM in house biotinylated histone H1 (Sigma # H-5505) substrate, 30 μM ATP (0.3 microCi P³³γ-ATP), 4 ng GST-Cyclin E/CDK2 complex, inhibitor in a final volume of 30 ml buffer (TRIS HCl 10 mM pH 7.5, MgCl₂ 10 mM, DTT 7.5 mM+0.2 mg/ml BSA) were added to each well of a 96 U bottom. After 60 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the plate and let stand 4 hours before radioactivity counting in the Top-Count instrument

IC50 determination: see above

Inhibition Assay of cdk1/Cyclin B1 Activity

Kinase reaction: 4 μm in house biotinylated histone H1 (Sigma # H-5505) substrate, 20 mM ATP (0.2 microCi P³³γ-ATP), 3 ng Cyclin B/CDK1 complex, inhibitor in a final volume of 30 ml buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the Optiplate and let stand 4 hours before radioactivity counting in the Top-Count instrument.

IC50 determination: see above

Inhibition Assay of cdk5/p25 Activity

The inhibition assay of cdk5/p25 activity was performed according to the following protocol.

Kinase reaction: 10 uM biotinylated histone H1 (Sigma # H-5505) substrate, 30 μM ATP (0.3 microCi P³³g-ATP), 15 ng CDK5/p25 complex, inhibitor in a final volume of 30 ml buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the plate and let stand 4 hours before radioactivity counting in the Top-Count instrument.

IC50 determination: see above

Inhibition Assay of cdk4/Cyclin D1 Activity

Kinase reaction: 0,4 uM mouse GST-Rb (769-921) (# sc-4112 from Santa Cruz) substrate, 10 μM ATP (0.5 μCi P³³γ-ATP), 100 ng of baculovirus expressed GST-cdk4/GST-Cyclin D1, suitable concentrations of inhibitor in a final volume of 50 ml buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 ml EDTA 120 mM.

Capture: 60 ml were transferred from each well to MultiScreen plate, to allow substrate binding to phosphocellulose filter. Plates were then washed 3 times with 150 ml/well PBS Ca⁺⁺/Mg⁺⁺ free and filtered by MultiScreen filtration system.

Detection: filters were allowed to dry at 37° C., then 100 ml/well scintillant were added and ³³P labeled Rb fragment was detected by radioactivity counting in the Top-Count instrument.

IC50 determination: see above

Inhibition Assay of MAPK Activity

Kinase reaction: 10 μM in house biotinylated MBP (Sigma # M-1891) substrate, 15 μM ATP (0.15 microCi P³³γ-ATP), 30 ng GST-MAPK (Upstate Biothecnology # 14-173), inhibitor in a final volume of 30 ml buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the Optiplate and let stand 4 hours before radioactivity counting in the Top-Count instrument.

IC50 determination: see above

Inhibition Assay of PKA Activity

Kinase reaction: 10 μM in house biotinylated histone H1 (Sigma # H-5505) substrate, 10 μM ATP (0.2 microM P³³γ-ATP), 0.45 U PKA (Sigma # 2645), inhibitor in a final volume of 30 ml buffer. (TRIS HCl 10 mM pH 7.5, MgCl₂ 10 mM, DTT 7.5 mM+0.2 mg/ml BSA) were added to each well of a 96 U bottom. After 90 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the Optiplate and let stand 4 hours before radioactivity counting in the Top-Count instrument.

IC50 determination: see above

Inhibition Assay of EGFR Activity

Kinase reaction: 10 μM in house biotinylated MBP (Sigma # M-1891) substrate, 2 mM ATP (0.04 microCi P³³γ-ATP), 36 ng insect cell expressed GST-EGFR, inhibitor in a final volume of 30 ml buffer (Hepes 50 mM pH 7.5, MgCl₂ 3 mM, MnCl₂ 3 mM, DTT 1 mM, NaVO₃ 3 μM+0.2 mg/ml BSA) were added to each well of a 96 U bottom. After 20 min at r.t. incubation, reaction was stopped by 100 ml PBS+32 mM EDTA+0.1% Triton X-100+500 mM ATP, containing 1 mg SPA beads. Then a volume of 110 ml is transferred to Optiplate.

After 20 min. incubation for substrate capture, 100 ml 5M CsCl were added to allow statification of beads to the top of the Optiplate and let stand 4 hours before radioactivity counting in the Top-Count instrument.

IC50 determination: see above

Inhibition Assay of IGF1-R Activity

The inhibition assay of IGF1-R activity was performed according to the following protocol.

Kinase reaction: 10 uM biotinylated MBP (Sigma cat. # M-1891) substrate, 0-20 mM inhibitor, 6 mM ATP, 1 microCi ³³P-ATP, and 22.5 ng GST-IGF1-R (pre-incubated for 30 min at room temperature with cold 60 mM cold ATP) in a final volume of 30 ml buffer (50 mM HEPES pH 7.9, 3 mM MnCl₂, 1 mM DTT, 3 mM NaVO₃) were added to each well of a 96 U bottom well plate. After incubation for 35 min at room temperature, the reaction was stopped by addition of 100 ml PBS buffer containing 32 mM EDTA, 500 mM cold ATP, 0.1% Triton X100 and 10 mg/ml streptavidin coated SPA beads. After 20 min incubation, 110 mL of suspension were withdrawn and transferred into 96-well OPTIPLATEs containing 100 ml of 5M CsCl. After 4 hours, the plates were read for 2 min in a Packard TOP-Count radioactivity reader.

Inhibition Assay of Aurora-2 Activity

Kinase reaction: 8 μM biotinylated peptide (4 repeats of LRRWSLG), 10 μM ATP (0.5 uCi P³³γ-ATP), 15 ng Aurora2, inhibitor in a final volume of 30 ml buffer (HEPES 50 mM pH 7.0, MgCl₂ 10 mM, 1 mM DTT, 0.2 mg/ml BSA, 3 mM orthovanadate) were added to each well of a 96 U bottom well plate. After 30 minutes at room temperature incubation, reaction was stopped and biotinylated peptide captured by adding 100 ml of bead suspension.

Stratification: 100 ml of CsCl2 5 M were added to each well and let stand 4 hour before radioactivity was counted in the Top-Count instrument.

IC50 determination: see above

Inhibition Assay of Cdc7/dbf4 Activity

The inhibition assay of Cdc7/dbf4 activity was performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:
10 ul substrate (biotinylated MCM2, 6 uM final concentration)
10 ul enzyme (Cdc7/Dbf4, 12.5 nM final concentration)
10 ul test compound (12 increasing concentrations in the nM to mM range to generate a dose-response curve)
10 ul of a mixture of cold ATP (10 uM final concentration) and radioactive ATP (1/2500 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 mM NaVO₃, 2 mM glycerophosphate and 0.2 mg/ml BSA. The solvent for test compounds also contained 10% DMSO.

After incubation for 20 minutes, the reaction was stopped by adding to each well 100 ul of PBS pH 7.4 containing 50 mM EDTA, 1 mM cold ATP, 0.1% Triton X100 and 10 mg/ml streptavidin coated SPA beads.

After 15 minutes of incubation at room temperature to allow the biotinylated MCM2-streptavidin SPA beads interaction to occur, beads were trapped in a 96 wells filter plate (Unifilter^{R} GF/B™) using a Packard Cell Harvester (Filtermate), washed with distilled water and then counted using a Top Count (Packard).

Counts were blank-subtracted and then the experimental data (each point in triplicate) were analyzed for IC50 determination using a non-linear regression analysis (Sigma Plot).

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from. about 10 to about 500 mg pro dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

As an example, the compounds of the invention can be administered in combination with one or more chemotherapeutic agents such as, for instance, exemestane, formestane, anastrozole, letrozole, fadrozole, taxane and derivatives such as paclitaxel and docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, parecoxib, rofecoxib, valecoxib, tamoxifen, raloxifen, JTE 5222, Sugen SU-5416, Sugen SU-6668, Herceptin, estramustine, and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

The present invention also includes pharmaceutical compositions comprising a compound of formula (I) or a pharmaceutically acceptable salt thereof in association with a pharmaceutically acceptable excipient (which can be a carrier or a diluent).

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic , magnesium or calcium stearate, and/or polyethylene glycols; binding agents, e.g. starches, arabic gum, gelatine, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulphates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerin and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

The following examples are herewith intended to better illustrate the present invention, without posing any limitation to it.

General Methods

Flash chromatography was performed on silica gel (Merck grade 9385, 60 Å). HPLC/MS was performed on a Waters X Terra RP 18 (4.6×50 mm, 3.5 mm) column using a Waters 2790 HPLC system equipped with a 996 Waters PDA detector and a Micromass mod. ZQ single quadrupole mass spectrometer, equipped with an electrospray (ESI) ion source. Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 min. UV detection at 220 nm and 254 nm. Flow rate 1 ml/min. Injection volume 10 μl. Full scan, mass range from 100 to 800 amu. Capillary voltage was 2.5 KV; Source temp. was 120° C.; Cone was 10 V. Retention Times (HPLC r.t.) are given in minutes at 220 nm or 254 nm. Mass is given as m/z ratio.

HPLC/MS was also (Method B) performed on a Hypersil C18 BDS (50×2.0 mm, 5 mm) column using a HPLC system equipped with Hewlett Packard 1312A binary pump with Gilson 215 autosampler fitted with a lml syringe with a Polymer Labs PL1000 Evaporative Light Scattering Detector (ELSD) and a Micromass ZMD mass spectrometer operating in Electrospray positive ionisation mode. Mobile phase A was water with 0.1% of trifluoroacetic acid, and mobile phase B was acetonitrile with 0.1% of trifluoroacetic acid. Gradient from 0% to 95% B in 1.8 minutes, hold 95% B 0.3 min. Flow rate 1 ml/min. Injection volume 3 μl. ELS Detector: Nebuliser Temperature 80° C.; Evaporation temperature 90° C.; Gas Flow 1.5 l/hr. Full scan, mass range from 150 to 800 amu (@ 0.5 secs/scan, 0.1 second interscan delay). Capillary voltage was 25 V; Source temp. was 140° C. Retention Times (HPLC Method B r.t.) are given in minutes (rev. ELSD); mass is given as mass found in MassLynx® Report (Waters).

When necessary compounds have been purified by Preparative HPLC on a Waters Symmetry C18 (19×50 mm, 5 um) column using a Waters preparative HPLC 600 equipped with a 996 Waters PDA detector and a Micromass mod. ZMD single quadrupole mass spectrometer, electrospray ionisation, positive mode. Mobile phase A was water 0.01% TFA, and Mobile phase B was acetonitrile. Gradient from 10 to 90% B in 8 min, hold 90% B 2 min. Flow rate 20 ml/m.

¹H-NMR spectroscopy was performed on a Mercury VX 400 operating at 400.45 MHz equipped with a 5 mm double resonance probe (1H {15N-31P} ID_PFG Varian).

### EXAMPLE 1

N-(4-tert-butylphenyl)cyanoacetamide

To 100 mg of cyanoacetic acid (1.17 mmol, 1.5 eq) in 8 ml of dichloromethane (DCM), 0.130 ml of 4-tert-butyl aniline (0.78 mmol) and 804 mg of N-cyclohexylcarbodiimide,N′-methylpolystyrene (1.94 mmol/g theoretical loading, 2 eq) were added. The suspension was shaken over-night then the resin was filtered off and washed with dichloromethane and dimethylformamide. The final product was obtained in 80% yield (135 mg) with 100% HPLC purity (according to HPLC analysis at 220 nm). [M+H]+=217; [M−H]−=215; HPLC r.t. 5,35.

¹H-NMR (DMSO-d₆), diagnostic signals (ppm): 10,2 (bs, 1H), 7,42 (d, 2H), 7,2 (d, 2H), 3,8 (s, 2H), 1,2 (s, 9H).

By working in an analogous way and by using the appropriate amine derivative, the following compounds were prepared:
N-(4-fluorophenyl)cyanoacetamide [M−H]−=177; HPLC r.t. 3,2.
N-(2,4-difluorophenyl)cyanoacetamide [M−H]−=195; HPLC r.t. 3,1.
N-(4-chlorophenyl)cyanoacetamide [M−H]−=193; HPLC r.t. 4,2.
N-(Benzyl)cyanoacetamide [M+H]+=175 [M−H]−=173; HPLC r.t. 2,6.
2-cyano-N-(5,6,7,8-tetrahydronaphthalen-1-yl)acetamide [M+H]+=215; [M−H]−=213; HPLC r.t. 4,5.
2-cyano-N-(2,3-dihydro-1H-inden-5-yl)acetamide [M−H]−=199; HPLC r.t. 4,5.
N-(4-(trifluoromethoxy)phenyl)cyanoacetamide [M−H]−=243; HPLC r.t. 4,9.
N-(1-naphthyl)cyanoacetamide [M+H]+=211 [M−H]−=209; HPLC r.t. 4,0.
N-(2-benzylphenyl)cyanoacetamide [M−H]−=249; HPLC r.t. 4,9.
2-cyano-N-(4-methylphenyl)acetamide [M+H]+=175; [M−H]−=173; HPLC r.t. 3,63.
2-cyano-N-[4-(dimethylamino)phenyl]acetamide [M+H]+=204; HPLC r.t. 3,06.
N-(3-benzyloxyphenyl)cyanoacetamide [M+H]+=267; [M−H]−=265; HPLC r.t. 5,4.
N-benzhydryl-2-cyanoacetamide [M−H]−=249; HPLC r.t. 4,9.
N-(2,4-dimethoxybenzyl)cyanoacetamide [M−H]−=233; HPLC r.t. 3,28.
N-(3,4,5-trimethoxyphenyl)cyanoacetamide [M+H]+=251; [M−H]−=249; HPLC r.t. 2,79.
2-cyano-N-(2-methoxydibenzo[b,d]furan-3-yl)acetamide [M+H]+=281 [M−H]−=279 HPLC r.t. 5,7.
2-cyano-N-quinolin-8-ylacetamide [M+H]+=212 [M−H]−=210 HPLC r.t. 3,97.
2-cyano-N-[2-oxo-4-(trifluoromethyl)-2H-chromen-7-yl]acetamide [M−H]−=295 HPLC r.t. 4,7.
N-(3,5-dimethoxyphenyl)cyanoacetamide [M+H]+=221 [M−H]−=219 HPLC r.t. 3,58.
Ethyl 3-[(cyanoacetyl)amino]benzoate [M−H]−=231 HPLC r.t. 4,1.
2-cyano-N-[2-methoxy-5-(trifluoromethyl)phenyl]acetamide [M−H]−=257 HPLC r.t. 4,2.
2-cyano-N-[(5-methyl-2-furyl)methyl]acetamide.
2-cyano-N-cyclobutylacetamide.
2-cyano-N-cyclohexylacetamide.
2-cyano-N-(2-methylcyclohexyl)acetamide.
2-cyano-N-(cyclohexylmethyl)acetamide.
2-cyano-N-(1,2,3,4-tetrahydronaphthalen-1-yl)acetamide.
2-cyano-N-(1-naphthylmethyl)acetamide.
2-cyano-N-cycloheptylacetamide.
2-cyano-N-(thien-2-ylmethyl)acetamide.
N-(1,3-benzodioxol-5-ylmethyl)-2-cyanoacetamide.
N-(1-benzylpiperidin-4-yl)-2-cyanoacetamide.
N-(tert-butyl)-2-cyanoacetamide.
2-cyano-N-(1,2-diphenylethyl)acetamide.
2-cyano-N-(1-phenylethyl)acetamide.
2-cyano-N-(1,2-dimethylpropyl)acetamide.
2-cyano-N-isopropylacetamide.
2-cyano-N-(2-methoxy-1-methylethyl)acetamide.
2-cyano-N-(1,3-dimethylbutyl)acetamide.
2-cyano-N-(1-methyl-3-phenylpropyl)acetamide.
2-cyano-N-(1,5-dimethylhexyl)acetamide.
N-(sec-butyl)-2-cyanoacetamide.
2-cyano-N-(1-ethylpropyl)acetamide.
2-cyano-N-(1-methylhexyl)acetamide.
2-cyano-N-(1-methylheptyl)acetamide.
N-benzyl-2-cyanoacetamide.
2-cyano-N-(2-fluorobenzyl)acetamide.
N-(2-chlorobenzyl)-2-cyanoacetamide.
2-cyano-N-(2-methylbenzyl)acetamide.
2-cyano-N-(3-fluorobenzyl)acetamide.
2-cyano-N-(3,4-dimethoxybenzyl)acetamide.
2-cyano-N-[3-(trifluoromethyl)benzyl]acetamide.
2-cyano-N-(3-methylbenzyl)acetamide.
2-cyano-N-(4-methoxybenzyl)acetamide.
2-cyano-N-(4-methylbenzyl)acetamide.
2-cyano-N-isobutylacetamide.
2-cyano-N-(2-ethylhexyl)acetamide.
2-cyano-N-(2-phenylethyl)acetamide.
2-cyano-N-prop-2-ynylacetamide.
N-allyl-2-cyanoacetamide.
2-cyano-N-(3,3-diphenylpropyl)acetamide.
2-cyano-N-isopentylacetamide.
2-cyano-N-propylacetamide.
2-cyano-N-[3-(dibutylamino)propyl]acetamide.
2-cyano-N-(3-phenylpropyl)acetamide.
2-cyano-N-(4-phenylbutyl)acetamide.
2-cyano-N-pentylacetamide.
2-cyano-N-hexylacetamide.
2-cyano-N-heptylacetamide.
2-cyano-N-octylacetamide.
N-[3,5-bis(trifluoromethyl)benzyl]-2-cyanoacetamide.
2-cyano-N-(3,4-difluorobenzyl)acetamide.
2-cyano-N-[4-(trifluoromethyl)benzyl]acetamide.
2-cyano-N-[2-(trifluoromethyl)benzyl]acetamide.
N-butyl-2-cyanoacetamide.
2-cyano-N-(2-cyclohex-1-en-1-ylethyl)acetamide.
N-(4-tert-butylcyclohexyl)-2-cyanoacetamide.
N-(2-bromobenzyl)-2-cyanoacetamide.
2-cyano-N-(cyclopropylmethyl)acetamide.
N-(3-chlorobenzyl)-2-cyanoacetamide.
2-cyano-N-[3-(methylthio)propyl]acetamide.
N-[2-(3-chlorophenyl)ethyl]-2-cyanoacetamide.
2-cyano-N-(2,4-dimethoxybenzyl)acetamide.
2-cyano-N-[4-(trifluoromethoxy)benzyl]acetamide.

### EXAMPLE 2

Cyanoacetic Acid Rink Amide

Rink amide (100 mg, Novabiochem, 0.59 mmol/g, 0.059 mmol) was suspended in 2 ml of piperidine 20% in dimethylformamide (DMF), after 20 minutes of shaking, the resin was washed with DMF, DCM and DMF and the procedure was repeated.

To the Fmoc-deprotected resin 2 ml of DCM/DMF 1:1, cyanoacetic acid (20 mg, 0.236 mmol, 4 eq.), HOBt (64 mg, 0.47 mmol, 8 eq.), DIPEA (80.8 μl, 0.47 mmol, 8 eq.) were added. The suspension was shaken 10 min and DIC (74 μl, 0.47 mmol, 8 eq.) was added. After 16 hrs the resin was washed with DCM (×2), DMF (×2), DCM (×2), DMF (×2) and DCM (×5); a second cycle was repeated. The unreacted amino groups were capped with acetic anhydride (3 eq). The resin was washed with DCM (×2), DMF (×2), DCM (×2), DMF (×2) and DCM (×5).

### EXAMPLE 3

5-Amino-3-thiophen-3-yl-isoxazole-4-carboxylic Acid Amide

Cyanacetamide (1.5 g, 18 mmol) and 3-thiophenylhydroxyaminomethyl chloride (3.46 g, 21 mmol) were suspended in dry THF (80 ml) and cooled to −20° C. Then lithium bis(trimethylsilyl)amide LiHMDS (41.02 ml of 1 M solution in THF, 41.02 mmol) was added, the resulting suspension was stirred at −20° C. for 15 min, allowed to warm to room temperature (23° C.) and stirred for 2 h. The reaction was quenched with water (50 ml) and stirred for 10′, the final mixture was extracted several times with ethyl acetate AcOEt and the organic phases were collected, dried over anhydrous Na₂SO₄ and solvents were removed under reduced pressure. The product was purified by crystallization from isopropanol and water or by flash chromatography over silica gel. [M+H]+=210. ¹H-NMR (DMSO-d₆), diagnostic signals (ppm): 7.9 (d, 1H), 7.7 (dd, 1H), 7.6 (bs, 2H), 7.3 (d, 1H).

By working in an analogous way and by using the appropriate chloroxime derivative and the appropriate cyanacetic acid derivative such as ester, primary amide or secondary amide, the following compounds were prepared:
5-Amino-3-thiophen-2-yl-isoxazole-4-carboxylic acid amide [M+H]+=210. ¹H-NMR (DMSO-d₆), diagnostic signals (ppm): 7.85 (d, 1H), 7.8 (bs, 3H), 7.2 (dd, 1H).
5-Amino-3-pyridin-2-yl-isoxazole-4-carboxylic acid amide. [M+H]+=205. ¹H-NMR (DMSO-d₆), diagnostic signals (ppm): 9,8 (bs, 1H), 8,7 (m, 1H), 8,05 (m, 2H), 7,95 (bs, 2H), 7,6 (m, 1H), 7,2 (bs, 1H).
5-Amino-3-pyridin-3-yl-isoxazole-4-carboxylic acid amide. [M+H]+=205. ¹H-NMR (DMSO-d₆), diagnostic signals (ppm): 8,7 (m, 2H), 7,95 (m, 1H), 7,6 (bs, 2H), 7,5 (m, 1H).
5-Amino-3-pyridin-4-yl-isoxazole-4-carboxylic acid amide. [M+H]+=205. ¹H-NMR (DMSO-d₆), diagnostic signals (ppm): 8,7 (d, 2H), 7,7 (bs, 2H), 7,5 (s, 2H).
5-Amino-3-thiazol-2-yl-isoxazole-4-carboxylic acid amide. [M+H]+=211. ¹H-NMR (DMSO-d₆), diagnostic signals (ppm): 9,5 (bs, 1H), 8,1 (s, 1H), 8,0 (bs, 2H), 7,9 (d, 1H).
5-Amino-3-(3,5-dimethyl-1-phenyl-1H-pyrazol-4-yl)-isoxazole-4-carboxylic acid amide. [M+H]+=298. ¹H-NMR (DMSO-d₆), diagnostic signals (ppm): 7,65 (bs, 2H), 7,4-7,5 (m, 5H), 2,2 (s, 3H), 2,1 (s, 3H).
5-Amino-3-quinolin-2-yl-isoxazole-4-carboxylic acid amide. [M+H]+=255. ¹H-NMR (DMSO-d₆), diagnostic signals (ppm): 10,4 (bs, 1H), 8,6 (d, 1H), 8,15 (d, 1H), 8,1 (dd, 1H) 8,15 (bs, 2H), 8,0 (dd, 1H), 7,9 (m, 1H), 7,75 (m, 1H).
5-Amino-3-thiophen-2-yl-isoxazole-4-carboxylic acid butylamide. [M+H]+=279. ¹H-NMR (DMSO-d₆), diagnostic signals (ppm): 7,8(d, 1H), 7,6 (d, 1H), 7,4 (bs, 2H), 7,2 (dd, 1H) 3,2 (m, 2H), 1,4 (m, 2H), 1,2 (m, 2H), 0,9 (m, 3H).
Methyl 5-amino-3-(2-pyridinyl)-4-isoxazolecarboxylate. [M+H]+=220. ¹H-NMR (DMSO-d₆), diagnostic signals (ppm): 8,6 (d, 1H), 7,9 (bs, 2H), 7,85 (dd, 1H), 7,55 (d, 1H) 7,45 (dd, 1H), 3,5 (s, 3H).
5-Amino-3-pyridin-4-yl-isoxazole-4-carboxylic acid (2,4-difluoro-phenyl)-amide. HPLC r.t. 4.1, [M+H]+=415.
5-Amino-3-pyridin-4-yl-isoxazole-4-carboxylic acid (4-chloro-phenyl)-amide. HPLC r.t. 4.58, [M+H]+=315.1.
5-Amino-3-pyridin-4-yl-isoxazole-4-carboxylic acid benzylamide. HPLC r.t. 3.55, [M+H]+=295.
5-Amino-3-pyridin-4-yl-isoxazole-4-carboxylic acid (5,6,7,8-tetrahydro-naphthalen-1-yl)-amide. HPLC r.t. 4.96, [M+H]+=335.
5-Amino-3-pyridin-4-yl-isoxazole-4-carboxylic acid (4-fluoro-phenyl)-amide. HPLC r.t. 3.86, [M+H]+=299.
5-Amino-3-pyridin-4-yl-isoxazole-4-carboxylic acid benzhydryl-amide. HPLC r.t. 5.02, [M+H]+=371.1.
5-Amino-3-thiophen-2-yl-isoxazole-4-carboxylic acid (2,4-difluoro-phenyl)-amide. HPLC r.t. 5.94, [M+H]+=322.
5-Amino-3-thiophen-2-yl-isoxazole-4-carboxylic acid (4-chloro-phenyl)-amide. HPLC r.t. 6.26, [M+H]+=438.
5-Amino-3-thiophen-2-yl-isoxazole-4-carboxylic acid benzylamide. HPLC r.t. 5.08, [M+H]+=300.
5-Amino-3-thiophen-2-yl-isoxazole-4-carboxylic acid (5,6,7,8-tetrahydro-naphthalen-1-yl)-amide. HPLC r.t. 6.7, [M+H]+=340.
5-Amino-3-thiophen-2-yl-isoxazole-4-carboxylic acid (4-fluoro-phenyl)-amide. HPLC r.t. 5.57, [M+H]+=304.
5-Amino-3-thiophen-2-yl-isoxazole-4-carboxylic acid benzhydryl-amide. HPLC r.t. 6.37, [M+H]+=375.9.
5-Amino-3-thiophen-3-yl-isoxazole-4-carboxylic acid (2,4-difluoro-phenyl)-amide. HPLC r.t. 5.8, [M+H]+=322.
5-Amino-3-thiophen-3-yl-isoxazole-4-carboxylic acid (4-chloro-phenyl)-amide. HPLC r.t. 6.15, [M+H]+=320.
5-Amino-3-thiophen-3-yl-isoxazole-4-carboxylic acid benzylamide. HPLC r.t. 4.96, [M+H]+=300.
5-Amino-3-thiophen-3-yl-isoxazole-4-carboxylic acid (5,6,7,8-tetrahydro-naphthalen-1-yl)-amide. HPLC r.t. 6.55, [M+H]+=340.
5-Amino-3-thiophen-3-yl-isoxazole-4-carboxylic acid (4-fluoro-phenyl)-amide. HPLC r.t. 5.48, [M+H]+=304.
5-Amino-3-thiophen-3-yl-isoxazole-4-carboxylic acid benzhydryl-amide. HPLC r.t. 6.24, [M+H]+=375.9.
5-Amino-3-thiazol-2-yl-isoxazole-4-carboxylic acid (2,4-difluoro-phenyl)-amide. HPLC r.t. 6.82, [M+H]+=323.
5-Amino-3-thiazol-2-yl-isoxazole-4-carboxylic acid (4-chloro-phenyl)-amide. HPLC r.t. 7.25, [M+H]+=321.
5-Amino-3-thiazol-2-yl-isoxazole-4-carboxylic acid benzylamide. HPLC r.t. 5.63, [M+H]+=301.
5-Amino-3-thiazol-2-yl-isoxazole-4-carboxylic acid (5,6,7,8-tetrahydro-naphthalen-1-yl)-amide. HPLC r.t. 7.21, [M+H]+=341.
5-Amino-3-thiazol-2-yl-isoxazole-4-carboxylic acid (4-fluoro-phenyl)-amide. HPLC r.t. 6.5, [M+H]+=305.
5-Amino-3-thiazol-2-yl-isoxazole-4-carboxylic acid benzhydryl-amide. HPLC r.t. 6.76, [M+H]+=376.8.
5-Amino-3-pyridin-2-yl-isoxazole-4-carboxylic acid (2,4-difluoro-phenyl)-amide. HPLC r.t. 6.79, [M+H]+=317.
5-Amino-3-pyridin-2-yl-isoxazole-4-carboxylic acid (4-chloro-phenyl)-amide. HPLC r.t. 7.07, [M+H]+=315.
5-Amino-3-pyridin-2-yl-isoxazole-4-carboxylic acid benzylamide. HPLC r.t. 5.44, [M+H]+=295.1.
5-Amino-3-pyridin-2-yl-isoxazole-4-carboxylic acid (5,6,7,8-tetrahydro-naphthalen-1-yl)-amide. HPLC r.t. 6.89, [M+H]+=335.
5-Amino-3-pyridin-2-yl-isoxazole-4-carboxylic acid (4-fluoro-phenyl)-amide. HPLC r.t. 6.32, [M+H]+=299.
5-Amino-3-pyridin-2-yl-isoxazole-4-carboxylic acid benzhydryl-amide. HPLC r.t. 6.64, [M+H]+=370.9.
5-Amino-3-pyridin-3-yl-isoxazole-4-carboxylic acid (2,4-difluoro-phenyl)-amide. HPLC r.t. 4.08, [M+H]+=317.1.
5-Amino-3-pyridin-3-yl-isoxazole-4-carboxylic acid (4-chloro-phenyl)-amide. HPLC r.t. 4.59, [M+H]+=315.1.
5-Amino-3-pyridin-3-yl-isoxazole-4-carboxylic acid benzylamide. HPLC r.t. 3.54, [M+H]+=295.1.
5-Amino-3-pyridin-3-yl-isoxazole-4-carboxylic acid (5,6,7,8-tetrahydro-naphthalen-1-yl)-amide. HPLC r.t. 5, [M+H]+=335.
5-Amino-3-pyridin-3-yl-isoxazole-4-carboxylic acid (4-fluoro-phenyl)-amide. HPLC r.t. 3.84, [M+H]+=299.1.
5-Amino-3-pyridin-3-yl-isoxazole-4-carboxylic acid benzhydryl-amide. HPLC r.t. 5.05, [M+H]+=371.2.
5-Amino-3-(3,5-dimethyl-1-phenyl-1H-pyrazol-4-yl)-isoxazole-4-carboxylic acid (2,4-difluoro-phenyl)-amide. HPLC r.t. 6.04, [M+H]+=410.
5-Amino-3-(3,5-dimethyl-1-phenyl-1H-pyrazol-4-yl)-isoxazole-4-carboxylic acid (4-chloro-phenyl)-amide. HPLC r.t. 6.39, [M+H]+=408.
5-Amino-3-(3,5-dimethyl-1-phenyl-1H-pyrazol-4-yl)-isoxazole-4-carboxylic acid (5,6,7,8-tetrahydro-naphthalen-1-yl)-amide. HPLC r.t. 6.73, [M+H]+=428.2.
5-Amino-3-(3,5-dimethyl-1-phenyl-1H-pyrazol-4-yl)-isoxazole-4-carboxylic acid (4-fluoro-phenyl)-amide. HPLC r.t. 5.78, [M+H]+=392.1.
5-Amino-3-(3,5-dimethyl-1-phenyl-1H-pyrazol-4-yl)-isoxazole-4-carboxylic acid benzhydryl-amide. HPLC r.t. 6.35, [M+H]+=464.2.
5-Amino-3-quinolin-2-yl-isoxazole-4-carboxylic acid (2,4-difluoro-phenyl)-amide. HPLC r.t. 7.36, [M+H]+=367.
5-Amino-3-quinolin-2-yl-isoxazole-4-carboxylic acid (4-chloro-phenyl)-amide. HPLC r.t. 8.18, [M+H]+=365.
5-Amino-3-quinolin-2-yl-isoxazole-4-carboxylic acid benzylamide. HPLC r.t. 6.77, [M+H]+=345.1.
5-Amino-3-quinolin-2-yl-isoxazole-4-carboxylic acid (5,6,7,8-tetrahydro-naphthalen-1-yl)-amide. HPLC r.t. 7.99, [M+H]+=385.1.
5-Amino-3-quinolin-2-yl-isoxazole-4-carboxylic acid (4-fluoro-phenyl)-amide. HPLC r.t. 7.45, [M+H]+=349.
5-Amino-3-quinolin-2-yl-isoxazole-4-carboxylic acid benzhydryl-amide. HPLC r.t. 7.79, [M+H]+=421.
5-Amino-3-pyridin-4-yl-isoxazole-4-carboxylic acid naphthalen-1-ylamide. HPLC r.t. 4.41, [M+H]+=331.1.
5-Amino-3-pyridin-4-yl-isoxazole-4-carboxylic acid p-tolylamide. HPLC r.t. 4.12, [M+H]+=295.
5-Amino-3-pyridin-4-yl-isoxazole-4-carboxylic acid (3-benzyloxy-phenyl)-amide. HPLC r.t. 5.52, [M+H]+=387.1.
5-Amino-3-pyridin-4-yl-isoxazole-4-carboxylic acid 2,4-dimethoxy-benzylamide. HPLC r.t. 3.85, [M+H]+=355.
5-Amino-3-pyridin-4-yl-isoxazole-4-carboxylic acid indan-5-ylamide. HPLC r.t. 4.77, [M+H]+=321.
5-Amino-3-pyridin-4-yl-isoxazole-4-carboxylic acid (3,5-dimethoxy-phenyl)-amide. HPLC r.t. 4, [M+H]+=341.
5-Amino-3-thiophen-2-yl-isoxazole-4-carboxylic acid naphthalen-1-ylamide. HPLC r.t. 6.25, [M+H]+=336.
5-Amino-3-thiophen-2-yl-isoxazole-4-carboxylic acid p-tolylamide. HPLC r.t. 5.87, [M+H]+=300.
5-Amino-3-thiophen-2-yl-isoxazole-4-carboxylic acid (3-benzyloxy-phenyl)-amide. HPLC r.t. 6.92, [M+H]+=392.
5-Amino-3-thiophen-2-yl-isoxazole-4-carboxylic acid 2,4-dimethoxy-benzylamide. HPLC r.t. 5.29, [M+H]+=359.9.
5-Amino-3-thiophen-2-yl-isoxazole-4-carboxylic acid indan-5-ylamide. HPLC r.t. 6.48, [M+H]+=326.
5-Amino-3-thiophen-2-yl-isoxazole-4-carboxylic acid (3,5-dimethoxy-phenyl)-amide. HPLC r.t. 5.64, [M+H]+=346.
5-Amino-3-thiophen-3-yl-isoxazole-4-carboxylic acid naphthalen-1-ylamide. HPLC r.t. 6.14, [M+H]+=336.
5-Amino-3-thiophen-3-yl-isoxazole-4-carboxylic acid p-tolylamide. HPLC r.t. 5.76, [M+H]+=300.
5-Amino-3-thiophen-3-yl-isoxazole-4-carboxylic acid (3-benzyloxy-phenyl)-amide. HPLC r.t. [M+H]+=392.
5-Amino-3-thiophen-3-yl-isoxazole-4-carboxylic acid 2,4-dimethoxy-benzylamide. HPLC r.t. 5.18, [M+H]+=359.9.
5-Amino-3-thiophen-3-yl-isoxazole-4-carboxylic acid indan-5-ylamide. HPLC r.t. 6.37, [M+H]+=326.
5-Amino-3-thiophen-3-yl-isoxazole-4-carboxylic acid (3,5-dimethoxy-phenyl)-amide. HPLC r.t. 5.52, [M+H]+=346.
5-Amino-3-thiazol-2-yl-isoxazole-4-carboxylic acid naphthalen-1-ylamide. HPLC r.t. 6.95, [M+H]+=337.
5-Amino-3-thiazol-2-yl-isoxazole-4-carboxylic acid p-tolylamide. HPLC r.t. 6.73, [M+H]+=301.
5-Amino-3-thiazol-2-yl-isoxazole-4-carboxylic acid (3-benzyloxy-phenyl)-amide. HPLC r.t. 7.7, [M+H]+=393.
5-Amino-3-thiazol-2-yl-isoxazole-4-carboxylic acid 2,4-dimethoxy-benzylamide. HPLC r.t. 5.64, [M+H]+=360.8.
5-Amino-3-thiazol-2-yl-isoxazole-4-carboxylic acid indan-5-ylamide. HPLC r.t. 7.38, [M+H]+=327.
5-Amino-3-thiazol-2-yl-isoxazole-4-carboxylic acid (3,5-dimethoxy-phenyl)-amide. HPLC r.t. 6.37, [M+H]+=347.
5-Amino-3-pyridin-2-yl-isoxazole-4-carboxylic acid naphthalen-1-ylamide. HPLC r.t. 6.75.
5-Amino-3-pyridin-2-yl-isoxazole-4-carboxylic acid p-tolylamide. HPLC r.t. 6.53, [M+H]+=295.
5-Amino-3-pyridin-2-yl-isoxazole-4-carboxylic acid (3-benzyloxy-phenyl)-amide. HPLC r.t. 7.55, [M+H]+=387.
5-Amino-3-pyridin-2-yl-isoxazole-4-carboxylic acid 2,4-dimethoxy-benzylamide. HPLC r.t. 5.39, [M+H]+=417.1.
5-Amino-3-pyridin-2-yl-isoxazole-4-carboxylic acid indan-5-ylamide. HPLC r.t. 7.17, [M+H]+=321.
5-Amino-3-pyridin-2-yl-isoxazole-4-carboxylic acid (3,5-dimethoxy-phenyl)-amide. HPLC r.t. 6.19, [M+H]+=341.
5-Amino-3-pyridin-3-yl-isoxazole-4-carboxylic acid naphthalen-1-ylamide. HPLC r.t. 4.44, [M+H]+=331.
5-Amino-3-pyridin-3-yl-isoxazole-4-carboxylic acid p-tolylamide. HPLC r.t. 4.13, [M+H]+=295.
5-Amino-3-pyridin-3-yl-isoxazole-4-carboxylic acid (3-benzyloxy-phenyl)-amide. HPLC r.t. 5.53, [M+H]+=387.1.
5-Amino-3-pyridin-3-yl-isoxazole-4-carboxylic acid 2,4-dimethoxy-benzylamide. HPLC r.t. 3.87, [M+H]+=355.
5-Amino-3-pyridin-3-yl-isoxazole-4-carboxylic acid indan-5-ylamide. HPLC r.t. 4.78, [M+H]+=321.1.
5-Amino-3-pyridin-3-yl-isoxazole-4-carboxylic acid (3,5-dimethoxy-phenyl)-amide. HPLC r.t. 3.99, [M+H]+=341.
5-Amino-3-(3,5-dimethyl-1-phenyl-1H-pyrazol-4-yl)-isoxazole-4-carboxylic acid naphthalen-1-ylamide. HPLC r.t. 6.37, [M+H]+=424.1.
5-Amino-3-(3,5-dimethyl-1-phenyl-1H-pyrazol-4-yl)-isoxazole-4-carboxylic acid p-tolylamide. HPLC r.t. 6.01, [M+H]+=388.1.
5-Amino-3-(3,5-dimethyl-1-phenyl-1H-pyrazol-4-yl)-isoxazole-4-carboxylic acid (3-benzyloxy-phenyl)-amide. HPLC r.t. 6.99, [M+H]+=480.1.
5-Amino-3-(3,5-dimethyl-1-phenyl-1H-pyrazol-4-yl)-isoxazole-4-carboxylic acid 2,4-dimethoxy-benzylamide. HPLC r.t. 5.31, [M+H]+=448.
5-Amino-3-(3,5-dimethyl-1-phenyl-1H-pyrazol-4-yl)-isoxazole-4-carboxylic acid indan-5-ylamide. HPLC r.t. 6.54, [M+H]+=414.1.
5-Amino-3-(3,5-dimethyl-1-phenyl-1H-pyrazol-4-yl)-isoxazole-4-carboxylic acid (3,5-dimethoxy-phenyl)-amide. HPLC r.t. 5.8, [M+H]+=434.1.
5-Amino-3-quinolin-2-yl-isoxazole-4-carboxylic acid naphthalen-1-ylamide. HPLC r.t. 7.66, [M+H]+=381.
5-Amino-3-quinolin-2-yl-isoxazole-4-carboxylic acid p-tolylamide. HPLC r.t. 7.69, [M+H]+=345.
5-Amino-3-quinolin-2-yl-isoxazole-4-carboxylic acid (3-benzyloxy-phenyl)-amide. HPLC r.t. 8.51, [M+H]+=437.1.
5-Amino-3-quinolin-2-yl-isoxazole-4-carboxylic acid 2,4-dimethoxy-benzylamide. HPLC r.t. 6.6, [M+H]+=405.
5-Amino-3-quinolin-2-yl-isoxazole-4-carboxylic acid indan-5-ylamide. HPLC r.t. 8.35, [M+H]+=371.
5-Amino-3-quinolin-2-yl-isoxazole-4-carboxylic acid (3,5-dimethoxy-phenyl)-amide. HPLC r.t. 7.34, [M+H]+=391.
5-Amino-3-pyridin-4-yl-isoxazole-4-carboxylic acid (2-methoxy-dibenzofuran-3-yl)-amide. HPLC r.t. 6.44, [M+H]+=401.
5-Amino-3-pyridin-4-yl-isoxazole-4-carboxylic acid (3,4,5-trimethoxy-phenyl)-amide. HPLC r.t. 3.27, [M+H]+=371.2.
5-Amino-3-pyridin-4-yl-isoxazole-4-carboxylic acid (2-methoxy-5-trifluoromethyl-phenyl)-amide. HPLC r.t. 5.66, [M+H]+=379.1.
5-Amino-3-pyridin-4-yl-isoxazole-4-carboxylic acid quinolin-8-ylamide. HPLC r.t. 5.08, [M+H]+=332.
5-Amino-3-pyridin-4-yl-isoxazole-4-carboxylic acid (4-tert-butyl-phenyl)-amide. HPLC r.t. 5.5, [M+H]+=337.1.
5-Amino-3-thiophen-2-yl-isoxazole-4-carboxylic acid (2-methoxy-dibenzofuran-3-yl)-amide. HPLC r.t. 7.8, [M+H]+=406.
5-Amino-3-thiophen-2-yl-isoxazole-4-carboxylic acid (3,4,5-trimethoxy-phenyl)-amide. HPLC r.t. 4.87, [M+H]+=376.
5-Amino-3-thiophen-2-yl-isoxazole-4-carboxylic acid quinolin-8-ylamide. HPLC r.t. 6.62, [M+H]+=336.9.
5-Amino-3-thiophen-2-yl-isoxazole-4-carboxylic acid (4-tert-butyl-phenyl)-amide. HPLC r.t. 7.04, [M+H]+=342.
5-Amino-3-thiophen-3-yl-isoxazole-4-carboxylic acid (2-methoxy-dibenzofuran-3-yl)-amide. HPLC r.t. 7.6, [M+H]+=406.
5-Amino-3-thiophen-3-yl-isoxazole-4-carboxylic acid (3,4,5-trimethoxy-phenyl)-amide. HPLC r.t. 4.78, [M+H]+=376.
5-Amino-3-thiophen-3-yl-isoxazole-4-carboxylic acid (2-methoxy-5-trifluoromethyl-phenyl)-amide. HPLC r.t. 6.84, [M+H]+=384.
5-Amino-3-thiophen-3-yl-isoxazole-4-carboxylic acid quinolin-8-ylamide. HPLC r.t. 6.39, [M+H]+=337.
5-Amino-3-thiophen-3-yl-isoxazole-4-carboxylic acid (4-tert-butyl-phenyl)-amide. HPLC r.t. 6.95, [M+H]+=342.
5-Amino-3-thiazol-2-yl-isoxazole-4-carboxylic acid (2-methoxy-dibenzofuran-3-yl)-amide. HPLC r.t. 8.21, [M+H]+=407.
5-Amino-3-thiazol-2-yl-isoxazole-4-carboxylic acid quinolin-8-ylamide. HPLC r.t. 6.71, [M+H]+=338.
5-Amino-3-thiazol-2-yl-isoxazole-4-carboxylic acid (4-tert-butyl-phenyl)-amide. HPLC r.t. 7.88, [M+H]+=343.
5-Amino-3-pyridin-2-yl-isoxazole-4-carboxylic acid (2-methoxy-dibenzofuran-3-yl)-amide. HPLC r.t. 7.98, [M+H]+=401.
5-Amino-3-pyridin-2-yl-isoxazole-4-carboxylic acid (3,4,5-trimethoxy-phenyl)-amide. HPLC r.t. 5.45, [M+H]+=371.
5-Amino-3-pyridin-2-yl-isoxazole-4-carboxylic acid (2-methoxy-5-trifluoromethyl-phenyl)-amide. HPLC r.t. 7.19, [M+H]+=379.
5-Amino-3-pyridin-2-yl-isoxazole-4-carboxylic acid quinolin-8-ylamide. HPLC r.t. 6.55, [M+H]+=332.
5-Amino-3-pyridin-2-yl-isoxazole-4-carboxylic acid (4-tert-butyl-phenyl)-amide. HPLC r.t. 7.68, [M+H]+=337.1.
5-Amino-3-pyridin-3-yl-isoxazole-4-carboxylic acid (2-methoxy-dibenzofuran-3-yl)-amide. HPLC r.t. 6.37, [M+H]+=401.1.
5-Amino-3-pyridin-3-yl-isoxazole-4-carboxylic acid quinolin-8-ylamide. HPLC r.t. 4.98, [M+H]+=332.
5-Amino-3-pyridin-3-yl-isoxazole-4-carboxylic acid (4-tert-butyl-phenyl)-amide. HPLC r.t. 5.52, [M+H]+=337.1.
5-Amino-3-(3,5-dimethyl-1-phenyl-1H-pyrazol-4-yl)-isoxazole-4-carboxylic acid (2-methoxy-dibenzofuran-3-yl)-amide. HPLC r.t. 7.76, [M+H]+=494.
5-Amino-3-(3,5-dimethyl-1-phenyl-1H-pyrazol-4-yl)-isoxazole-4-carboxylic acid (2-methoxy-5-trifluoromethyl-phenyl)-amide. HPLC r.t. 7.03, [M+H]+=472.
5-Amino-3-(3,5-dimethyl-1-phenyl-1H-pyrazol-4-yl)-isoxazole-4-carboxylic acid (4-tert-butyl-phenyl)-amide. HPLC r.t. 7.07, [M+H]+=430.2.
5-Amino-3-quinolin-2-yl-isoxazole-4-carboxylic acid (2-methoxy-dibenzofuran-3-yl)-amide. HPLC r.t. 8.77, [M+H]+=451.
5-Amino-3-quinolin-2-yl-isoxazole-4-carboxylic acid (3,4,5-trimethoxy-phenyl)-amide. HPLC r.t. 6.61, [M+H]+=421.
5-Amino-3-quinolin-2-yl-isoxazole-4-carboxylic acid quinolin-8-ylamide. HPLC r.t. 7.14, [M+H]+=382.
5-Amino-3-quinolin-2-yl-isoxazole-4-carboxylic acid (4-tert-butyl-phenyl)-amide. HPLC r.t. 8.75, [M+H]+=387.1.
5-Amino-3-pyridin-4-yl-isoxazole-4-carboxylic acid (2-benzyl-phenyl)-amide. HPLC r.t. 5.13, [M+H]+=371.
3-[(5-Amino-3-pyridin-4-yl-isoxazole-4-carbonyl)-amino]-benzoic acid ethyl ester. HPLC r.t. 4.44, [M+H]+=353.
5-Amino-3-pyridin-4-yl-isoxazole-4-carboxylic acid (4-trifluoromethoxy-phenyl)-amide. HPLC r.t. 5.13, [M+H]+=365.
5-Amino-3-thiophen-2-yl-isoxazole-4-carboxylic acid (2-benzyl-phenyl)-amide. HPLC r.t. 6.6, [M+H]+=376.
3-[(5-Amino-3-thiophen-2-yl-isoxazole-4-carbonyl)-amino]-benzoic acid ethyl ester. HPLC r.t. 6.02, [M+H]+=358.
5-Amino-3-thiophen-2-yl-isoxazole-4-carboxylic acid (4-trifluoromethoxy-phenyl)-amide. HPLC r.t. 6.59, [M+H]+=370.
5-Amino-3-thiophen-3-yl-isoxazole-4-carboxylic acid (4-dimethylamino-phenyl)-amide. HPLC r.t. 5.32, [M+H]+=329.
5-Amino-3-thiophen-3-yl-isoxazole-4-carboxylic acid (2-benzyl-phenyl)-amide. HPLC r.t. 6.47, [M+H]+=376.
3-[(5-Amino-3-thiophen-3-yl-isoxazole-4-carbonyl)-amino]-benzoic acid ethyl ester. HPLC r.t. 5.92, [M+H]+=357.9.
5-Amino-3-thiophen-3-yl-isoxazole-4-carboxylic acid (4-trifluoromethoxy-phenyl)-amide. HPLC r.t. 6.49, [M+H]+=370.
5-Amino-3-thiazol-2-yl-isoxazole-4-carboxylic acid (4-dimethylamino-phenyl)-amide. HPLC r.t. 6.23, [M+H]+=330.1.
5-Amino-3-thiazol-2-yl-isoxazole-4-carboxylic acid (2-benzyl-phenyl)-amide. HPLC r.t. 7.04, [M+H]+=377.
3-[(5-Amino-3-thiazol-2-yl-isoxazole-4-carbonyl)-amino]-benzoic acid ethyl ester. HPLC r.t. 6.97, [M+H]+=358.9.
5-Amino-3-thiazol-2-yl-isoxazole-4-carboxylic acid (4-trifluoromethoxy-phenyl)-amide. HPLC r.t. 7.5, [M+H]+=371.
5-Amino-3-pyridin-2-yl-isoxazole-4-carboxylic acid (4-dimethylamino-phenyl)-amide. HPLC r.t. 5.98.
5-Amino-3-pyridin-2-yl-isoxazole-4-carboxylic acid (2-benzyl-phenyl)-amide. HPLC r.t. 6.74, [M+H]+=371.
3-[(5-Amino-3-pyridin-2-yl-isoxazole-4-carbonyl)-amino]-benzoic acid ethyl ester. HPLC r.t. 6.84, [M+H]+=353.
5-Amino-3-pyridin-2-yl-isoxazole-4-carboxylic acid (4-trifluoromethoxy-phenyl)-amide. HPLC r.t. 7.34, [M+H]+=365.
5-Amino-3-pyridin-3-yl-isoxazole-4-carboxylic acid (4-dimethylamino-phenyl)-amide. HPLC r.t. 3.66, [M+H]+=324.
5-Amino-3-pyridin-3-yl-isoxazole-4-carboxylic acid (2-benzyl-phenyl)-amide. HPLC r.t. 5.16, [M+H]+=371.2.
3-[(5-Amino-3-pyridin-3-yl-isoxazole-4-carbonyl)-amino]-benzoic acid ethyl ester. HPLC r.t. 4.45, [M+H]+=353.1.
5-Amino-3-pyridin-3-yl-isoxazole-4-carboxylic acid (4-trifluoromethoxy-phenyl)-amide. HPLC r.t. 5.15, [M+H]+=365.
5-Amino-3-(3,5-dimethyl-1-phenyl-1H-pyrazol-4-yl)-isoxazole-4-carboxylic acid (2-benzyl-phenyl)-amide. HPLC r.t. 6.72, [M+H]+=464.2.
3-{[5-Amino-3-(3,5-dimethyl-1-phenyl-1H-pyrazol-4-yl)-isoxazole-4-carbonyl]-amino}-benzoic acid ethyl ester. HPLC r.t. 6.18, [M+H]+=446.1.
5-Amino-3-(3,5-dimethyl-1-phenyl-1H-pyrazol-4-yl)-isoxazole-4-carboxylic acid (4-trifluoromethoxy-phenyl)-amide. HPLC r.t. 6.69, [M+H]+=458.1.
5-Amino-3-quinolin-2-yl-isoxazole-4-carboxylic acid (4-dimethylamino-phenyl)-amide. HPLC r.t. 7.27, [M+H]+=374.1.
5-Amino-3-quinolin-2-yl-isoxazole-4-carboxylic acid (2-benzyl-phenyl)-amide. HPLC r.t. 7.6, [M+H]+=421.
3-[(5-Amino-3-quinolin-2-yl-isoxazole-4-carbonyl)-amino]-benzoic acid ethyl ester. HPLC r.t. 7.99, [M+H]+=403.
5-Amino-3-quinolin-2-yl-isoxazole-4-carboxylic acid (4-trifluoromethoxy-phenyl)-amide. HPLC r.t. 8.33.
5-amino-N-benzyl-3-{5-[3-(trifluoromethyl)phenyl]-2-furyl}isoxazole-4-carboxamide. HPLC (Method B) r.t. 1.62, mass found=427.11.
5-amino-N-(2-furylmethyl)-3-{5-[3-(trifluoromethyl)phenyl]-2-furyl}isoxazole-4-carboxamide. HPLC (Method B) r.t. 1.55, mass found=417.09.
5-amino-N-(2-phenylethyl)-3-{5-[3-(trifluoromethyl)phenyl]-2-furyl}isoxazole-4-carboxamide. HPLC (Method B) r.t. 1.64, mass found=441.13.
5-amino-N-cyclohexyl-3-{5-[3-(trifluoromethyl)phenyl]-2-furyl}isoxazole-4-carboxamide. HPLC (Method B) r.t. 1.69, mass found=419.15.
5-amino-N-(4-methoxybenzyl)-3-{5-[3-(trifluoromethyl)phenyl]-2-furyl}isoxazole-4-carboxamide. HPLC (Method B) r.t. 1.59, mass found=457.12.
5-amino-N-hexyl-3-{5-[3-(trifluoromethyl)phenyl]-2-furyl}isoxazole-4-carboxamide. HPLC (Method B) r.t. 1.76, mass found=421.16.
5-amino-3-{5-[3-(trifluoromethyl)phenyl]-2-furyl}-N-(tetrahydrofuran-2-ylmethyl)isoxazole-4-carboxamide. HPLC (Method B) r.t. 1.51, mass found=421.12.
5-amino-3-{5-[3-(trifluoromethyl)phenyl]-2-furyl}-N-(thien-2-ylmethyl)isoxazole-4-carboxamide. HPLC (Method B) r.t. 1.61, mass found=433.07.
5-amino-N-cyclopentyl-3-{5-[3-(trifluoromethyl)phenyl]-2-furyl}isoxazole-4-carboxamide. HPLC (Method B) r.t. 1.62, mass found=405.13.
5-amino-N-(1,5-dimethylhexyl)-3-{5-[3-(trifluoromethyl)phenyl]-2-furyl}isoxazole-4-carboxamide. HPLC (Method B) r.t. 1.87, mass found=449.19.
5-amino-N-isopentyl-3-{5-[3-(trifluoromethyl)phenyl]-2-furyl}isoxazole-4-carboxamide. HPLC (Method B) r.t. 1.67, mass found=407.15.
5-amino-N-(2-ethylhexyl)-3-{5-[3-(trifluoromethyl)phenyl]-2-furyl}isoxazole-4-carboxamide. HPLC (Method B) r.t. 1.87, mass found=449.19.
5-amino-3-{5-[3-(trifluoromethyl)phenyl]-2-furyl}-N-(4-phenylbutyl)isoxazole-4-carboxamide. HPLC (Method B) r.t. 1.74, mass found=469.16.
5-amino-N-(2-methylcyclohexyl)-3-{5-[3-(trifluoromethyl)phenyl]-2-furyl}isoxazole-4-carboxamide. HPLC (Method B) r.t. 1.74, mass found=433.16.
5-amino-N-[3,5-bis(trifluoromethyl)benzyl]-3-{5-[3-(trifluoromethyl)phenyl]-2-furyl}isoxazole-4-carboxamide. HPLC (Method B) r.t. 1.78, mass found=563.09.
5-amino-3-{5-[3-(trifluoromethyl)phenyl]-2-furyl}-N-[3-(trifluoromethyl)benzyl]isoxazole-4-carboxamide. HPLC (Method B) r.t. 1.69, mass found=495.1.
5-amino-3-{5-[3-(trifluoromethyl)phenyl]-2-furyl}-N-(3-phenylpropyl)isoxazole-4-carboxamide. HPLC (Method B) r.t. 1.69, mass found=455.15.
5-amino-N-isobutyl-3-{5-[3-(trifluoromethyl)phenyl]-2-furyl}isoxazole-4-carboxamide. HPLC (Method B) r.t. 1.61, mass found=393.13.
5-amino-3-{5-[3-(trifluoromethyl)phenyl]-2-furyl}-N-[2-(trifluoromethyl)benzyl]isoxazole-4-carboxamide. HPLC (Method B) r.t. 1.69, mass found=495.1.
5-amino-N-(2-methoxybenzyl)-3-{5-[3-(trifluoromethyl)phenyl]-2-furyl}isoxazole-4-carboxamide. HPLC (Method B) r.t. 1.64, mass found=457.12.
5-amino-N-(2-fluorobenzyl)-3-{5-[3-(trifluoromethyl)phenyl]-2-furyl}isoxazole-4-carboxamide. HPLC (Method B) r.t. 1.63, mass found=445.1.
5-amino-3-{5-[3-(trifluoromethyl)phenyl]-2-furyl}-N-[4-(trifluoromethyl)benzyl]isoxazole-4-carboxamide. HPLC (Method B) r.t. 1.71, mass found=495.1.
5-amino-N-(3,4-difluorobenzyl)-3-{5-[3-(trifluoromethyl)phenyl]-2-furyl}isoxazole-4-carboxamide. HPLC (Method B) r.t. 1.62, mass found=463.1.
5-amino-N-(3-chlorobenzyl)-3-{5-[3-(trifluoromethyl)phenyl]-2-furyl}isoxazole-4-carboxamide. HPLC (Method B) r.t. 1.68, mass found=461.08.
5-amino-N-(2-chlorobenzyl)-3-{5-[3-(trifluoromethyl)phenyl]-2-furyl}isoxazole-4-carboxamide. HPLC (Method B) r.t. 1.67, mass found=461.08.
5-amino-N-(3-methylbenzyl)-3-{5-[3-(trifluoromethyl)phenyl]-2-furyl}isoxazole-4-carboxamide. HPLC (Method B) r.t. 1.67, mass found=441.13.
5-amino-3-{5-[3-(trifluoromethyl)phenyl]-2-furyl}-N-(1-naphthylmethyl)isoxazole-4-carboxamide. HPLC (Method B) r.t. 1.71, mass found=477.13.
5-amino-N-(cyclopropylmethyl)-3-{5-[3-(trifluoromethyl)phenyl]-2-furyl}isoxazole-4-carboxamide. HPLC (Method B) r.t. 1.57, mass found=391.11.
5-amino-N-cycloheptyl-3-{5-[3-(trifluoromethyl)phenyl]-2-furyl}isoxazole-4-carboxamide. HPLC (Method B) r.t. 1.75, mass found=433.16.
5-amino-N-[2-(4-methoxyphenyl)ethyl]-3-{5-[3-(trifluoromethyl)phenyl]-2-furyl}isoxazole-4-carboxamide. HPLC (Method B) r.t. 1.61, mass found=471.14.
5-amino-N-(2-cyclohex-1-en-1-ylethyl)-3-{5-[3-(trifluoromethyl)phenyl]-2-furyl}isoxazole-4-carboxamide. HPLC (Method B) r.t. 1.77, mass found=445.16.
5-amino-N-(1,2-dimethylpropyl)-3-{5-[3-(trifluoromethyl)phenyl]-2-furyl}isoxazole-4-carboxamide. HPLC (Method B) r.t. 1.65, mass found=407.15.
5-amino-N-(3,3-diphenylpropyl)-3-{5-[3-(trifluoromethyl)phenyl]-2-furyl}isoxazole-4-carboxamide. HPLC (Method B) r.t. 1.8, mass found=531.18.
5-amino-N-(1,2-diphenylethyl)-3-{5-[3-(trifluoromethyl)phenyl]-2-furyl}isoxazole-4-carboxamide. HPLC (Method B) r.t. 1.77, mass found=517.16.
5-amino-N-(2,4-dimethoxybenzyl)-3-{5-[3-(trifluoromethyl)phenyl]-2-furyl}isoxazole-4-carboxamide. HPLC (Method B) r.t. 1.59, mass found=487.14.
5-amino-N-[3-(dibutylamino)propyl]-3-{5-[3-(trifluoromethyl)phenyl]-2-furyl}isoxazole-4-carboxamide. HPLC (Method B) r.t. 1.44, mass found=506.25.
5-amino-N-butyl-3-{5-[3-(trifluoromethyl)phenyl]-2-furyl}isoxazole-4-carboxamide. HPLC (Method B) r.t. 1.62, mass found=393.13.
5-amino-3-{5-[3-(trifluoromethyl)phenyl]-2-furyl}-N-[4-(trifluoromethoxy)benzyl]isoxazole-4-carboxamide. HPLC (Method B) r.t. 1.73, mass found=511.1.
5-amino-N-(1-methylhexyl)-3-{5-[3-(trifluoromethyl)phenyl]-2-furyl}isoxazole-4-carboxamide. HPLC (Method B) r.t. 1.82, mass found=435.18.
5-amino-N-(2-methoxy-1-methylethyl)-3-{5-[3-(trifluoromethyl)phenyl]-2-furyl}isoxazole-4-carboxamide. HPLC (Method B) r.t. 1.53, mass found=409.12.
5-amino-N-(1-methylbutyl)-3-{5-[3-(trifluoromethyl)phenyl]-2-furyl}isoxazole-4-carboxamide. HPLC (Method B) r.t. 1.69, mass found=407.15.
5-amino-N-(3-fluorobenzyl)-3-{5-[3-(trifluoromethyl)phenyl]-2-furyl}isoxazole-4-carboxamide. HPLC (Method B) r.t. 1.63, mass found=445.1.
5-amino-N-(4-methylbenzyl)-3-{5-[3-(trifluoromethyl)phenyl]-2-furyl}isoxazole-4-carboxamide. HPLC (Method B) r.t. 1.69, mass found=441.13.
5-amino-N-(1,3-dimethylbutyl)-3-{5-[3-(trifluoromethyl)phenyl]-2-furyl}isoxazole-4-carboxamide. HPLC (Method B) r.t. 1.74, mass found=421.16.
5-amino-3-{5-[3-(trifluoromethyl)phenyl]-2-furyl}-N-(1-methyl-3-phenylpropyl)isoxazole-4-carboxamide. HPLC (Method B) r.t. 1.75, mass found=469.16.
5-amino-3-{5-[3-(trifluoromethyl)phenyl]-2-furyl}-N-[3-(methylthio)propyl]isoxazole-4-carboxamide. HPLC (Method B) r.t. 1.57, mass found=425.1.
5-amino-N-(1,3-benzodioxol-5-ylmethyl)-3-{5-[3-(trifluoromethyl)phenyl]-2-furyl}isoxazole-4-carboxamide. HPLC (Method B) r.t. 1.57, mass found=471.1.
5-amino-3-{5-[3-(trifluoromethyl)phenyl]-2-furyl}-N-pentylisoxazole-4-carboxamide. HPLC (Method B) r.t. 1.69, mass found=407.15.
5-amino-N-(cyclohexylmethyl)-3-{5-[3-(trifluoromethyl)phenyl]-2-furyl}isoxazole-4-carboxamide. HPLC (Method B) r.t. 1.77, mass found=433.16.
5-amino-N-[2-(3-chlorophenyl)ethyl]-3-{5-[3-(trifluoromethyl)phenyl]-2-furyl}isoxazole-4-carboxamide. HPLC (Method B) r.t. 1.71, mass found=475.09.
5-amino-N-(1-ethylpropyl)-3-{5-[3-(trifluoromethyl)phenyl]-2-furyl}isoxazole-4-carboxamide. HPLC (Method B) r.t. 1.67, mass found=407.15.
5-amino-3-{5-[3-(trifluoromethyl)phenyl]-2-furyl}-N-(1-phenylethyl)isoxazole-4-carboxamide. HPLC (Method B) r.t. 1.67, mass found=441.13.
5-amino-N-(2-methylbenzyl)-3-{5-[3-(trifluoromethyl)phenyl]-2-furyl}isoxazole-4-carboxamide. HPLC (Method B) r.t. 1.67, mass found=441.13.
5-amino-N-cyclobutyl-3-{5-[3-(trifluoromethyl)phenyl]-2-furyl}isoxazole-4-carboxamide. HPLC (Method B) r.t. 1.58, mass found=391.11.
5-amino-3-{5-[3-(trifluoromethyl)phenyl]-2-furyl}-N-octylisoxazole-4-carboxamide. HPLC (Method B) r.t. 1.92, mass found=449.19.
5-amino-N-(2-bromobenzyl)-3-{5-[3-(trifluoromethyl)phenyl]-2-furyl}isoxazole-4-carboxamide. HPLC (Method B) r.t. 1.7, mass found=505.02.
5-amino-N-(1-methylheptyl)-3-{5-[3-(trifluoromethyl)phenyl]-2-furyl}isoxazole-4-carboxamide. HPLC (Method B) r.t. 1.89, mass found=449.19.
5-amino-N-heptyl-3-{5-[3-(trifluoromethyl)phenyl]-2-furyl}isoxazole-4-carboxamide. HPLC (Method B) r.t. 1.84, mass found=435.18.
5-amino-N-(tert-butyl)-3-{5-[3-(trifluoromethyl)phenyl]-2-furyl}isoxazole-4-carboxamide. HPLC (Method B) r.t. 1.66, mass found=393.13.
5-amino-N-(3,4-dimethoxybenzyl)-3-{5-[3-(trifluoromethyl)phenyl]-2-furyl}isoxazole-4-carboxamide. HPLC (Method B) r.t. 1.51, mass found=487.14.
5-amino-N-(2,3-dihydro-1H-inden-1-yl)-3-{5-[3-(trifluoromethyl)phenyl]-2-furyl}isoxazole-4-carboxamide. HPLC (Method B) r.t. 1.72, mass found=453.13.
5-amino-N-(1,2,3,4-tetrahydronaphthalen-1-yl)-3-{5-[3-(trifluoromethyl)phenyl]-2-furyl}isoxazole-4-carboxamide. HPLC (Method B) r.t. 1.74, mass found=467.15.
5-amino-N-benzyl-3-(5-bromothien-2-yl)isoxazole-4-carboxamide. HPLC (Method B) r.t. 1.46, mass found=376.98.
5-amino-3-(5-bromothien-2-yl)-N-(2-furylmethyl)isoxazole-4-carboxamide. HPLC (Method B) r.t. 1.38, mass found=366.96.
5-amino-3-(5-bromothien-2-yl)-N-(2-phenylethyl)isoxazole-4-carboxamide. HPLC (Method B) r.t. 1.52, mass found=391.
5-amino-3-(5-bromothien-2-yl)-N-(sec-butyl)isoxazole-4-carboxamide. HPLC (Method B) r.t. 1.45, mass found=343.
5-amino-3-(5-bromothien-2-yl)-N-cyclohexylisoxazole-4-carboxamide. HPLC (Method B) r.t. 1.57, mass found=369.01.
5-amino-3-(5-bromothien-2-yl)-N-(4-methoxybenzyl)isoxazole-4-carboxamide. HPLC (Method B) r.t. 1.44, mass found=406.99.
5-amino-3-(5-bromothien-2-yl)-N-isopropylisoxazole-4-carboxamide. HPLC (Method B) r.t. 1.37, mass found=328.98.
5-amino-3-(5-bromothien-2-yl)-N-hexylisoxazole-4-carboxamide. HPLC (Method B) r.t. 1.64, mass found=371.03.
N-allyl-5-amino-3-(5-bromothien-2-yl)isoxazole-4-carboxamide. HPLC (Method B) r.t. 1.32, mass found=326.97.
5-amino-3-(5-bromothien-2-yl)-N-(tetrahydrofuran-2-ylmethyl)isoxazole-4-carboxamide. HPLC (Method B) r.t. 1.29, mass found=370.99.
5-amino-3-(5-bromothien-2-yl)-N-(thien-2-ylmethyl)isoxazole-4-carboxamide. HPLC (Method B) r.t. 1.44, mass found=382.94.
5-amino-3-(5-bromothien-2-yl)-N-cyclopentylisoxazole-4-carboxamide. HPLC (Method B) r.t. 1.47, mass found=355.
5-amino-3-(5-bromothien-2-yl)-N-(1,5-dimethylhexyl)isoxazole-4-carboxamide. HPLC (Method B) r.t. 1.78, mass found=399.06.
5-amino-3-(5-bromothien-2-yl)-N-isopentylisoxazole-4-carboxamide. HPLC (Method B) r.t. 1.54, mass found=357.01.
5-amino-3-(5-bromothien-2-yl)-N-(2-ethylhexyl)isoxazole-4-carboxamide. HPLC (Method B) r.t. 1.79, mass found=399.06.
5-amino-3-(5-bromothien-2-yl)-N-(4-phenylbutyl)isoxazole-4-carboxamide. HPLC (Method B) r.t. 1.64, mass found=419.03.
5-amino-3-(5-bromothien-2-yl)-N-(2-methylcyclohexyl)isoxazole-4-carboxamide. HPLC (Method B) r.t. 1.64, mass found=383.03.
5-amino-N-[3,5-bis(trifluoromethyl)benzyl]-3-(5-bromothien-2-yl)isoxazole-4-carboxamide. HPLC (Method B) r.t. 1.69, mass found=512.96.
5-amino-3-(5-bromothien-2-yl)-N-[3-(trifluoromethyl)benzyl]isoxazole-4-carboxamide. HPLC (Method B) r.t. 1.57, mass found=444.97.
5-amino-3-(5-bromothien-2-yl)-N-(3-phenylpropyl)isoxazole-4-carboxamide. HPLC (Method B) r.t. 1.57, mass found=405.01.
5-amino-3-(5-bromothien-2-yl)-N-isobutylisoxazole-4-carboxamide. HPLC (Method B) r.t. 1.44, mass found=343.
5-amino-3-(5-bromothien-2-yl)-N-[2-(trifluoromethyl)benzyl]isoxazole-4-carboxamide. HPLC (Method B) r.t. 1.58, mass found=444.97.
5-amino-3-(5-bromothien-2-yl)-N-(2-methoxybenzyl)isoxazole-4-carboxamide. HPLC (Method B) r.t. 1.49, mass found=406.99.
5-amino-3-(5-bromothien-2-yl)-N-(2-fluorobenzyl)isoxazole-4-carboxamide. HPLC (Method B) r.t. 1.47, mass found=394.97.
5-amino-3-(5-bromothien-2-yl)-N-[4-(trifluoromethyl)benzyl]isoxazole-4-carboxamide. HPLC (Method B) r.t. 1.58, mass found=444.97.
5-amino-3-(5-bromothien-2-yl)-N-(3,4-difluorobenzyl)isoxazole-4-carboxamide. HPLC (Method B) r.t. 1.49, mass found=412.96.
5-amino-3-(5-bromothien-2-yl)-N-(3-chlorobenzyl)isoxazole-4-carboxamide. HPLC (Method B) r.t. 1.55, mass found=410.94.
5-amino-3-(5-bromothien-2-yl)-N-(2-chlorobenzyl)isoxazole-4-carboxamide. HPLC (Method B) r.t. 1.56, mass found=410.94.
5-amino-3-(5-bromothien-2-yl)-N-(3-methylbenzyl)isoxazole-4-carboxamide. HPLC (Method B) r.t. 1.54, mass found=391.
5-amino-3-(5-bromothien-2-yl)-N-(1-naphthylmethyl)isoxazole-4-carboxamide. HPLC (Method B) r.t. 1.6, mass found=427.
5-amino-3-(5-bromothien-2-yl)-N-(cyclopropylmethyl)isoxazole-4-carboxamide. HPLC (Method B) r.t. 1.39, mass found=340.98.
5-amino-3-(5-bromothien-2-yl)-N-cycloheptylisoxazole-4-carboxamide. HPLC (Method B) r.t. 1.64, mass found=383.03.
5-amino-3-(5-bromothien-2-yl)-N-[2-(4-methoxyphenyl)ethyl]isoxazole-4-carboxamide. HPLC (Method B) r.t. 1.48, mass found=421.01.
5-amino-3-(5-bromothien-2-yl)-N-(2-cyclohex-1-en-1-ylethyl)isoxazole-4-carboxamide. HPLC (Method B) r.t. 1.67, mass found=395.03.
5-amino-3-(5-bromothien-2-yl)-N-prop-2-ynylisoxazole-4-carboxamide. HPLC (Method B) r.t. 1.27, mass found=324.95.
5-amino-3-(5-bromothien-2-yl)-N-(1,2-dimethylpropyl)isoxazole-4-carboxamide. HPLC (Method B) r.t. 1.52, mass found=357.01.
5-amino-3-(5-bromothien-2-yl)-N-(3,3-diphenylpropyl)isoxazole-4-carboxamide. HPLC (Method B) r.t. 1.68, mass found=481.05.
5-amino-3-(5-bromothien-2-yl)-N-(1,2-diphenylethyl)isoxazole-4-carboxamide. HPLC (Method B) r.t. 1.68, mass found=467.03.
5-amino-3-(5-bromothien-2-yl)-N-[3-(dibutylamino)propyl]isoxazole-4-carboxamide. HPLC (Method B) r.t. 1.27, mass found=456.12.
5-amino-3-(5-bromothien-2-yl)-N-butylisoxazole-4-carboxamide. HPLC (Method B) r.t. 1.45, mass found=343.
5-amino-3-(5-bromothien-2-yl)-N-[4-(trifluoromethoxy)benzyl]isoxazole-4-carboxamide. HPLC (Method B) r.t. 1.6, mass found=460.97.
5-amino-3-(5-bromothien-2-yl)-N-[(5-methyl-2-furyl)methyl]isoxazole-4-carboxamide. HPLC (Method B) r.t. 1.45, mass found=380.98.
5-amino-3-(5-bromothien-2-yl)-N-(1-methylhexyl)isoxazole-4-carboxamide. HPLC (Method B) r.t. 1.7, mass found=385.05.
5-amino-3-(5-bromothien-2-yl)-N-(2-methoxy-1-methylethyl)isoxazole-4-carboxamide. HPLC (Method B) r.t. 1.32, mass found=358.99.
5-amino-3-(5-bromothien-2-yl)-N-(4-tert-butylcyclohexyl)isoxazole-4-carboxamide. HPLC (Method B) r.t. 1.88, mass found=425.08.
5-amino-3-(5-bromothien-2-yl)-N-(1-methylbutyl)isoxazole-4-carboxamide. HPLC (Method B) r.t. 1.54, mass found=357.01.
5-amino-3-(5-bromothien-2-yl)-N-(3-fluorobenzyl)isoxazole-4-carboxamide. HPLC (Method B) r.t. 1.47, mass found=394.97.
5-amino-3-(5-bromothien-2-yl)-N-(4-methylbenzyl)isoxazole-4-carboxamide. HPLC (Method B) r.t. 1.54, mass found=391.
5-amino-3-(5-bromothien-2-yl)-N-(1,3-dimethylbutyl)isoxazole-4-carboxamide. HPLC (Method B) r.t. 1.61, mass found=371.03.
5-amino-3-(5-bromothien-2-yl)-N-(1-methyl-3-phenylpropyl)isoxazole-4-carboxamide. HPLC (Method B) r.t. 1.62, mass found=419.03.
5-amino-3-(5-bromothien-2-yl)-N-[3-(methylthio)propyl]isoxazole-4-carboxamide. HPLC (Method B) r.t. 1.38, mass found=374.97.
5-amino-N-(1,3-benzodioxol-5-ylmethyl)-3-(5-bromothien-2-yl)isoxazole-4-carboxamide. HPLC (Method B) r.t. 1.42, mass found=420.97.
5-amino-3-(5-bromothien-2-yl)-N-pentylisoxazole-4-carboxamide. HPLC (Method B) r.t. 1.53, mass found=357.01.
5-amino-3-(5-bromothien-2-yl)-N-(cyclohexylmethyl)isoxazole-4-carboxamide. HPLC (Method B) r.t. 1.64, mass found=383.03.
ethyl 4-({[5-amino-3-(5-bromothien-2-yl)isoxazol-4-yl]carbonyl}amino)piperidine-1-carboxylate. HPLC (Method B) r.t. 1.35, mass found=442.03.
5-amino-3-(5-bromothien-2-yl)-N-[2-(3-chlorophenyl)ethyl]isoxazole-4-carboxamide. HPLC (Method B) r.t. 1.59, mass found=424.96.
5-amino-3-(5-bromothien-2-yl)-N-(1-ethylpropyl)isoxazole-4-carboxamide. HPLC (Method B) r.t. 1.52, mass found=357.01.
5-amino-3-(5-bromothien-2-yl)-N-(1-phenylethyl)isoxazole-4-carboxamide. HPLC (Method B) r.t. 1.53, mass found=391.
5-amino-3-(5-bromothien-2-yl)-N-propylisoxazole-4-carboxamide. HPLC (Method B) r.t. 1.36, mass found=328.98.
5-amino-3-(5-bromothien-2-yl)-N-(2-methylbenzyl)isoxazole-4-carboxamide. HPLC (Method B) r.t. 1.53, mass found=391.
5-amino-3-(5-bromothien-2-yl)-N-cyclobutylisoxazole-4-carboxamide. HPLC (Method B) r.t. 1.41, mass found=340.98.
5-amino-3-(5-bromothien-2-yl)-N-(3-methoxypropyl)isoxazole-4-carboxamide. HPLC (Method B) r.t. 1.27, mass found=358.99.
5-amino-3-(5-bromothien-2-yl)-N-octylisoxazole-4-carboxamide. HPLC (Method B) r.t. 1.82, mass found=399.06.
5-amino-N-(2-bromobenzyl)-3-(5-bromothien-2-yl)isoxazole-4-carboxamide. HPLC (Method B) r.t. 1.58, mass found=454.89.
5-amino-3-(5-bromothien-2-yl)-N-(1-methylheptyl)isoxazole-4-carboxamide. HPLC (Method B) r.t. 1.8, mass found=399.06.
5-amino-3-(5-bromothien-2-yl)-N-heptylisoxazole-4-carboxamide. HPLC (Method B) r.t. 1.73, mass found=385.05.
5-amino-3-(5-bromothien-2-yl)-N-(tert-butyl)isoxazole-4-carboxamide. HPLC (Method B) r.t. 1.51, mass found=343.
5-amino-3-(5-bromothien-2-yl)-N-(2,3-dihydro-1H-inden-1-yl)isoxazole-4-carboxamide. HPLC (Method B) r.t. 1.57, mass found=403.
5-amino-3-(5-bromothien-2-yl)-N-(1,2,3,4-tetrahydronaphthalen-1-yl)isoxazole-4-carboxamide. HPLC (Method B) r.t. 1.62, mass found=417.01.
5-amino-N-benzyl-3-(5-chlorothien-2-yl)isoxazole-4-carboxamide. HPLC (Method B) r.t. 1.46, mass found=333.03.
5-amino-3-(5-chlorothien-2-yl)-N-(2-furylmethyl)isoxazole-4-carboxamide. HPLC (Method B) r.t. 1.37, mass found=323.01.
5-amino-3-(5-chlorothien-2-yl)-N-(2-phenylethyl)isoxazole-4-carboxamide. HPLC (Method B) r.t. 1.52, mass found=347.05.
5-amino-N-(sec-butyl)-3-(5-chlorothien-2-yl)isoxazole-4-carboxamide. HPLC (Method B) r.t. 1.45, mass found=299.05.
5-amino-3-(5-chlorothien-2-yl)-N-cyclohexylisoxazole-4-carboxamide. HPLC (Method B) r.t. 1.57, mass found=325.07.
5-amino-3-(5-chlorothien-2-yl)-N-(4-methoxybenzyl)isoxazole-4-carboxamide. HPLC (Method B) r.t. 1.44, mass found=363.04.
5-amino-3-(5-chlorothien-2-yl)-N-isopropylisoxazole-4-carboxamide. HPLC (Method B) r.t. 1.36, mass found=285.03.
5-amino-3-(5-chlorothien-2-yl)-N-hexylisoxazole-4-carboxamide. HPLC (Method B) r.t. 1.65, mass found=327.08.
N-allyl-5-amino-3-(5-chlorothien-2-yl)isoxazole-4-carboxamide. HPLC (Method B) r.t. 1.32, mass found=283.02.
5-amino-3-(5-chlorothien-2-yl)-N-(thien-2-ylmethyl)isoxazole-4-carboxamide. HPLC (Method B) r.t. 1.44, mass found=338.99.
5-amino-3-(5-chlorothien-2-yl)-N-cyclopentylisoxazole-4-carboxamide. HPLC (Method B) r.t. 1.47, mass found=311.05.
5-amino-3-(5-chlorothien-2-yl)-N-isopentylisoxazole-4-carboxamide. HPLC (Method B) r.t. 1.54, mass found=313.07.
5-amino-3-(5-chlorothien-2-yl)-N-(2-ethylhexyl)isoxazole-4-carboxamide. HPLC (Method B) r.t. 1.81, mass found=355.11.
5-amino-3-(5-chlorothien-2-yl)-N-(4-phenylbutyl)isoxazole-4-carboxamide. HPLC (Method B) r.t. 1.64, mass found=375.08.
5-amino-3-(5-chlorothien-2-yl)-N-(2-methylcyclohexyl)isoxazole-4-carboxamide. HPLC (Method B) r.t. 1.64, mass found=339.08.
5-amino-3-(5-chlorothien-2-yl)-N-[3-(trifluoromethyl)benzyl]isoxazole-4-carboxamide. HPLC (Method B) r.t. 1.58, mass found=401.02.
5-amino-3-(5-chlorothien-2-yl)-N-(2-fluorobenzyl)isoxazole-4-carboxamide. HPLC (Method B) r.t. 1.48, mass found=351.02.
5-amino-3-(5-chlorothien-2-yl)-N-(3,4-difluorobenzyl)isoxazole-4-carboxamide. HPLC (Method B) r.t. 1.5, mass found=369.02.
5-amino-N-(3-chlorobenzyl)-3-(5-chlorothien-2-yl)isoxazole-4-carboxamide. HPLC (Method B) r.t. 1.56, mass found=366.99.
5-amino-N-(2-chlorobenzyl)-3-(5-chlorothien-2-yl)isoxazole-4-carboxamide. HPLC (Method B) r.t. 1.56, mass found=366.99.
5-amino-3-(5-chlorothien-2-yl)-N-(1-naphthylmethyl)isoxazole-4-carboxamide. HPLC (Method B) r.t. 1.6, mass found=383.05.
5-amino-3-(5-chlorothien-2-yl)-N-(cyclopropylmethyl)isoxazole-4-carboxamide. HPLC (Method B) r.t. 1.39, mass found=297.03.
5-amino-3-(5-chlorothien-2-yl)-N-cycloheptylisoxazole-4-carboxamide. HPLC (Method B) r.t. 1.65, mass found=339.08.
5-amino-3-(5-chlorothien-2-yl)-N-[2-(4-methoxyphenyl)ethyl]isoxazole-4-carboxamide. HPLC (Method B) r.t. 1.49, mass found=377.06.
5-amino-3-(5-chlorothien-2-yl)-N-(2-cyclohex-1-en-1-ylethyl)isoxazole-4-carboxamide. HPLC (Method B) r.t. 1.67, mass found=351.08.
5-amino-3-(5-chlorothien-2-yl)-N-(3,3-diphenylpropyl)isoxazole-4-carboxamide. HPLC (Method B) r.t. 1.7, mass found=437.1.
5-amino-3-(5-chlorothien-2-yl)-N-(1,2-diphenylethyl)isoxazole-4-carboxamide. HPLC (Method B) r.t. 1.71, mass found=423.08.
5-amino-3-(5-chlorothien-2-yl)-N-[3-(dibutylamino)propyl]isoxazole-4-carboxamide. HPLC (Method B) r.t. 1.29, mass found=412.17.
5-amino-N-butyl-3-(5-chlorothien-2-yl)isoxazole-4-carboxamide. HPLC (Method B) r.t. 1.46, mass found=299.05.
5-amino-3-(5-chlorothien-2-yl)-N-[4-(trifluoromethoxy)benzyl]isoxazole-4-carboxamide. HPLC (Method B) r.t. 1.62, mass found=417.02.
5-amino-3-(5-chlorothien-2-yl)-N-[(5-methyl-2-furyl)methyl]isoxazole-4-carboxamide. HPLC (Method B) r.t. 1.45, mass found=337.03.
5-amino-3-(5-chlorothien-2-yl)-N-(1-methylhexyl)isoxazole-4-carboxamide. HPLC (Method B) r.t. 1.73, mass found=341.1.
5-amino-N-(1-benzylpiperidin-4-yl)-3-(5-chlorothien-2-yl)isoxazole-4-carboxamide. HPLC (Method B) r.t. 1.16, mass found=416.11.
5-amino-3-(5-chlorothien-2-yl)-N-(2-methoxy-1-methylethyl)isoxazole-4-carboxamide. HPLC (Method B) r.t. 1.32, mass found=315.04.
5-amino-3-(5-chlorothien-2-yl)-N-(1-methylbutyl)isoxazole-4-carboxamide. HPLC (Method B) r.t. 1.54, mass found=313.07.
5-amino-3-(5-chlorothien-2-yl)-N-(3-fluorobenzyl)isoxazole-4-carboxamide. HPLC (Method B) r.t. 1.48, mass found=351.02.
5-amino-3-(5-chlorothien-2-yl)-N-(1,3-dimethylbutyl)isoxazole-4-carboxamide. HPLC (Method B) r.t. 1.63, mass found=327.08.
5-amino-3-(5-chlorothien-2-yl)-N-(1-methyl-3-phenylpropyl)isoxazole-4-carboxamide. HPLC (Method B) r.t. 1.65, mass found=375.08.
5-amino-3-(5-chlorothien-2-yl)-N-[3-(methylthio)propyl]isoxazole-4-carboxamide. HPLC (Method B) r.t. 1.39, mass found=331.02.
5-amino-N-(1,3-benzodioxol-5-ylmethyl)-3-(5-chlorothien-2-yl)isoxazole-4-carboxamide. HPLC (Method B) r.t. 1.43, mass found=377.02.
5-amino-3-(5-chlorothien-2-yl)-N-pentylisoxazole-4-carboxamide. HPLC (Method B) r.t. 1.56, mass found=313.07.
5-amino-3-(5-chlorothien-2-yl)-N-(cyclohexylmethyl)isoxazole-4-carboxamide. HPLC (Method B) r.t. 1.65, mass found=339.08.
ethyl 4-({[5-amino-3-(5-chlorothien-2-yl)isoxazol-4-yl]carbonyl}amino)piperidine-1-carboxylate. HPLC (Method B) r.t. 1.35, mass found=398.08.
5-amino-N-[2-(3-chlorophenyl)ethyl]-3-(5-chlorothien-2-yl)isoxazole-4-carboxamide. HPLC (Method B) r.t. 1.59, mass found=381.01.
5-amino-3-(5-chlorothien-2-yl)-N-(1-ethylpropyl)isoxazole-4-carboxamide. HPLC (Method B) r.t. 1.53, mass found=313.07.
5-amino-3-(5-chlorothien-2-yl)-N-(1-phenylethyl)isoxazole-4-carboxamide. HPLC (Method B) r.t. 1.54, mass found=347.05.
5-amino-3-(5-chlorothien-2-yl)-N-propylisoxazole-4-carboxamide. HPLC (Method B) r.t. 1.36, mass found=285.03.
5-amino-3-(5-chlorothien-2-yl)-N-(2-methylbenzyl)isoxazole-4-carboxamide. HPLC (Method B) r.t. 1.54, mass found=347.05.
5-amino-3-(5-chlorothien-2-yl)-N-cyclobutylisoxazole-4-carboxamide. HPLC (Method B) r.t. 1.41, mass found=297.03.
5-amino-3-(5-chlorothien-2-yl)-N-(3-methoxypropyl)isoxazole-4-carboxamide. HPLC (Method B) r.t. 1.26, mass found=315.04.
5-amino-N-(2-bromobenzyl)-3-(5-chlorothien-2-yl)isoxazole-4-carboxamide. HPLC (Method B) r.t. 1.59, mass found=410.94.
5-amino-3-(5-chlorothien-2-yl)-N-(1-methylheptyl)isoxazole-4-carboxamide. HPLC (Method B) r.t. 1.82, mass found=355.11.
5-amino-3-(5-chlorothien-2-yl)-N-heptylisoxazole-4-carboxamide. HPLC (Method B) r.t. 1.74, mass found=341.1.
5-amino-N-(tert-butyl)-3-(5-chlorothien-2-yl)isoxazole-4-carboxamide. HPLC (Method B) r.t. 1.51, mass found=299.05.
5-amino-3-(5-chlorothien-2-yl)-N-(3,4-dimethoxybenzyl)isoxazole-4-carboxamide. HPLC (Method B) r.t. 1.34, mass found=393.05.
5-amino-3-(5-chlorothien-2-yl)-N-(2,3-dihydro-1H-inden-1-yl)isoxazole-4-carboxamide. HPLC (Method B) r.t. 1.57, mass found=359.05.
5-amino-3-(5-chlorothien-2-yl)-N-(1,2,3,4-tetrahydronaphthalen-1-yl)isoxazdle-4-carboxamide. HPLC (Method B) r.t. 1.63, mass found=373.07.
5-amino-N-benzyl-3-(5-methylthien-2-yl)isoxazole-4-carboxamide. HPLC (Method B) r.t. 1.39, mass found=313.09.
5-amino-N-(2-furylmethyl)-3-(5-methylthien-2-yl)isoxazole-4-carboxamide. HPLC (Method B) r.t. 1.3, mass found=303.07.
5-amino-3-(5-methylthien-2-yl)-N-(2-phenylethyl)isoxazole-4-carboxamide. HPLC (Method B) r.t. 1.44, mass found=327.1.
5-amino-N-(sec-butyl)-3-(5-methylthien-2-yl)isoxazole-4-carboxamide. HPLC (Method B) r.t. 1.38, mass found=279.1.
5-amino-N-cyclohexyl-3-(5-methylthien-2-yl)isoxazole-4-carboxamide. HPLC (Method B) r.t. 1.49, mass found=305.12.
5-amino-N-(4-methoxybenzyl)-3-(5-methylthien-2-yl)isoxazole-4-carboxamide. HPLC (Method B) r.t. 1.38, mass found=343.1.
5-amino-N-isopropyl-3-(5-methylthien-2-yl)isoxazole-4-carboxamide. HPLC (Method B) r.t. 1.28, mass found=265.09.
5-amino-N-hexyl-3-(5-methylthien-2-yl)isoxazole-4-carboxamide. HPLC (Method B) r.t. 1.59, mass found=307.14.
N-allyl-5-amino-3-(5-methylthien-2-yl)isoxazole-4-carboxamide. HPLC (Method B) r.t. 1.23, mass found=263.07.
5-amino-3-(5-methylthien-2-yl)-N-(thien-2-ylmethyl)isoxazole-4-carboxamide. HPLC (Method B) r.t. 1.36, mass found=319.04.
5-amino-N-cyclopentyl-3-(5-methylthien-2-yl)isoxazole-4-carboxamide. HPLC (Method B) r.t. 1.39, mass found=291.1.
5-amino-N-(1,5-dimethylhexyl)-3-(5-methylthien-2-yl)isoxazole-4-carboxamide. HPLC (Method B) r.t. 1.73, mass found=335.17.
5-amino-N-isopentyl-3-(5-methylthien-2-yl)isoxazole-4-carboxamide. HPLC (Method B) r.t. 1.47, mass found=293.12.
5-amino-N-(2-ethylhexyl)-3-(5-methylthien-2-yl)isoxazole-4-carboxamide. HPLC (Method B) r.t. 1.73, mass found=335.17.
5-amino-3-(5-methylthien-2-yl)-N-(4-phenylbutyl)isoxazole-4-carboxamide. HPLC (Method B) r.t. 1.6, mass found=355.14.
5-amino-N-(2-methylcyclohexyl)-3-(5-methylthien-2-yl)isoxazole-4-carboxamide. HPLC (Method B) r.t. 1.56, mass found=319.14.
5-amino-N-[3,5-bis(trifluoromethyl)benzyl]-3-(5-methylthien-2-yl)isoxazole-4-carboxamide. HPLC (Method B) r.t. 1.64, mass found=449.06.
5-amino-3-(5-methylthien-2-yl)-N-[3-(trifluoromethyl)benzyl]isoxazole-4-carboxamide. HPLC (Method B) r.t. 1.52, mass found=381.08.
5-amino-3-(5-methylthien-2-yl)-N-(3-phenylpropyl)isoxazole-4-carboxamide. HPLC (Method B) r.t. 1.52, mass found=341.12.
5-amino-N-isobutyl-3-(5-methylthien-2-yl)isoxazole-4-carboxamide. HPLC (Method B) r.t. 1.37, mass found=279.1.
5-amino-3-(5-methylthien-2-yl)-N-[2-(trifluoromethyl)benzyl]isoxazole-4-carboxamide. HPLC (Method B) r.t. 1.53, mass found=381.08.
5-amino-N-(2-methoxybenzyl)-3-(5-methylthien-2-yl)isoxazole-4-carboxamide. HPLC (Method B) r.t. 1.42, mass found=343.1.
5-amino-N-(2-fluorobenzyl)-3-(5-methylthien-2-yl)isoxazole-4-carboxamide. HPLC (Method B) r.t. 1.41, mass found=331.08.
5-amino-N-(3,4-difluorobenzyl)-3-(5-methylthien-2-yl)isoxazole-4-carboxamide. HPLC (Method B) r.t. 1.44, mass found=349.07.
5-amino-N-(3-chlorobenzyl)-3-(5-methylthien-2-yl)isoxazole-4-carboxamide. HPLC (Method B) r.t. 1.5, mass found=347.05.
5-amino-N-cycloheptyl-3-(5-methylthien-2-yl)isoxazole-4-carboxamide. HPLC (Method B) r.t. 1.58, mass found=319.14.
5-amino-N-(1,2-dimethylpropyl)-3-(5-methylthien-2-yl)isoxazole-4-carboxamide. HPLC (Method B) r.t. 1.46, mass found=293.12.
5-amino-N-(3,3-diphenylpropyl)-3-(5-methylthien-2-yl)isoxazole-4-carboxamide. HPLC (Method B) r.t. 1.64, mass found=417.15.
5-amino-N-(1,2-diphenylethyl)-3-(5-methylthien-2-yl)isoxazole-4-carboxamide. HPLC (Method B) r.t. 1.63, mass found=403.14.
5-amino-N-(2,4-dimethoxybenzyl)-3-(5-methylthien-2-yl)isoxazole-4-carboxamide. HPLC (Method B) r.t. 1.42, mass found=373.11.
5-amino-N-[3-(dibutylamino)propyl]-3-(5-methylthien-2-yl)isoxazole-4-carboxamide. HPLC (Method B) r.t. 1.22, mass found=392.22.
5-amino-N-butyl-3-(5-methylthien-2-yl)isoxazole-4-carboxamide. HPLC (Method B) r.t. 1.39, mass found=279.1.
5-amino-3-(5-methylthien-2-yl)-N-[4-(trifluoromethoxy)benzyl]isoxazole-4-carboxamide. HPLC (Method B) r.t. 1.56, mass found=397.07.
5-amino-N-[(5-methyl-2-furyl)methyl]-3-(5-methylthien-2-yl)isoxazole-4-carboxamide. HPLC (Method B) r.t. 1.38, mass found=317.08.
5-amino-N-(1-methylhexyl)-3-(5-methylthien-2-yl)isoxazole-4-carboxamide. HPLC (Method B) r.t. 1.65, mass found=321.15.
5-amino-N-(2-methoxy-1-methylethyl)-3-(5-methylthien-2-yl)isoxazole-4-carboxamide. HPLC (Method B) r.t. 1.23, mass found=295.1.
5-amino-N-(4-tert-butylcyclohexyl)-3-(5-methylthien-2-yl)isoxazole-4-carboxamide. HPLC (Method B) r.t. 1.83, mass found=361.18.
5-amino-N-(1-methylbutyl)-3-(5-methylthien-2-yl)isoxazole-4-carboxamide. HPLC (Method B) r.t. 1.48, mass found=293.12.
5-amino-N-(3-fluorobenzyl)-3-(5-methylthien-2-yl)isoxazole-4-carboxamide. HPLC (Method B) r.t. 1.42, mass found=331.08.
5-amino-N-(4-methylbenzyl)-3-(5-methylthien-2-yl)isoxazole-4-carboxamide. HPLC (Method B) r.t. 1.48, mass found=327.1.
5-amino-N-(1-methyl-3-phenylpropyl)-3-(5-methylthien-2-yl)isoxazole-4-carboxamide. HPLC (Method B) r.t. 1.57, mass found=355.14.
5-amino-N-(1,3-benzodioxol-5-ylmethyl)-3-(5-methylthien-2-yl)isoxazole-4-carboxamide. HPLC (Method B) r.t. 1.36, mass found=357.08.
5-amino-3-(5-methylthien-2-yl)-N-pentylisoxazole-4-carboxamide. HPLC (Method B) r.t. 1.49, mass found=293.12.
5-amino-N-(cyclohexylmethyl)-3-(5-methylthien-2-yl)isoxazole-4-carboxamide. HPLC (Method B) r.t. 1.56, mass found=319.14.
5-amino-3-(5-methylthien-2-yl)-N-(1-phenylethyl)isoxazole-4-carboxamide. HPLC (Method B) r.t. 1.47, mass found=327.1.
5-amino-3-(5-methylthien-2-yl)-N-propylisoxazole-4-carboxamide. HPLC (Method B) r.t. 1.28, mass found=265.09.
5-amino-N-(2-methylbenzyl)-3-(5-methylthien-2-yl)isoxazole-4-carboxamide. HPLC (Method B) r.t. 1.47, mass found=327.1.
5-amino-N-cyclobutyl-3-(5-methylthien-2-yl)isoxazole-4-carboxamide. HPLC (Method B) r.t. 1.33, mass found=277.09.
5-amino-N-(3-methoxypropyl)-3-(5-methylthien-2-yl)isoxazole-4-carboxamide. HPLC (Method B) r.t. 1.17, mass found=295.1.
5-amino-3-(5-methylthien-2-yl)-N-octylisoxazole-4-carboxamide. HPLC (Method B) r.t. 1.76, mass found=335.17.
5-amino-N-(1-methylheptyl)-3-(5-methylthien-2-yl)isoxazole-4-carboxamide. HPLC (Method B) r.t. 1.74, mass found=335.17.
5-amino-N-heptyl-3-(5-methylthien-2-yl)isoxazole-4-carboxamide. HPLC (Method B) r.t. 1.66, mass found=321.15.
5-amino-N-(tert-butyl)-3-(5-methylthien-2-yl)isoxazole-4-carboxamide. HPLC (Method B) r.t. 1.43, mass found=279.1.
5-amino-N-(3,4-dimethoxybenzyl)-3-(5-methylthien-2-yl)isoxazole-4-carboxamide. HPLC (Method B) r.t. 1.27, mass found=373.11.
5-amino-N-(2,3-dihydro-1H-inden-1-yl)-3-(5-methylthien-2-yl)isoxazole-4-carboxamide. HPLC (Method B) r.t. 1.5, mass found=339.1.
5-amino-3-(5-methylthien-2-yl)-N-(1,2,3,4-tetrahydronaphthalen-1-yl)isoxazole-4-carboxamide. HPLC (Method B) r.t. 1.55, mass found=353.12.
5-amino-N-benzyl-3-(3-methylthien-2-yl)isoxazole-4-carboxamide. HPLC (Method B) r.t. 1.37, mass found=313.09.
5-amino-N-(2-furylmethyl)-3-(3-methylthien-2-yl)isoxazole-4-carboxamide. HPLC (Method B) r.t. 1.27, mass found=303.07.
5-amino-3-(3-methylthien-2-yl)-N-(2-phenylethyl)isoxazole-4-carboxamide. HPLC (Method B) r.t. 1.41, mass found=327.1.
5-amino-N-(sec-butyl)-3-(3-methylthien-2-yl)isoxazole-4-carboxamide. HPLC (Method B) r.t. 1.34, mass found=279.1.
5-amino-N-cyclohexyl-3-(3-methylthien-2-yl)isoxazole-4-carboxamide. HPLC (Method B) r.t. 1.46, mass found=305.12.
5-amino-N-(4-methoxybenzyl)-3-(3-methylthien-2-yl)isoxazole-4-carboxamide. HPLC (Method B) r.t. 1.34, mass found=343.1.
5-amino-N-isopropyl-3-(3-methylthien-2-yl)isoxazole-4-carboxamide. HPLC (Method B) r.t. 1.25, mass found=265.09.
5-amino-N-hexyl-3-(3-methylthien-2-yl)isoxazole-4-carboxamide. HPLC (Method B) r.t. 1.54, mass found=307.14.
N-allyl-5-amino-3-(3-methylthien-2-yl)isoxazole-4-carboxamide. HPLC (Method B) r.t. 1.19, mass found=263.07.
5-amino-3-(3-methylthien-2-yl)-N-(tetrahydrofuran-2-ylmethyl)isoxazole-4-carboxamide. HPLC (Method B) r.t. 1.14, mass found=307.1.
5-amino-N-cyclopentyl-3-(3-methylthien-2-yl)isoxazole-4-carboxamide. HPLC (Method B) r.t. 1.36, mass found=291.1.
5-amino-N-(1,5-dimethylhexyl)-3-(3-methylthien-2-yl)isoxazole-4-carboxamide. HPLC (Method B) r.t. 1.7, mass found=335.17.
5-amino-N-isopentyl-3-(3-methylthien-2-yl)isoxazole-4-carboxamide. HPLC (Method B) r.t. 1.43, mass found=293.12.
5-amino-N-(2-ethylhexyl)-3-(3-methylthien-2-yl)isoxazole-4-carboxamide. HPLC (Method B) r.t. 1.7, mass found=335.17.
5-amino-3-(3-methylthien-2-yl)-N-(4-phenylbutyl)isoxazole-4-carboxamide. HPLC (Method B) r.t. 1.54, mass found=355.14.
5-amino-N-(2-methylcyclohexyl)-3-(3-methylthien-2-yl)isoxazole-4-carboxamide. HPLC (Method B) r.t. 1.54, mass found=319.14.
5-amino-N-[3,5-bis(trifluoromethyl)benzyl]-3-(3-methylthien-2-yl)isoxazole-4-carboxamide. HPLC (Method B) r.t. 1.62, mass found=449.06.
5-amino-3-(3-methylthien-2-yl)-N-[3-(trifluoromethyl)benzyl]isoxazole-4-carboxamide. HPLC (Method B) r.t. 1.49, mass found=381.08.
5-amino-3-(3-methylthien-2-yl)-N-(3-phenylpropyl)isoxazole-4-carboxamide. HPLC (Method B) r.t. 1.48, mass found=341.12.
5-amino-N-isobutyl-3-(3-methylthien-2-yl)isoxazole-4-carboxamide. HPLC (Method B) r.t. 1.34, mass found=279.1.
5-amino-3-(3-methylthien-2-yl)-N-[2-(trifluoromethyl)benzyl]isoxazole-4-carboxamide. HPLC (Method B) r.t. 1.51, mass found=381.08.
5-amino-N-(2-methoxybenzyl)-3-(3-methylthien-2-yl)isoxazole-4-carboxamide. HPLC (Method B) r.t. 1.39, mass found=343.1.
5-amino-N-(2-fluorobenzyl)-3-(3-methylthien-2-yl)isoxazole-4-carboxamide. HPLC (Method B) r.t. 1.39, mass found=331.08.
5-amino-3-(3-methylthien-2-yl)-N-[4-(trifluoromethyl)benzyl]isoxazole-4-carboxamide. HPLC (Method B) r.t. 1.5, mass found=381.08.
5-amino-N-(3,4-difluorobenzyl)-3-(3-methylthien-2-yl)isoxazole-4-carboxamide. HPLC (Method B) r.t. 1.42, mass found=349.07.
5-amino-N-(3-chlorobenzyl)-3-(3-methylthien-2-yl)isoxazole-4-carboxamide. HPLC (Method B) r.t. 1.47, mass found=347.05.
5-amino-N-(2-chlorobenzyl)-3-(3-methylthien-2-yl)isoxazole-4-carboxamide. HPLC (Method B) r.t. 1.47, mass found=347.05.
5-amino-N-(3-methylbenzyl)-3-(3-methylthien-2-yl)isoxazole-4-carboxamide. HPLC (Method B) r.t. 1.44, mass found=327.1.
5-amino-3-(3-methylthien-2-yl)-N-(1-naphthylmethyl)isoxazole-4-carboxamide. HPLC (Method B) r.t. 1.51, mass found=363.1.
5-amino-N-(cyclopropylmethyl)-3-(3-methylthien-2-yl)isoxazole-4-carboxamide. HPLC (Method B) r.t. 1.27, mass found=277.09.
5-amino-N-cycloheptyl-3-(3-methylthien-2-yl)isoxazole-4-carboxamide. HPLC (Method B) r.t. 1.53, mass found=319.14.
5-amino-N-[2-(4-methoxyphenyl)ethyl]-3-(3-methylthien-2-yl)isoxazole-4-carboxamide. HPLC (Method B) r.t. 1.38, mass found=357.11.
5-amino-N-(2-cyclohex-1-en-1-ylethyl)-3-(3-methylthien-2-yl)isoxazole-4-carboxamide. HPLC (Method B) r.t. 1.55, mass found=331.14.
5-amino-N-(1,2-dimethylpropyl)-3-(3-methylthien-2-yl)isoxazole-4-carboxamide. HPLC (Method B) r.t. 1.42, mass found=293.12.
5-amino-N-(3,3-diphenylpropyl)-3-(3-methylthien-2-yl)isoxazole-4-carboxamide. HPLC (Method B) r.t. 1.61, mass found=417.15.
5-amino-N-(2,4-dimethoxybenzyl)-3-(3-methylthien-2-yl)isoxazole-4-carboxamide. HPLC (Method B) r.t. 1.38, mass found=373.11.
5-amino-N-[3-(dibutylamino)propyl]-3-(3-methylthien-2-yl)isoxazole-4-carboxamide. HPLC (Method B) r.t. 1.17, mass found=392.22.
5-amino-N-butyl-3-(3-methylthien-2-yl)isoxazole-4-carboxamide. HPLC (Method B) r.t. 1.34, mass found=279.1.
5-amino-3-(3-methylthien-2-yl)-N-[4-(trifluoromethoxy)benzyl]isoxazole-4-carboxamide. HPLC (Method B) r.t. 1.53, mass found=397.07.
5-amino-N-[(5-methyl-2-furyl)methyl]-3-(3-methylthien-2-yl)isoxazole-4-carboxamide. HPLC (Method B) r.t. 1.35, mass found=317.08.
5-amino-N-(1-benzylpiperidin-4-yl)-3-(3-methylthien-2-yl)isoxazole-4-carboxamide. HPLC (Method B) r.t. 1.05, mass found=396.16.
5-amino-N-(2-methoxy-1-methylethyl)-3-(3-methylthien-2-yl)isoxazole-4-carboxamide. HPLC (Method B) r.t. 1.19, mass found=295.1.
5-amino-N-(4-tert-butylcyclohexyl)-3-(3-methylthien-2-yl)isoxazole-4-carboxamide. HPLC (Method B) r.t. 1.8, mass found=361.18.
5-amino-N-(1-methylbutyl)-3-(3-methylthien-2-yl)isoxazole-4-carboxamide. HPLC (Method B) r.t. 1.44, mass found=293.12.
5-amino-N-(3-fluorobenzyl)-3-(3-methylthien-2-yl)isoxazole-4-carboxamide. HPLC (Method B) r.t. 1.39, mass found=331.08.
5-amino-N-(4-methylbenzyl)-3-(3-methylthien-2-yl)isoxazole-4-carboxamide. HPLC (Method B) r.t. 1.45, mass found=327.1.
5-amino-N-(1,3-dimethylbutyl)-3-(3-methylthien-2-yl)isoxazole-4-carboxamide. HPLC (Method B) r.t. 1.52, mass found=307.14.
5-amino-N-(1-methyl-3-phenylpropyl)-3-(3-methylthien-2-yl)isoxazole-4-carboxamide. HPLC (Method B) r.t. 1.55, mass found=355.14.
5-amino-3-(3-methylthien-2-yl)-N-[3-(methylthio)propyl]isoxazole-4-carboxamide. HPLC (Method B) r.t. 1.28, mass found=311.08.
5-amino-N-(1,3-benzodioxol-5-ylmethyl)-3-(3-methylthien-2-yl)isoxazole-4-carboxamide. HPLC (Method B) r.t. 1.33, mass found=357.08.
5-amino-3-(3-methylthien-2-yl)-N-pentylisoxazole-4-carboxamide. HPLC (Method B) r.t. 1.44, mass found=293.12.
5-amino-N-(cyclohexylmethyl)-3-(3-methylthien-2-yl)isoxazole-4-carboxamide. HPLC (Method B) r.t. 1.54, mass found=319.14.
ethyl 4-({[5-amino-3-(3-methylthien-2-yl)isoxazol-4-yl]carbonyl}amino)piperidine-1-carboxylate. HPLC (Method B) r.t. 1.24, mass found=378.14.
5-amino-N-[2-(3-chlorophenyl)ethyl]-3-(3-methylthien-2-yl)isoxazole-4-carboxamide. HPLC (Method B) r.t. 1.5, mass found=361.07.
5-amino-N-(1-ethylpropyl)-3-(3-methylthien-2-yl)isoxazole-4-carboxamide. HPLC (Method B) r.t. 1.42, mass found=293.12.
5-amino-3-(3-methylthien-2-yl)-N-(1-phenylethyl)isoxazole-4-carboxamide. HPLC (Method B) r.t. 1.44, mass found=327.1.
5-amino-N-(2-methylbenzyl)-3-(3-methylthien-2-yl)isoxazole-4-carboxamide. HPLC (Method B) r.t. 1.44, mass found=327.1.
5-amino-N-cyclobutyl-3-(3-methylthien-2-yl)isoxazole-4-carboxamide. HPLC (Method B) r.t. 1.29, mass found=277.09.
5-amino-N-benzhydryl-3-(3-methylthien-2-yl)isoxazole-4-carboxamide. HPLC (Method B) r.t. 1.57, mass found=389.12.
5-amino-3-(3-methylthien-2-yl)-N-octylisoxazole-4-carboxamide. HPLC (Method B) r.t. 1.74, mass found=335.17.
5-amino-N-(2-bromobenzyl)-3-(3-methylthien-2-yl)isoxazole-4-carboxamide. HPLC (Method B) r.t. 1.49, mass found=391.
5-amino-N-(1-methylheptyl)-3-(3-methylthien-2-yl)isoxazole-4-carboxamide. HPLC (Method B) r.t. 1.72, mass found=335.17.
5-amino-N-heptyl-3-(3-methylthien-2-yl)isoxazole-4-carboxamide. HPLC (Method B) r.t. 1.64, mass found=321.15.
5-amino-N-(tert-butyl)-3-(3-methylthien-2-yl)isoxazole-4-carboxamide. HPLC (Method B). r.t. 1.39, mass found=279.1.
5-amino-N-(3,4-dimethoxybenzyl)-3-(3-methylthien-2-yl)isoxazole-4-carboxamide. HPLC (Method B) r.t. 1.24, mass found=373.11.
5-amino-N-(2,3-dihydro-1H-inden-1-yl)-3-(3-methylthien-2-yl)isoxazole-4-carboxamide. HPLC (Method B) r.t. 1.47, mass found=339.1.
5-amino-3-(3-methylthien-2-yl)-N-(1,2,3,4-tetrahydronaphthalen-1-yl)isoxazole-4-carboxamide. HPLC (Method B) r.t. 1.53, mass found=353.12.
5-amino-3-[5-(3-chlorophenyl)-2-furyl]-N-(2-furylmethyl)isoxazole-4-carboxamide. HPLC (Method B) r.t. 1.54, mass found=383.07.
5-amino-3-[5-(3-chlorophenyl)-2-furyl]-N-(2-phenylethyl)isoxazole-4-carboxamide. HPLC (Method B) r.t. 1.65, mass found=407.1.
5-amino-N-(sec-butyl)-3-[5-(3-chlorophenyl)-2-furyl]isoxazole-4-carboxamide. HPLC (Method B) r.t. 1.62, mass found=359.1.
5-amino-3-[5-(3-chlorophenyl)-2-furyl]-N-cyclohexylisoxazole-4-carboxamide. HPLC (Method B) r.t. 1.71, mass found=385.12.
5-amino-3-[5-(3-chlorophenyl)-2-furyl]-N-(4-methoxybenzyl)isoxazole-4-carboxamide. HPLC (Method B) r.t. 1.59, mass found=423.1.
5-amino-3-[5-(3-chlorophenyl)-2-furyl]-N-isopropylisoxazole-4-carboxamide. HPLC (Method B) r.t. 1.55, mass found=345.09.
5-amino-3-[5-(3-chlorophenyl)-2-furyl]-N-hexylisoxazole-4-carboxamide. HPLC (Method B) r.t. 1.79, mass found=387.14.
5-amino-3-[5-(3-chlorophenyl)-2-furyl]-N-(tetrahydrofuran-2-ylmethyl)isoxazole-4-carboxamide. HPLC (Method B) r.t. 1.49, mass found=387.1.
5-amino-3-[5-(3-chlorophenyl)-2-furyl]-N-cyclopentylisoxazole-4-carboxamide. HPLC (Method B) r.t. 1.63, mass found=371.1.
5-amino-3-[5-(3-chlorophenyl)-2-furyl]-N-(1,5-dimethylhexyl)isoxazole-4-carboxamide. HPLC (Method B) r.t. 1.9, mass found=415.17.
5-amino-3-[5-(3-chlorophenyl)-2-furyl]-N-isopentylisoxazole-4-carboxamide. HPLC (Method B) r.t. 1.69, mass found=373.12.
5-amino-3-[5-(3-chlorophenyl)-2-furyl]-N-(2-ethylhexyl)isoxazole-4-carboxamide. HPLC (Method B) r.t. 1.91, mass found=415.17.
5-amino-3-[5-(3-chlorophenyl)-2-furyl]-N-(4-phenylbutyl)isoxazole-4-carboxamide. HPLC (Method B) r.t. 1.77, mass found=435.14.
5-amino-3-[5-(3-chlorophenyl)-2-furyl]-N-(2-methylcyclohexyl)isoxazole-4-carboxamide. HPLC (Method B) r.t. 1.78, mass found=399.14.
5-amino-N-[3,5-bis(trifluoromethyl)benzyl]-3-[5-(3-chlorophenyl)-2-furyl]isoxazole-4-carboxamide. HPLC (Method B) r.t. 1.79, mass found=529.06.
5-amino-3-[5-(3-chlorophenyl)-2-furyl]-N-[3-(trifluoromethyl)benzyl]isoxazole-4-carboxamide. HPLC (Method B) r.t. 1.71, mass found=461.08.
5-amino-3-[5-(3-chlorophenyl)-2-furyl]-N-(3-phenylpropyl)isoxazole-4-carboxamide. HPLC (Method B) r.t. 1.7, mass found=421.12.
5-amino-3-[5-(3-chlorophenyl)-2-furyl]-N-isobutylisoxazole-4-carboxamide. HPLC (Method B) r.t. 1.62, mass found=359.1.
5-amino-3-[5-(3-chlorophenyl)-2-furyl]-N-[2-(trifluoromethyl)benzyl]isoxazole-4-carboxamide. HPLC (Method B) r.t. 1.7, mass found=461.08.
5-amino-3-[5-(3-chlorophenyl)-2-furyl]-N-(2-methoxybenzyl)isoxazole-4-carboxamide. HPLC (Method B) r.t. 1.63, mass found=423.1.
5-amino-3-[5-(3-chlorophenyl)-2-furyl]-N-(2-fluorobenzyl)isoxazole-4-carboxamide. HPLC (Method B) r.t. 1.62, mass found=411.08.
5-amino-3-[5-(3-chlorophenyl)-2-furyl]-N-[4-(trifluoromethyl)benzyl]isoxazole-4-carboxamide. HPLC (Method B) r.t. 1.71, mass found=461.08.
5-amino-3-[5-(3-chlorophenyl)-2-furyl]-N-(3,4-difluorobenzyl)isoxazole-4-carboxamide. HPLC (Method B) r.t. 1.63, mass found=429.07.
5-amino-N-(3-chlorobenzyl)-3-[5-(3-chlorophenyl)-2-furyl]isoxazole-4-carboxamide. HPLC (Method B) r.t. 1.7, mass found=427.05.
5-amino-N-(2-chlorobenzyl)-3-[5-(3-chlorophenyl)-2-furyl]isoxazole-4-carboxamide. HPLC (Method B) r.t. 1.69, mass found=427.05.
5-amino-3-[5-(3-chlorophenyl)-2-furyl]-N-(3-methylbenzyl)isoxazole-4-carboxamide. HPLC (Method B) r.t. 1.69, mass found=407.1.
5-amino-3-[5-(3-chlorophenyl)-2-furyl]-N-(1-naphthylmethyl)isoxazole-4-carboxamide. HPLC (Method B) r.t. 1.72, mass found=443.1.
5-amino-3-[5-(3-chlorophenyl)-2-furyl]-N-(cyclopropylmethyl)isoxazole-4-carboxamide. HPLC (Method B) r.t. 1.56, mass found=357.09.
5-amino-3-[5-(3-chlorophenyl)-2-furyl]-N-cycloheptylisoxazole-4-carboxamide. HPLC (Method B) r.t. 1.78, mass found=399.14.
5-amino-3-[5-(3-chlorophenyl)-2-furyl]-N-[2-(4-methoxyphenyl)ethyl]isoxazole-4-carboxamide. HPLC (Method B) r.t. 1.62, mass found=437.11.
5-amino-3-[5-(3-chlorophenyl)-2-furyl]-N-(2-cyclohex-1-en-1-ylethyl)isoxazole-4-carboxamide. HPLC (Method B) r.t. 1.79, mass found=411.14.
5-amino-3-[5-(3-chlorophenyl)-2-furyl]-N-(1,2-dimethylpropyl)isoxazole-4-carboxamide. HPLC (Method B) r.t. 1.67, mass found=373.12.
5-amino-3-[5-(3-chlorophenyl)-2-furyl]-N-(3,3-diphenylpropyl)isoxazole-4-carboxamide. HPLC (Method B) r.t. 1.8, mass found=497.15.
5-amino-3-[5-(3-chlorophenyl)-2-furyl]-N-(1,2-diphenylethyl)isoxazole-4-carboxamide. HPLC (Method B) r.t. 1.78, mass found=483.14.
5-amino-3-[5-(3-chlorophenyl)-2-furyl]-N-(2,4-dimethoxybenzyl)isoxazole-4-carboxamide. HPLC (Method B) r.t. 1.6, mass found=453.11.
5-amino-3-[5-(3-chlorophenyl)-2-furyl]-N-[3-(dibutylamino)propyl]isoxazole-4-carboxamide. HPLC (Method B) r.t. 1.41, mass found=472.22.
5-amino-N-butyl-3-[5-(3-chlorophenyl)-2-furyl]isoxazole-4-carboxamide. HPLC (Method B) r.t. 1.62, mass found=359.1.
5-amino-3-[5-(3-chlorophenyl)-2-furyl]-N-[4-(trifluoromethoxy)benzyl]isoxazole-4-carboxamide. HPLC (Method B) r.t. 1.74, mass found=477.07.
5-amino-3-[5-(3-chlorophenyl)-2-furyl]-N-[(5-methyl-2-furyl)methyl]isoxazole-4-carboxamide. HPLC (Method B) r.t. 1.61, mass found=397.08.
5-amino-3-[5-(3-chlorophenyl)-2-furyl]-N-(1-methylhexyl)isoxazole-4-carboxamide. HPLC (Method B) r.t. 1.84, mass found=401.15.
5-amino-N-(1-benzylpiperidin-4-yl)-3-[5-(3-chlorophenyl)-2-furyl]isoxazole-4-carboxamide. HPLC (Method B) r.t. 1.29, mass found=476.16.
5-amino-3-[5-(3-chlorophenyl)-2-furyl]-N-(1,3-dimethylbutyl)isoxazole-4-carboxamide. HPLC (Method B) r.t. 1.76, mass found=387.14.
5-amino-3-[5-(3-chlorophenyl)-2-furyl]-N-(1-methyl-3-phenylpropyl)isoxazole-4-carboxamide. HPLC (Method B) r.t. 1.77, mass found=435.14.
5-amino-3-[5-(3-chlorophenyl)-2-furyl]-N-[3-(methylthio)propyl]isoxazole-4-carboxamide. HPLC (Method B) r.t. 1.55, mass found=391.08.
5-amino-3-[5-(3-chlorophenyl)-2-furyl]-N-pentylisoxazole-4-carboxamide. HPLC (Method B) r.t. 1.71, mass found=373.12.
ethyl 4-[({5-amino-3-[5-(3-chlorophenyl)-2-furyl]isoxazol-4-yl}carbonyl)amino]piperidine-1-carboxylate. HPLC (Method B) r.t. 1.48, mass found=458.14.
5-amino-N-[2-(3-chlorophenyl)ethyl]-3-[5-(3-chlorophenyl)-2-furyl]isoxazole-4-carboxamide. HPLC (Method B) r.t. 1.72, mass found=441.06.
5-amino-3-[5-(3-chlorophenyl)-2-furyl]-N-(1-ethylpropyl)isoxazole-4-carboxamide. HPLC (Method B) r.t. 1.68, mass found=373.12.
5-amino-3-[5-(3-chlorophenyl)-2-furyl]-N-(1-phenylethyl)isoxazole-4-carboxamide. HPLC (Method B) r.t. 1.67, mass found=407.1.
5-amino-3-[5-(3-chlorophenyl)-2-furyl]-N-(2-methylbenzyl)isoxazole-4-carboxamide. HPLC (Method B) r.t. 1.68, mass found=407.1.
5-amino-3-[5-(3-chlorophenyl)-2-furyl]-N-cyclobutylisoxazole-4-carboxamide. HPLC (Method B) r.t. 1.58, mass found=357.09.
5-amino-N-benzhydryl-3-[5-(3-chlorophenyl)-2-furyl]isoxazole-4-carboxamide. HPLC (Method B) r.t. 1.76, mass found=469.12.
5-amino-3-[5-(3-chlorophenyl)-2-furyl]-N-octylisoxazole-4-carboxamide. HPLC (Method B) r.t. 1.96, mass found=415.17.
5-amino-N-(2-bromobenzyl)-3-[5-(3-chlorophenyl)-2-furyl]isoxazole-4-carboxamide. HPLC (Method B) r.t. 1.71, mass found=471.
5-amino-3-[5-(3-chlorophenyl)-2-furyl]-N-(1-methylheptyl)isoxazole-4-carboxamide. HPLC (Method B) r.t. 1.93, mass found=415.17.
5-amino-3-[5-(3-chlorophenyl)-2-furyl]-N-heptylisoxazole-4-carboxamide. HPLC (Method B) r.t. 1.87, mass found=401.15.
5-amino-N-(tert-butyl)-3-[5-(3-chlorophenyl)-2-furyl]isoxazole-4-carboxamide. HPLC (Method B) r.t. 1.66, mass found=359.1.
5-amino-3-[5-(3-chlorophenyl)-2-furyl]-N-(3,4-dimethoxybenzyl)isoxazole-4-carboxamide. HPLC (Method B) r.t. 1.49, mass found=453.11.
5-amino-3-[5-(3-chlorophenyl)-2-furyl]-N-(2,3-dihydro-1H-inden-1-yl)isoxazole-4-carboxamide. HPLC (Method B) r.t. 1.72, mass found=419.1.
5-amino-3-[5-(3-chlorophenyl)-2-furyl]-N-(1,2,3,4-tetrahydronaphthalen-1-yl)isoxazole-4-carboxamide. HPLC (Method B) r.t. 1.78, mass found=433.12.
5-amino-N-benzyl-3-(5-methyl-2-furyl)isoxazole-4-carboxamide. HPLC (Method B) r.t. 1.35, mass found=297.11.
5-amino-N-(2-furylmethyl)-3-(5-methyl-2-furyl)isoxazole-4-carboxamide. HPLC (Method B) r.t. 1.25, mass found=287.09.
5-amino-3-(5-methyl-2-furyl)-N-(2-phenylethyl)isoxazole-4-carboxamide. HPLC (Method B) r.t. 1.38, mass found=311.13.
5-amino-N-(sec-butyl)-3-(5-methyl-2-furyl)isoxazole-4-carboxamide. HPLC (Method B) r.t. 1.29, mass found=263.13.
5-amino-N-cyclohexyl-3-(5-methyl-2-furyl)isoxazole-4-carboxamide. HPLC (Method B) r.t. 1.42, mass found=289.14.
5-amino-N-(4-methoxybenzyl)-3-(5-methyl-2-furyl)isoxazole-4-carboxamide. HPLC (Method B) r.t. 1.32, mass found=327.12.
5-amino-N-hexyl-3-(5-methyl-2-furyl)isoxazole-4-carboxamide. HPLC (Method B) r.t. 1.52, mass found=291.16.
5-amino-3-(5-methyl-2-furyl)-N-(tetrahydrofuran-2-ylmethyl)isoxazole-4-carboxamide. HPLC (Method B) r.t. 1.14, mass found=291.12.
5-amino-3-(5-methyl-2-furyl)-N-(thien-2-ylmethyl)isoxazole-4-carboxamide. HPLC (Method B) r.t. 1.31, mass found=303.07.
5-amino-N-cyclopentyl-3-(5-methyl-2-furyl)isoxazole-4-carboxamide. HPLC (Method B) r.t. 1.32, mass found=275.13.
5-amino-N-(1,5-dimethylhexyl)-3-(5-methyl-2-furyl)isoxazole-4-carboxamide. HPLC (Method B) r.t. 1.67, mass found=319.19.
5-amino-N-isopentyl-3-(5-methyl-2-furyl)isoxazole-4-carboxamide. HPLC (Method B) r.t. 1.4, mass found=277.14.
5-amino-N-(2-ethylhexyl)-3-(5-methyl-2-furyl)isoxazole-4-carboxamide. HPLC (Method B) r.t. 1.68, mass found=319.19.
5-amino-3-(5-methyl-2-furyl)-N-(4-phenylbutyl)isoxazole-4-carboxamide. HPLC (Method B) r.t. 1.52, mass found=339.16.
5-amino-N-(2-methylcyclohexyl)-3-(5-methyl-2-furyl)isoxazole-4-carboxamide. HPLC (Method B) r.t. 1.48, mass found=303.16.
5-amino-3-(5-methyl-2-furyl)-N-[3-(trifluoromethyl)benzyl]isoxazole-4-carboxamide. HPLC (Method B) r.t. 1.47, mass found=365.1.
5-amino-3-(5-methyl-2-furyl)-N-(3-phenylpropyl)isoxazole-4-carboxamide. HPLC (Method B) r.t. 1.45, mass found=325.14.
5-amino-N-isobutyl-3-(5-methyl-2-furyl)isoxazole-4-carboxamide. HPLC (Method B) r.t. 1.3, mass found=263.13.
5-amino-3-(5-methyl-2-furyl)-N-[2-(trifluoromethyl)benzyl]isoxazole-4-carboxamide. HPLC (Method B) r.t. 1.48, mass found=365.1.
5-amino-N-(2-methoxybenzyl)-3-(5-methyl-2-furyl)isoxazole-4-carboxamide. HPLC (Method B) r.t. 1.37, mass found=327.12.
5-amino-N-(2-fluorobenzyl)-3-(5-methyl-2-furyl)isoxazole-4-carboxamide. HPLC (Method B) r.t. 1.37, mass found=315.1.
5-amino-3-(5-methyl-2-furyl)-N-[4-(trifluoromethyl)benzyl]isoxazole-4-carboxamide. HPLC (Method B) r.t. 1.48, mass found=365.1.
5-amino-N-(3,4-difluorobenzyl)-3-(5-methyl-2-furyl)isoxazole-4-carboxamide. HPLC (Method B) r.t. 1.39, mass found=333.09.
5-amino-N-(3-chlorobenzyl)-3-(5-methyl-2-furyl)isoxazole-4-carboxamide. HPLC (Method B) r.t. 1.44, mass found=331.07.
5-amino-N-(2-chlorobenzyl)-3-(5-methyl-2-furyl)isoxazole-4-carboxamide. HPLC (Method B) r.t. 1.43, mass found=331.07.
5-amino-N-(3-methylbenzyl)-3-(5-methyl-2-furyl)isoxazole-4-carboxamide. HPLC (Method B) r.t. 1.43, mass found=311.13.
5-amino-3-(5-methyl-2-furyl)-N-(1-naphthylmethyl)isoxazole-4-carboxamide. HPLC (Method B) r.t. 1.49, mass found=347.13.
5-amino-N-(cyclopropylmethyl)-3-(5-methyl-2-furyl)isoxazole-4-carboxamide. HPLC (Method B) r.t. 1.24, mass found=261.11.
5-amino-N-cycloheptyl-3-(5-methyl-2-furyl)isoxazole-4-carboxamide. HPLC (Method B) r.t. 1.5, mass found=303.16.
5-amino-N-[2-(4-methoxyphenyl)ethyl]-3-(5-methyl-2-furyl)isoxazole-4-carboxamide. HPLC (Method B) r.t. 1.35, mass found=341.14.
5-amino-N-(2-cyclohex-1-en-1-ylethyl)-3-(5-methyl-2-furyl)isoxazole-4-carboxamide. HPLC (Method B) r.t. 1.52, mass found=315.16.
5-amino-N-(1,2-dimethylpropyl)-3-(5-methyl-2-furyl)isoxazole-4-carboxamide. HPLC (Method B) r.t. 1.38, mass found=277.14.
5-amino-N-(3,3-diphenylpropyl)-3-(5-methyl-2-furyl)isoxazole-4-carboxamide. HPLC (Method B) r.t. 1.59, mass found=401.17.
5-amino-N-(1,2-diphenylethyl)-3-(5-methyl-2-furyl)isoxazole-4-carboxamide. HPLC (Method B) r.t. 1.58, mass found=387.16.
5-amino-N-(2,4-dimethoxybenzyl)-3-(5-methyl-2-furyl)isoxazole-4-carboxamide. HPLC (Method B) r.t. 1.35, mass found=357.13.
5-amino-N-[3-(dibutylamino)propyl]-3-(5-methyl-2-furyl)isoxazole-4-carboxamide. HPLC (Method B) r.t. 1.16, mass found=376.25.
5-amino-N-butyl-3-(5-methyl-2-furyl)isoxazole-4-carboxamide. HPLC (Method B) r.t. 1.31, mass found=263.13.
5-amino-3-(5-methyl-2-furyl)-N-[4-(trifluoromethoxy)benzyl]isoxazole-4-carboxamide. HPLC (Method B) r.t. 1.52, mass found=381.09.
5-amino-3-(5-methyl-2-furyl)-N-[(5-methyl-2-furyl)methyl]isoxazole-4-carboxamide. HPLC (Method B) r.t. 1.33, mass found=301.11.
5-amino-3-(5-methyl-2-furyl)-N-(1-methylhexyl)isoxazole-4-carboxamide. HPLC (Method B) r.t. 1.59, mass found=305.17.
5-amino-N-(1-benzylpiperidin-4-yl)-3-(5-methyl-2-furyl)isoxazole-4-carboxamide. HPLC (Method B) r.t. 1.03, mass found=380.18.
5-amino-N-(2-methoxy-1-methylethyl)-3-(5-methyl-2-furyl)isoxazole-4-carboxamide. HPLC (Method B) r.t. 1.17, mass found=279.12.
5-amino-N-(1-methylbutyl)-3-(5-methyl-2-furyl)isoxazole-4-carboxamide. HPLC (Method B) r.t. 1.39, mass found=277.14.
5-amino-N-(3-fluorobenzyl)-3-(5-methyl-2-furyl)isoxazole-4-carboxamide. HPLC (Method B) r.t. 1.37, mass found=315.1.
5-amino-N-(4-methylbenzyl)-3-(5-methyl-2-furyl)isoxazole-4-carboxamide. HPLC (Method B) r.t. 1.43, mass found=311.13.
5-amino-N-(1,3-dimethylbutyl)-3-(5-methyl-2-furyl)isoxazole-4-carboxamide. HPLC (Method B) r.t. 1.48, mass found=291.16.
5-amino-3-(5-methyl-2-furyl)-N-(1-methyl-3-phenylpropyl)isoxazole-4-carboxamide. HPLC (Method B) r.t. 1.52, mass found=339.16.
5-amino-3-(5-methyl-2-furyl)-N-[3-(methylthio)propyl]isoxazole-4-carboxamide. HPLC (Method B) r.t. 1.24, mass found=295.1.
5-amino-N-(1,3-benzodioxol-5-ylmethyl)-3-(5-methyl-2-furyl)isoxazole-4-carboxamide. HPLC (Method B) r.t. 1.31, mass found=341.1.
5-amino-3-(5-methyl-2-furyl)-N-pentylisoxazole-4-carboxamide. HPLC (Method B) r.t. 1.42, mass found=277.14.
5-amino-N-(cyclohexylmethyl)-3-(5-methyl-2-furyl)isoxazole-4-carboxamide. HPLC (Method B) r.t. 1.52, mass found=303.16.
ethyl 4-({[5-amino-3-(5-methyl-2-furyl)isoxazol-4-yl]carbonyl}amino)piperidine-1-carboxylate. HPLC (Method B) r.t. 1.21, mass found=362.16.
5-amino-N-[2-(3-chlorophenyl)ethyl]-3-(5-methyl-2-furyl)isoxazole-4-carboxamide. HPLC (Method B) r.t. 1.47, mass found=345.09.
5-amino-N-(1-ethylpropyl)-3-(5-methyl-2-furyl)isoxazole-4-carboxamide. HPLC (Method B) r.t. 1.37, mass found=277.14.
5-amino-3-(5-methyl-2-furyl)-N-(1-phenylethyl)isoxazole-4-carboxamide. HPLC (Method B) r.t. 1.41, mass found=311.13.
5-amino-N-(2-methylbenzyl)-3-(5-methyl-2-furyl)isoxazole-4-carboxamide. HPLC (Method B) r.t. 1.42, mass found=311.13.
5-amino-N-cyclobutyl-3-(5-methyl-2-furyl)isoxazole-4-carboxamide. HPLC (Method B) r.t. 1.26, mass found=261.11.
5-amino-N-benzhydryl-3-(5-methyl-2-furyl)isoxazole-4-carboxamide. HPLC (Method B) r.t. 1.58, mass found=373.14.
5-amino-3-(5-methyl-2-furyl)-N-octylisoxazole-4-carboxamide. HPLC (Method B) r.t. 1.72, mass found=319.19.
5-amino-N-(2-bromobenzyl)-3-(5-methyl-2-furyl)isoxazole-4-carboxamide. HPLC (Method B) r.t. 1.45, mass found=375.02.
5-amino-3-(5-methyl-2-furyl)-N-(1-methylheptyl)isoxazole-4-carboxamide. HPLC (Method B) r.t. 1.69, mass found=319.19.
5-amino-N-heptyl-3-(5-methyl-2-furyl)isoxazole-4-carboxamide. HPLC (Method B) r.t. 1.62, mass found=305.17.
5-amino-N-(tert-butyl)-3-(5-methyl-2-furyl)isoxazole-4-carboxamide. HPLC (Method B) r.t. 1.35, mass found=263.13.
5-amino-N-(3,4-dimethoxybenzyl)-3-(5-methyl-2-furyl)isoxazole-4-carboxamide. HPLC (Method B) r.t. 1.22, mass found=357.13.
5-amino-N-(2,3-dihydro-1H-inden-1-yl)-3-(5-methyl-2-furyl)isoxazole-4-carboxamide. HPLC (Method B) r.t. 1.46, mass found=323.13.
5-amino-3-(5-methyl-2-furyl)-N-(1,2,3,4-tetrahydronaphthalen-1-yl)isoxazole-4-carboxamide. HPLC (Method B) r.t. 1.52, mass found=337.14.
5-amino-N-benzyl-3-(2-furyl)isoxazole-4-carboxamide. HPLC (Method B) r.t. 1.26, mass found=283.1.
5-amino-3-(2-furyl)-N-(2-furylmethyl)isoxazole-4-carboxamide. HPLC (Method B) r.t. 1.15, mass found=273.08.
5-amino-3-(2-furyl)-N-(2-phenylethyl)isoxazole-4-carboxamide. HPLC (Method B) r.t. 1.3, mass found=297.11.
5-amino-N-(sec-butyl)-3-(2-furyl)isoxazole-4-carboxamide. HPLC (Method B) r.t. 1.19, mass found=249.11.
5-amino-3-(2-furyl)-N-(4-methoxybenzyl)isoxazole-4-carboxamide. HPLC (Method B) r.t. 1.24, mass found=313.11.
5-amino-3-(2-furyl)-N-hexylisoxazole-4-carboxamide. HPLC (Method B) r.t. 1.43, mass found=277.14.
5-amino-3-(2-furyl)-N-(tetrahydrofuran-2-ylmethyl)isoxazole-4-carboxamide. HPLC (Method B) r.t. 1.02, mass found=277.11.
5-amino-3-(2-furyl)-N-(thien-2-ylmethyl)isoxazole-4-carboxamide. HPLC (Method B) r.t. 1.22, mass found=289.05.
5-amino-N-cyclopentyl-3-(2-furyl)isoxazole-4-carboxamide. HPLC (Method B) r.t. 1.22, mass found=261.11.
5-amino-N-(1,5-dimethylhexyl)-3-(2-furyl)isoxazole-4-carboxamide. HPLC (Method B) r.t. 1.59, mass found=305.17.
5-amino-3-(2-furyl)-N-isopentylisoxazole-4-carboxamide. HPLC (Method B) r.t. 1.31, mass found=263.13.
5-amino-N-(2-ethylhexyl)-3-(2-furyl)isoxazole-4-carboxamide. HPLC (Method B) r.t. 1.6, mass found=305.17.
5-amino-3-(2-furyl)-N-(4-phenylbutyl)isoxazole-4-carboxamide. HPLC (Method B) r.t. 1.45, mass found=325.14.
5-amino-3-(2-furyl)-N-(2-methylcyclohexyl)isoxazole-4-carboxamide. HPLC (Method B) r.t. 1.4, mass found=289.14.
5-amino-N-[3,5-bis(trifluoromethyl)benzyl]-3-(2-furyl)isoxazole-4-carboxamide. HPLC (Method B) r.t. 1.54, mass found=419.07.
5-amino-3-(2-furyl)-N-[3-(trifluoromethyl)benzyl]isoxazole-4-carboxamide. HPLC (Method B) r.t. 1.41, mass found=351.08.
5-amino-3-(2-furyl)-N-(3-phenylpropyl)isoxazole-4-carboxamide. HPLC (Method B) r.t. 1.38, mass found=311.13.
5-amino-3-(2-furyl)-N-isobutylisoxazole-4-carboxamide. HPLC (Method B) r.t. 1.21, mass found=249.11.
5-amino-3-(2-furyl)-N-[2-(trifluoromethyl)benzyl]isoxazole-4-carboxamide. HPLC (Method B) r.t. 1.42, mass found=351.08.
5-amino-3-(2-furyl)-N-(2-methoxybenzyl)isoxazole-4-carboxamide. HPLC (Method B) r.t. 1.29, mass found=313.11.
5-amino-N-(2-fluorobenzyl)-3-(2-furyl)isoxazole-4-carboxamide. HPLC (Method B) r.t. 1.28, mass found=301.09.
5-amino-3-(2-furyl)-N-[4-(trifluoromethyl)benzyl]isoxazole-4-carboxamide. HPLC (Method B) r.t. 1.42, mass found=351.08.
5-amino-N-(3,4-difluorobenzyl)-3-(2-furyl)isoxazole-4-carboxamide. HPLC (Method B) r.t. 1.31, mass found=319.08.
5-amino-N-(3-chlorobenzyl)-3-(2-furyl)isoxazole-4-carboxamide. HPLC (Method B) r.t. 1.36, mass found=317.06.
5-amino-N-(2-chlorobenzyl)-3-(2-furyl)isoxazole-4-carboxamide. HPLC (Method B) r.t. 1.36, mass found=317.06.
5-amino-3-(2-furyl)-N-(3-methylbenzyl)isoxazole-4-carboxamide. HPLC (Method B) r.t. 1.34, mass found=297.11.
5-amino-3-(2-furyl)-N-(1-naphthylmethyl)isoxazole-4-carboxamide. HPLC (Method B) r.t. 1.41, mass found=333.11.
5-amino-N-(cyclopropylmethyl)-3-(2-furyl)isoxazole-4-carboxamide. HPLC (Method B) r.t. 1.13, mass found=247.1.
5-amino-N-cycloheptyl-3-(2-furyl)isoxazole-4-carboxamide. HPLC (Method B) r.t. 1.42, mass found=289.14.
5-amino-3-(2-furyl)-N-[2-(4-methoxyphenyl)ethyl]isoxazole-4-carboxamide. HPLC (Method B) r.t. 1.29, mass found=327.12.
5-amino-N-(2-cyclohex-1-en-1-ylethyl)-3-(2-furyl)isoxazole-4-carboxamide. HPLC (Method B) r.t. 1.44, mass found=301.14.
5-amino-N-(1,2-dimethylpropyl)-3-(2-furyl)isoxazole-4-carboxamide. HPLC (Method B) r.t. 1.29, mass found=263.13.
5-amino-N-(3,3-diphenylpropyl)-3-(2-furyl)isoxazole-4-carboxamide. HPLC (Method B) r.t. 1.53, mass found=387.16.
5-amino-N-(1,2-diphenylethyl)-3-(2-furyl)isoxazole-4-carboxamide. HPLC (Method B) r.t. 1.51, mass found=373.14.
5-amino-N-(2,4-dimethoxybenzyl)-3-(2-furyl)isoxazole-4-carboxamide. HPLC (Method B) r.t. 1.29, mass found=343.12.
5-amino-N-[3-(dibutylamino)propyl]-3-(2-furyl)isoxazole-4-carboxamide. HPLC (Method B) r.t. 1.09, mass found=362.23.
5-amino-N-butyl-3-(2-furyl)isoxazole-4-carboxamide. HPLC (Method B) r.t. 1.22, mass found=249.11.
5-amino-3-(2-furyl)-N-[4-(trifluoromethoxy)benzyl]isoxazole-4-carboxamide. HPLC (Method B) r.t. 1.45, mass found=367.08.
5-amino-3-(2-furyl)-N-[(5-methyl-2-furyl)methyl]isoxazole-4-carboxamide. HPLC (Method B) r.t. 1.24, mass found=287.09.
5-amino-3-(2-furyl)-N-(1-methylhexyl)isoxazole-4-carboxamide. HPLC (Method B) r.t. 1.51, mass found=291.16.
5-amino-N-(1-benzylpiperidin-4-yl)-3-(2-furyl)isoxazole-4-carboxamide. HPLC (Method B) r.t. 0.97, mass found=366.17.
5-amino-3-(2-furyl)-N-(2-methoxy-1-methylethyl)isoxazole-4-carboxamide. HPLC (Method B) r.t. 1.05, mass found=265.11.
5-amino-N-(4-tert-butylcyclohexyl)-3-(2-furyl)isoxazole-4-carboxamide. HPLC (Method B) r.t. 1.68, mass found=331.19.
5-amino-3-(2-furyl)-N-(1-methylbutyl)isoxazole-4-carboxamide. HPLC (Method B) r.t. 1.31, mass found=263.13.
5-amino-N-(3-fluorobenzyl)-3-(2-furyl)isoxazole-4-carboxamide. HPLC (Method B) r.t. 1.28, mass found=301.09.
5-amino-3-(2-furyl)-N-(4-methylbenzyl)isoxazole-4-carboxamide. HPLC (Method B) r.t. 1.34, mass found=297.11.
5-amino-N-(1,3-dimethylbutyl)-3-(2-furyl)isoxazole-4-carboxamide. HPLC (Method B) r.t. 1.39, mass found=277.14.
5-amino-3-(2-furyl)-N-(1-methyl-3-phenylpropyl)isoxazole-4-carboxamide. HPLC (Method B) r.t. 1.44, mass found=325.14.
5-amino-3-(2-furyl)-N-[3-(methylthio)propyl]isoxazole-4-carboxamide. HPLC (Method B) r.t. 1.16, mass found=281.08.
5-amino-N-(1,3-benzodioxol-5-ylmethyl)-3-(2-furyl)isoxazole-4-carboxamide. HPLC (Method B) r.t. 1.23, mass found=327.09.
5-amino-3-(2-furyl)-N-pentylisoxazole-4-carboxamide. HPLC (Method B) r.t. 1.33, mass found=263.13.
5-amino-N-(cyclohexylmethyl)-3-(2-furyl)isoxazole-4-carboxamide. HPLC (Method B) r.t. 1.43, mass found=289.14.
ethyl 4-({[5-amino-3-(2-furyl)isoxazol-4-yl]carbonyl}amino)piperidine-1-carboxylate. HPLC (Method B) r.t. 1.14, mass found=348.14.
5-amino-N-[2-(3-chlorophenyl)ethyl]-3-(2-furyl)isoxazole-4-carboxamide. HPLC (Method B) r.t. 1.39, mass found=331.07.
5-amino-N-(1-ethylpropyl)-3-(2-furyl)isoxazole-4-carboxamide. HPLC (Method B) r.t. 1.29, mass found=263.13.
5-amino-3-(2-furyl)-N-(1-phenylethyl)isoxazole-4-carboxamide. HPLC (Method B) r.t. 1.33, mass found=297.11.
5-amino-3-(2-furyl)-N-(2-methylbenzyl)isoxazole-4-carboxamide. HPLC (Method B) r.t. 1.33, mass found=297.11.
5-amino-N-cyclobutyl-3-(2-furyl)isoxazole-4-carboxamide. HPLC (Method B) r.t. 1.15, mass found=247.1.
5-amino-N-benzhydryl-3-(2-furyl)isoxazole-4-carboxamide. HPLC (Method B) r.t. 1.49, mass found=359.13.
5-amino-3-(2-furyl)-N-octylisoxazole-4-carboxamide. HPLC (Method B) r.t. 1.64, mass found=305.17.
5-amino-N-(2-bromobenzyl)-3-(2-furyl)isoxazole-4-carboxamide. HPLC (Method B) r.t. 1.39, mass found=361.01.
5-amino-3-(2-furyl)-N-(1-methylheptyl)isoxazole-4-carboxamide. HPLC (Method B) r.t. 1.61, mass found=305.17.
5-amino-3-(2-furyl)-N-heptylisoxazole-4-carboxamide. HPLC (Method B) r.t. 1.54, mass found=291.16.
5-amino-N-(tert-butyl)-3-(2-furyl)isoxazole-4-carboxamide. HPLC (Method B) r.t. 1.25, mass found=249.11.
5-amino-N-(3,4-dimethoxybenzyl)-3-(2-furyl)isoxazole-4-carboxamide. HPLC (Method B) r.t. 1.14, mass found=343.12.
5-amino-N-(2,3-dihydro-1H-inden-1-yl)-3-(2-furyl)isoxazole-4-carboxamide. HPLC (Method B) r.t. 1.37, mass found=309.11.
5-amino-3-(2-furyl)-N-(1,2,3,4-tetrahydronaphthalen-1-yl)isoxazole-4-carboxamide. HPLC (Method B) r.t. 1.43, mass found=323.13.
5-amino-3-(1-benzofuran-3-yl)-N-benzylisoxazole-4-carboxamide. HPLC (Method B) r.t. 1.47, mass found=333.11.
5-amino-3-(1-benzofuran-3-yl)-N-(2-furylmethyl)isoxazole-4-carboxamide. HPLC (Method B) r.t. 1.39, mass found=323.09.
5-amino-3-(1-benzofuran-3-yl)-N-(2-phenylethyl)isoxazole-4-carboxamide. HPLC (Method B) r.t. 1.51, mass found=347.13.
5-amino-3-(1-benzofuran-3-yl)-N-(sec-butyl)isoxazole-4-carboxamide. HPLC (Method B) r.t. 1.45, mass found=299.13.
5-amino-3-(1-benzofuran-3-yl)-N-cyclohexylisoxazole-4-carboxamide. HPLC (Method B) r.t. 1.57, mass found=325.14.
5-amino-3-(1-benzofuran-3-yl)-N-(4-methoxybenzyl)isoxazole-4-carboxamide. HPLC (Method B) r.t. 1.44, mass found=363.12.
5-amino-3-(1-benzofuran-3-yl)-N-hexylisoxazole-4-carboxamide. HPLC (Method B) r.t. 1.64, mass found=327.16.
5-amino-3-(1-benzofuran-3-yl)-N-(tetrahydrofuran-2-ylmethyl)isoxazole-4-carboxamide. HPLC (Method B) r.t. 1.32, mass found=327.12.
5-amino-3-(1-benzofuran-3-yl)-N-(thien-2-ylmethyl)isoxazole-4-carboxamide. HPLC (Method B) r.t. 1.44, mass found=339.07.
5-amino-3-(1-benzofuran-3-yl)-N-cyclopentylisoxazole-4-carboxamide. HPLC (Method B) r.t. 1.47, mass found=311.13.
5-amino-3-(1-benzofuran-3-yl)-N-(1,5-dimethylhexyl)isoxazole-4-carboxamide. HPLC (Method B) r.t. 1.78, mass found=355.19.
5-amino-3-(1-benzofuran-3-yl)-N-isopentylisoxazole-4-carboxamide. HPLC (Method B) r.t. 1.54, mass found=313.14.
5-amino-3-(1-benzofuran-3-yl)-N-(2-ethylhexyl)isoxazole-4-carboxamide. HPLC (Method B) r.t. 1.8, mass found=355.19.
5-amino-3-(1-benzofuran-3-yl)-N-(4-phenylbutyl)isoxazole-4-carboxamide. HPLC (Method B) r.t. 1.63, mass found=375.16.
5-amino-3-(1-benzofuran-3-yl)-N-(2-methylcyclohexyl)isoxazole-4-carboxamide. HPLC (Method B) r.t. 1.62, mass found=339.16.
5-amino-3-(1-benzofuran-3-yl)-N-[3,5-bis(trifluoromethyl)benzyl]isoxazole-4-carboxamide. HPLC (Method B) r.t. 1.68, mass found=469.09.
5-amino-3-(1-benzofuran-3-yl)-N-[3-(trifluoromethyl)benzyl]isoxazole-4-carboxamide. HPLC (Method B) r.t. 1.57, mass found=401.1.
5-amino-3-(1-benzoturan-3-yl)-N-(3-phenylpropyl)isoxazole-4-carboxamide. HPLC (Method B) r.t. 1.57, mass found=361.14.
5-amino-3-(1-benzofuran-3-yl)-N-isobutylisoxazole-4-carboxamide. HPLC (Method B) r.t. 1.45, mass found=299.13.
5-amino-3-(1-benzofuran-3-yl)-N-[2-(trifluoromethyl)benzyl]isoxazole-4-carboxamide. HPLC (Method B) r.t. 1.59, mass found=401.1.
5-amino-3-(1-benzofuran-3-yl)-N-(2-methoxybenzyl)isoxazole-4-carboxamide. HPLC (Method B) r.t. 1.49, mass found=363.12.
5-amino-3-(1-benzofuran-3-yl)-N-(2-fluorobenzyl)isoxazole-4-carboxamide. HPLC (Method B) r.t. 1.49, mass found=351.1.
5-amino-3-(1-benzofuran-3-yl)-N-[4-(trifluoromethyl)benzyl]isoxazole-4-carboxamide. HPLC (Method B) r.t. 1.59, mass found=401.1.
5-amino-3-(1-benzofuran-3-yl)-N-(3,4-difluorobenzyl)isoxazole-4-carboxamide. HPLC (Method B) r.t. 1.5, mass found=369.09.
5-amino-3-(1-benzofuran-3-yl)-N-(3-chlorobenzyl)isoxazole-4-carboxamide. HPLC (Method B) r.t. 1.56, mass found=367.07.
5-amino-3-(1-benzofuran-3-yl)-N-(2-chlorobenzyl)isoxazole-4-carboxamide. HPLC (Method B) r.t. 1.56, mass found=367.07.
5-amino-3-(1-benzofuran-3-yl)-N-(3-methylbenzyl)isoxazole-4-carboxamide. HPLC (Method B) r.t. 1.55, mass found=347.13.
5-amino-3-(1-benzofuran-3-yl)-N-(1-naphthylmethyl)isoxazole-4-carboxamide. HPLC (Method B) r.t. 1.6, mass found=383.13.
5-amino-3-(1-benzofuran-3-yl)-N-(cyclopropylmethyl)isoxazole-4-carboxamide. HPLC (Method B) r.t. 1.39, mass found=297.11.
5-amino-3-(1-benzofuran-3-yl)-N-cycloheptylisoxazole-4-carboxamide. HPLC (Method B) r.t. 1.64, mass found=339.16.
5-amino-3-(1-benzofuran-3-yl)-N-[2-(4-methoxyphenyl)ethyl]isoxazole-4-carboxamide. HPLC (Method B) r.t. 1.48, mass found=377.14.
5-amino-3-(1-benzofuran-3-yl)-N-(2-cyclohex-1-en-1-ylethyl)isoxazole-4-carboxamide. HPLC (Method B) r.t. 1.65, mass found=351.16.
5-amino-3-(1-benzofuran-3-yl)-N-(1,2-dimethylpropyl)isoxazole-4-carboxamide. HPLC (Method B) r.t. 1.53, mass found=313.14.
5-amino-3-(1-benzofuran-3-yl)-N-(3,3-diphenylpropyl)isoxazole-4-carboxamide. HPLC (Method B) r.t. 1.69, mass found=437.17.
5-amino-3-(1-benzofuran-3-yl)-N-(1,2-diphenylethyl)isoxazole-4-carboxamide. HPLC (Method B) r.t. 1.69, mass found=423.16.
5-amino-3-(1-benzofuran-3-yl)-N-(2,4-dimethoxybenzyl)isoxazole-4-carboxamide. HPLC (Method B) r.t. 1.47, mass found=393.13.
5-amino-3-(1-benzofuran-3-yl)-N-[3-(dibutylamino)propyl]isoxazole-4-carboxamide. HPLC (Method B) r.t. 1.28, mass found=412.25.
5-amino-3-(1-benzofuran-3-yl)-N-butylisoxazole-4-carboxamide. HPLC (Method B) r.t. 1.46, mass found=299.13.
5-amino-3-(1-benzofuran-3-yl)-N-[4-(trifluoromethoxy)benzyl]isoxazole-4-carboxamide. HPLC (Method B) r.t. 1.62, mass found=417.09.
5-amino-3-(1-benzofuran-3-yl)-N-[(5-methyl-2-furyl)methyl]isoxazole-4-carboxamide. HPLC (Method B) r.t. 1.47, mass found=337.11.
5-amino-3-(1-benzofuran-3-yl)-N-(1-methylhexyl)isoxazole-4-carboxamide. HPLC (Method B) r.t. 1.72, mass found=341.17.
5-amino-3-(1-benzofuran-3-yl)-N-(1-benzylpiperidin-4-yl)isoxazole-4-carboxamide. HPLC (Method B) r.t. 1.16, mass found=416.18.
5-amino-3-(1-benzofuran-3-yl)-N-(2-methoxy-1-methylethyl)isoxazole-4-carboxamide. HPLC (Method B) r.t. 1.34, mass found=315.12.
5-amino-3-(1-benzofuran-3-yl)-N-(1-methylbutyl)isoxazole-4-carboxamide. HPLC (Method B) r.t. 1.55, mass found=313.14.
5-amino-3-(1-benzofuran-3-yl)-N-(3-fluorobenzyl)isoxazole-4-carboxamide. HPLC (Method B) r.t. 1.48, mass found=351.1.
5-amino-3-(1-benzofuran-3-yl)-N-(1,3-dimethylbutyl)isoxazole-4-carboxamide. HPLC (Method B) r.t. 1.62, mass found=327.16.
5-amino-3-(1-benzofuran-3-yl)-N-(1-methyl-3-phenylpropyl)isoxazole-4-carboxamide. HPLC (Method B) r.t. 1.64, mass found=375.16.
5-amino-3-(1-benzofuran-3-yl)-N-[3-(methylthio)propyl]isoxazole-4-carboxamide. HPLC (Method B) r.t. 1.39, mass found=331.1.
5-amino-N-(1,3-benzodioxol-5-ylmethyl)-3-(1-benzofuran-3-yl)isoxazole-4-carboxamide. HPLC (Method B) r.t. 1.42, mass found=377.1.
5-amino-3-(1-benzofuran-3-yl)-N-pentylisoxazole-4-carboxamide. HPLC (Method B) r.t. 1.55, mass found=313.14.
5-amino-3-(1-benzofuran-3-yl)-N-(cyclohexylmethyl)isoxazole-4-carboxamide. HPLC (Method B) r.t. 1.65, mass found=339.16.
ethyl 4-({[5-amino-3-(1-benzofuran-3-yl)isoxazol-4-yl]carbonyl}amino)piperidine-1-carboxylate. HPLC (Method B) r.t. 1.34, mass found=398.16.
5-amino-3-(1-benzofuran-3-yl)-N-[2-(3-chlorophenyl)ethyl]isoxazole-4-carboxamide. HPLC (Method B) r.t. 1.56, mass found=381.09.
5-amino-3-(i-benzofuran-3-yl)-N-(1-ethylpropyl)isoxazole-4-carboxamide. HPLC (Method B) r.t. 1.49, mass found=313.14.
5-amino-3-(1-benzofuran-3-yl)-N-(1-phenylethyl)isoxazole-4-carboxamide. HPLC (Method B) r.t. 1.54, mass found=347.13.
5-amino-3-(1-benzofuran-3-yl)-N-(2-methylbenzyl)isoxazole-4-carboxamide. HPLC (Method B) r.t. 1.54, mass found=347.13.
5-amino-3-(1-benzofuran-3-yl)-N-cyclobutylisoxazole-4-carboxamide. HPLC (Method B) r.t. 1.41, mass found=297.11.
5-amino-N-benzhydryl-3-(1-benzofuran-3-yl)isoxazole-4-carboxamide. HPLC (Method B) r.t. 1.67, mass found=409.14.
5-amino-3-(1-benzofuran-3-yl)-N-octylisoxazole-4-carboxamide. HPLC (Method B) r.t. 1.83, mass found=355.19.
5-amino-3-(1-benzofuran-3-yl)-N-(2-bromobenzyl)isoxazole-4-carboxamide. HPLC (Method B) r.t. 1.57, mass found=411.02.
5-amino-3-(1-benzofuran-3-yl)-N-(1-methylheptyl)isoxazole-4-carboxamide. HPLC (Method B) r.t. 1.8, mass found=355.19.
5-amino-3-(1-benzofuran-3-yl)-N-heptylisoxazole-4-carboxamide. HPLC (Method B) r.t. 1.74, mass found=341.17.
5-amino-3-(1-benzofuran-3-yl)-N-(tert-butyl)isoxazole-4-carboxamide. HPLC (Method B) r.t. 1.51, mass found=299.13.
5-amino-3-(1-benzofuran-3-yl)-N-(3,4-dimethoxybenzyl)isoxazole-4-carboxamide. HPLC (Method B) r.t. 1.34, mass found=393.13.
5-amino-3-(1-benzofuran-3-yl)-N-(2,3-dihydro-1H-inden-1-yl)isoxazole-4-carboxamide. HPLC (Method B) r.t. 1.58, mass found=359.13.
5-amino-3-(1-benzofuran-3-yl)-N-(1,2,3,4-tetrahydronaphthalen-1-yl)isoxazole-4-carboxamide. HPLC (Method B) r.t. 1.64, mass found=373.14.
5-amino-N-benzyl-3-(5-ethylthien-2-yl)isoxazole-4-carboxamide. HPLC (Method B) r.t. 1.49, mass found=327.1.
5-amino-3-(5-ethylthien-2-yl)-N-(2-furylmethyl)isoxazole-4-carboxamide. HPLC (Method B) r.t. 1.4, mass found=317.08.
5-amino-3-(5-ethylthien-2-yl)-N-(2-phenylethyl)isoxazole-4-carboxamide. HPLC (Method B) r.t. 1.53, mass found=341.12.
5-amino-N-(sec-butyl)-3-(5-ethylthien-2-yl)isoxazole-4-carboxamide. HPLC (Method B) r.t. 1.48, mass found=293.12.
5-amino-N-cyclohexyl-3-(5-ethylthien-2-yl)isoxazole-4-carboxamide. HPLC (Method B) r.t. 1.59, mass found=319.14.
5-amino-3-(5-ethylthien-2-yl)-N-(4-methoxybenzyl)isoxazole-4-carboxamide. HPLC (Method B) r.t. 1.46, mass found=357.11.
5-amino-3-(5-ethylthien-2-yl)-N-hexylisoxazole-4-carboxamide. HPLC (Method B) r.t. 1.67, mass found=321.15.
5-amino-3-(5-ethylthien-2-yl)-N-(thien-2-ylmethyl)isoxazole-4-carboxamide. HPLC (Method B) r.t. 1.46, mass found=333.06.
5-amino-N-cyclopentyl-3-(5-ethylthien-2-yl)isoxazole-4-carboxamide. HPLC (Method B) r.t. 1.5, mass found=305.12.
5-amino-N-(1,5-dimethylhexyl)-3-(5-ethylthien-2-yl)isoxazole-4-carboxamide. HPLC (Method B) r.t. 1.83, mass found=349.18.
5-amino-3-(5-ethylthien-2-yl)-N-isopentylisoxazole-4-carboxamide. HPLC (Method B) r.t. 1.56, mass found=307.14.
5-amino-N-(2-ethylhexyl)-3-(5-ethylthien-2-yl)isoxazole-4-carboxamide. HPLC (Method B) r.t. 1.83, mass found=349.18.
5-amino-3-(5-ethylthien-2-yl)-N-(4-phenylbutyl)isoxazole-4-carboxamide. HPLC (Method B) r.t. 1.66, mass found=369.15.
5-amino-3-(5-ethylthien-2-yl)-N-(2-methylcyclohexyl)isoxazole-4-carboxamide. HPLC (Method B) r.t. 1.66, mass found=333.15.
5-amino-N-[3,5-bis(trifluoromethyl)benzyl]-3-(5-ethylthien-2-yl)isoxazole-4-carboxamide. HPLC (Method B) r.t. 1.71, mass found=463.08.
5-amino-3-(5-ethylthien-2-yl)-N-[3-(trifluoromethyl)benzyl]isoxazole-4-carboxamide. HPLC (Method B) r.t. 1.6, mass found=395.09.
5-amino-3-(5-ethylthien-2-yl)-N-(3-phenylpropyl)isoxazole-4-carboxamide. HPLC (Method B) r.t. 1.59, mass found=355.14.
5-amino-3-(5-ethylthien-2-yl)-N-isobutylisoxazole-4-carboxamide. HPLC (Method B) r.t. 1.48, mass found=293.12.
5-amino-3-(5-ethylthien-2-yl)-N-[2-(trifluoromethyl)benzyl]isoxazole-4-carboxamide. HPLC (Method B) r.t. 1.62, mass found=395.09.
5-amino-3-(5-ethylthien-2-yl)-N-(2-methoxybenzyl)isoxazole-4-carboxamide. HPLC (Method B) r.t. 1.51, mass found=357.11.
5-amino-3-(5-ethylthien-2-yl)-N-(2-fluorobenzyl)isoxazole-4-carboxamide. HPLC (Method B) r.t. 1.5, mass found=345.09.
5-amino-N-(3,4-difluorobenzyl)-3-(5-ethylthien-2-yl)isoxazole-4-carboxamide. HPLC (Method B) r.t. 1.52, mass found=363.09.
5-amino-N-(3-chlorobenzyl)-3-(5-ethylthien-2-yl)isoxazole-4-carboxamide. HPLC (Method B) r.t. 1.58, mass found=361.07.
5-amino-N-(2-chlorobenzyl)-3-(5-ethylthien-2-yl)isoxazole-4-carboxamide. HPLC (Method B) r.t. 1.58, mass found=361.07.
5-amino-3-(5-ethylthien-2-yl)-N-(3-methylbenzyl)isoxazole-4-carboxamide. HPLC (Method B) r.t. 1.56, mass found=341.12.
5-amino-3-(5-ethylthien-2-yl)-N-(1-naphthylmethyl)isoxazole-4-carboxamide. HPLC (Method B) r.t. 1.62, mass found=377.12.
5-amino-N-(cyclopropylmethyl)-3-(5-ethylthien-2-yl)isoxazole-4-carboxamide. HPLC (Method B) r.t. 1.41, mass found=291.1.
5-amino-N-cycloheptyl-3-(5-ethylthien-2-yl)isoxazole-4-carboxamide. HPLC (Method B) r.t. 1.66, mass found=333.15.
5-amino-3-(5-ethylthien-2-yl)-N-[2-(4-methoxyphenyl)ethyl]isoxazole-4-carboxamide. HPLC (Method B) r.t. 1.5, mass found=371.13.
5-amino-N-(2-cyclohex-1-en-1-ylethyl)-3-(5-ethylthien-2-yl)isoxazole-4-carboxamide. HPLC (Method B) r.t. 1.69, mass found=345.15.
5-amino-N-(1,2-dimethylpropyl)-3-(5-ethylthien-2-yl)isoxazole-4-carboxamide. HPLC (Method B) r.t. 1.55, mass found=307.14.
5-amino-N-(3,3-diphenylpropyl)-3-(5-ethylthien-2-yl)isoxazole-4-carboxamide. HPLC (Method B) r.t. 1.72, mass found=431.17.
5-amino-N-(1,2-diphenylethyl)-3-(5-ethylthien-2-yl)isoxazole-4-carboxamide. HPLC (Method B) r.t. 1.71, mass found=417.15.
5-amino-N-(2,4-dimethoxybenzyl)-3-(5-ethylthien-2-yl)isoxazole-4-carboxamide. HPLC (Method B) r.t. 1.5, mass found=387.13.
5-amino-N-[3-(dibutylamino)propyl]-3-(5-ethylthien-2-yl)isoxazole-4-carboxamide. HPLC (Method B) r.t. 1.28, mass found=406.24.
5-amino-N-butyl-3-(5-ethylthien-2-yl)isoxazole-4-carboxamide. HPLC (Method B) r.t. 1.48, mass found=293.12.
5-amino-3-(5-ethylthien-2-yl)-N-[4-(trifluoromethoxy)benzyl]isoxazole-4-carboxamide. HPLC (Method B) r.t. 1.63, mass found=411.09.
5-amino-3-(5-ethylthien-2-yl)-N-[(5-methyl-2-furyl)methyl]isoxazole-4-carboxamide. HPLC (Method B) r.t. 1.47, mass found=331.1.
5-amino-3-(5-ethylthien-2-yl)-N-(1-methylhexyl)isoxazole-4-carboxamide. HPLC (Method B) r.t. 1.75, mass found=335.17.
5-amino-3-(5-ethylthien-2-yl)-N-(2-methoxy-1-methylethyl)isoxazole-4-carboxamide. HPLC (Method B) r.t. 1.34, mass found=309.11.
5-amino-N-(4-tert-butylcyclohexyl)-3-(5-ethylthien-2-yl)isoxazole-4-carboxamide. HPLC (Method B) r.t. 1.92, mass found=375.2.
5-amino-3-(5-ethylthien-2-yl)-N-(1-methylbutyl)isoxazole-4-carboxamide. HPLC (Method B) r.t. 1.57, mass found=307.14.
5-amino-3-(5-ethylthien-2-yl)-N-(3-fluorobenzyl)isoxazole-4-carboxamide. HPLC (Method B) r.t. 1.5, mass found=345.09.
5-amino-3-(5-ethylthien-2-yl)-N-(4-methylbenzyl)isoxazole-4-carboxamide. HPLC (Method B) r.t. 1.56, mass found=341.12.
5-amino-N-(1,3-dimethylbutyl)-3-(5-ethylthien-2-yl)isoxazole-4-carboxamide. HPLC (Method B) r.t. 1.65, mass found=321.15.
5-amino-3-(5-ethylthien-2-yl)-N-(1-methyl-3-phenylpropyl)isoxazole-4-carboxamide. HPLC (Method B) r.t. 1.66, mass found=369.15.
5-amino-3-(5-ethylthien-2-yl)-N-[3-(methylthio)propyl]isoxazole-4-carboxamide. HPLC (Method B) r.t. 1.41, mass found=325.09.
5-amino-N-(1,3-benzodioxol-5-ylmethyl)-3-(5-ethylthien-2-yl)isoxazole-4-carboxamide. HPLC (Method B) r.t. 1.44, mass found=371.09.
5-amino-3-(5-ethylthien-2-yl)-N-pentylisoxazole-4-carboxamide. HPLC (Method B) r.t. 1.58, mass found=307.14.
5-amino-N-(cyclohexylmethyl)-3-(5-ethylthien-2-yl)isoxazole-4-carboxamide. HPLC (Method B) r.t. 1.67, mass found=333.15.
ethyl 4-({[5-amino-3-(5-ethylthien-2-yl)isoxazol-4-yl]carbonyl}amino)piperidine-1-carboxylate. HPLC (Method B) r.t. 1.36, mass found=392.15.
5-amino-N-[2-(3-chlorophenyl)ethyl]-3-(5-ethylthien-2-yl)isoxazole-4-carboxamide. HPLC (Method B) r.t. 1.6, mass found=375.08.
5-amino-N-(1-ethylpropyl)-3-(5-ethylthien-2-yl)isoxazole-4-carboxamide. HPLC (Method B) r.t. 1.55, mass found=307.14.
5-amino-3-(5-ethylthien-2-yl)-N-(1-phenylethyl)isoxazole-4-carboxamide. HPLC (Method B) r.t. 1.56, mass found=341.12.
5-amino-3-(5-ethylthien-2-yl)-N-(2-methylbenzyl)isoxazole-4-carboxamide. HPLC (Method B) r.t. 1.55, mass found=341.12.
5-amino-N-cyclobutyl-3-(5-ethylthien-2-yl)isoxazole-4-carboxamide. HPLC (Method B) r.t. 1.43, mass found=291.1.
5-amino-N-benzhydryl-3-(5-ethylthien-2-yl)isoxazole-4-carboxamide. HPLC (Method B) r.t. 1.68, mass found=403.14.
5-amino-3-(5-ethylthien-2-yl)-N-octylisoxazole-4-carboxamide. HPLC (Method B) r.t. 1.86, mass found=349.18.
5-amino-N-(2-bromobenzyl)-3-(5-ethylthien-2-yl)isoxazole-4-carboxamide. HPLC (Method B) r.t. 1.6, mass found=405.01.
5-amino-3-(5-ethylthien-2-yl)-N-(1-methylheptyl)isoxazole-4-carboxamide. HPLC (Method B) r.t. 1.85, mass found=349.18.
5-amino-3-(5-ethylthien-2-yl)-N-heptylisoxazole-4-carboxamide. HPLC (Method B) r.t. 1.77, mass found=335.17.
5-amino-N-(tert-butyl)-3-(5-ethylthien-2-yl)isoxazole-4-carboxamide. HPLC (Method B) r.t. 1.53, mass found=293.12.
5-amino-N-(3,4-dimethoxybenzyl)-3-(5-ethylthien-2-yl)isoxazole-4-carboxamide. HPLC (Method B) r.t. 1.36, mass found=387.13.
5-amino-N-(2,3-dihydro-1H-inden-1-yl)-3-(5-ethylthien-2-yl)isoxazole-4-carboxamide. HPLC (Method B) r.t. 1.59, mass found=353.12.
5-amino-3-(5-ethylthien-2-yl)-N-(1,2,3,4-tetrahydronaphthalen-1-yl)isoxazole-4-carboxamide. HPLC (Method B) r.t. 1.64, mass found=367.14.
5-amino-N-benzyl-3-(5-chloro-2-furyl)isoxazole-4-carboxamide. HPLC (Method B) r.t. 1.39, mass found=317.06.
5-amino-3-(5-chloro-2-furyl)-N-(2-furylmethyl)isoxazole-4-carboxamide. HPLC (Method B) r.t. 1.3, mass found=307.04.
5-amino-3-(5-chloro-2-furyl)-N-(2-phenylethyl)isoxazole-4-carboxamide. HPLC (Method B) r.t. 1.43, mass found=331.07.
5-amino-N-(sec-butyl)-3-(5-chloro-2-furyl)isoxazole-4-carboxamide. HPLC (Method B) r.t. 1.37, mass found=283.07.
5-amino-3-(5-chloro-2-furyl)-N-cyclohexylisoxazole-4-carboxamide. HPLC (Method B) r.t. 1.49, mass found=309.09.
5-amino-3-(5-chloro-2-furyl)-N-(4-methoxybenzyl)isoxazole-4-carboxamide. HPLC (Method B) r.t. 1.37, mass found=347.07.
5-amino-3-(5-chloro-2-furyl)-N-hexylisoxazole-4-carboxamide. HPLC (Method B) r.t. 1.58, mass found=311.1.
5-amino-3-(5-chloro-2-furyl)-N-cyclopentylisoxazole-4-carboxamide. HPLC (Method B) r.t. 1.4, mass found=295.07.
5-amino-3-(5-chloro-2-furyl)-N-(1,5-dimethylhexyl)isoxazole-4-carboxamide. HPLC (Method B) r.t. 1.73, mass found=339.14.
5-amino-3-(5-chloro-2-furyl)-N-isopentylisoxazole-4-carboxamide. HPLC (Method B) r.t. 1.46, mass found=297.09.
5-amino-3-(5-chloro-2-furyl)-N-(2-ethylhexyl)isoxazole-4-carboxamide. HPLC (Method B) r.t. 1.74, mass found=339.14.
5-amino-3-(5-chloro-2-furyl)-N-(4-phenylbutyl)isoxazole-4-carboxamide. HPLC (Method B) r.t. 1.57, mass found=359.1.
5-amino-3-(5-chloro-2-furyl)-N-(2-methylcyclohexyl)isoxazole-4-carboxamide. HPLC (Method B) r.t. 1.56, mass found=323.1.
5-amino-N-[3,5-bis(trifluoromethyl)benzyl]-3-(5-chloro-2-furyl)isoxazole-4-carboxamide. HPLC (Method B) r.t. 1.63, mass found=453.03.
5-amino-3-(5-chloro-2-furyl)-N-[3-(trifluoromethyl)benzyl]isoxazole-4-carboxamide. HPLC (Method B) r.t. 1.51, mass found=385.04.
5-amino-3-(5-chloro-2-furyl)-N-(3-phenylpropyl)isoxazole-4-carboxamide. HPLC (Method B) r.t. 1.5, mass found=345.09.
5-amino-3-(5-chloro-2-furyl)-N-isobutylisoxazole-4-carboxamide. HPLC (Method B) r.t. 1.37, mass found=283.07.
5-amino-3-(5-chloro-2-furyl)-N-[2-(trifluoromethyl)benzyl]isoxazole-4-carboxamide. HPLC (Method B) r.t. 1.52, mass found=385.04.
5-amino-3-(5-chloro-2-furyl)-N-(2-methoxybenzyl)isoxazole-4-carboxamide. HPLC (Method B) r.t. 1.42, mass found=347.07.
5-amino-3-(5-chloro-2-furyl)-N-(2-fluorobenzyl)isoxazole-4-carboxamide. HPLC (Method B) r.t. 1.41, mass found=335.05.
5-amino-3-(5-chloro-2-furyl)-N-[4-(trifluoromethyl)benzyl]isoxazole-4-carboxamide. HPLC (Method B) r.t. 1.52, mass found=385.04.
5-amino-3-(5-chloro-2-furyl)-N-(3,4-difluorobenzyl)isoxazole-4-carboxamide. HPLC (Method B) r.t. 1.43, mass found=353.04.
5-amino-N-(3-chlorobenzyl)-3-(5-chloro-2-furyl)isoxazole-4-carboxamide. HPLC (Method B) r.t. 1.48, mass found=351.02.
5-amino-N-(2-chlorobenzyl)-3-(5-chloro-2-furyl)isoxazole-4-carboxamide. HPLC (Method B) r.t. 1.48, mass found=351.02.
5-amino-3-(5-chloro-2-furyl)-N-(3-methylbenzyl)isoxazole-4-carboxamide. HPLC (Method B) r.t. 1.47, mass found=331.07.
5-amino-3-(5-chloro-2-furyl)-N-(1-naphthylmethyl)isoxazole-4-carboxamide. HPLC (Method B) r.t. 1.53, mass found=367.07.
5-amino-3-(5-chloro-2-furyl)-N-(cyclopropylmethyl)isoxazole-4-carboxamide. HPLC (Method B) r.t. 1.31, mass found=281.06.
5-amino-3-(5-chloro-2-furyl)-N-cycloheptylisoxazole-4-carboxamide. HPLC (Method B) r.t. 1.58, mass found=323.1.
5-amino-3-(5-chloro-2-furyl)-N-[2-(4-methoxyphenyl)ethyl]isoxazole-4-carboxamide. HPLC (Method B) r.t. 1.41, mass found=361.08.
5-amino-3-(5-chloro-2-furyl)-N-(2-cyclohex-1-en-1-ylethyl)isoxazole-4-carboxamide. HPLC (Method B) r.t. 1.59, mass found=335.1.
5-amino-3-(5-chloro-2-furyl)-N-(1,2-dimethylpropyl)isoxazole-4-carboxamide. HPLC (Method B) r.t. 1.46, mass found=297.09.
5-amino-3-(5-chloro-2-furyl)-N-(3,3-diphenylpropyl)isoxazole-4-carboxamide. HPLC (Method B) r.t. 1.63, mass found=421.12.
5-amino-3-(5-chloro-2-furyl)-N-(1,2-diphenylethyl)isoxazole-4-carboxamide. HPLC (Method B) r.t. 1.64, mass found=407.1.
5-amino-3-(5-chloro-2-furyl)-N-(2,4-dimethoxybenzyl)isoxazole-4-carboxamide. HPLC (Method B) r.t. 1.4, mass found=377.08.
5-amino-3-(5-chloro-2-furyl)-N-[3-(dibutylamino)propyl]isoxazole-4-carboxamide. HPLC (Method B) r.t. 1.19, mass found=396.19.
5-amino-N-butyl-3-(5-chloro-2-furyl)isoxazole-4-carboxamide. HPLC (Method B) r.t. 1.38, mass found=283.07.
5-amino-3-(5-chloro-2-furyl)-N-[4-(trifluoromethoxy)benzyl]isoxazole-4-carboxamide. HPLC (Method B) r.t. 1.55, mass found=401.04.
5-amino-3-(5-chloro-2-furyl)-N-[(5-methyl-2-furyl)methyl]isoxazole-4-carboxamide. HPLC (Method B) r.t. 1.38, mass found=321.05.
5-amino-3-(5-chloro-2-furyl)-N-(1-methylhexyl)isoxazole-4-carboxamide. HPLC (Method B) r.t. 1.67, mass found=325.12.
5-amino-N-(1-benzylpiperidin-4-yl)-3-(5-chloro-2-furyl)isoxazole-4-carboxamide. HPLC (Method B) r.t. 1.08, mass found=400.13.
5-amino-3-(5-chloro-2-furyl)-N-(2-methoxy-1-methylethyl)isoxazole-4-carboxamide. HPLC (Method B) r.t. 1.22, mass found=299.07.
5-amino-N-(4-tert-butylcyclohexyl)-3-(5-chloro-2-furyl)isoxazole-4-carboxamide. HPLC (Method B) r.t. 1.83, mass found=365.15.
5-amino-3-(5-chloro-2-furyl)-N-(1-methylbutyl)isoxazole-4-carboxamide. HPLC (Method B) r.t. 1.47, mass found=297.09.
5-amino-3-(5-chloro-2-furyl)-N-(3-fluorobenzyl)isoxazole-4-carboxamide. HPLC (Method B) r.t. 1.4, mass found=335.05.
5-amino-3-(5-chloro-2-furyl)-N-(4-methylbenzyl)isoxazole-4-carboxamide. HPLC (Method B) r.t. 1.47, mass found=331.07.
5-amino-3-(5-chloro-2-furyl)-N-(1,3-dimethylbutyl)isoxazole-4-carboxamide. HPLC (Method B) r.t. 1.55, mass found=311.1.
5-amino-3-(5-chloro-2-furyl)-N-(1-methyl-3-phenylpropyl)isoxazole-4-carboxamide. HPLC (Method B) r.t. 1.58, mass found=359.1.
5-amino-3-(5-chloro-2-furyl)-N-[3-(methylthio)propyl]isoxazole-4-carboxamide. HPLC (Method B) r.t. 1.3, mass found=315.04.
5-amino-N-(1,3-benzodioxol-5-ylmethyl)-3-(5-chloro-2-furyl)isoxazole-4-carboxamide. HPLC (Method B) r.t. 1.35, mass found=361.05.
5-amino-3-(5-chloro-2-furyl)-N-pentylisoxazole-4-carboxamide. HPLC (Method B) r.t. 1.48, mass found=297.09.
5-amino-3-(5-chloro-2-furyl)-N-(cyclohexylmethyl)isoxazole-4-carboxamide. HPLC (Method B) r.t. 1.58, mass found=323.1.
ethyl 4-({[5-amino-3-(5-chloro-2-furyl)isoxazol-4-yl]carbonyl}amino)piperidine-1-carboxylate. HPLC (Method B) r.t. 1.26, mass found=382.1.
5-amino-3-(5-chloro-2-furyl)-N-[2-(3-chlorophenyl)ethyl]isoxazole-4-carboxamide. HPLC (Method B) r.t. 1.52, mass found=365.03.
5-amino-3-(5-chloro-2-furyl)-N-(1-ethylpropyl)isoxazole-4-carboxamide. HPLC (Method B) r.t. 1.45, mass found=297.09.
5-amino-3-(5-chloro-2-furyl)-N-(1-phenylethyl)isoxazole-4-carboxamide. HPLC (Method B) r.t. 1.47, mass found=331.07.
5-amino-3-(5-chloro-2-furyl)-N-(2-methylbenzyl)isoxazole-4-carboxamide. HPLC (Method B) r.t. 1.46, mass found=331.07.
5-amino-3-(5-chloro-2-furyl)-N-cyclobutylisoxazole-4-carboxamide. HPLC (Method B) r.t. 1.32, mass found=281.06.
5-amino-N-benzhydryl-3-(5-chloro-2-furyl)isoxazole-4-carboxamide. HPLC (Method B) r.t. 1.61, mass found=393.09.
5-amino-3-(5-chloro-2-furyl)-N-octylisoxazole-4-carboxamide. HPLC (Method B) r.t. 1.77, mass found=339.14.
5-amino-N-(2-bromobenzyl)-3-(5-chloro-2-furyl)isoxazole-4-carboxamide. HPLC (Method B) r.t. 1.51, mass found=394.97.
5-amino-3-(5-chloro-2-furyl)-N-(1-methylheptyl)isoxazole-4-carboxamide. HPLC (Method B) r.t. 1.76, mass found=339.14.
5-amino-3-(5-chloro-2-furyl)-N-heptylisoxazole-4-carboxamide. HPLC (Method B) r.t. 1.68, mass found=325.12.
5-amino-N-(tert-butyl)-3-(5-chloro-2-furyl)isoxazole-4-carboxamide. HPLC (Method B) r.t. 1.44, mass found=283.07.
5-amino-3-(5-chloro-2-furyl)-N-(3,4-dimethoxybenzyl)isoxazole-4-carboxamide. HPLC (Method B) r.t. 1.26, mass found=377.08.
5-amino-3-(5-chloro-2-furyl)-N-(2,3-dihydro-1H-inden-1-yl)isoxazole-4-carboxamide. HPLC (Method B) r.t. 1.5, mass found=343.07.
5-amino-3-(5-chloro-2-furyl)-N-(1,2,3,4-tetrahydronaphthalen-1-yl)isoxazole-4-carboxamide. HPLC (Method B) r.t. 1.56, mass found=357.09.
5-amino-N-benzyl-3-(6-methoxypyridin-3-yl)isoxazole-4-carboxamide. HPLC (Method B) r.t. 1.23, mass found=324.12.
5-amino-N-(2-furylmethyl)-3-(6-methoxypyridin-3-yl)isoxazole-4-carboxamide. HPLC (Method B) r.t. 1.12, mass found=314.1.
5-amino-3-(6-methoxypyridin-3-yl)-N-(2-phenylethyl)isoxazole-4-carboxamide. HPLC (Method B) r.t. 1.29, mass found=338.14.
5-amino-N-(sec-butyl)-3-(6-methoxypyridin-3-yl)isoxazole-4-carboxamide. HPLC (Method B) r.t. 1.17, mass found=290.14.
5-amino-N-cyclohexyl-3-(6-methoxypyridin-3-yl)isoxazole-4-carboxamide. HPLC (Method B) r.t. 1.29, mass found=316.15.
5-amino-N-(4-methoxybenzyl)-3-(6-methoxypyridin-3-yl)isoxazole-4-carboxamide. HPLC (Method B) r.t. 1.22, mass found=354.13.
5-amino-N-hexyl-3-(6-methoxypyridin-3-yl)isoxazole-4-carboxamide. HPLC (Method B) r.t. 1.41, mass found=318.17.
5-amino-3-(6-methoxypyridin-3-yl)-N-(thien-2-ylmethyl)isoxazole-4-carboxamide. HPLC (Method B) r.t. 1.19, mass found=330.08.
5-amino-N-cyclopentyl-3-(6-methoxypyridin-3-yl)isoxazole-4-carboxamide. HPLC (Method B) r.t. 1.2, mass found=302.14.
5-amino-N-(1,5-dimethylhexyl)-3-(6-methoxypyridin-3-yl)isoxazole-4-carboxamide. HPLC (Method B) r.t. 1.58, mass found=346.2.
5-amino-N-isopentyl-3-(6-methoxypyridin-3-yl)isoxazole-4-carboxamide. HPLC (Method B) r.t. 1.29, mass found=304.15.
5-amino-N-(2-ethylhexyl)-3-(6-methoxypyridin-3-yl)isoxazole-4-carboxamide. HPLC (Method B) r.t. 1.58, mass found=346.2.
5-amino-3-(6-methoxypyridin-3-yl)-N-(4-phenylbutyl)isoxazole-4-carboxamide. HPLC (Method B) r.t. 1.44, mass found=366.17.
5-amino-3-(6-methoxypyridin-3-yl)-N-(2-methylcyclohexyl)isoxazole-4-carboxamide. HPLC (Method B) r.t. 1.38, mass found=330.17.
5-amino-N-[3,5-bis(trifluoromethyl)benzyl]-3-(6-methoxypyridin-3-yl)isoxazole-4-carboxamide. HPLC (Method B) r.t. 1.54, mass found=460.1.
5-amino-3-(6-methoxypyridin-3-yl)-N-[3-(trifluoromethyl)benzyl]isoxazole-4-carboxamide. HPLC (Method B) r.t. 1.39, mass found=392.11.
5-amino-3-(6-methoxypyridin-3-yl)-N-(3-phenylpropyl)isoxazole-4-carboxamide. HPLC (Method B) r.t. 1.37, mass found=352.15.
5-amino-N-isobutyl-3-(6-methoxypyridin-3-yl)isoxazole-4-carboxamide. HPLC (Method B) r.t. 1.18, mass found=290.14.
5-amino-3-(6-methoxypyridin-3-yl)-N-[2-(trifluoromethyl)benzyl]isoxazole-4-carboxamide. HPLC (Method B) r.t. 1.39, mass found=392.11.
5-amino-N-(2-methoxybenzyl)-3-(6-methoxypyridin-3-yl)isoxazole-4-carboxamide. HPLC (Method B) r.t. 1.28, mass found=354.13.
5-amino-N-(2-fluorobenzyl)-3-(6-methoxypyridin-3-yl)isoxazole-4-carboxamide. HPLC (Method B) r.t. 1.25, mass found=342.11.
5-amino-3-(6-methoxypyridin-3-yl)-N-[4-(trifluoromethyl)benzyl]isoxazole-4-carboxamide. HPLC (Method B) r.t. 1.4, mass found=392.11.
5-amino-N-(3,4-difluorobenzyl)-3-(6-methoxypyridin-3-yl)isoxazole-4-carboxamide. HPLC (Method B) r.t. 1.3, mass found=360.1.
5-amino-N-(3-chlorobenzyl)-3-(6-methoxypyridin-3-yl)isoxazole-4-carboxamide. HPLC (Method B) r.t. 1.34, mass found=358.08.
5-amino-N-(2-chlorobenzyl)-3-(6-methoxypyridin-3-yl)isoxazole-4-carboxamide. HPLC (Method B) r.t. 1.33, mass found=358.08.
5-amino-3-(6-methoxypyridin-3-yl)-N-(3-methylbenzyl)isoxazole-4-carboxamide. HPLC (Method B) r.t. 1.33, mass found=338.14.
5-amino-3-(6-methoxypyridin-3-yl)-N-(1-naphthylmethyl)isoxazole-4-carboxamide. HPLC (Method B) r.t. 1.38, mass found=374.14.
5-amino-N-(cyclopropylmethyl)-3-(6-methoxypyridin-3-yl)isoxazole-4-carboxamide. HPLC (Method B) r.t. 1.11, mass found=288.12.
5-amino-N-cycloheptyl-3-(6-methoxypyridin-3-yl)isoxazole-4-carboxamide. HPLC (Method B) r.t. 1.38, mass found=330.17.
5-amino-N-[2-(4-methoxyphenyl)ethyl]-3-(6-methoxypyridin-3-yl)isoxazole-4-carboxamide. HPLC (Method B) r.t. 1.27, mass found=368.15.
5-amino-N-(2-cyclohex-1-en-1-ylethyl)-3-(6-methoxypyridin-3-yl)isoxazole-4-carboxamide. HPLC (Method B) r.t. 1.44, mass found=342.17.
5-amino-N-(1,2-dimethylpropyl)-3-(6-methoxypyridin-3-yl)isoxazole-4-carboxamide. HPLC (Method B) r.t. 1.27, mass found=304.15.
5-amino-N-(3,3-diphenylpropyl)-3-(6-methoxypyridin-3-yl)isoxazole-4-carboxamide. HPLC (Method B) r.t. 1.52, mass found=428.18.
5-amino-N-(1,2-diphenylethyl)-3-(6-methoxypyridin-3-yl)isoxazole-4-carboxamide. HPLC (Method B) r.t. 1.52, mass found=414.17.
5-amino-N-(2,4-dimethoxybenzyl)-3-(6-methoxypyridin-3-yl)isoxazole-4-carboxamide. HPLC (Method B) r.t. 1.27, mass found=384.14.
5-amino-N-[3-(dibutylamino)propyl]-3-(6-methoxypyridin-3-yl)isoxazole-4-carboxamide. HPLC (Method B) r.t. 1.11, mass found=403.26.
5-amino-N-butyl-3-(6-methoxypyridin-3-yl)isoxazole-4-carboxamide. HPLC (Method B) r.t. 1.19, mass found=290.14.
5-amino-3-(6-methoxypyridin-3-yl)-N-[4-(trifluoromethoxy)benzyl]isoxazole-4-carboxamide. HPLC (Method B) r.t. 1.43, mass found=408.1.
5-amino-3-(6-methoxypyridin-3-yl)-N-[(5-methyl-2-furyl)methyl]isoxazole-4-carboxamide. HPLC (Method B) r.t. 1.22, mass found=328.12.
5-amino-3-(6-methoxypyridin-3-yl)-N-(1-methylhexyl)isoxazole-4-carboxamide. HPLC (Method B) r.t. 1.49, mass found=332.18.
5-amino-N-(1-benzylpiperidin-4-yl)-3-(6-methoxypyridin-3-yl)isoxazole-4-carboxamide. HPLC (Method B) r.t. 0.97, mass found=407.2.
5-amino-N-(2-methoxy-1-methylethyl)-3-(6-methoxypyridin-3-yl)isoxazole-4-carboxamide. HPLC (Method B) r.t. 1.03, mass found=306.13.
5-amino-N-(4-tert-butylcyclohexyl)-3-(6-methoxypyridin-3-yl)isoxazole-4-carboxamide. HPLC (Method B) r.t. 1.65, mass found=372.22.
5-amino-3-(6-methoxypyridin-3-yl)-N-(1-methylbutyl)isoxazole-4-carboxamide. HPLC (Method B) r.t. 1.28, mass found=304.15.
5-amino-N-(3-fluorobenzyl)-3-(6-methoxypyridin-3-yl)isoxazole-4-carboxamide. HPLC (Method B) r.t. 1.27, mass found=342.11.
5-amino-3-(6-methoxypyridin-3-yl)-N-(4-methylbenzyl)isoxazole-4-carboxamide. HPLC (Method B) r.t. 1.32, mass found=338.14.
5-amino-N-(1,3-dimethylbutyl)-3-(6-methoxypyridin-3-yl)isoxazole-4-carboxamide. HPLC (Method B) r.t. 1.37, mass found=318.17.
5-amino-3-(6-methoxypyridin-3-yl)-N-(1-methyl-3-phenylpropyl)isoxazole-4-carboxamide. HPLC (Method B) r.t. 1.43, mass found=366.17.
5-amino-3-(6-methoxypyridin-3-yl)-N-[3-(methylthio)propyl]isoxazole-4-carboxamide. HPLC (Method B) r.t. 1.13, mass found=322.11.
5-amino-N-(1,3-benzodioxol-5-ylmethyl)-3-(6-methoxypyridin-3-yl)isoxazole-4-carboxamide. HPLC (Method B) r.t. 1.21, mass found=368.11.
5-amino-3-(6-methoxypyridin-3-yl)-N-pentylisoxazole-4-carboxamide. HPLC (Method B) r.t. 1.3, mass found=304.15.
5-amino-N-(cyclohexylmethyl)-3-(6-methoxypyridin-3-yl)isoxazole-4-carboxamide. HPLC (Method B) r.t. 1.4, mass found=330.17.
ethyl 4-({[5-amino-3-(6-methoxypyridin-3-yl)isoxazol-4-yl]carbonyl}amino)piperidine-1-carboxylate. HPLC (Method B) r.t. 1.11, mass found=389.17.
5-amino-N-[2-(3-chlorophenyl)ethyl]-3-(6-methoxypyridin-3-yl)isoxazole-4-carboxamide. HPLC (Method B) r.t. 1.38, mass found=372.1.
5-amino-N-(1-ethylpropyl)-3-(6-methoxypyridin-3-yl)isoxazole-4-carboxamide. HPLC (Method B) r.t. 1.27, mass found=304.15.
5-amino-3-(6-methoxypyridin-3-yl)-N-(1-phenylethyl)isoxazole-4-carboxamide. HPLC (Method B) r.t. 1.3, mass found=338.14.
5-amino-3-(6-methoxypyridin-3-yl)-N-(2-methylbenzyl)isoxazole-4-carboxamide. HPLC (Method B) r.t. 1.3, mass found=338.14.
5-amino-N-cyclobutyl-3-(6-methoxypyridin-3-yl)isoxazole-4-carboxamide. HPLC (Method B) r.t. 1.12, mass found=288.12.
5-amino-N-benzhydryl-3-(6-methoxypyridin-3-yl)isoxazole-4-carboxamide. HPLC (Method B) r.t. 1.47, mass found=400.15.
5-amino-3-(6-methoxypyridin-3-yl)-N-octylisoxazole-4-carboxamide. HPLC (Method B) r.t. 1.62, mass found=346.2.
5-amino-N-(2-bromobenzyl)-3-(6-methoxypyridin-3-yl)isoxazole-4-carboxamide. HPLC (Method B) r.t. 1.35, mass found=402.03.
5-amino-3-(6-methoxypyridin-3-yl)-N-(1-methylheptyl)isoxazole-4-carboxamide. HPLC (Method B) r.t. 1.59, mass found=346.2.
5-amino-N-heptyl-3-(6-methoxypyridin-3-yl)isoxazole-4-carboxamide. HPLC (Method B) r.t. 1.51, mass found=332.18.
5-amino-N-(tert-butyl)-3-(6-methoxypyridin-3-yl)isoxazole-4-carboxamide. HPLC (Method B) r.t. 1.23, mass found=290.14.
5-amino-N-(3,4-dimethoxybenzyl)-3-(6-methoxypyridin-3-yl)isoxazole-4-carboxamide. HPLC (Method B) r.t. 1.13, mass found=384.14.
5-amino-N-(2,3-dihydro-1H-inden-1-yl)-3-(6-methoxypyridin-3-yl)isoxazole-4-carboxamide. HPLC (Method B) r.t. 1.33, mass found=350.14.
5-amino-3-(6-methoxypyridin-3-yl)-N-(1,2,3,4-tetrahydronaphthalen-1-yl)isoxazole-4-carboxamide. HPLC (Method B) r.t. 1.39, mass found=364.15.
5-amino-N-benzyl-3-pyridin-3-ylisoxazole-4-carboxamide. HPLC (Method B) r.t. 0.87, mass found=294.11.
5-amino-N-(2-furylmethyl)-3-pyridin-3-ylisoxazole-4-carboxamide. HPLC (Method B) r.t. 0.74, mass found=284.09.
5-amino-N-(2-phenylethyl)-3-pyridin-3-ylisoxazole-4-carboxamide. HPLC (Method B) r.t. 0.93, mass found=308.13.
5-amino-N-cyclohexyl-3-pyridin-3-ylisoxazole-4-carboxamide. HPLC (Method B) r.t. 0.89, mass found=286.14.
5-amino-N-(4-methoxybenzyl)-3-pyridin-3-ylisoxazole-4-carboxamide. HPLC (Method B) r.t. 0.89, mass found=324.12.
5-amino-N-hexyl-3-pyridin-3-ylisoxazole-4-carboxamide. HPLC (Method B) r.t. 1.03, mass found=288.16.
5-amino-3-pyridin-3-yl-N-(thien-2-ylmethyl)isoxazole-4-carboxamide. HPLC (Method B) r.t. 0.83, mass found=300.07.
5-amino-N-cyclopentyl-3-pyridin-3-ylisoxazole-4-carboxamide. HPLC (Method B) r.t. 0.79, mass found=272.13.
5-amino-N-(1,5-dimethylhexyl)-3-pyridin-3-ylisoxazole-4-carboxamide. HPLC (Method B) r.t. 1.19, mass found=316.19.
5-amino-N-isopentyl-3-pyridin-3-ylisoxazole-4-carboxamide. HPLC (Method B) r.t. 0.9, mass found=274.14.
5-amino-N-(2-ethylhexyl)-3-pyridin-3-ylisoxazole-4-carboxamide. HPLC (Method B) r.t. 1.21, mass found=316.19.
5-amino-N-(4-phenylbutyl)-3-pyridin-3-ylisoxazole-4-carboxamide. HPLC (Method B) r.t. 1.1, mass found=336.16.
5-amino-N-(2-methylcyclohexyl)-3-pyridin-3-ylisoxazole-4-carboxamide. HPLC (Method B) r.t. 0.97, mass found=300.16.
5-amino-N-[3,5-bis(trifluoromethyl)benzyl]-3-pyridin-3-ylisoxazole-4-carboxamide. HPLC (Method B) r.t. 1.24, mass found=430.09.
5-amino-3-pyridin-3-yl-N-[3-(trifluoromethyl)benzyl]isoxazole-4-carboxamide. HPLC (Method B) r.t. 1.08, mass found=362.1.
5-amino-N-(3-phenylpropyl)-3-pyridin-3-ylisoxazole-4-carboxamide. HPLC (Method B) r.t. 1.03, mass found=322.14.
5-amino-N-isobutyl-3-pyridin-3-ylisoxazole-4-carboxamide. HPLC (Method B) r.t. 0.79, mass found=260.13.
5-amino-3-pyridin-3-yl-N-[2-(trifluoromethyl)benzyl]isoxazole-4-carboxamide. HPLC (Method B) r.t. 1.05, mass found=362.1.
5-amino-N-(2-methoxybenzyl)-3-pyridin-3-ylisoxazole-4-carboxamide. HPLC (Method B) r.t. 0.92, mass found=324.12.
5-amino-N-(2-fluorobenzyl)-3-pyridin-3-ylisoxazole-4-carboxamide. HPLC (Method B) r.t. 0.89, mass found=312.1.
5-amino-3-pyridin-3-yl-N-[4-(trifluoromethyl)benzyl]isoxazole-4-carboxamide. HPLC (Method B) r.t. 1.1, mass found=362.1.
5-amino-N-(3,4-difluorobenzyl)-3-pyridin-3-ylisoxazole-4-carboxamide. HPLC (Method B) r.t. 0.97, mass found=330.09.
5-amino-N-(3-chlorobenzyl)-3-pyridin-3-ylisoxazole-4-carboxamide. HPLC (Method B) r.t. 1, mass found=328.07.
5-amino-N-(2-chlorobenzyl)-3-pyridin-3-ylisoxazole-4-carboxamide. HPLC (Method B) r.t. 0.97, mass found=328.07.
5-amino-N-(3-methylbenzyl)-3-pyridin-3-ylisoxazole-4-carboxamide. HPLC (Method B) r.t. 0.97, mass found=308.13.
5-amino-N-(1-naphthylmethyl)-3-pyridin-3-ylisoxazole-4-carboxamide. HPLC (Method B) r.t. 1.04, mass found=344.13.
5-amino-N-(cyclopropylmethyl)-3-pyridin-3-ylisoxazole-4-carboxamide. HPLC (Method B) r.t. 0.72, mass found=258.11.
5-amino-N-cycloheptyl-3-pyridin-3-ylisoxazole-4-carboxamide. HPLC (Method B) r.t. 0.98, mass found=300.16.
5-amino-N-[2-(4-methoxyphenyl)ethyl]-3-pyridin-3-ylisoxazole-4-carboxamide. HPLC (Method B) r.t. 0.94, mass found=338.14.
5-amino-N-(2-cyclohex-1-en-1-ylethyl)-3-pyridin-3-ylisoxazole-4-carboxamide. HPLC (Method B) r.t. 1.07, mass found=312.16.
5-amino-N-(1,2-dimethylpropyl)-3-pyridin-3-ylisoxazole-4-carboxamide. HPLC (Method B) r.t. 0.86, mass found=274.14.
5-amino-N-(3,3-diphenylpropyl)-3-pyridin-3-ylisoxazole-4-carboxamide. HPLC (Method B) r.t. 1.23, mass found=398.17.
5-amino-N-(1,2-diphenylethyl)-3-pyridin-3-ylisoxazole-4-carboxamide. HPLC (Method B) r.t. 1.19, mass found=384.16.
5-amino-N-(2,4-dimethoxybenzyl)-3-pyridin-3-ylisoxazole-4-carboxamide. HPLC (Method B) r.t. 0.95, mass found=354.13.
5-amino-N-[3-(dibutylamino)propyl]-3-pyridin-3-ylisoxazole-4-carboxamide. HPLC (Method B) r.t. 0.86, mass found=373.25.
5-amino-N-butyl-3-pyridin-3-ylisoxazole-4-carboxamide. HPLC (Method B) r.t. 0.79, mass found=260.13.
5-amino-3-pyridin-3-yl-N-[4-(trifluoromethoxy)benzyl]isoxazole-4-carboxamide. HPLC (Method B) r.t. 1.13, mass found=378.09.
5-amino-N-[(5-methyl-2-furyl)methyl]-3-pyridin-3-ylisoxazole-4-carboxamide. HPLC (Method B) r.t. 0.84, mass found=298.11.
5-amino-N-(1-methylhexyl)-3-pyridin-3-ylisoxazole-4-carboxamide. HPLC (Method B) r.t. 1.1, mass found=302.17.
5-amino-N-(1-methylbutyl)-3-pyridin-3-ylisoxazole-4-carboxamide. HPLC (Method B) r.t. 0.87, mass found=274.14.
5-amino-N-(3-fluorobenzyl)-3-pyridin-3-ylisoxazole-4-carboxamide. HPLC (Method B) r.t. 0.92, mass found=312.1.
5-amino-N-(4-methylbenzyl)-3-pyridin-3-ylisoxazole-4-carboxamide. HPLC (Method B) r.t. 0.97, mass found=308.13.
5-amino-N-(1,3-dimethylbutyl)-3-pyridin-3-ylisoxazole-4-carboxamide. HPLC (Method B) r.t. 0.97, mass found=288.16.
5-amino-N-(1-methyl-3-phenylpropyl)-3-pyridin-3-ylisoxazole-4-carboxamide. HPLC (Method B) r.t. 1.09, mass found=336.16.
5-amino-N-[3-(methylthio)propyl]-3-pyridin-3-ylisoxazole-4-carboxamide. HPLC (Method B) r.t. 0.79, mass found=292.1.
5-amino-N-(1,3-benzodioxol-5-ylmethyl)-3-pyridin-3-ylisoxazole-4-carboxamide. HPLC (Method B) r.t. 0.9, mass found=338.1.
5-amino-N-pentyl-3-pyridin-3-ylisoxazole-4-carboxamide. HPLC (Method B) r.t. 0.92, mass found=274.14.
5-amino-N-(cyclohexylmethyl)-3-pyridin-3-ylisoxazole-4-carboxamide. HPLC (Method B) r.t. 1.02, mass found=300.16.
ethyl 4-{[(5-amino-3-pyridin-3-ylisoxazol-4-yl)carbonyl]amino}piperidine-1-carboxylate. HPLC (Method B) r.t. 0.84, mass found=359.16.
5-amino-N-[2-(3-chlorophenyl)ethyl]-3-pyridin-3-ylisoxazole-4-carboxamide. HPLC (Method B) r.t. 1.05, mass found=342.09.
5-amino-N-(1-ethylpropyl)-3-pyridin-3-ylisoxazole-4-carboxamide. HPLC (Method B) r.t. 0.86, mass found=274.14.
5-amino-N-(1-phenylethyl)-3-pyridin-3-ylisoxazole-4-carboxamide. HPLC (Method B) r.t. 0.94, mass found=308.13.
5-amino-N-(2-methylbenzyl)-3-pyridin-3-ylisoxazole-4-carboxamide. HPLC (Method B) r.t. 0.95, mass found=308.13.
5-amino-N-cyclobutyl-3-pyridin-3-ylisoxazole-4-carboxamide. HPLC (Method B) r.t. 0.72, mass found=258.11.
5-amino-N-benzhydryl-3-pyridin-3-ylisoxazole-4-carboxamide. HPLC (Method B) r.t. 1.15, mass found=370.14.
5-amino-N-octyl-3-pyridin-3-ylisoxazole-4-carboxamide. HPLC (Method B) r.t. 1.24, mass found=316.19.
5-amino-N-(2-bromobenzyl)-3-pyridin-3-ylisoxazole-4-carboxamide. HPLC (Method B) r.t. 0.99, mass found=372.02.
5-amino-N-(1-methylheptyl)-3-pyridin-3-ylisoxazole-4-carboxamide. HPLC (Method B) r.t. 1.21, mass found=316.19.
5-amino-N-heptyl-3-pyridin-3-ylisoxazole-4-carboxamide. HPLC (Method B) r.t. 1.14, mass found=302.17.
5-amino-N-(tert-butyl)-3-pyridin-3-ylisoxazole-4-carboxamide. HPLC (Method B) r.t. 0.8, mass found=260.13.
5-amino-N-(3,4-dimethoxybenzyl)-3-pyridin-3-ylisoxazole-4-carboxamide. HPLC (Method B) r.t. 0.84, mass found=354.13.
5-amino-N-(2,3-dihydro-1H-inden-1-yl)-3-pyridin-3-ylisoxazole-4-carboxamide. HPLC (Method B) r.t. 0.98, mass found=320.13.
5-amino-3-pyridin-3-yl-N-(1,2,3,4-tetrahydronaphthalen-1-yl)isoxazole-4-carboxamide. HPLC (Method B) r.t. 1.04, mass found=334.14.

### EXAMPLE 4

4-(3-Methoxy-4-phenylaminomethyl-phenoxy)-butyryl AM Resin

4-(4-Formyl-3-methoxyphenoxy)butyryl AM resin [copoly(styrene-1% dvb) 100-200 mesh] (1,5 g, 1 eq, loading 0,94 mmol/g) is swollen in DCM and then filtered. A mixture of THF/DCM (4:1, 15 ml), aniline (6 eq.) and AcOH (6 eq.) were added. After 15 min NaBH(OAc)₃ (3 eq) was added and the reaction was shaken over night at room temperature. After filtration, the resin was washed with methanol (×3), DMF/DCM (1:1) (×3) and DCM (×5).

### EXAMPLE 5

4-(4-{[(2-Cyano-acetyl)-phenyl-amino]-methyl}-3-methoxy-phenoxy)-butyryl AM Resin

Diisopropyl carbodimide (1.1 ml, 7.08 mmol) was added to a suspension of cyanoacetic acid (1.34 g, 15.79 mmol) in DCM (18.9 ml) at 0° C., the resulting solution was stirred for 30 min, then was added to 1.4 g 4-(3-Methoxy-4-phenylaminomethyl-phenoxy)-butyryl AM resin (1.32 mmol). The suspension was shaken for 16 hours, then filtered and washed with dichloromethane (2×), dimethylformamide (2×) and dichloromethane (3×). A second cycle with the same procedure was performed for 16 h, then the solution was drained and the resin washed with dichloromethane (2×), dimethylformamide (2×), methanol (2×), dichloromethane (2×), dimethylformamide (2×), dichloromethane (5×) and dried in vacuum overnight.

### EXAMPLE 6

5-Amino-3-pyridin-2-yl-isoxazole-4-carboxylic Acid 4-(3-methoxy-4-phenylaminomethyl-phenoxy)-butyryl AM Resin Amide

4-(4-{[(2-Cyano-acetyl)-phenyl-amino]-methyl}-3-methoxy-phenoxy)-butyryl AM resin (0.75 g, 0.443 mmol) and pyridine-2-hydroxyaminomethyl chloride (208 mg, 1.328 mmol) were suspended in dry THF (8 ml) and cooled to −20° C. Then lithium bis(trimethylsilyl)amide, LiHMDS, (2.2 ml of 1 M solution in THF, 2.2 mmol) was added, the resulting suspension was stirred at −20° C. for 15 min, allowed to warm to room temperature (23° C.) and shaken for 2 h and drained. The resin was washed with dimethylformamide, dichloromethane, dimethylformamide (2×) and dichloromethane (3×) and dried under vacuum.

By working in an analogous way starting from cyanoacetic acid Rink amide the following compounds were prepared:
5-Amino-3-thiophen-3-yl-isoxazole-4-carboxylic acid Rink amide.
5-Amino-3-thiophen-2-yl-isoxazole-4-carboxylic acid Rink amide.
5-Amino-3-pyridin-2-yl-isoxazole-4-carboxylic acid Rink amide.
5-Amino-3-pyridin-3-yl-isoxazole-4-carboxylic acid Rink amide).
5-Amino-3-pyridin-4-yl-isoxazole-4-carboxylic acid Rink amide).
5-Amino-3-thiazol-2-yl-isoxazole-4-carboxylic acid Rink amide.
5-Amino-3-(3,5-dimethyl-1-phenyl-1H-pyrazol-4-yl)-isoxazole-4-carboxylic acid Rink amide.
5-Amino-3-quinolin-2-yl-isoxazole-4-carboxylic acid Rink amide.

### EXAMPLE 7

5-Amino-3-pyridin-2-yl-isoxazole-4-carboxylic Acid Phenylamide

5-Amino-3-pyridin-2-yl-isoxazole-4-carboxylic acid 4-(3-methoxy-4-phenylaminomethyl-phenoxy)-butyryl AM resin amide (0.75 mmol) was treated with a solution of TFA 20% in anhydrous dichloromethane (10 ml). The resulting suspension was gently stirred or shaken at 22° C. for 1 h, then washed with anhydrous dichloromethane, methanol, dichloromethane and dried under vacuum to give a crude solid, that after purification by flash chromatography gave 120 mg of the desired compound.

Analogously, by using the appropriate chloroxime derivative and the appropriate cyanacetamide derivative supported on resin, the following compounds were prepared after purification by flash chromatography or preparative HPLC, when necessary:
5-Amino-3-(3,5-dimethyl-1-phenyl-1H-pyrazol-4-yl)-isoxazole-4-carboxylic acid (9H-fluoren-2-yl)-amide.
5-Amino-3-(3,5-dimethyl-1-phenyl-1H-pyrazol-4-yl)-isoxazole-4-carboxylic acid (pyridin-4-ylmethyl)-amide.
5-Amino-3-(3,5-dimethyl-1-phenyl-1H-pyrazol-4-yl)-isoxazole-4-carboxylic acid (3-methoxy-phenyl)-amide.
5-Amino-3-thiazol-2-yl-isoxazole-4-carboxylic acid (2,3-dihydro-benzo[1,4]dioxin-6-yl)-amide.
5-Amino-3-(3,5-dimethyl-1-phenyl-1H-pyrazol-4-yl)-isoxazole-4-carboxylic acid (2,3-dihydro-benzo[1,4]dioxin-6-yl)-amide.
5-Amino-3-pyridin-3-yl-isoxazole-4-carboxylic acid (9H-fluoren-2-yl)-amide.
5-Amino-3-thiophen-2-yl-isoxazole-4-carboxylic acid (9H-fluoren-2-yl)-amide.
5-Amino-3-thiophen-2-yl-isoxazole-4-carboxylic acid (4-morpholin-4-yl-phenyl)-amide.
5-Amino-3-thiazol-2-yl-isoxazole-4-carboxylic acid (4-morpholin-4-yl-phenyl)-amide.
5-Amino-3-(3,5-dimethyl-1-phenyl-1H-pyrazol-4-yl)-isoxazole-4-carboxylic acid (4-morpholin-4-yl-phenyl)-amide.
5-Amino-3-pyridin-2-yl-isoxazole-4-carboxylic acid (pyridin-4-ylmethyl)-amide.
5-Amino-3-thiophen-2-yl-isoxazole-4-carboxylic acid phenylamide.
5-Amino-3-thiazol-2-yl-isoxazole-4-carboxylic acid phenylamide.
5-Amino-3-(3,5-dimethyl-1-phenyl-1H-pyrazol-4-yl)-isoxazole-4-carboxylic acid (3-methoxy-propyl)-amide.
5-Amino-3-pyridin-4-yl-isoxazole-4-carboxylic acid (4-morpholin-4-yl-phenyl)-amide.
5-Amino-3-pyridin-2-yl-isoxazole-4-carboxylic acid phenylamide.
5-Amino-3-thiazol-2-yl-isoxazole-4-carboxylic acid (3-methoxy-phenyl)-amide.
5-Amino-3-(3,5-dimethyl-1-phenyl-1H-pyrazol-4-yl)-isoxazole-4-carboxylic acid (3-phenoxy-phenyl)-amide.
5-Amino-3-thiophen-3-yl-isoxazole-4-carboxylic acid (4-morpholin-4-yl-phenyl)-amide.
4-{[5-Amino-3-(3,5-dimethyl-1-phenyl-1H-pyrazol-4-yl)-isoxazole-4-carbonyl]-amino}-piperidine-1-carboxylic acid ethyl ester.
5-Amino-3-(3,5-dimethyl-1-phenyl-1H-pyrazol-4-yl)-isoxazole-4-carboxylic acid (tetrahydro-furan-2-ylmethyl)-amide.
5-Amino-3-thiophen-3-yl-isoxazole-4-carboxylic acid phenylamide.
5-Amino-3-thiophen-3-yl-isoxazole-4-carboxylic acid 2-methoxy-benzylamide.
5-Amino-3-pyridin-4-yl-isoxazole-4-carboxylic acid 4-chloro-benzylamide.
5-Amino-3-pyridin-2-yl-isoxazole-4-carboxylic acid 2-methoxy-benzylamide.
5-Amino-3-thiophen-2-yl-isoxazole-4-carboxylic acid 4-fluoro-benzylamide.
5-Amino-3-pyridin-4-yl-isoxazole-4-carboxylic acid (3,4-dimethyl-phenyl)-amide.
5-Amino-3-(3,5-dimethyl-1-phenyl-1H-pyrazol-4-yl)-isoxazole-4-carboxylic acid (2-dimethylamino-ethyl)-amide.
5-Amino-3-thiophen-3-yl-isoxazole-4-carboxylic acid (9H-fluoren-2-yl)-amide.
5-Amino-3-thiazol-2-yl-isoxazole-4-carboxylic acid (9H-fluoren-2-yl)-amide.
5-Amino-3-thiophen-3-yl-isoxazole-4-carboxylic acid (3-methoxy-propyl)-amide.
5-Amino-3-pyridin-4-yl-isoxazole-4-carboxylic acid (4-diethylamino-phenyl)-amide.
5-Amino-3-pyridin-2-yl-isoxazole-4-carboxylic acid (4-diethylamino-phenyl)-amide.
5-Amino-3-(3,5-dimethyl-1-phenyl-1H-pyrazol-4-yl)-isoxazole-4-carboxylic acid (4-diethylamino-phenyl)-amide.
5-Amino-3-pyridin-2-yl-isoxazole-4-carboxylic acid (4-butoxy-phenyl)-amide.
5-Amino-3-(3,5-dimethyl-1-phenyl-1H-pyrazol-4-yl)-isoxazole-4-carboxylic acid (4-butoxy-phenyl)-amide.
5-Amino-3-(3,5-dimethyl-1-phenyl-1H-pyrazol-4-yl)-isoxazole-4-carboxylic acid (1-ethyl-propyl)-amide.
5-Amino-3-thiophen-3-yl-isoxazole-4-carboxylic acid (3,4-dimethyl-phenyl)-amide.
5-Amino-3-thiophen-3-yl-isoxazole-4-carboxylic acid (4-fluoro-2-methyl-phenyl)-amide.
5-Amino-3-thiazol-2-yl-isoxazole-4-carboxylic acid [4-(acetyl-methyl-amino)-phenyl]-amide.
5-Amino-3-thiophen-2-yl-isoxazole-4-carboxylic acid 2-methoxy-benzylamide.
5-Amino-3-pyridin-4-yl-isoxazole-4-carboxylic acid [2-(4-methoxy-phenyl)-ethyl]-amide.
5-Amino-3-pyridin-2-yl-isoxazole-4-carboxylic acid (3-methoxy-propyl)-amide.
5-Amino-3-pyridin-3-yl-isoxazole-4-carboxylic acid 4-chloro-benzylamide.
5-Amino-3-(3,5-dimethyl-1-phenyl-1H-pyrazol-4-yl)-isoxazole-4-carboxylic acid [2-(4-methoxy-phenyl)-ethyl]-amide.
5-Amino-3-thiophen-3-yl-isoxazole-4-carboxylic acid (3-phenoxy-phenyl)-amide.
5-Amino-3-thiazol-2-yl-isoxazole-4-carboxylic acid (pyridin-4-ylmethyl)-amide.
5-Amino-3-thiophen-2-yl-isoxazole-4-carboxylic acid (2,3-dihydro-benzo[1,4]dioxin-6-yl)-amide.
5-Amino-3-pyridin-3-yl-isoxazole-4-carboxylic acid phenylamide.
5-Amino-3-thiazol-2-yl-isoxazole-4-carboxylic acid (2-methylsulfanyl-phenyl)-amide.
5-Amino-3-thiophen-2-yl-isoxazole-4-carboxylic acid (3-chloro-4-methyl-phenyl)-amide.
5-Amino-3-thiophen-3-yl-isoxazole-4-carboxylic acid (3-chloro-4-methyl-phenyl)-amide.
5-Amino-3-thiophen-2-yl-isoxazole-4-carboxylic acid (3-methoxy-phenyl)-amide.
5-Amino-3-pyridin-2-yl-isoxazole-4-carboxylic acid (3-methoxy-phenyl)-amide.
5-Amino-3-pyridin-2-yl-isoxazole-4-carboxylic acid (3-trifluoromethyl-phenyl)-amide.
5-Amino-3-thiazol-2-yl-isoxazole-4-carboxylic acid (3-methoxy-propyl)-amide.
5-Amino-3-(3,5-dimethyl-1-phenyl-1H-pyrazol-4-yl)-isoxazole-4-carboxylic acid cyclopropylamide.
5-Amino-3-thiophen-3-yl-isoxazole-4-carboxylic acid (3-trifluoromethyl-phenyl)-amide.
4-[(5-Amino-3-thiophen-3-yl-isoxazole-4-carbonyl)-amino]-piperidine-1-carboxylic acid ethyl ester.
4-[(5-Amino-3-pyridin-2-yl-isoxazole-4-carbonyl)-amino]-piperidine-1-carboxylic acid ethyl ester.
5-Amino-3-thiophen-3-yl-isoxazole-4-carboxylic acid (2,3-dihydro-benzo[1,4]dioxin-6-yl)-amide.
5-Amino-3-thiophen-2-yl-isoxazole-4-carboxylic acid [2-(4-methoxy-phenyl)-ethyl]-amide.
5-Amino-3-thiophen-2-yl-isoxazole-4-carboxylic acid (furan-2-ylmethyl)-amide.
5-Amino-3-thiophen-3-yl-isoxazole-4-carboxylic acid (furan-2-ylmethyl)-amide.
5-Amino-3-(3,5-dimethyl-1-phenyl-1H-pyrazol-4-yl)-isoxazole-4-carboxylic acid (furan-2-ylmethyl)-amide.
5-Amino-3-thiophen-3-yl-isoxazole-4-carboxylic acid (4-diethylamino-phenyl)-amide.
5-Amino-3-thiazol-2-yl-isoxazole-4-carboxylic acid (4-diethylamino-phenyl)-amide.
5-Amino-3-pyridin-4-yl-isoxazole-4-carboxylic acid (9H-fluoren-2-yl)-amide.
5-Amino-3-pyridin-2-yl-isoxazole-4-carboxylic acid (9H-fluoren-2-yl)-amide.
5-Amino-3-pyridin-4-yl-isoxazole-4-carboxylic acid (3-trifluoromethyl-phenyl)-amide.
5-Amino-3-pyridin-4-yl-isoxazole-4-carboxylic acid 4-fluoro-benzylamide.
5-Amino-3-thiophen-3-yl-isoxazole-4-carboxylic acid (4-butoxy-phenyl)-amide.
5-Amino-3-thiophen-3-yl-isoxazole-4-carboxylic acid (4-difluoromethoxy-phenyl)-amide.
5-Amino-3-thiazol-2-yl-isoxazole-4-carboxylic acid [4-(toluene-4-sulfonylamino)-phenyl]-amide.
5-Amino-3-thiophen-3-yl-isoxazole-4-carboxylic acid (2-methylsulfanyl-phenyl)-amide.
5-Amino-3-(3,5-dimethyl-1-phenyl-1H-pyrazol-4-yl)-isoxazole-4-carboxylic acid phenylamide.
5-Amino-3-thiophen-3-yl-isoxazole-4-carboxylic acid (1-ethyl-propyl)-amide.
5-Amino-3-pyridin-4-yl-isoxazole-4-carboxylic acid (3-methoxy-phenyl)-amide.
5-Amino-3-thiophen-3-yl-isoxazole-4-carboxylic acid [4-(toluene-4-sulfonylamino)-phenyl]-amide.
5-Amino-3-thiophen-2-yl-isoxazole-4-carboxylic acid (2-pyrrolidin-1-yl-ethyl)-amide.
5-Amino-3-(3,5-dimethyl-1-phenyl-1H-pyrazol-4-yl)-isoxazole-4-carboxylic acid (3-chloro-4-methyl-phenyl)-amide.
5-Amino-3-thiophen-2-yl-isoxazole-4-carboxylic acid (4-diethylamino-phenyl)-amide.
5-Amino-3-pyridin-2-yl-isoxazole-4-carboxylic acid (2-dimethylamino-ethyl)-amide.
5-Amino-3-thiazol-2-yl-isoxazole-4-carboxylic acid (3-trifluoromethyl-phenyl)-amide.
5-Amino-3-thiophen-2-yl-isoxazole-4-carboxylic acid (pyridin-4-ylmethyl)-amide.
5-Amino-3-pyridin-2-yl-isoxazole-4-carboxylic acid (4-morpholin-4-yl-phenyl)-amide.
5-Amino-3-thiophen-3-yl-isoxazole-4-carboxylic acid (3-methoxy-phenyl)-amide.
5-Amino-3-pyridin-4-yl-isoxazole-4-carboxylic acid (4-butoxy-phenyl)-amide.

### EXAMPLE 8

5-Benzoylamino-3-pyridin-4-yl-isoxazole-4-carboxylic Acid Amide

To a suspension of 5-amino-3-pyridin-4-yl-isoxazole-4-carboxylic acid amide (25 mg, 0.123 mmol) in dichloromethane (0.25 ml), triethyl amine (0.051 ml, 0.368 mmol), 4-dimethyl aminopyridine (3 mg, 0.025 mmol) and benzoyl chloride (0.021 ml, 0.184 mmol) were added. The final mixture was stirred at room temperature (23° C.) for 16 hours, afterward volatiles were removed under reduced pressure to give a crude solid, that was purified by flash chromatography (yield 62%). HPLC r.t. 3.24, [M+H]+=309. ¹H-NMR (DMSO-d₆), diagnostic signals (ppm): 11,5 (bs, 1H), 8,7 (d, 2H), 8 (m, 2H) 7,7(m, 2H), 7,6 (m, 1H), 7,52(bt, 2H).

By working in an analogous way and by using the appropriate isoxazole derivative and the appropriate acyl chloride derivative, the following compounds were prepared after purification by flash chromatography or preparative HPLC when necessary:
5-Benzoylamino-3-thiophen-3-yl-isoxazole-4-carboxylic acid amide. HPLC r.t. 4.52, [M+H]+=314. ¹H-NMR (DMSO-d₆), diagnostic signals (ppm): 9,05 (bs, 1H), 8,2 (bs, 2H), 8 (d, 2H) 7,6 (m, 2H), 7,5 (m, 2H), 7,22(m, 2H).
5-(4-Methyl-benzoylamino)-3-pyridin-2-yl-isoxazole-4-carboxylic acid amide. HPLC r.t. 5.85, [M+H]+=323.
5-(4-Chloro-butyrylamino)-3-pyridin-2-yl-isoxazole-4-carboxylic acid amide. HPLC r.t. 4.64, [M+H]+=309.
5-(4-tert-Butyl-benzoylamino)-3-pyridin-2-yl-isoxazole-4-carboxylic acid amide. HPLC r.t. 7.08, [M+H]+=365.
5-Pentanoylamino-3-pyridin-2-yl-isoxazole-4-carboxylic acid amide. HPLC r.t. 4.95, [M+H]+=289.
5-(4-Methyl-benzoylamino)-3-pyridin-4-yl-isoxazole-4-carboxylic acid amide. HPLC r.t. 3.81, [M+H]+=323.
5-(4-Chloro-benzoylamino)-3-pyridin-4-yl-isoxazole-4-carboxylic acid amide. HPLC r.t. 3.84, [M+H]+=343.
5-Pentanoylamino-3-pyridin-4-yl-isoxazole-4-carboxylic acid amide. HPLC r.t. 2.96, [M+H]+=289.
5-(3-Phenyl-propionylamino)-3-pyridin-4-yl-isoxazole-4-carboxylic acid amide. HPLC r.t. 3.74, [M+H]+=337.
5-(4-tert-Butyl-benzoylamino)-3-thiophen-2-yl-isoxazole-4-carboxylic acid amide. HPLC r.t. 6.34, [M+H]+=370.
5-Pentanoylamino-3-thiophen-2-yl-isoxazole-4-carboxylic acid amide. HPLC r.t. 4.18, [M+H]+=294.
5-(3-Phenyl-propionylamino)-3-thiophen-2-yl-isoxazole-4-carboxylic acid amide. HPLC r.t. 4.89, [M+H]+=342.
5-(4-Methyl-benzoylamino)-3-thiophen-3-yl-isoxazole-4-carboxylic acid amide. HPLC r.t. 5.07, [M+H]+=328.
5-(4-tert-Butyl-benzoylamino)-3-thiophen-3-yl-isoxazole-4-carboxylic acid amide. HPLC r.t. 6.33, [M+H]+=370.
5-Pentanoylamino-3-thiophen-3-yl-isoxazole-4-carboxylic acid amide. HPLC r.t. 4.12, [M+H]+=294.
5-(3-Phenyl-propionylamino)-3-thiophen-3-yl-isoxazole-4-carboxylic acid amide. HPLC r.t. 4.83, [M+H]+=342.
5-(4-Chloro-butyrylamino)-3-thiazol-2-yl-isoxazole-4-carboxylic acid amide. HPLC r.t. 4.83, [M+H]+=315.
5-(4-tert-Butyl-benzoylamino)-3-thiazol-2-yl-isoxazole-4-carboxylic acid amide. HPLC r.t. 7.25, [M+H]+=371.
5-(4-Chloro-benzoylamino)-3-thiazol-2-yl-isoxazole-4-carboxylic acid amide. HPLC r.t. 6.3, [M+H]+=349.
5-(3-Phenyl-propionylamino)-3-thiazol-2-yl-isoxazole-4-carboxylic acid amide. HPLC r.t. 5.69, [M+H]+=343.
5-(4-Methyl-benzoylamino)-3-pyridin-3-yl-isoxazole-4-carboxylic acid amide. HPLC r.t. 3.76, [M+H]+=323.
5-(4-tert-Butyl-benzoylamino)-3-pyridin-3-yl-isoxazole-4-carboxylic acid amide. HPLC r.t. 5.04, [M+H]+=365.
5-(4-Chloro-benzoylamino)-3-pyridin-3-yl-isoxazole-4-carboxylic acid amide. HPLC r.t. 3.81, [M+H]+=343.
5-Pentanoylamino-3-pyridin-3-yl-isoxazole-4-carboxylic acid amide. HPLC r.t. 2.92, [M+H]+=289.
5-(4-tert-Butyl-benzoylamino)-3-(3,5-dimethyl-1-phenyl-1H-pyrazol-4-yl)-isoxazole-4-carboxylic acid amide. HPLC r.t. 6.79, [M+H]+=458.
5-(4-Chloro-benzoylamino)-3-(3,5-dimethyl-1-phenyl-1H-pyrazol-4-yl)-isoxazole-4-carboxylic acid amide. HPLC r.t. 5.9, [M+H]+=436.
3-(3,5-Dimethyl-1-phenyl-1H-pyrazol-4-yl)-5-pentanoylamino-isoxazole-4-carboxylic acid amide. HPLC r.t. 5, [M+H]+=382.
3-(3,5-Dimethyl-1-phenyl-1H-pyrazol-4-yl)-5-(3-phenyl-propionylamino)-isoxazole-4-carboxylic acid amide. HPLC r.t. 5.5, [M+H]+=430.
5-(4-Chloro-butyrylamino)-3-quinolin-2-yl-isoxazole-4-carboxylic acid amide. HPLC r.t. 6.14, [M+H]+=359.
5-Pentanoylamino-3-quinolin-2-yl-isoxazole-4-carboxylic acid amide. HPLC r.t. 6.47, [M+H]+=339.
5-(3-Phenyl-propionylamino)-3-quinolin-2-yl-isoxazole-4-carboxylic acid amide. HPLC r.t. 6.84, [M+H]+=387.
5-(3-Fluoro-benzoylamino)-3-pyridin-2-yl-isoxazole-4-carboxylic acid amide. HPLC r.t. 5.65, [M+H]+=327.
5-(3-Methoxy-benzoylamino)-3-pyridin-2-yl-isoxazole-4-carboxylic acid amide. HPLC r.t. 5.59, [M+H]+=339.
3-Pyridin-2-yl-5-(4-trifluoromethyl-benzoylamino)-isoxazole-4-carboxylic acid amide. HPLC r.t. 5.63, [M+H]+=377.
5-(3-Fluoro-benzoylamino)-3-pyridin-4-yl-isoxazole-4-carboxylic acid amide. HPLC r.t. 3.33, [M+H]+=327.
5-(2-Methoxy-acetylamino)-3-pyridin-4-yl-isoxazole-4-carboxylic acid amide. HPLC r.t. 1.76, [M+H]+=277.
5-(3-Methoxy-benzoylamino)-3-pyridin-4-yl-isoxazole-4-carboxylic acid amide. HPLC r.t. 3.49, [M+H]+=339.
3-Pyridin-4-yl-5-(4-trifluoromethyl-benzoylamino)-isoxazole-4-carboxylic acid amide. HPLC r.t. 3.4, [M+H]+=377.
5-(3-Bromo-benzoylamino)-3-pyridin-4-yl-isoxazole-4-carboxylic acid amide. HPLC r.t. 3.9, [M+H]+=387.
5-(3-Fluoro-benzoylamino)-3-thiophen-2-yl-isoxazole-4-carboxylic acid amide. HPLC r.t. 4.64, [M+H]+=332.
5-(2-Methoxy-acetylamino)-3-thiophen-2-yl-isoxazole-4-carboxylic acid amide. HPLC r.t. 2.85, [M+H]+=282.
5-Benzoylamino-3-thiophen-2-yl-isoxazole-4-carboxylic acid amide. HPLC r.t. 4.52, [M+H]+=314.
5-(3-Methoxy-benzoylamino)-3-thiophen-2-yl-isoxazole-4-carboxylic acid amide. HPLC r.t. 4.77, [M+H]+=344.
3-Thiophen-2-yl-5-(4-trifluoromethyl-benzoylamino)-isoxazole-4-carboxylic acid amide. HPLC r.t. 4.64, [M+H]+=382.
5-(2-Methoxy-acetylamino)-3-thiophen-3-yl-isoxazole-4-carboxylic acid amide. HPLC r.t. 2.79, [M+H]+=282.
5-(3-Methoxy-benzoylamino)-3-thiophen-3-yl-isoxazole-4-carboxylic acid amide. HPLC r.t. 4.78, [M+H]+=344.
3-Thiophen-3-yl-5-(4-trifluoromethyl-benzoylamino)-isoxazole-4-carboxylic acid amide. HPLC r.t. 3.7, [M+H]+=382.
5-(2-Methoxy-acetylamino)-3-thiazol-2-yl-isoxazole-4-carboxylic acid amide. HPLC r.t. 3.56, [M+H]+=283.
5-Benzoylamino-3-thiazol-2-yl-isoxazole-4-carboxylic acid amide. HPLC r.t. 5.52, [M+H]+=315.
5-(3-Methoxy-benzoylamino)-3-thiazol-2-yl-isoxazole-4-carboxylic acid amide. HPLC r.t. 5.76, [M+H]+=345.
3-Thiazol-2-yl-5-(4-trifluoromethyl-benzoylamino)-isoxazole-4-carboxylic acid amide. HPLC r.t. 5.77, [M+H]+=383.
5-(3-Bromo-benzoylamino)-3-thiazol-2-yl-isoxazole-4-carboxylic acid amide. HPLC r.t. 6.5, [M+H]+=393.
5-(2-Methoxy-acetylamino)-3-pyridin-3-yl-isoxazole-4-carboxylic acid amide. HPLC r.t. 1.71, [M+H]+=277.
5-(3-Methoxy-benzoylamino)-3-pyridin-3-yl-isoxazole-4-carboxylic acid amide. HPLC r.t. 3.47, [M+H]+=339.
3-Pyridin-3-yl-5-(4-trifluoromethyl-benzoylamino)-isoxazole-4-carboxylic acid amide. HPLC r.t. 3.36, [M+H]+=377.
5-(3-Bromo-benzoylamino)-3-pyridin-3-yl-isoxazole-4-carboxylic acid amide. HPLC r.t. 3.85, [M+H]+=387.
3-(3,5-Dimethyl-1-phenyl-1H-pyrazol-4-yl)-5-(3-fluoro-benzoylamino)-isoxazole-4-carboxylic acid amide. HPLC r.t. 5.5, [M+H]+=420.
3-(3,5-Dimethyl-1-phenyl-1H-pyrazol-4-yl)-5-(2-methoxy-acetylamino)-isoxazole-4-carboxylic acid amide. HPLC r.t. 3.88, [M+H]+=370.
5-(3-Bromo-benzoylamino)-3-(3,5-dimethyl-1-phenyl-1H-pyrazol-4-yl)-isoxazole-4-carboxylic acid amide. HPLC r.t. 5.92, [M+H]+=480.
3-Quinolin-2-yl-5-(4-trifluoromethyl-benzoylamino)-isoxazole-4-carboxylic acid amide. HPLC r.t. 6.93, [M+H]+=427.
5-(Cyclopropanecarbonyl-amino)-3-pyridin-2-yl-isoxazole-4-carboxylic acid amide. HPLC r.t. 3.89, [M+H]+=273.
5-(2-Phenoxy-acetylamino)-3-pyridin-4-yl-isoxazole-4-carboxylic acid amide. HPLC r.t. 3.6, [M+H]+=339.
5-(2-Chloro-benzoylamino)-3-pyridin-4-yl-isoxazole-4-carboxylic acid amide. HPLC r.t. 3.19, [M+H]+=343.
5-(Cyclopropanecarbonyl-amino)-3-pyridin-4-yl-isoxazole-4-carboxylic acid amide. HPLC r.t. 2, [M+H]+=273.
5-(3-Methyl-but-2-enoylamino)-3-pyridin-4-yl-isoxazole-4-carboxylic acid amide. HPLC r.t. 2.81, [M+H]+=287.
5-(2,4-Difluoro-benzoylamino)-3-pyridin-4-yl-isoxazole-4-carboxylic acid amide. HPLC r.t. 3.51, [M+H]+=345.
5-[2-(3-Methoxy-phenyl)-acetylamino]-3-thiophen-2-yl-isoxazole-4-carboxylic acid amide. HPLC r.t. 4.53, [M+H]+=358.
5-(2-Phenoxy-acetylamino)-3-thiophen-2-yl-isoxazole-4-carboxylic acid amide. HPLC r.t. 4.87, [M+H]+=344.
5-(2-Chloro-benzoylamino)-3-thiophen-2-yl-isoxazole-4-carboxylic acid amide. HPLC r.t. 4.51, [M+H]+=348.
5-(Cyclopropanecarbonyl-amino)-3-thiophen-2-yl-isoxazole-4-carboxylic acid amide. HPLC r.t. 3.09, [M+H]+=278.
5-(3-Methyl-but-2-enoylamino)-3-thiophen-2-yl-isoxazole-4-carboxylic acid amide. HPLC r.t. 4.02, [M+H]+=292.
5-(2,4-Difluoro-benzoylamino)-3-thiophen-2-yl-isoxazole-4-carboxylic acid amide. HPLC r.t. 4.85, [M+H]+=350.
5-[2-(3-Methoxy-phenyl)-acetylamino]-3-thiophen-3-yl-isoxazole-4-carboxylic acid amide. HPLC r.t. 4.49, [M+H]+=358.
5-(2-Chloro-benzoylamino)-3-thiophen-3-yl-isoxazole-4-carboxylic acid amide. HPLC r.t. 4.53, [M+H]+=348.
5-(Cyclopropanecarbonyl-amino)-3-thiophen-3-yl-isoxazole-4-carboxylic acid amide. HPLC r.t. 3.03, [M+H]+=278.
5-(3-Methyl-but-2-enoylamino)-3-thiophen-3-yl-isoxazole-4-carboxylic acid amide. HPLC r.t. 3.94, [M+H]+=292.
5-(2,4-Difluoro-benzoylamino)-3-thiophen-3-yl-isoxazole-4-carboxylic acid amide. HPLC r.t. 4.87, [M+H]+=350.
5-(2-Phenoxy-acetylamino)-3-thiazol-2-yl-isoxazole-4-carboxylic acid amide. HPLC r.t. 5.52, [M+H]+=345.
5-(Cyclopropanecarbonyl-amino)-3-thiazol-2-yl-isoxazole-4-carboxylic acid amide. HPLC r.t. 4.1, [M+H]+=277.
5-(3-Methyl-but-2-enoylamino)-3-thiazol-2-yl-isoxazole-4-carboxylic acid amide. HPLC r.t. 4.95, [M+H]+=293.
5-(2,4-Difluoro-benzoylamino)-3-thiazol-2-yl-isoxazole-4-carboxylic acid amide. HPLC r.t. 5.62, [M+H]+=351.
5-(2-Phenoxy-acetylamino)-3-pyridin-3-yl-isoxazole-4-carboxylic acid amide. HPLC r.t. 3.56, [M+H]+=339.
5-(2-Chloro-benzoylamino)-3-pyridin-3-yl-isoxazole-4-carboxylic acid amide. HPLC r.t. 3.16, [M+H]+=343.
5-(Cyclopropanecarbonyl-amino)-3-pyridin-3-yl-isoxazole-4-carboxylic acid amide. HPLC r.t. 1.95, [M+H]+=273.
5-(2,4-Difluoro-benzoylamino)-3-pyridin-3-yl-isoxazole-4-carboxylic acid amide. HPLC r.t. 3.46, [M+H]+=345.
3-(3,5-Dimethyl-1-phenyl-1H-pyrazol-4-yl)-5-(2-phenoxy-acetylamino)-isoxazole-4-carboxylic acid amide. HPLC r.t. 5.45, [M+H]+=432.
5-(2-Chloro-benzoylamino)-3-(3,5-dimethyl-1-phenyl-1H-pyrazol-4-yl)-isoxazole-4-carboxylic acid amide. HPLC r.t. 5.32, [M+H]+=436.
5-(Cyclopropanecarbonyl-amino)-3-(3,5-dimethyl-1-phenyl-1H-pyrazol-4-yl)-isoxazole-4-carboxylic acid amide. HPLC r.t. 4.12, [M+H]+=366.
3-(3,5-Dimethyl-1-phenyl-1H-pyrazol-4-yl)-5-(3-methyl-but-2-enoylamino)-isoxazole-4-carboxylic acid amide. HPLC r.t. 4.81, [M+H]+=380.
5-(2,4-Difluoro-benzoylamino)-3-(3,5-dimethyl-1-phenyl-1H-pyrazol-4-yl)-isoxazole-4-carboxylic acid amide. HPLC r.t. 5.46, [M+H]+=438.
5-(Cyclopropanecarbonyl-amino)-3-quinolin-2-yl-isoxazole-4-carboxylic acid amide. HPLC r.t. 5.6, [M+H]+=323.
5-(4-Fluoro-benzoylamino)-3-pyridin-2-yl-isoxazole-4-carboxylic acid amide. HPLC r.t. 5.56, [M+H]+=327.
3-Pyridin-2-yl-5-(4-trifluoromethoxy-benzoylamino)-isoxazole-4-carboxylic acid amide. HPLC r.t. 6.52, [M+H]+=393.
5-(2-Methyl-benzoylamino)-3-pyridin-2-yl-isoxazole-4-carboxylic acid amide. HPLC r.t. 5.61, [M+H]+=323.
5-(4-Ethyl-benzoylamino)-3-pyridin-2-yl-isoxazole-4-carboxylic acid amide. HPLC r.t. 6.38, [M+H]+=337.
5-[(Naphthalene-1-carbonyl)-amino]-3-pyridin-2-yl-isoxazole-4-carboxylic acid amide. HPLC r.t. 6.27, [M+H]+=359.
5-(4-Fluoro-benzoylamino)-3-pyridin-4-yl-isoxazole-4-carboxylic acid amide. HPLC r.t. 3.38, [M+H]+=327.
3-Pyridin-4-yl-5-(4-trifluoromethoxy-benzoylamino)-isoxazole-4-carboxylic acid amide. HPLC r.t. 4.3, [M+H]+=393.
5-(2-Methyl-benzoylamino)-3-pyridin-4-yl-isoxazole-4-carboxylic acid amide. HPLC r.t. 3.5, [M+H]+=323.
3-Pyridin-4-yl-5-(3-trifluoromethyl-benzoylamino)-isoxazole-4-carboxylic acid amide. HPLC r.t. 3.97, [M+H]+=377.
5-(4-Ethyl-benzoylamino)-3-pyridin-4-yl-isoxazole-4-carboxylic acid amide. HPLC r.t. 4.32, [M+H]+=337.
5-[(Naphthalene-1-carbonyl)-amino]-3-pyridin-4-yl-isoxazole-4-carboxylic acid amide. HPLC r.t. 4.2, [M+H]+=359.
5-(4-Fluoro-benzoylamino)-3-thiophen-2-yl-isoxazole-4-carboxylic acid amide. HPLC r.t. 4.66, [M+H]+=332.
3-Thiophen-2-yl-5-(4-trifluoromethoxy-benzoylamino)-isoxazole-4-carboxylic acid amide. HPLC r.t. 5.53, [M+H]+=398.
5-(2-Methyl-benzoylamino)-3-thiophen-2-yl-isoxazole-4-carboxylic acid amide. HPLC r.t. 4.79, [M+H]+=328.
3-Thiophen-2-yl-5-(3-trifluoromethyl-benzoylamino)-isoxazole-4-carboxylic acid amide. HPLC r.t. 5.21, [M+H]+=382.
5-(4-Ethyl-benzoylamino)-3-thiophen-2-yl-isoxazole-4-carboxylic acid amide. HPLC r.t. 5.64, [M+H]+=342.
5-[(Naphthalene-1-carbonyl)-amino]-3-thiophen-2-yl-isoxazole-4-carboxylic acid amide. HPLC r.t. 5.44, [M+H]+=364.
5-(4-Fluoro-benzoylamino)-3-thiophen-3-yl-isoxazole-4-carboxylic acid amide. HPLC r.t. 4.66, [M+H]+=332.
3-Thiophen-3-yl-5-(4-trifluoromethoxy-benzoylamino)-isoxazole-4-carboxylic acid amide. HPLC r.t. 5.59, [M+H]+=398.
5-(2-Methyl-benzoylamino)-3-thiophen-3-yl-isoxazole-4-carboxylic acid amide. HPLC r.t. 4.77, [M+H]+=328.
3-Thiophen-3-yl-5-(3-trifluoromethyl-benzoylamino)-isoxazole-4-carboxylic acid amide. HPLC r.t. 5.29, [M+H]+=382.
5-(4-Ethyl-benzoylamino)-3-thiophen-3-yl-isoxazole-4-carboxylic acid amide. HPLC r.t. 5.58, [M+H]+=342.
5-[(Naphthalene-1-carbonyl)-amino]-3-thiophen-3-yl-isoxazole-4-carboxylic acid amide. HPLC r.t. 5.43, [M+H]+=364.
5-(4-Fluoro-benzoylamino)-3-thiazol-2-yl-isoxazole-4-carboxylic acid amide. HPLC r.t. 5.72, [M+H]+=333.
3-Thiazol-2-yl-5-(4-trifluoromethoxy-benzoylamino)-isoxazole-4-carboxylic acid amide. HPLC r.t. 6.66, [M+H]+=398.
5-(2-Methyl-benzoylamino)-3-thiazol-2-yl-isoxazole-4-carboxylic acid amide. HPLC r.t. 5.8, [M+H]+=329.
3-Thiazol-2-yl-5-(3-trifluoromethyl-benzoylamino)-isoxazole-4-carboxylic acid amide. HPLC r.t. 6.47, [M+H]+=383.
5-(4-Ethyl-benzoylamino)-3-thiazol-2-yl-isoxazole-4-carboxylic acid amide. HPLC r.t. 6.6, [M+H]+=343.
5-(4-Fluoro-benzoylamino)-3-pyridin-3-yl-isoxazole-4-carboxylic acid amide. HPLC r.t. 3.34, [M+H]+=327.
3-Pyridin-3-yl-5-(4-trifluoromethoxy-benzoylamino)-isoxazole-4-carboxylic acid amide. HPLC r.t. 4.27, [M+H]+=393.
5-(2-Methyl-benzoylamino)-3-pyridin-3-yl-isoxazole-4-carboxylic acid amide. HPLC r.t. 3.45, [M+H]+=323.
3-Pyridin-3-yl-5-(3-trifluoromethyl-benzoylamino)-isoxazole-4-carboxylic acid amide. HPLC r.t. 3.95, [M+H]+=377.
5-(4-Ethyl-benzoylamino)-3-pyridin-3-yl-isoxazole-4-carboxylic acid amide. HPLC r.t. 4.33, [M+H]+=337.
5-[(Naphthalene-1-carbonyl)-amino]-3-pyridin-3-yl-isoxazole-4-carboxylic acid amide. HPLC r.t. 4.2, [M+H]+=359.
3-(3,5-Dimethyl-1-phenyl-1H-pyrazol-4-yl)-5-(4-fluoro-benzoylamino)-isoxazole-4-carboxylic acid amide. HPLC r.t. 5.4, [M+H]+=420.
3-(3,5-Dimethyl-1-phenyl-1H-pyrazol-4-yl)-5(2-methyl-benzoylamino)-isoxazole-4-carboxylic acid amide. HPLC r.t. 5.51, [M+H]+=416.
3-(3,5-Dimethyl-1-phenyl-1H-pyrazol-4-yl)-5-(4-ethyl-benzoylamino)-isoxazole-4-carboxylic acid amide. HPLC r.t. 6.21, [M+H]+=430.
3-(3,5-Dimethyl-1-phenyl-1H-pyrazol-4-yl)-5-[(naphthalene-1-carbonyl)-amino]-isoxazole-4-carboxylic acid amide. HPLC r.t. 6.04, [M+H]+=452.
5-(2-Fluoro-benzoylamino)-3-pyridin-2-yl-isoxazole-4-carboxylic acid amide. HPLC r.t. 5.22, [M+H]+=327.
5-(3-Methyl-benzoylamino)-3-pyridin-2-yl-isoxazole-4-carboxylic acid amide. HPLC r.t. 5.83, [M+H]+=323.
5-[2-(4-Fluoro-phenyl)-acetylamino]-3-pyridin-4-yl-isoxazole-4-carboxylic acid amide. HPLC r.t. 3.48, [M+H]+=341.
5-(3,5-Difluoro-benzoylamino)-3-pyridin-4-yl-isoxazole-4-carboxylic acid amide. HPLC r.t. 3.32, [M+H]+=345.
5-(3-Chloro-benzoylamino)-3-pyridin-4-yl-isoxazole-4-carboxylic acid amide. HPLC r.t. 3.74, [M+H]+=343.
5-(2-Fluoro-benzoylamino)-3-pyridin-4-yl-isoxazole-4-carboxylic acid amide. HPLC r.t. 3.36, [M+H]+=327.
5-(3-Methyl-benzoylamino)-3-pyridin-4-yl-isoxazole-4-carboxylic acid amide. HPLC r.t. 3.78, [M+H)+=323.
5-(3,5-Difluoro-benzoylamino)-3-thiophen-2-yl-isoxazole-4-carboxylic acid amide. HPLC r.t. 4.49, [M+H]+=350.
5-(3-Chloro-benzoylamino)-3-thiophen-2-yl-isoxazole-4-carboxylic acid amide. HPLC r.t. 5.05, [M+H]+=348.
5-(3,5-Difluoro-benzoylamino)-3-thiophen-3-yl-isoxazole-4-carboxylic acid amide. HPLC r.t. 4.58, (M+H]+=350.
5-(3-Methyl-benzoylamino)-3-thiophen-3-yl-isoxazole-4-carboxylic acid amide. HPLC r.t. 5.05, [M+H]+=328.
5-(3-Chloro-benzoylamino)-3-thiazol-2-yl-isoxazole-4-carboxylic acid amide. HPLC r.t. 6.33, [M+H]+=349.
5-(3-Chloro-benzoylamino)-3-pyridin-3-yl-isoxazole-4-carboxylic acid amide. HPLC r.t. 3.71, [M+H]+=343.
5-(2-Fluoro-benzoylamino)-3-pyridin-3-yl-isoxazole-4-carboxylic acid amide. HPLC r.t. 3.3, [M+H]+=327.
5-(3-Methyl-benzoylamino)-3-pyridin-3-yl-isoxazole-4-carboxylic acid amide. HPLC r.t. 3.73, [M+H]+=323.
5-(3-Chloro-benzoylamino)-3-(3,5-dimethyl-1-phenyl-1H-pyrazol-4-yl)-isoxazole-4-carboxylic acid amide. HPLC r.t. 5.78, [M+H]+=436.
5-Butyrylamino-3-pyridin-2-yl-isoxazole-4-carboxylic acid amide. HPLC r.t. 4.22, [M+H]+=275.
5-[(E)-(3-Phenyl-acryloyl)amino]-3-pyridin-2-yl-isoxazole-4-carboxylic acid amide. HPLC r.t. 5.74, [M+H]+=335.
5-(3,3-Dimethyl-butyrylamino)-3-pyridin-2-yl-isoxazole-4-carboxylic acid amide. HPLC r.t. 5.2, [M+H]+=303.
5-(2-Chloro-2-phenyl-acetylamino)-3-pyridin-4-yl-isoxazole-4-carboxylic acid amide. HPLC r.t. 3.37, [M+H]+=357.
5-Butyrylamino-3-pyridin-4-yl-isoxazole-4-carboxylic acid amide. HPLC r.t. 2.23, [M+H]+=275.
5-[(Biphenyl-4-carbonyl)-amino]-3-pyridin-4-yl-isoxazole-4-carboxylic acid amide. HPLC r.t. 4.94, [M+H]+=385.
5-(4-Chloromethyl-benzoylamino)-3-pyridin-4-yl-isoxazole-4-carboxylic acid amide. HPLC r.t. 3.91, [M+H]+=355.
3-Pyridin-4-yl-5-(3,4,5-trimethoxy-benzoylamino)-isoxazole-4-carboxylic acid amide. HPLC r.t. 3.28, [M+H]+=399.
5-[(E)-(3-Phenyl-acryloyl)amino]-3-pyridin-4-yl-isoxazole-4-carboxylic acid amide. HPLC r.t. 3.91, [M+H]+=335.
5-(3,3-Dimethyl-butyrylamino)-3-pyridin-4-yl-isoxazole-4-carboxylic acid amide. HPLC r.t. 3.25, [M+H]+=303.
5-(2-Chloro-2-phenyl-acetylamino)-3-thiophen-2-yl-isoxazole-4-carboxylic acid amide. HPLC r.t. 4.6, [M+H]+=362.
5-(4-Nitro-benzoylamino)-3-thiophen-2-yl-isoxazole-4-carboxylic acid amide. HPLC r.t. 4.23, [M+H]+=359.
5-Butyrylamino-3-thiophen-2-yl-isoxazole-4-carboxylic acid amide. HPLC r.t. 3.38, [M+H]+=280.
5-[(Biphenyl-4-carbonyl)-amino]-3-thiophen-2-yl-isoxazole-4-carboxylic acid amide. HPLC r.t. 6.19, [M+H]+=390.
5-(4-Chloromethyl-benzoylamino)-3-thiophen-2-yl-isoxazole-4-carboxylic acid amide. HPLC r.t. 5.17, [M+H]+=362.
3-Thiophen-2-yl-5-(3,4,5-trimethoxy-benzoylamino)-isoxazole-4-carboxylic acid amide. HPLC r.t. 4.47, [M+H]+=404.
5-[(E)-(3-Phenyl-acryloyl)amino]-3-thiophen-2-yl-isoxazole-4-carboxylic acid amide. HPLC r.t. 5.06, [M+H]+=340.
5-(3,3-Dimethyl-butyrylamino)-3-thiophen-2-yl-isoxazole-4-carboxylic acid amide. HPLC r.t. 4.5, [M+H]+=308.
5-(2-Chloro-2-phenyl-acetylamino)-3-thiophen-3-yl-isoxazole-4-carboxylic acid amide. HPLC r.t. 4.77, [M+H]+=362.
5-Butyrylamino-3-thiophen-3-yl-isoxazole-4-carboxylic acid amide. HPLC r.t. 3.35, [M+H]+=380.
5-(3,3-Dimethyl-butyrylamino)-3-thiophen-3-yl-isoxazole-4-carboxylic acid amide. HPLC r.t. 4.44, [M+H]+=308.
5-(4-Chloromethyl-benzoylamino)-3-thiazol-2-yl-isoxazole-4-carboxylic acid amide. HPLC r.t. 6.13, [M+H]+=363.
5-(3,3-Dimethyl-butyrylamino)-3-thiazol-2-yl-isoxazole-4-carboxylic acid amide. HPLC r.t. 5.41, [M+H]+=309.
5-(3,3-Dimethyl-butyrylamino)-3-pyridin-3-yl-isoxazole-4-carboxylic acid amide. HPLC r.t. 3.23, [M+H]+=303.
5-Butyrylamino-3-(3,5-dimethyl-1-phenyl-1H-pyrazol-4-yl)-isoxazole-4-carboxylic acid amide. HPLC r.t. 4.42, [M+H]+=368.
3-(3,5-Dimethyl-1-phenyl-1H-pyrazol-4-yl)-5-[(E)-(3-phenyl-acryloyl)amino]-isoxazole-4-carboxylic acid amide. HPLC r.t. 5.67, [M+H]+=428.
5-(3,3-Dimethyl-butyrylamino)-3-(3,5-dimethyl-1-phenyl-1H-pyrazol-4-yl)-isoxazole-4-carboxylic acid amide. HPLC r.t. 5.22, [M+H]+=396.
5-Butyrylamino-3-quinolin-2-yl-isoxazole-4-carboxylic acid amide. HPLC r.t. 5.89, [M+H]+=325.
5-(3,3-Dimethyl-butyrylamino)-3-quinolin-2-yl-isoxazole-4-carboxylic acid amide. HPLC r.t. 6.72, [M+H]+=353.
5-[(Furan-2-carbonyl)-amino]-3-pyridin-2-yl-isoxazole-4-carboxylic acid amide. HPLC r.t. 4.45, [M+H]+=299.
5-[(Naphthalene-2-carbonyl)-amino]-3-pyridin-4-yl-isoxazole-4-carboxylic acid amide. HPLC r.t. 4.41, [M+H]+=359.
5-(4-Methoxy-benzoylamino)-3-pyridin-4-yl-isoxazole-4-carboxylic acid amide. HPLC r.t. 3.51, [M+H]+=339.
5-[(Furan-2-carbonyl)-amino]-3-pyridin-4-yl-isoxazole-4-carboxylic acid amide. HPLC r.t. 2.42, [M+H]+=299.
5-(3-Cyclopentyl-propionylamino)-3-pyridin-4-yl-isoxazole-4-carboxylic acid amide. HPLC r.t. 4.32, [M+H]+=329.
5-[(Naphthalene-2-carbonyl)-amino]-3-thiophen-2-yl-isoxazole-4-carboxylic acid amide. HPLC r.t. 5.71, [M+H]+=364.
5-(4-Methoxy-benzoylamino)-3-thiophen-2-yl-isoxazole-4-carboxylic acid amide. HPLC r.t. 4.75, [M+H]+=344.
5-(2-Benzyloxy-acetylamino)-3-thiophen-2-yl-isoxazole-4-carboxylic acid amide. HPLC r.t. 5.06, [M+H]+=358.
5-(2,4-Dimethoxy-benzoylamino)-3-thiophen-2-yl-isoxazole-4-carboxylic acid amide. HPLC r.t. 5.16, [M+H]+=374.
5-[(Furan-2-carbonyl)-amino]-3-thiophen-2-yl-isoxazole-4-carboxylic acid amide. HPLC r.t. 3.67, [M+H]+=304.
5-(3-Cyclopentyl-propionylamino)-3-thiophen-2-yl-isoxazole-4-carboxylic acid amide. HPLC r.t. 5.5, [M+H]+=334.
5-(4-Bromo-benzoylamino)-3-thiophen-2-yl-isoxazole-4-carboxylic acid amide. HPLC r.t. 5.34, [M+H]+=392.
5-[(Naphthalene-2-carbonyl)-amino]-3-thiophen-3-yl-isoxazole-4-carboxylic acid amide. HPLC r.t. 5.71, [M+H]+=364.
5-(4-Methoxy-benzoylamino)-3-thiophen-3-yl-isoxazole-4-carboxylic acid amide. HPLC r.t. 4.72, [M+H]+=344.
5-(2,4-Dimethoxy-benzoylamino)-3-thiophen-3-yl-isoxazole-4-carboxylic acid amide. HPLC r.t. 5.02, [M+H]+=374.
5-[(Furan-2-carbonyl)-amino]-3-thiophen-3-yl-isoxazole-4-carboxylic acid amide. HPLC r.t. 3.69, [M+H]+=304.
5-(3-Cyclopentyl-propionylamino)-3-thiophen-3-yl-isoxazole-4-carboxylic acid amide. HPLC r.t. 5.45, [M+H]+=334.
5-(4-Methoxy-benzoylamino)-3-thiazol-2-yl-isoxazole-4-carboxylic acid amide. HPLC r.t. 5.63, [M+H]+=345.
5-(2,4-Dimethoxy-benzoylamino)-3-thiazol-2-yl-isoxazole-4-carboxylic acid amide. HPLC r.t. 5.66, [M+H]+=375.
5-[(Furan-2-carbonyl)-amino]-3-thiazol-2-yl-isoxazole-4-carboxylic acid amide. HPLC r.t. 4.61, [M+H]+=305.
5-(3-Cyclopentyl-propionylamino)-3-thiazol-2-yl-isoxazole-4-carboxylic acid amide. HPLC r.t. 6.36, [M+H]+=335.
5-[(Furan-2-carbonyl)-amino]-3-pyridin-3-yl-isoxazole-4-carboxylic acid amide. HPLC r.t. 2.39, [M+H]+=299.
3-(3,5-Dimethyl-1-phenyl-1H-pyrazol-4-yl)-5-(4-methoxy-benzoylamino)-isoxazole-4-carboxylic acid amide. HPLC r.t. 5.4, [M+H]+=432.
5-(2,4-Dimethoxy-benzoylamino)-3-(3,5-dimethyl-1-phenyl-1H-pyrazol-4-yl)-isoxazole-4-carboxylic acid amide. HPLC r.t. 5.5, [M+H]+=462.
3-(3,5-Dimethyl-1-phenyl-1H-pyrazol-4-yl)-5-[(furan-2-carbonyl)-amino]-isoxazole-4-carboxylic acid amide. HPLC r.t. 4.58, [M+H]+=392.
5-(3-Cyclopentyl-propionylamino)-3-(3,5-dimethyl-1-phenyl-1H-pyrazol-4-yl)-isoxazole-4-carboxylic acid amide. HPLC r.t. 6.05, [M+H]+=422.
5-(3,4-Dimethoxy-benzoylamino)-3-(3,5-dimethyl-1-phenyl-1H-pyrazol-4-yl)-isoxazole-4-carboxylic acid amide. HPLC r.t. 5.02, [M+H]+=462.
5-(4-Bromo-benzoylamino)-3-(3,5-dimethyl-1-phenyl-1H-pyrazol-4-yl)-isoxazole-4-carboxylic acid amide. HPLC r.t. 5.99, [M+H]+=480.
5-(3-Cyclopentyl-propionylamino)-3-quinolin-2-yl-isoxazole-4-carboxylic acid amide. HPLC r.t. 7.56, [M+H]+=379.
5-(2-Ethyl-butyrylamino)-3-pyridin-2-yl-isoxazole-4-carboxylic acid amide. HPLC r.t. 5.23, [M+H]+=303.
5-(2-Propyl-pentanoylamino)-3-pyridin-2-yl-isoxazole-4-carboxylic acid amide. HPLC r.t. 6.35, [M+H]+=331.
5-(3-Methyl-butyrylamino)-3-pyridin-4-yl-isoxazole-4-carboxylic acid amide. HPLC r.t. 2.79, [M+H]+=289.
5-[(Benzo[1,3]dioxole-5-carbonyl)-amino]-3-pyridin-4-yl-isoxazole-4-carboxylic acid amide. HPLC r.t. 3.38, [M+H]+=353.
5-(2-Ethyl-butyrylamino)-3-pyridin-4-yl-isoxazole-4-carboxylic acid amide. HPLC r.t. 3.17, [M+H]+=303.
5-(2-Propyl-pentanoylamino)-3-pyridin-4-yl-isoxazole-4-carboxylic acid amide. HPLC r.t. 4.42, [M+H]+=331.
5-(3-Methyl-butyrylamino)-3-thiophen-2-yl-isoxazole-4-carboxylic acid amide. HPLC r.t. 3.98, [M+H]+=294.
5-[(Benzo[1,3]dioxole-5-carbonyl)-amino]-3-thiophen-2-yl-isoxazole-4-carboxylic acid amide. HPLC r.t. 4.63, [M+H]+=358.
5-(2-Ethyl-butyrylamino)-3-thiophen-2-yl-isoxazole-4-carboxylic acid amide. HPLC r.t. 4.42, [M+H]+=308.
5-(2-Propyl-pentanoylamino)-3-thiophen-2-yl-isoxazole-4-carboxylic acid amide. HPLC r.t. 5.65, [M+H]+=336.
5-[2-(3,4-Dimethoxy-phenyl)-acetylamino]-3-thiophen-2-yl-isoxazole-4-carboxylic acid amide. HPLC r.t. 4.08, [M+H]+=388.
5-(3-Methyl-butyrylamino)-3-thiophen-3-yl-isoxazole-4-carboxylic acid amide. HPLC r.t. 3.93, [M+H]+=294.
5-[(Benzo[1,3]dioxole-5-carbonyl)-amino]-3-thiophen-3-yl-isoxazole-4-carboxylic acid amide. HPLC r.t. 4.61, [M+H]+=358.
5-(2-Ethyl-butyrylamino)-3-thiophen-3-yl-isoxazole-4-carboxylic acid amide. HPLC r.t. 4.38, [M+H]+=308.
5-(2-Propyl-pentanoylamino)-3-thiophen-3-yl-isoxazole-4-carboxylic acid amide. HPLC r.t. 5.6, [M+H]+=336.
5-(3-Methyl-butyrylamino)-3-thiazol-2-yl-isoxazole-4-carboxylic acid amide. HPLC r.t. 5.01, [M+H]+=295.
5-[(Benzo[1,3]dioxole-5-carbonyl)-amino]-3-thiazol-2-yl-isoxazole-4-carboxylic acid amide. HPLC r.t. 5.56, [M+H]+=359.
5-(2-Ethyl-butyrylamino)-3-thiazol-2-yl-isoxazole-4-carboxylic acid amide. HPLC r.t. 5.42, [M+H]+=309.
5-(2-Propyl-pentanoylamino)-3-thiazol-2-yl-isoxazole-4-carboxylic acid amide. HPLC r.t. 6.53, [M+H]+=337.
5-(2-Ethyl-butyrylamino)-3-pyridin-3-yl-isoxazole-4-carboxylic acid amide. HPLC r.t. 3.14, [M+H]+=303.
5-(2-Propyl-pentanoylamino)-3-pyridin-3-yl-isoxazole-4-carboxylic acid amide. HPLC r.t. 4.38, [M+H]+=331.
3-(3,5-Dimethyl-1-phenyl-1H-pyrazol-4-yl)-5-(3-methyl-butyrylamino)-isoxazole-4-carboxylic acid amide. HPLC r.t. 4.85, [M+H]+=382.
5-[(Benzo[1,3]dioxole-5-carbonyl)-amino]-3-(3,5-dimethyl-1-phenyl-1H-pyrazol-4-yl)-isoxazole-4-carboxylic acid amide. HPLC r.t. 5.3, [M+H]+=446.
3-(3,5-Dimethyl-1-phenyl-1H-pyrazol-4-yl)-5-(2-ethyl-butyrylamino)-isoxazole-4-carboxylic acid amide. HPLC r.t. 5.21, [M+H]+=396.
3-(3,5-Dimethyl-1-phenyl-1H-pyrazol-4-yl)-5-(2-propyl-pentanoylamino)-isoxazole-4-carboxylic acid amide. HPLC r.t. 6.16, [M+H]+=424.
5-(3-Methyl-butyrylamino)-3-quinolin-2-yl-isoxazole-4-carboxylic acid amide. HPLC r.t. 6.36, [M+H]+=339.
5-(2-Ethyl-butyrylamino)-3-quinolin-2-yl-isoxazole-4-carboxylic acid amide. HPLC r.t. 6.74, [M+H]+=353.

### EXAMPLE 9

5-Acetylamino-3-thiophen-2-yl-isoxazole-4-carboxylic Acid 4-(3-methoxy-4-phenylaminomethyl-phenoxy)-butyryl AM Resin Amide

To a suspension of 5-amino-3-thiophen-2-yl-isoxazole-4-carboxylic acid 4-(3-methoxy-4-phenylaminomethyl-phenoxy)-butyryl AM resin amide (160 mg, 0.094 mmol) in dichloromethane (1.6 ml), triethyl amine (0.040 ml, 0.285 mmol), 4-dimethyl aminopyridine (5.8 mg, 0.048 mmol) and acetyl chloride (0.142 mmol) were added. The final mixture was shaken at room temperature (23° C.) for 16 hours, afterward the resin was filtered, washed with N,N-dimethylformamide, dichloromethane, methanol, and dichloromethane (3×) dried under vacuum.

### EXAMPLE 10

5-Acetylamino-3-thiophen-2-yl-isoxazole-4-carboxylic Acid (4-methoxy-phenyl)-amide

5-Acetylamino-3-thiophen-2-yl-isoxazole-4-carboxylic acid 4-(3-methoxy-4-phenylaminomethyl-phenoxy)-butyryl AM resin amide (0.75 mmol) was treated with a solution of TFA 20% in anhydrous dichloromethane (10 ml). The resulting suspension was gently stirred at 22° C. for 1 h, then washed with anhydrous dichloromethane, methanol, dichloromethane and dried under vacuum to give a crude solid, that after purification by flash chromatography gave 130 mg of the desired compound. [M+H]+=358. ¹H-NMR (DMSO-d₆), diagnostic signals (ppm): 11,4 (bs, 1H), 10,1 (bs, 1H), 7,7 (d, 1H), 7,6 (d, 1H) 7,5 (d, 2H), 7,2 (dd, 1H), 6,9 (d, 2H), 3,7 (s, 3H), 2,1 (s, 3H).

By working in an analogous way and by using the appropriate isoxazole resin bound derivatives and the appropriate acyl chloride the following compound has been prepared, after purification by flash chromatography or preparative HPLC when necessary:
5-Phenylacetylamino-3-thiophen-2-yl-isoxazole-4-carboxylic acid butylamide. [M+H]+=384. ¹H-NMR (DMSO-d₆), diagnostic signals (ppm): 11,4 (bs, 1H), 8,1 (bs, 1H), 7,7 (d, 1H) 7,6 (d, 1H), 7,3 (m, 5H), 7,1 (d, 1H), 3,7 (s, 2H), 3,1 (m, 2H) 1,4 (m, 2H), 1,2 (m, 2H), 0,9 (m, 3H).

### EXAMPLE 11

5-(3-Phenyl-ureido)-3-thiophen-2-yl-isoxazole-4-carboxylic Acid Amide

To a suspension of 5-amino-3-thiophen-2-yl-isoxazole-4-carboxylic acid amide (25 mg, 0.12 mmol) in dichloromethane (0.25 ml), phenyl isocyanate (0.130 ml, 1.2 mmol) was added. The final mixture was stirred at room temperature (23° C.) for 16 hours, afterward volatiles were removed under reduced pressure to give a crude solid, that was purified by flash chromatography (45% yield). [M+H]+=329, [M−H]−=327. HPLC r.t. 4.6.

### EXAMPLE 12

5-(4-Acetylamino-benzenesulfonylamino)-3-pyridin-2-yl-isoxazole-4-carboxylic Acid Rink Amide

To a suspension of 5-Amino-3-pyridin-2-yl-isoxazole-4-carboxylic acid Rink amide (50 mg, 0.030 mmol) in dichloromethane (0.5 ml), diisopropylethyl amine (15.2 μl, 0.089 mmol), 4-dimethyl aminopyridine (1.8 mg, 0.015 mmol) and N-acetylbenzenesulfonyl chloride (10.3 mg, 0.044 mmol) were added. The final mixture was shaken at room temperature (23° C.) for 16 hours, afterward the resin was filtered, washed with N,N-dimethylformamide, dichloromethane, methanol, dichloromethane (3×) and dried under vacuum.

### EXAMPLE 13

5-(4-Acetylamino-benzenesulfonylamino)-3-pyridin-2-yl-isoxazole-4-carboxylic Acid Amide

5-(4-Acetylamino-benzenesulfonylamino)-3-pyridin-2-yl-isoxazole-4-carboxylic acid Rink amide (0.75 mmol) was treated with a solution of TFA 20% in anhydrous dichloromethane (10 ml). The resulting suspension was gently stirred or shaken at 22° C. for 1 h, then washed with anhydrous dichloromethane, methanol, dichloromethane and dried under vacuum to give a crude solid, that after purification by flash chromatography gave 154 mg of the desired compound. [M+H]+=402. HPLC r.t. 3.0.

1. A method for treating diseases caused by and/or associated with an altered protein kinase activity which comprises administering to a mammal in need thereof an effective amount of an aminoisoxazole derivative represented by formula (I): 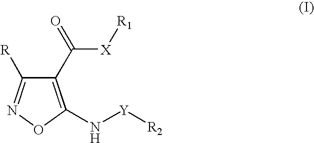
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;
R₂ and R₃ have, each independently, the meanings above reported for R₁ or represent an optionally substituted straight or branched C₂-C₆ alkenyl or alkynyl group; and the pharmaceutically acceptable salts thereof.

2. The method of claim 1 wherein the disease caused by and/or associated with an altered protein kinase activity is a cell proliferative disorder selected from the group consisting of cancer, Alzheimer's disease, viral infections, auto-immune diseases and neurodegenerative disorders.

3. The method of claim 2 wherein the cancer is selected from carcinoma, squamous cell carcinoma, hematopoietic tumors of lymphoid or myeloid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

4. The method of claim 1 wherein the cell proliferative disorder is selected from benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. The method of claim 1 wherein, within the compounds of formula (I), Y is a single bond, R₂ is a hydrogen atom, and R, R₁ and X are as above defined.

9. The method of claim 1 wherein, within the compounds of formula (I), Y is a —CO— group and R, R₁, R₂ and X are as above defined.

10. The method of claim 1 wherein, within the compounds of formula (I), Y is a —CONH— group and R, R₁, R₂ and X are as above defined.

11. The method of claim 1 wherein, within the compounds of formula (I), Y is a —SO₂— group and R, R₁, R₂ and X are as above defined.

12. A method for inhibiting protein kinase activity which comprises contacting the said kinase with an effective amount of a compound as defined in claim 1.

13. An aminoisoxazole derivative represented by formula (I) 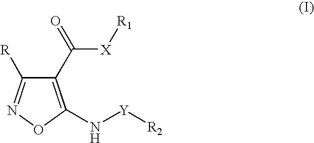
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆ cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;
R₂ and R₃ have, each independently, the meanings above reported for R₁ or represent an optionally substituted straight or branched C₂-C₆ alkenyl or alkynyl group; and the pharmaceutically acceptable salts thereof; provided that:
a) R is other than nitrofuryl; and
b) 5-amino-3-(2-aminofuryl-5-yl)-4-methoxycarbonyl-isoxazole and 5-amino-4-ethoxycarbonyl-3-(indol-3-yl)-isoxazole, optionally further substituted at the indole moiety, being excluded.

14. A compound of formula (I) according to claim 13 wherein Y is a single bond, R₂ is a hydrogen atom, and R, R₁ and X are as defined in claim 13.

15. The compound of claim 14 wherein X is a —N(R₃)— group and R₃ is as defined in claim 13.

16. A compound of formula (I) according to claim 13 wherein Y is a —CO— group and R, R₁, R₂ and X are as defined in claim 13.

17. The compound of claim 16 wherein X is —N(R₃)— and R₁ and R₃ are both hydrogen atoms.

18. A compound of formula (I) according to claim 13 wherein Y is a —CONH— group and R, R₁, R₂ and X are as above defined.

19. The compound of claim 18 wherein X is —N(R₃)— and R₁ and R₃ are both hydrogen atoms.

20. A compound of formula (I) according to claim 13 wherein Y is a —SO₂— group and R, R₁, R₂ and X are as above defined.

21. The compound of claim 20 wherein X is —N(R₃)— and R₁ and R₃ are both hydrogen atoms.

22. A compound of formula (I) according to claim 13 wherein any heteroaryl or heterocyclyl group has 1 or 2 heteroatoms selected among nitrogen, oxygen or sulfur.

23. A compound of formula (I) as defined in claim 13, optionally in the form of a pharmaceutically acceptable salt, selected from:
1) 5-amino-3-thiophen-3-yl-isoxazole-4-carboxylic acid amide;
2) 5-amino-3-thiophen-2-yl-isoxazole-4-carboxylic acid amide;
3) 5-amino-3-pyridin-2-yl-isoxazole-4-carboxylic acid amide;
4) 5-amino-3-pyridin-3-yl-isoxazole-4-carboxylic acid amide;
5) 5-amino-3-pyridin-4-yl-isoxazole-4-carboxylic acid amide;
6) 5-amino-3-thiazol-2-yl-isoxazole-4-carboxylic acid amide;
7) 5-amino-3-(3,5-dimethyl-1-phenyl-1H-pyrazol-4-yl)-isoxazole-4-carboxylic acid amide;
8) 5-amino-3-quinolin-2-yl-isoxazole-4-carboxylic acid amide;
9) 5-amino-3-thiophen-2-yl-isoxazole-4-carboxylic acid butylamide;
10) 5-acetylamino-3-thiophen-2-yl-isoxazole-4-carboxylic acid (4-methoxy-phenyl)-amide;
11) methyl 5-amino-3-(2-pyridinyl)-4-isoxazolecarboxylate;
12) 5-amino-3-pyridin-4-yl-isoxazole-4-carboxylic acid (2,4-difluoro-phenyl)-amide;
13) 5-amino-3-pyridin-4-yl-isoxazole-4-carboxylic acid (4-chloro-phenyl)-amide;
14) 5-amino-3-pyridin-4-yl-isoxazole-4-carboxylic acid benzylamide;
15) 5-amino-3-pyridin-4-yl-isoxazole-4-carboxylic acid (5,6,7,8-tetrahydro-naphthalen-1-yl)-amide;
16) 5-amino-3-pyridin-4-yl-isoxazole-4-carboxylic acid (4-fluoro-phenyl)-amide;
17) 5-amino-3-pyridin-4-yl-isoxazole-4-carboxylic acid benzhydryl-amide;
18) 5-amino-3-thiophen-2-yl-isoxazole-4-carboxylic acid (2,4-difluoro-phenyl)-amide;
19) 5-amino-3-thiophen-2-yl-isoxazole-4-carboxylic acid (4-chloro-phenyl)-amide;
20) 5-amino-3-thiophen-2-yl-isoxazole-4-carboxylic acid benzylamide;
21) 5-amino-3-thiophen-2-yl-isoxazole-4-carboxylic acid (5,6,7,8-tetrahydro-naphthalen-1-yl)-amide;
22) 5-amino-3-thiophen-2-yl-isoxazole-4-carboxylic acid (4-fluoro-phenyl)-amide;
23) 5-amino-3-thiophen-2-yl-isoxazole-4-carboxylic acid benzhydryl-amide;
24) 5-amino-3-thiophen-3-yl-isoxazole-4-carboxylic acid (2,4-difluoro-phenyl)-amide;
25) 5-amino-3-thiophen-3-yl-isoxazole-4-carboxylic acid (4-chloro-phenyl)-amide;
26) 5-amino-3-thiophen-3-yl-isoxazole-4-carboxylic acid benzylamide;
27) 5-amino-3-thiophen-3-yl-isoxazole-4-carboxylic acid (5,6,7,8-tetrahydro-naphthalen-1-yl)-amide;
28) 5-amino-3-thiophen-3-yl-isoxazole-4-carboxylic acid (4-fluoro-phenyl)-amide;
29) 5-amino-3-thiophen-3-yl-isoxazole-4-carboxylic acid benzhydryl-amide;
30) 5-amino-3-thiazol-2-yl-isoxazole-4-carboxylic acid (2,4-difluoro-phenyl)-amide;
31) 5-amino-3-thiazol-2-yl-isoxazole-4-carboxylic acid (4-chloro-phenyl)-amide;
32) 5-amino-3-thiazol-2-yl-isoxazole-4-carboxylic acid benzylamide;
33) 5-amino-3-thiazol-2-yl-isoxazole-4-carboxylic acid (5,6,7,8-tetrahydro-naphthalen-1-yl)-amide;
34) 5-amino-3-thiazol-2-yl-isoxazole-4-carboxylic acid (4-fluoro-phenyl)-amide;
35) 5-amino-3-thiazol-2-yl-isoxazole-4-carboxylic acid benzhydryl-amide;
36) 5-amino-3-pyridin-2-yl-isoxazole-4-carboxylic acid (2,4-difluoro-phenyl)-amide;
37) 5-amino-3-pyridin-2-yl-isoxazole-4-carboxylic acid (4-chloro-phenyl)-amide;
38) 5-amino-3-pyridin-2-yl-isoxazole-4-carboxylic acid benzylamide;
39) 5-amino-3-pyridin-2-yl-isoxazole-4-carboxylic acid (5,6,7,8-tetrahydro-naphthalen-1-yl)-amide;
40) 5-amino-3-pyridin-2-yl-isoxazole-4-carboxylic acid (4-fluoro-phenyl)-amide;
41) 5-amino-3-pyridin-2-yl-isoxazole-4-carboxylic acid benzhydryl-amide;
42) 5-amino-3-pyridin-3-yl-isoxazole-4-carboxylic acid (2,4-difluoro-phenyl)-amide;
43) 5-amino-3-pyridin-3-yl-isoxazole-4-carboxylic acid (4-chloro-phenyl)-amide;
44) 5-amino-3-pyridin-3-yl-isoxazole-4-carboxylic acid benzylamide;
45) 5-amino-3-pyridin-3-yl-isoxazole-4-carboxylic acid (5,6,7,8-tetrahydro-naphthalen-1-yl)-amide;
46) 5-amino-3-pyridin-3-yl-isoxazole-4-carboxylic acid (4-fluoro-phenyl)-amide;
47) 5-amino-3-pyridin-3-yl-isoxazole-4-carboxylic acid benzhydryl-amide;
48) 5-amino-3-(3,5-dimethyl-1-phenyl-1H-pyrazol-4-yl)-isoxazole-4-carboxylic acid (2,4-difluoro-phenyl)-amide;
49) 5-amino-3-(3,5-dimethyl-1-phenyl-1H-pyrazol-4-yl)-isoxazole-4-carboxylic acid (4-chloro-phenyl)-amide;
50) 5-amino-3-(3,5-dimethyl-1-phenyl-1H-pyrazol-4-yl)-isoxazole-4-carboxylic acid (5,6,7,8-tetrahydro-naphthalen-1-yl)-amide;
51) 5-amino-3-(3,5-dimethyl-1-phenyl-1H-pyrazol-4-yl)-isoxazole-4-carboxylic acid (4-fluoro-phenyl)-amide;
52) 5-amino-3-(3,5-dimethyl-1-phenyl-1H-pyrazol-4-yl)-isoxazole-4-carboxylic acid benzhydryl-amide;
53) 5-amino-3-quinolin-2-yl-isoxazole-4-carboxylic acid (2,4-difluoro-phenyl)-amide;
54) 5-amino-3-quinolin-2-yl-isoxazole-4-carboxylic acid (4-chloro-phenyl)-amide;
55) 5-amino-3-quinolin-2-yl-isoxazole-4-carboxylic acid benzylamide;
56) 5-amino-3-quinolin-2-yl-isoxazole-4-carboxylic acid (5,6,7,8-tetrahydro-naphthalen-1-yl)-amide;
57) 5-amino-3-quinolin-2-yl-isoxazole-4-carboxylic acid (4-fluoro-phenyl)-amide;
58) 5-amino-3-quinolin-2-yl-isoxazole-4-carboxylic acid benzhydryl-amide;
59) 5-amino-3-pyridin-4-yl-isoxazole-4-carboxylic acid naphthalen-1-ylamide;
60) 5-amino-3-pyridin-4-yl-isoxazole-4-carboxylic acid p-tolylamide;
61) 5-amino-3-pyridin-4-yl-isoxazole-4-carboxylic acid (3-benzyloxy-phenyl)-amide;
62) 5-amino-3-pyridin-4-yl-isoxazole-4-carboxylic acid 2,4-dimethoxy-benzylamide;
63) 5-amino-3-pyridin-4-yl-isoxazole-4-carboxylic acid indan-5-ylamide;
64) 5-amino-3-pyridin-4-yl-isoxazole-4-carboxylic acid (3,5-dimethoxy-phenyl)-amide;
65) 5-amino-3-thiophen-2-yl-isoxazole-4-carboxylic acid naphthalen-1-ylamide;
66) 5-amino-3-thiophen-2-yl-isoxazole-4-carboxylic acid p-tolylamide;
67) 5-amino-3-thiophen-2-yl-isoxazole-4-carboxylic acid (3-benzyloxy-phenyl)-amide;
68) 5-amino-3-thiophen-2-yl-isoxazole-4-carboxylic acid 2,4-dimethoxy-benzylamide;
69) 5-amino-3-thiophen-2-yl-isoxazole-4-carboxylic acid indan-5-ylamide;
70) 5-amino-3-thiophen-2-yl-isoxazole-4-carboxylic acid (3,5-dimethoxy-phenyl)-amide;
71) 5-amino-3-thiophen-3-yl-isoxazole-4-carboxylic acid naphthalen-1-ylamide;
72) 5-amino-3-thiophen-3-yl-isoxazole-4-carboxylic acid p-tolylamide;
73) 5-amino-3-thiophen-3-yl-isoxazole-4-carboxylic acid (3-benzyloxy-phenyl)-amide;
74) 5-amino-3-thiophen-3-yl-isoxazole-4-carboxylic acid 2,4-dimethoxy-benzylamide;
75) 5-amino-3-thiophen-3-yl-isoxazole-4-carboxylic acid indan-5-ylamide;
76) 5-amino-3-thiophen-3-yl-isoxazole-4-carboxylic acid (3,5-dimethoxy-phenyl)-amide;
77) 5-amino-3-thiazol-2-yl-isoxazole-4-carboxylic acid naphthalen-1-ylamide;
78) 5-amino-3-thiazol-2-yl-isoxazole-4-carboxylic acid p-tolylamide;
79) 5-amino-3-thiazol-2-yl-isoxazole-4-carboxylic acid (3-benzyloxy-phenyl)-amide;
80) 5-amino-3-thiazol-2-yl-isoxazole-4-carboxylic acid 2,4-dimethoxy-benzylamide;
81) 5-amino-3-thiazol-2-yl-isoxazole-4-carboxylic acid indan-5-ylamide;
82) 5-amino-3-thiazol-2-yl-isoxazole-4-carboxylic acid (3,5-dimethoxy-phenyl)-amide;
83) 5-amino-3-pyridin-2-yl-isoxazole-4-carboxylic acid naphthalen-1-ylamide;
84) 5-amino-3-pyridin-2-yl-isoxazole-4-carboxylic acid p-tolylamide;
85) 5-amino-3-pyridin-2-yl-isoxazole-4-carboxylic acid (3-benzyloxy-phenyl)-amide;
86) 5-amino-3-pyridin-2-yl-isoxazole-4-carboxylic acid 2,4-dimethoxy-benzylamide;
87) 5-amino-3-pyridin-2-yl-isoxazole-4-carboxylic acid indan-5-ylamide;
88) 5-amino-3-pyridin-2-yl-isoxazole-4-carboxylic acid (3,5-dimethoxy-phenyl)-amide;
89) 5-amino-3-pyridin-3-yl-isoxazole-4-carboxylic acid naphthalen-1-ylamide;
90) 5-amino-3-pyridin-3-yl-isoxazole-4-carboxylic acid p-tolylamide;
91) 5-amino-3-pyridin-3-yl-isoxazole-4-carboxylic acid (3-benzyloxy-phenyl)-amide;
92) 5-amino-3-pyridin-3-yl-isoxazole-4-carboxylic acid 2,4-dimethoxy-benzylamide;
93) 5-amino-3-pyridin-3-yl-isoxazole-4-carboxylic acid indan-5-ylamide;
94) 5-amino-3-pyridin-3-yl-isoxazole-4-carboxylic acid (3,5-dimethoxy-phenyl)-amide;
95) 5-amino-3-(3,5-dimethyl-1-phenyl-1H-pyrazol-4-yl)-isoxazole-4-carboxylic acid naphthalen-1-ylamide;
96) 5-amino-3-(3,5-dimethyl-1-phenyl-1H-pyrazol-4-yl)-isoxazole-4-carboxylic acid p-tolylamide;
97) 5-amino-3-(3,5-dimethyl-1-phenyl-1H-pyrazol-4-yl)-isoxazole-4-carboxylic acid (3-benzyloxy-phenyl)-amide;
98) 5-amino-3-(3,5-dimethyl-1-phenyl-1H-pyrazol-4-yl)-isoxazole-4-carboxylic acid 2,4-dimethoxy-benzylamide;
99) 5-amino-3-(3,5-dimethyl-1-phenyl-1H-pyrazol-4-yl)-isoxazole-4-carboxylic acid indan-5-ylamide;
100) 5-amino-3-(3,5-dimethyl-1-phenyl-1H-pyrazol-4-yl)-isoxazole-4-carboxylic acid (3,5-dimethoxy-phenyl)-amide;
101) 5-amino-3-quinolin-2-yl-isoxazole-4-carboxylic acid naphthalen-1-ylamide;
102) 5-amino-3-quinolin-2-yl-isoxazole-4-carboxylic acid p-tolylamide;
103) 5-amino-3-quinolin-2-yl-isoxazole-4-carboxylic acid (3-benzyloxy-phenyl)-amide;
104) 5-amino-3-quinolin-2-yl-isoxazole-4-carboxylic acid 2,4-dimethoxy-benzylamide;
105) 5-amino-3-quinolin-2-yl-isoxazole-4-carboxylic acid indan-5-ylamide;
106) 5-amino-3-quinolin-2-yl-isoxazole-4-carboxylic acid (3,5-dimethoxy-phenyl)-amide;
107) 5-amino-3-pyridin-4-yl-isoxazole-4-carboxylic acid (2-methoxy-dibenzofuran-3-yl)-amide;
108) 5-amino-3-pyridin-4-yl-isoxazole-4-carboxylic acid (3,4,5-trimethoxy-phenyl)-amide;
109) 5-amino-3-pyridin-4-yl-isoxazole-4-carboxylic acid (2-methoxy-5-trifluoromethyl-phenyl)-amide;
110) 5-amino-3-pyridin-4-yl-isoxazole-4-carboxylic acid quinolin-8-ylamide;
111) 5-amino-3-pyridin-4-yl-isoxazole-4-carboxylic acid (4-tert-butyl-phenyl)-amide;
112) 5-amino-3-thiophen-2-yl-isoxazole-4-carboxylic acid (2-methoxy-dibenzofuran-3-yl)-amide;
113) 5-amino-3-thiophen-2-yl-isoxazole-4-carboxylic acid (3,4,5-trimethoxy-phenyl)-amide;
114) 5-amino-3-thiophen-2-yl-isoxazole-4-carboxylic acid quinolin-8-ylamide;
115) 5-amino-3-thiophen-2-yl-isoxazole-4-carboxylic acid (4-tert-butyl-phenyl)-amide;
116) 5-amino-3-thiophen-3-yl-isoxazole-4-carboxylic acid (2-methoxy-dibenzofuran-3-yl)-amide;
117) 5-amino-3-thiophen-3-yl-isoxazole-4-carboxylic acid (3,4,5-trimethoxy-phenyl)-amide;
118) 5-amino-3-thiophen-3-yl-isoxazole-4-carboxylic acid (2-methoxy-5-trifluoromethyl-phenyl)-amide;
119) 5-amino-3-thiophen-3-yl-isoxazole-4-carboxylic acid quinolin-8-ylamide;
120) 5-amino-3-thiophen-3-yl-isoxazole-4-carboxylic acid (4-tert-butyl-phenyl)-amide;
121) 5-amino-3-thiazol-2-yl-isoxazole-4-carboxylic acid (2-methoxy-dibenzofuran-3-yl)-amide;
122) 5-amino-3-thiazol-2-yl-isoxazole-4-carboxylic acid quinolin-8-ylamide;
123) 5-amino-3-thiazol-2-yl-isoxazole-4-carboxylic acid (4-tert-butyl-phenyl)-amide;
124) 5-amino-3-pyridin-2-yl-isoxazole-4-carboxylic acid (2-methoxy-dibenzofuran-3-yl)-amide;
125) 5-amino-3-pyridin-2-yl-isoxazole-4-carboxylic acid (3,4,5-trimethoxy-phenyl)-amide;
126) 5-amino-3-pyridin-2-yl-isoxazole-4-carboxylic acid (2-methoxy-5-trifluoromethyl-phenyl)-amide;
127) 5-amino-3-pyridin-2-yl-isoxazole-4-carboxylic acid quinolin-8-ylamide;
128) 5-amino-3-pyridin-2-yl-isoxazole-4-carboxylic acid (4-tert-butyl-phenyl)-amide;
129) 5-amino-3-pyridin-3-yl-isoxazole-4-carboxylic acid (2-methoxy-dibenzofuran-3-yl)-amide;
130) 5-amino-3-pyridin-3-yl-isoxazole-4-carboxylic acid quinolin-8-ylamide;
131) 5-amino-3-pyridin-3-yl-isoxazole-4-carboxylic acid (4-tert-butyl-phenyl)-amide;
132) 5-amino-3-(3,5-dimethyl-1-phenyl-1H-pyrazol-4-yl)-isoxazole-4-carboxylic acid (2-methoxy-dibenzofuran-3-yl)-amide;
133) 5-amino-3-(3,5-dimethyl-1-phenyl-1H-pyrazol-4-yl)-isoxazole-4-carboxylic acid (2-methoxy-5-trifluoromethyl-phenyl)-amide;
134) 5-amino-3-(3,5-dimethyl-1-phenyl-1H-pyrazol-4-yl)-isoxazole-4-carboxylic acid (4-tert-butyl-phenyl)-amide;
135) 5-amino-3-quinolin-2-yl-isoxazole-4-carboxylic acid (2-methoxy-dibenzofuran-3-yl)-amide;
136) 5-amino-3-quinolin-2-yl-isoxazole-4-carboxylic acid (3,4,5-trimethoxy-phenyl)-amide;
137) 5-amino-3-quinolin-2-yl-isoxazole-4-carboxylic acid quinolin-8-ylamide;
138) 5-amino-3-quinolin-2-yl-isoxazole-4-carboxylic acid (4-tert-butyl-phenyl)-amide;
139) 5-amino-3-pyridin-4-yl-isoxazole-4-carboxylic acid (2-benzyl-phenyl)-amide;
140) 3-[(5-amino-3-pyridin-4-yl-isoxazole-4-carbonyl)-amino]-benzoic acid ethyl ester;
141) 5-amino-3-pyridin-4-yl-isoxazole-4-carboxylic acid (4-trifluoromethoxy-phenyl)-amide;
142) 5-amino-3-thiophen-2-yl-isoxazole-4-carboxylic acid (2-benzyl-phenyl)-amide;
143) 3-[(5-amino-3-thiophen-2-yl-isoxazole-4-carbonyl)-amino]-benzoic acid ethyl ester;
144) 5-amino-3-thiophen-2-yl-isoxazole-4-carboxylic acid (4-trifluoromethoxy-phenyl)-amide;
145) 5-amino-3-thiophen-3-yl-isoxazole-4-carboxylic acid (4-dimethylamino-phenyl)-amide;
146) 5-amino-3-thiophen-3-yl-isoxazole-4-carboxylic acid (2-benzyl-phenyl)-amide;
147) 3-[(5-amino-3-thiophen-3-yl-isoxazole-4-carbonyl)-amino]-benzoic acid ethyl ester;
148) 5-amino-3-thiophen-3-yl-isoxazole-4-carboxylic acid (4-trifluoromethoxy-phenyl)-amide;
149) 5-amino-3-thiazol-2-yl-isoxazole-4-carboxylic acid (4-dimethylamino-phenyl)-amide;
150) 5-amino-3-thiazol-2-yl-isoxazole-4-carboxylic acid (2-benzyl-phenyl)-amide;
151) 3-[(5-amino-3-thiazol-2-yl-isoxazole-4-carbonyl)-amino]-benzoic acid ethyl ester;
152) 5-amino-3-thiazol-2-yl-isoxazole-4-carboxylic acid (4-trifluoromethoxy-phenyl)-amide;
153) 5-amino-3-pyridin-2-yl-isoxazole-4-carboxylic acid (4-dimethylamino-phenyl)-amide;
154) 5-amino-3-pyridin-2-yl-isoxazole-4-carboxylic acid (2-benzyl-phenyl)-amide;
155) 3-[(5-amino-3-pyridin-2-yl-isoxazole-4-carbonyl)-amino]-benzoic acid ethyl ester;
156) 5-amino-3-pyridin-2-yl-isoxazole-4-carboxylic acid (4-trifluoromethoxy-phenyl)-amide;
157) 5-amino-3-pyridin-3-yl-isoxazole-4-carboxylic acid (4-dimethylamino-phenyl)-amide;
158) 5-amino-3-pyridin-3-yl-isoxazole-4-carboxylic acid (2-benzyl-phenyl)-amide;
159) 3-[(5-amino-3-pyridin-3-yl-isoxazole-4-carbonyl)-amino]-benzoic acid ethyl ester;
160) 5-amino-3-pyridin-3-yl-isoxazole-4-carboxylic acid (4-trifluoromethoxy-phenyl)-amide;
161) 5-amino-3-(3,5-dimethyl-1-phenyl-1H-pyrazol-4-yl)-isoxazole-4-carboxylic acid (2-benzyl-phenyl)-amide;
162) 3-{[5-amino-3-(3,5-dimethyl-1-phenyl-1H-pyrazol-4-yl)-isoxazole-4-carbonyl]-amino}-benzoic acid ethyl ester;
163) 5-amino-3-(3,5-dimethyl-1-phenyl-1H-pyrazol-4-yl)-isoxazole-4-carboxylic acid (4-trifluoromethoxy-phenyl)-amide;
164) 5-amino-3-quinolin-2-yl-isoxazole-4-carboxylic acid (4-dimethylamino-phenyl)-amide;
165) 5-amino-3-quinolin-2-yl-isoxazole-4-carboxylic acid (2-benzyl-phenyl)-amide;
166) 3-[(5-amino-3-quinolin-2-yl-isoxazole-4-carbonyl)-amino]-benzoic acid ethyl ester;
167) 5-amino-3-quinolin-2-yl-isoxazole-4-carboxylic acid (4-trifluoromethoxy-phenyl)-amide;
168) 5-amino-3-pyridin-2-yl-isoxazole-4-phenyl carboxamide;
169) 5-amino-3-(3,5-dimethyl-1-phenyl-1H-pyrazol-4-yl)-isoxazole-4-carboxylic acid (9H-fluoren-2-yl)-amide;
170) 5-amino-3-(3,5-dimethyl-1-phenyl-1H-pyrazol-4-yl)-isoxazole-4-carboxylic acid (pyridin-4-ylmethyl)-amide;
171) 5-amino-3-(3,5-dimethyl-1-phenyl-1H-pyrazol-4-yl)-isoxazole-4-carboxylic acid (3-methoxy-phenyl)-amide;
172) 5-amino-3-thiazol-2-yl-isoxazole-4-carboxylic acid (2,3-dihydro-benzo[1,4]dioxin-6-yl)-amide;
173) 5-amino-3-(3,5-dimethyl-1-phenyl-1H-pyrazol-4-yl)-isoxazole-4-carboxylic acid (2,3-dihydro-benzo[1,4]dioxin-6-yl)-amide;
174) 5-amino-3-pyridin-3-yl-isoxazole-4-carboxylic acid (9H-fluoren-2-yl)-amide;
175) 5-amino-3-thiophen-2-yl-isoxazole-4-carboxylic acid (9H-fluoren-2-yl)-amide;
176) 5-amino-3-thiophen-2-yl-isoxazole-4-carboxylic acid (4-morpholin-4-yl-phenyl)-amide;
177) 5-amino-3-thiazol-2-yl-isoxazole-4-carboxylic acid (4-morpholin-4-yl-phenyl)-amide;
178) 5-amino-3-(3,5-dimethyl-1-phenyl-1H-pyrazol-4-yl)-isoxazole-4-carboxylic acid (4-morpholin-4-yl-phenyl)-amide;
179) 5-amino-3-pyridin-2-yl-isoxazole-4-carboxylic acid (pyridin-4-ylmethyl)-amide;
180) 5-amino-3-thiophen-2-yl-isoxazole-4-carboxylic acid phenylamide;
181) 5-amino-3-thiazol-2-yl-isoxazole-4-carboxylic acid phenylamide;
182) 5-amino-3-(3,5-dimethyl-1-phenyl-1H-pyrazol-4-yl)-isoxazole-4-carboxylic acid (3-methoxy-propyl)-amide;
183) 5-amino-3-pyridin-4-yl-isoxazole-4-carboxylic acid (4-morpholin-4-yl-phenyl)-amide;
184) 5-amino-3-pyridin-2-yl-isoxazole-4-carboxylic acid phenylamide;
185) 5-amino-3-thiazol-2-yl-isoxazole-4-carboxylic acid (3-methoxy-phenyl)-amide;
186) 5-amino-3-(3,5-dimethyl-1-phenyl-1H-pyrazol-4-yl)-isoxazole-4-carboxylic acid (3-phenoxy-phenyl)-amide;
187) 5-amino-3-thiophen-3-yl-isoxazole-4-carboxylic acid (4-morpholin-4-yl-phenyl)-amide;
188) 4-{[5-amino-3-(3,5-dimethyl-1-phenyl-1H-pyrazol-4-yl)-isoxazole-4-carbonyl]-amino}-piperidine-1-carboxylic acid ethyl ester;
189) 5-amino-3-(3,5-dimethyl-1-phenyl-1H-pyrazol-4-yl)-isoxazole-4-carboxylic acid (tetrahydro-furan-2-ylmethyl)-amide;
190) 5-amino-3-thiophen-3-yl-isoxazole-4-carboxylic acid phenylamide;
191) 5-amino-3-thiophen-3-yl-isoxazole-4-carboxylic acid 2-methoxy-benzylamide;
192) 5-amino-3-pyridin-4-yl-isoxazole-4-carboxylic acid 4-chloro-benzylamide;
193) 5-amino-3-pyridin-2-yl-isoxazole-4-carboxylic acid 2-methoxy-benzylamide;
194) 5-amino-3-thiophen-2-yl-isoxazole-4-carboxylic acid 4-fluoro-benzylamide;
195) 5-amino-3-pyridin-4-yl-isoxazole-4-carboxylic acid (3,4-dimethyl-phenyl)-amide;
196) 5-amino-3-(3,5-dimethyl-1-phenyl-1H-pyrazol-4-yl)-isoxazole-4-carboxylic acid (2-dimethylamino-ethyl)-amide;
197) 5-amino-3-thiophen-3-yl-isoxazole-4-carboxylic acid (9H-fluoren-2-yl)-amide;
198) 5-amino-3-thiazol-2-yl-isoxazole-4-carboxylic acid (9H-fluoren-2-yl)-amide;
199) 5-amino-3-thiophen-3-yl-isoxazole-4-carboxylic acid (3-methoxy-propyl)-amide;
200) 5-amino-3-pyridin-4-yl-isoxazole-4-carboxylic acid (4-diethylamino-phenyl)-amide;
201) 5-amino-3-pyridin-2-yl-isoxazole-4-carboxylic acid (4-diethylamino-phenyl)-amide;
202) 5-amino-3-(3,5-dimethyl-1-phenyl-1H-pyrazol-4-yl)-isoxazole-4-carboxylic acid (4-diethylamino-phenyl)-amide;
203) 5-amino-3-pyridin-2-yl-isoxazole-4-carboxylic acid (4-butoxy-phenyl)-amide;
204) 5-amino-3-(3,5-dimethyl-1-phenyl-1H-pyrazol-4-yl)-isoxazole-4-carboxylic acid (4-butoxy-phenyl)-amide;
205) 5-amino-3-(3,5-dimethyl-1-phenyl-1H-pyrazol-4-yl)-isoxazole-4-carboxylic acid (1-ethyl-propyl)-amide;
206) 5-amino-3-thiophen-3-yl-isoxazole-4-carboxylic acid (3,4-dimethyl-phenyl)-amide;
207) 5-amino-3-thiophen-3-yl-isoxazole-4-carboxylic acid (4-fluoro-2-methyl-phenyl)-amide;
208) 5-amino-3-thiazol-2-yl-isoxazole-4-carboxylic acid [4-(acetyl-methyl-amino)-phenyl]-amide;
209) 5-amino-3-thiophen-2-yl-isoxazole-4-carboxylic acid 2-methoxy-benzylamide;
210) 5-amino-3-pyridin-4-yl-isoxazole-4-carboxylic acid [2-(4-methoxy-phenyl)-ethyl]-amide;
211) 5-amino-3-pyridin-2-yl-isoxazole-4-carboxylic acid (3-methoxy-propyl)-amide;
212) 5-amino-3-pyridin-3-yl-isoxazole-4-carboxylic acid 4-chloro-benzylamide;
213) 5-amino-3-(3,5-dimethyl-1-phenyl-1H-pyrazol-4-yl)-isoxazole-4-carboxylic acid [2-(4-methoxy-phenyl)-ethyl]-amide;
214) 5-amino-3-thiophen-3-yl-isoxazole-4-carboxylic acid (3-phenoxy-phenyl)-amide;
215) 5-amino-3-thiazol-2-yl-isoxazole-4-carboxylic acid (pyridin-4-ylmethyl)-amide;
216) 5-amino-3-thiophen-2-yl-isoxazole-4-carboxylic acid (2,3-dihydro-benzo[1,4]dioxin-6-yl)-amide;
217) 5-amino-3-pyridin-3-yl-isoxazole-4-carboxylic acid phenylamide;
218) 5-amino-3-thiazol-2-yl-isoxazole-4-carboxylic acid (2-methylsulfanyl-phenyl)-amide;
219) 5-amino-3-thiophen-2-yl-isoxazole-4-carboxylic acid (3-chloro-4-methyl-phenyl)-amide;
220) 5-amino-3-thiophen-3-yl-isoxazole-4-carboxylic acid (3-chloro-4-methyl-phenyl)-amide;
221) 5-amino-3-thiophen-2-yl-isoxazole-4-carboxylic acid (3-methoxy-phenyl)-amide;
222) 5-amino-3-pyridin-2-yl-isoxazole-4-carboxylic acid (3-methoxy-phenyl)-amide;
223) 5-amino-3-pyridin-2-yl-isoxazole-4-carboxylic acid (3-trifluoromethyl-phenyl)-amide;
224) 5-amino-3-thiazol-2-yl-isoxazole-4-carboxylic acid (3-methoxy-propyl)-amide;
225) 5-amino-3-(3,5-dimethyl-1-phenyl-1H-pyrazol-4-yl)-isoxazole-4-carboxylic acid cyclopropylamide;
226) 5-amino-3-thiophen-3-yl-isoxazole-4-carboxylic acid (3-trifluoromethyl-phenyl)-amide;
227) 4-[(5-amino-3-thiophen-3-yl-isoxazole-4-carbonyl)-amino]-piperidine-1-carboxylic acid ethyl ester;
228) 4-[(5-amino-3-pyridin-2-yl-isoxazole-4-carbonyl)-amino]-piperidine-1-carboxylic acid ethyl ester;
229) 5-amino-3-thiophen-3-yl-isoxazole-4-carboxylic acid (2,3-dihydro-benzo[1,4]dioxin-6-yl)-amide;
230) 5-amino-3-thiophen-2-yl-isoxazole-4-carboxylic acid [2-(4-methoxy-phenyl)-ethyl]-amide;
231) 5-amino-3-thiophen-2-yl-isoxazole-4-carboxylic acid (furan-2-ylmethyl)-amide;
232) 5-amino-3-thiophen-3-yl-isoxazole-4-carboxylic acid (furan-2-ylmethyl)-amide;
233) 5-amino-3-(3,5-dimethyl-1-phenyl-1H-pyrazol-4-yl)-isoxazole-4-carboxylic acid (furan-2-ylmethyl)-amide;
234) 5-amino-3-thiophen-3-yl-isoxazole-4-carboxylic acid (4-diethylamino-phenyl)-amide;
235) 5-amino-3-thiazol-2-yl-isoxazole-4-carboxylic acid (4-diethylamino-phenyl)-amide;
236) 5-amino-3-pyridin-4-yl-isoxazole-4-carboxylic acid (9H-fluoren-2-yl)-amide;
237) 5-amino-3-pyridin-2-yl-isoxazole-4-carboxylic acid (9H-fluoren-2-yl)-amide;
238) 5-amino-3-pyridin-4-yl-isoxazole-4-carboxylic acid (3-trifluoromethyl-phenyl)-amide;
239) 5-amino-3-pyridin-4-yl-isoxazole-4-carboxylic acid 4-fluoro-benzylamide;
240) 5-amino-3-thiophen-3-yl-isoxazole-4-carboxylic acid (4-butoxy-phenyl)-amide;
241) 5-amino-3-thiophen-3-yl-isoxazole-4-carboxylic acid (4-difluoromethoxy-phenyl)-amide;
242) 5-amino-3-thiazol-2-yl-isoxazole-4-carboxylic acid [4-(toluene-4-sulfonylamino)-phenyl]-amide;
243) 5-amino-3-thiophen-3-yl-isoxazole-4-carboxylic acid (2-methylsulfanyl-phenyl)-amide;
244) 5-amino-3-(3,5-dimethyl-1-phenyl-1H-pyrazol-4-yl)-isoxazole-4-carboxylic acid phenylamide;
245) 5-amino-3-thiophen-3-yl-isoxazole-4-carboxylic acid (1-ethyl-propyl)-amide;
246) 5-amino-3-pyridin-4-yl-isoxazole-4-carboxylic acid (3-methoxy-phenyl)-amide;
247) 5-amino-3-thiophen-3-yl-isoxazole-4-carboxylic acid [4-(toluene-4-sulfonylamino)-phenyl]-amide;
248) 5-amino-3-thiophen-2-yl-isoxazole-4-carboxylic acid (2-pyrrolidin-1-yl-ethyl)-amide;
249) 5-amino-3-(3,5-dimethyl-1-phenyl-1H-pyrazol-4-yl)-isoxazole-4-carboxylic acid (3-chloro-4-methyl-phenyl)-amide;
250) 5-amino-3-thiophen-2-yl-isoxazole-4-carboxylic acid (4-diethylamino-phenyl)-amide;
251) 5-amino-3-pyridin-2-yl-isoxazole-4-carboxylic acid (2-dimethylamino-ethyl)-amide;
252) 5-amino-3-thiazol-2-yl-isoxazole-4-carboxylic acid (3-trifluoromethyl-phenyl)-amide;
253) 5-amino-3-thiophen-2-yl-isoxazole-4-carboxylic acid (pyridin-4-ylmethyl)-amide;
254) 5-amino-3-pyridin-2-yl-isoxazole-4-carboxylic acid (4-morpholin-4-yl-phenyl)-amide;
255) 5-amino-3-thiophen-3-yl-isoxazole-4-carboxylic acid (3-methoxy-phenyl)-amide;
256) 5-amino-3-pyridin-4-yl-isoxazole-4-carboxylic acid (4-butoxy-phenyl)-amide;
257) 5-benzoylamino-3-pyridin-2-yl-isoxazole-4-carboxylic acid phenylamide;
258) 5-phenylacetylamino-3-thiophen-2-yl-isoxazole-4-carboxylic acid butylamide;
259) 5-(4-methyl-benzoylamino)-3-pyridin-2-yl-isoxazole-4-carboxylic acid amide;
260) 5-(4-chloro-butyrylamino)-3-pyridin-2-yl-isoxazole-4-carboxylic acid amide;
261) 5-(4-tert-butyl-benzoylamino)-3-pyridin-2-yl-isoxazole-4-carboxylic acid amide;
262) 5-pentanoylamino-3-pyridin-2-yl-isoxazole-4-carboxylic acid amide;
263) 5-(4-methyl-benzoylamino)-3-pyridin-4-yl-isoxazole-4-carboxylic acid amide;
264) 5-(4-chloro-benzoylamino)-3-pyridin-4-yl-isoxazole-4-carboxylic acid amide;
265) 5-pentanoylamino-3-pyridin-4-yl-isoxazole-4-carboxylic acid amide;
266) 5-(3-phenyl-propionylamino)-3-pyridin-4-yl-isoxazole-4-carboxylic acid amide;
267) 5-(4-tert-butyl-benzoylamino)-3-thiophen-2-yl-isoxazole-4-carboxylic acid amide;
268) 5-pentanoylamino-3-thiophen-2-yl-isoxazole-4-carboxylic acid amide;
269) 5-(3-phenyl-propionylamino)-3-thiophen-2-yl-isoxazole-4-carboxylic acid amide;
270) 5-(4-methyl-benzoylamino)-3-thiophen-3-yl-isoxazole-4-carboxylic acid amide;
271) 5-(4-tert-butyl-benzoylamino)-3-thiophen-3-yl-isoxazole-4-carboxylic acid amide;
272) 5-pentanoylamino-3-thiophen-3-yl-isoxazole-4-carboxylic acid amide;
273) 5-(3-phenyl-propionylamino)-3-thiophen-3-yl-isoxazole-4-carboxylic acid amide;
274) 5-(4-chloro-butyrylamino)-3-thiazol-2-yl-isoxazole-4-carboxylic acid amide;
275) 5-(4-tert-butyl-benzoylamino)-3-thiazol-2-yl-isoxazole-4-carboxylic acid amide;
276) 5-(4-chloro-benzoylamino)-3-thiazol-2-yl-isoxazole-4-carboxylic acid amide;
277) 5-(3-phenyl-propionylamino)-3-thiazol-2-yl-isoxazole-4-carboxylic acid amide;
278) 5-(4-methyl-benzoylamino)-3-pyridin-3-yl-isoxazole-4-carboxylic acid amide;
279) 5-(4-tert-butyl-benzoylamino)-3-pyridin-3-yl-isoxazole-4-carboxylic acid amide;
280) 5-(4-chloro-benzoylamino)-3-pyridin-3-yl-isoxazole-4-carboxylic acid amide;
281) 5-pentanoylamino-3-pyridin-3-yl-isoxazole-4-carboxylic acid amide;
282) 5-(4-tert-butyl-benzoylamino)-3-(3,5-dimethyl-1-phenyl-1H-pyrazol-4-yl)-isoxazole-4-carboxylic acid amide;
283) 5-(4-chloro-benzoylamino)-3-(3,5-dimethyl-1-phenyl-1H-pyrazol-4-yl)-isoxazole-4-carboxylic acid amide;
284) 3-(3,5-dimethyl-1-phenyl-1H-pyrazol-4-yl)-5-pentanoylamino-isoxazole-4-carboxylic acid amide;
285) 3-(3,5-dimethyl-1-phenyl-1H-pyrazol-4-yl)-5-(3-phenyl-propionylamino)-isoxazole-4-carboxylic acid amide;
286) 5-(4-chloro-butyrylamino)-3-quinolin-2-yl-isoxazole-4-carboxylic acid amide;
287) 5-pentanoylamino-3-quinolin-2-yl-isoxazole-4-carboxylic acid amide;
288) 5-(3-phenyl-propionylamino)-3-quinolin-2-yl-isoxazole-4-carboxylic acid amide;
289) 5-(3-fluoro-benzoylamino)-3-pyridin-2-yl-isoxazole-4-carboxylic acid amide;
290) 5-(3-methoxy-benzoylamino)-3-pyridin-2-yl-isoxazole-4-carboxylic acid amide;
291) 3-pyridin-2-yl-5-(4-trifluoromethyl-benzoylamino)-isoxazole-4-carboxylic acid amide;
292) 5-(3-fluoro-benzoylamino)-3-pyridin-4-yl-isoxazole-4-carboxylic acid amide;
293) 5-(2-methoxy-acetylamino)-3-pyridin-4-yl-isoxazole-4-carboxylic acid amide;
294) 5-benzoylamino-3-pyridin-4-yl-isoxazole-4-carboxylic acid amide;
295) 5-(3-methoxy-benzoylamino)-3-pyridin-4-yl-isoxazole-4-carboxylic acid amide;
296) 3-pyridin-4-yl-5-(4-trifluoromethyl-benzoylamino)-isoxazole-4-carboxylic acid amide;
297) 5-(3-bromo-benzoylamino)-3-pyridin-4-yl-isoxazole-4-carboxylic acid amide;
298) 5-(3-fluoro-benzoylamino)-3-thiophen-2-yl-isoxazole-4-carboxylic acid amide;
299) 5-(2-methoxy-acetylamino)-3-thiophen-2-yl-isoxazole-4-carboxylic acid amide;
300) 5-benzoylamino-3-thiophen-2-yl-isoxazole-4-carboxylic acid amide;
301) 5-(3-methoxy-benzoylamino)-3-thiophen-2-yl-isoxazole-4-carboxylic acid amide;
302) 3-thiophen-2-yl-5-(4-trifluoromethyl-benzoylamino)-isoxazole-4-carboxylic acid amide;
303) 5-(2-methoxy-acetylamino)-3-thiophen-3-yl-isoxazole-4-carboxylic acid amide;
304) 5-benzoylamino-3-thiophen-3-yl-isoxazole-4-carboxylic acid amide;
305) 5-(3-methoxy-benzoylamino)-3-thiophen-3-yl-isoxazole-4-carboxylic acid amide;
306) 3-thiophen-3-yl-5-(4-trifluoromethyl-benzoylamino)-isoxazole-4-carboxylic acid amide;
307) 5-(2-methoxy-acetylamino)-3-thiazol-2-yl-isoxazole-4-carboxylic acid amide;
308) 5-benzoylamino-3-thiazol-2-yl-isoxazole-4-carboxylic acid amide;
309) 5-(3-methoxy-benzoylamino)-3-thiazol-2-yl-isoxazole-4-carboxylic acid amide;
310) 3-thiazol-2-yl-5-(4-trifluoromethyl-benzoylamino)-isoxazole-4-carboxylic acid amide;
311) 5-(3-bromo-benzoylamino)-3-thiazol-2-yl-isoxazole-4-carboxylic acid amide;
312) 5-(2-methoxy-acetylamino)-3-pyridin-3-yl-isoxazole-4-carboxylic acid amide;
313) 5-(3-methoxy-benzoylamino)-3-pyridin-3-yl-isoxazole-4-carboxylic acid amide;
314) 3-pyridin-3-yl-5-(4-trifluoromethyl-benzoylamino)-isoxazole-4-carboxylic acid amide;
315) 5-(3-bromo-benzoylamino)-3-pyridin-3-yl-isoxazole-4-carboxylic acid amide;
316) 3-(3,5-dimethyl-1-phenyl-1H-pyrazol-4-yl)-5-(3-fluoro-benzoylamino)-isoxazole-4-carboxylic acid amide;
317) 3-(3,5-dimethyl-1-phenyl-1H-pyrazol-4-yl)-5-(2-methoxy-acetylamino)-isoxazole-4-carboxylic acid amide;
318) 5-(3-bromo-benzoylamino)-3-(3,5-dimethyl-1-phenyl-1H-pyrazol-4-yl)-isoxazole-4-carboxylic acid amide;
319) 3-quinolin-2-yl-5-(4-trifluoromethyl-benzoylamino)-isoxazole-4-carboxylic acid amide;
320) 5-(cyclopropanecarbonyl-amino)-3-pyridin-2-yl-isoxazole-4-carboxylic acid amide;
321) 5-(2-phenoxy-acetylamino)-3-pyridin-4-yl-isoxazole-4-carboxylic acid amide;
322) 5-(2-chloro-benzoylamino)-3-pyridin-4-yl-isoxazole-4-carboxylic acid amide;
323) 5-(cyclopropanecarbonyl-amino)-3-pyridin-4-yl-isoxazole-4-carboxylic acid amide;
324) 5-(3-methyl-but-2-enoylamino)-3-pyridin-4-yl-isoxazole-4-carboxylic acid amide;
325) 5-(2,4-difluoro-benzoylamino)-3-pyridin-4-yl-isoxazole-4-carboxylic acid amide;
326) 5-[2-(3-methoxy-phenyl)-acetylamino]-3-thiophen-2-yl-isoxazole-4-carboxylic acid amide;
327) 5-(2-phenoxy-acetylamino)-3-thiophen-2-yl-isoxazole-4-carboxylic acid amide;
328) 5-(2-chloro-benzoylamino)-3-thiophen-2-yl-isoxazole-4-carboxylic acid amide;
329) 5-(cyclopropanecarbonyl-amino)-3-thiopheh-2-yl-isoxazole-4-carboxylic acid amide;
330) 5-(3-methyl-but-2-enoylamino)-3-thiophen-2-yl-isoxazole-4-carboxylic acid amide;
331) 5-(2,4-difluoro-benzoylamino)-3-thiophen-2-yl-isoxazole-4-carboxylic acid amide;
332) 5-[2-(3-methoxy-phenyl)-acetylamino]-3-thiophen-3-yl-isoxazole-4-carboxylic acid amide;
333) 5-(2-chloro-benzoylamino)-3-thiophen-3-yl-isoxazole-4-carboxylic acid amide;
334) 5-(cyclopropanecarbonyl-amino)-3-thiophen-3-yl-isoxazole-4-carboxylic acid amide;
335) 5-(3-methyl-but-2-enoylamino)-3-thiophen-3-yl-isoxazole-4-carboxylic acid amide;
336) 5-(2,4-difluoro-benzoylamino)-3-thiophen-3-yl-isoxazole-4-carboxylic acid amide;
337) 5-(2-phenoxy-acetylamino)-3-thiazol-2-yl-isoxazole-4-carboxylic acid amide;
338) 5-(cyclopropanecarbonyl-amino)-3-thiazol-2-yl-isoxazole-4-carboxylic acid amide;
339) 5-(3-methyl-but-2-enoylamino)-3-thiazol-2-yl-isoxazole-4-carboxylic acid amide;
340) 5-(2,4-difluoro-benzoylamino)-3-thiazol-2-yl-isoxazole-4-carboxylic acid amide;
341) 5-(2-phenoxy-acetylamino)-3-pyridin-3-yl-isoxazole-4-carboxylic acid amide;
342) 5-(2-chloro-benzoylamino)-3-pyridin-3-yl-isoxazole-4-carboxylic acid amide;
343) 5-(cyclopropanecarbonyl-amino)-3-pyridin-3-yl-isoxazole-4-carboxylic acid amide;
344) 5-(2,4-difluoro-benzoylamino)-3-pyridin-3-yl-isoxazole-4-carboxylic acid amide;
345) 3-(3,5-dimethyl-1-phenyl-1H-pyrazol-4-yl)-5-(2-phenoxy-acetylamino)-isoxazole-4-carboxylic acid amide;
346) 5-(2-chloro-benzoylamino)-3-(3,5-dimethyl-1-phenyl-1H-pyrazol-4-yl)-isoxazole-4-carboxylic acid amide;
347) 5-(cyclopropanecarbonyl-amino)-3-(3,5-dimethyl-1-phenyl-1H-pyrazol-4-yl)-isoxazole-4-carboxylic acid amide;
348) 3-(3,5-dimethyl-1-phenyl-1H-pyrazol-4-yl)-5-(3-methyl-but-2-enoylamino)-isoxazole-4-carboxylic acid amide;
349) 5-(2,4-difluoro-benzoylamino)-3-(3,5-dimethyl-1-phenyl-1H-pyrazol-4-yl)-isoxazole-4-carboxylic acid amide;
350) 5-(cyclopropanecarbonyl-amino)-3-quinolin-2-yl-isoxazole-4-carboxylic acid amide;
351) 5-(4-fluoro-benzoylamino)-3-pyridin-2-yl-isoxazole-4-carboxylic acid amide;
352) 3-pyridin-2-yl-5-(4-trifluoromethoxy-benzoylamino)-isoxazole-4-carboxylic acid amide;
353) 5-(2-methyl-benzoylamino)-3-pyridin-2-yl-isoxazole-4-carboxylic acid amide;
354) 5-(4-ethyl-benzoylamino)-3-pyridin-2-yl-isoxazole-4-carboxylic acid amide;
355) 5-[(naphthalene-1-carbonyl)-amino]-3-pyridin-2-yl-isoxazole-4-carboxylic acid amide;
356) 5-(4-fluoro-benzoylamino)-3-pyridin-4-yl-isoxazole-4-carboxylic acid amide;
357) 3-pyridin-4-yl-5-(4-trifluoromethoxy-benzoylamino)-isoxazole-4-carboxylic acid amide;
358) 5-(2-methyl-benzoylamino)-3-pyridin-4-yl-isoxazole-4-carboxylic acid amide;
359) 3-pyridin-4-yl-5-(3-trifluoromethyl-benzoylamino)-isoxazole-4-carboxylic acid amide;
360) 5-(4-ethyl-benzoylamino)-3-pyridin-4-yl-isoxazole-4-carboxylic acid amide;
361) 5-[(naphthalene-1-carbonyl)-amino]-3-pyridin-4-yl-isoxazole-4-carboxylic acid amide;
362) 5-(4-fluoro-benzoylamino)-3-thiophen-2-yl-isoxazole-4-carboxylic acid amide;
363) 3-thiophen-2-yl-5-(4-trifluoromethoxy-benzoylamino)-isoxazole-4-carboxylic acid amide;
364) 5-(2-methyl-benzoylamino)-3-thiophen-2-yl-isoxazole-4-carboxylic acid amide;
365) 3-thiophen-2-yl-5-(3-trifluoromethyl-benzoylamino)-isoxazole-4-carboxylic acid amide;
366) 5-(4-ethyl-benzoylamino)-3-thiophen-2-yl-isoxazole-4-carboxylic acid amide;
367) 5-[(naphthalene-1-carbonyl)-amino]-3-thiophen-2-yl-isoxazole-4-carboxylic acid amide;
368) 5-(4-fluoro-benzoylamino)-3-thiophen-3-yl-isoxazole-4-carboxylic acid amide;
369) 3-thiophen-3-yl-5-(4-trifluoromethoxy-benzoylamino)-isoxazole-4-carboxylic acid amide;
370) 5-(2-methyl-benzoylamino)-3-thiophen-3-yl-isoxazole-4-carboxylic acid amide;
371) 3-thiophen-3-yl-5-(3-trifluoromethyl-benzoylamino)-isoxazole-4-carboxylic acid amide;
372) 5-(4-ethyl-benzoylamino)-3-thiophen-3-yl-isoxazole-4-carboxylic acid amide;
373) 5-[(naphthalene-1-carbonyl)-amino]-3-thiophen-3-yl-isoxazole-4-carboxylic acid amide;
374) 5-(4-fluoro-benzoylamino)-3-thiazol-2-yl-isoxazole-4-carboxylic acid amide;
375) 3-thiazol-2-yl-5-(4-trifluoromethoxy-benzoylamino)-isoxazole-4-carboxylic acid amide;
376) 5-(2-methyl-benzoylamino)-3-thiazol-2-yl-isoxazole-4-carboxylic acid amide;
377) 3-thiazol-2-yl-5-(3-trifluoromethyl-benzoylamino)-isoxazole-4-carboxylic acid amide;
378) 5-(4-ethyl-benzoylamino)-3-thiazol-2-yl-isoxazole-4-carboxylic acid amide;
379) 5-(4-fluoro-benzoylamino)-3-pyridin-3-yl-isoxazole-4-carboxylic acid amide;
380) 3-pyridin-3-yl-5-(4-trifluoromethoxy-benzoylamino)-isoxazole-4-carboxylic acid amide;
381) 3-pyridin-3-yl-5-(3-trifluoromethyl-benzoylamino)-isoxazole-4-carboxylic acid amide;
382) 5-(2-methyl-benzoylamino)-3-pyridin-3-yl-isoxazole-4-carboxylic acid amide;
383) 5-(4-ethyl-benzoylamino)-3-pyridin-3-yl-isoxazole-4-carboxylic acid amide;
384) 5-[(naphthalene-1-carbonyl)-amino]-3-pyridin-3-yl-isoxazole-4-carboxylic acid amide;
385) 3-(3,5-dimethyl-1-phenyl-1H-pyrazol-4-yl)-5-(4-fluoro-benzoylamino)-isoxazole-4-carboxylic acid amide;
386) 3-(3,5-dimethyl-1-phenyl-1H-pyrazol-4-yl)-5-(2-methyl-benzoylamino)-isoxazole-4-carboxylic acid amide;
387) 3-(3,5-dimethyl-1-phenyl-1H-pyrazol-4-yl)-5-(4-ethyl-benzoylamino)-isoxazole-4-carboxylic acid amide;
388) 3-(3,5-dimethyl-1-phenyl-1H-pyrazol-4-yl)-5-[(naphthalene-1-carbonyl)-amino]-isoxazole-4-carboxylic acid amide;
389) 5-(2-fluoro-benzoylamino)-3-pyridin-2-yl-isoxazole-4-carboxylic acid amide;
390) 5-(3-methyl-benzoylamino)-3-pyridin-2-yl-isoxazole-4-carboxylic acid amide;
391) 5-[2-(4-fluoro-phenyl)-acetylamino]-3-pyridin-4-yl-isoxazole-4-carboxylic acid amide;
392) 5-(3,5-difluoro-benzoylamino)-3-pyridin-4-yl-isoxazole-4-carboxylic acid amide;
393) 5-(3-chloro-benzoylamino)-3-pyridin-4-yl-isoxazole-4-carboxylic acid amide;
394) 5-(2-fluoro-benzoylamino)-3-pyridin-4-yl-isoxazole-4-carboxylic acid amide;
395) 5-(3-methyl-benzoylamino)-3-pyridin-4-yl-isoxazole-4-carboxylic acid amide;
396) 5-(3,5-difluoro-benzoylamino)-3-thiophen-2-yl-isoxazole-4-carboxylic acid amide;
397) 5-(3-chloro-benzoylamino)-3-thiophen-2-yl-isoxazole-4-carboxylic acid amide;
398) 5-(3,5-difluoro-benzoylamino)-3-thiophen-3-yl-isoxazole-4-carboxylic acid amide;
399) 5-(3-methyl-benzoylamino)-3-thiophen-3-yl-isoxazole-4-carboxylic acid amide;
400) 5-(3-chloro-benzoylamino)-3-thiazol-2-yl-isoxazole-4-carboxylic acid amide;
401) 5-(3-chloro-benzoylamino)-3-pyridin-3-yl-isoxazole-4-carboxylic acid amide;
402) 5-(2-fluoro-benzoylamino)-3-pyridin-3-yl-isoxazole-4-carboxylic acid amide;
403) 5-(3-methyl-benzoylamino)-3-pyridin-3-yl-isoxazole-4-carboxylic acid amide;
404) 5-(3-chloro-benzoylamino)-3-(3,5-dimethyl-1-phenyl-1H-pyrazol-4-yl)-isoxazole-4-carboxylic acid amide;
405) 5-butyrylamino-3-pyridin-2-yl-isoxazole-4-carboxylic acid amide;
406) 5-[(E)-(3-phenyl-acryloyl)amino]-3-pyridin-2-yl-isoxazole-4-carboxylic acid amide;
407) 5-(3,3-dimethyl-butyrylamino)-3-pyridin-2-yl-isoxazole-4-carboxylic acid amide;
408) 5-(2-chloro-2-phenyl-acetylamino)-3-pyridin-4-yl-isoxazole-4-carboxylic acid amide;
409) 5-butyrylamino-3-pyridin-4-yl-isoxazole-4-carboxylic acid amide;
410) 5-[(biphenyl-4-carbonyl)-amino]-3-pyridin-4-yl-isoxazole-4-carboxylic acid amide;
411) 5-(4-chloromethyl-benzoylamino)-3-pyridin-4-yl-isoxazole-4-carboxylic acid amide;
412) 3-pyridin-4-yl-5-(3,4,5-trimethoxy-benzoylamino)-isoxazole-4-carboxylic acid amide;
413) 5-[(E)-(3-phenyl-acryloyl)amino]-3-pyridin-4-yl-isoxazole-4-carboxylic acid amide;
414) 5-(3,3-dimethyl-butyrylamino)-3-pyridin-4-yl-isoxazole-4-carboxylic acid amide;
415) 5-(2-chloro-2-phenyl-acetylamino)-3-thiophen-2-yl-isoxazole-4-carboxylic acid amide;
416) 5-(4-nitro-benzoylamino)-3-thiophen-2-yl-isoxazole-4-carboxylic acid amide;
417) 5-butyrylamino-3-thiophen-2-yl-isoxazole-4-carboxylic acid amide;
418) 5-[(biphenyl-4-carbonyl)-amino]-3-thiophen-2-yl-isoxazole-4-carboxylic acid amide;
419) 5-(4-chloromethyl-benzoylamino)-3-thiophen-2-yl-isoxazole-4-carboxylic acid amide;
420) 3-thiophen-2-yl-5-(3,4,5-trimethoxy-benzoylamino)-isoxazole-4-carboxylic acid amide;
421) 5-[(E)-(3-phenyl-acryloyl)amino]-3-thiophen-2-yl-isoxazole-4-carboxylic acid amide;
422) 5-(3,3-dimethyl-butyrylamino)-3-thiophen-2-yl-isoxazole-4-carboxylic acid amide;
423) 5-(2-chloro-2-phenyl-acetylamino)-3-thiophen-3-yl-isoxazole-4-carboxylic acid amide;
424) 5-butyrylamino-3-thiophen-3-yl-isoxazole-4-carboxylic acid amide;
425) 5-(3,3-dimethyl-butyrylamino)-3-thiophen-3-yl-isoxazole-4-carboxylic acid amide;
426) 5-(4-chloromethyl-benzoylamino)-3-thiazol-2-yl-isoxazole-4-carboxylic acid amide;
427) 5-(3,3-dimethyl-butyrylamino)-3-thiazol-2-yl-isoxazole-4-carboxylic acid amide;
428) 5-(3,3-dimethyl-butyrylamino)-3-pyridin-3-yl-isoxazole-4-carboxylic acid amide;
429) 3-(3,5-dimethyl-1-phenyl-1H-pyrazol-4-yl)-5-[(E)-(3-phenyl-acryloyl)amino]-isoxazole-4-carboxylic acid amide;
430) 5-butyrylamino-3-(3,5-dimethyl-1-phenyl-1H-pyrazol-4-yl)-isoxazole-4-carboxylic acid amide;
431) 5-(3,3-dimethyl-butyrylamino)-3-(3,5-dimethyl-1-phenyl-1H-pyrazol-4-yl)-isoxazole-4-carboxylic acid amide;
432) 5-butyrylamino-3-quinolin-2-yl-isoxazole-4-carboxylic acid amide;
433) 5-(3,3-dimethyl-butyrylamino)-3-quinolin-2-yl-isoxazole-4-carboxylic acid amide;
434) 5-[(furan-2-carbonyl)-amino]-3-pyridin-2-yl-isoxazole-4-carboxylic acid amide;
435) 5-[(naphthalene-2-carbonyl)-amino]-3-pyridin-4-yl-isoxazole-4-carboxylic acid amide;
436) 5-(4-methoxy-benzoylamino)-3-pyridin-4-yl-isoxazole-4-carboxylic acid amide;
437) 5-[(furan-2-carbonyl)-amino]-3-pyridin-4-yl-isoxazole-4-carboxylic acid amide;
438) 5-(3-cyclopentyl-propionylamino)-3-pyridin-4-yl-isoxazole-4-carboxylic acid amide;
439) 5-[(naphthalene-2-carbonyl)-amino]-3-thiophen-2-yl-isoxazole-4-carboxylic acid amide;
440) 5-(4-methoxy-benzoylamino)-3-thiophen-2-yl-isoxazole-4-carboxylic acid amide;
441) 5-(2-benzyloxy-acetylamino)-3-thiophen-2-yl-isoxazole-4-carboxylic acid amide;
442) 5-(2,4-dimethoxy-benzoylamino)-3-thiophen-2-yl-isoxazole-4-carboxylic acid amide;
443) 5-[(furan-2-carbonyl)-amino]-3-thiophen-2-yl-isoxazole-4-carboxylic acid amide;
444) 5-(3-cyclopentyl-propionylamino)-3-thiophen-2-yl-isoxazole-4-carboxylic acid amide;
445) 5-(4-bromo-benzoylamino)-3-thiophen-2-yl-isoxazole-4-carboxylic acid amide;
446) 5-[(naphthalene-2-carbonyl)-amino]-3-thiophen-3-yl-isoxazole-4-carboxylic acid amide;
447) 5-(4-methoxy-benzoylamino)-3-thiophen-3-yl-isoxazole-4-carboxylic acid amide;
448) 5-(2,4-dimethoxy-benzoylamino)-3-thiophen-3-yl-isoxazole-4-carboxylic acid amide;
449) 5-[(furan-2-carbonyl)-amino]-3-thiophen-3-yl-isoxazole-4-carboxylic acid amide;
450) 5-(3-cyclopentyl-propionylamino)-3-thiophen-3-yl-isoxazole-4-carboxylic acid amide;
451) 5-(4-methoxy-benzoylamino)-3-thiazol-2-yl-isoxazole-4-carboxylic acid amide;
452) 5-(2,4-dimethoxy-benzoylamino)-3-thiazol-2-yl-isoxazole-4-carboxylic acid amide;
453) 5-[(furan-2-carbonyl)-amino]-3-thiazol-2-yl-isoxazole-4-carboxylic acid amide;
454) 5-(3-cyclopentyl-propionylamino)-3-thiazol-2-yl-isoxazole-4-carboxylic acid amide;
455) 5-[(furan-2-carbonyl)-amino]-3-pyridin-3-yl-isoxazole-4-carboxylic acid amide;
456) 3-(3,5-dimethyl-1-phenyl-1H-pyrazol-4-yl)-5-(4-methoxy-benzoylamino)-isoxazole-4-carboxylic acid amide;
457) 5-(2,4-dimethoxy-benzoylamino)-3-(3,5-dimethyl-1-phenyl-1H-pyrazol-4-yl)-isoxazole-4-carboxylic acid amide;
458) 3-(3,5-dimethyl-1-phenyl-1H-pyrazol-4-yl)-5-[(furan-2-carbonyl)-amino]-isoxazole-4-carboxylic acid amide;
459) 5-(3-cyclopentyl-propionylamino)-3-(3,5-dimethyl-1-phenyl-1H-pyrazol-4-yl)-isoxazole-4-carboxylic acid amide;
460) 5-(3,4-dimethoxy-benzoylamino)-3-(3,5-dimethyl-1-phenyl-1H-pyrazol-4-yl)-isoxazole-4-carboxylic acid amide;
461) 5-(4-bromo-benzoylamino)-3-(3,5-dimethyl-1-phenyl-1H-pyrazol-4-yl)-isoxazole-4-carboxylic acid amide;
462) 5-(3-cyclopentyl-propionylamino)-3-quinolin-2-yl-isoxazole-4-carboxylic acid amide;
463) 5-(2-ethyl-butyrylamino)-3-pyridin-2-yl-isoxazole-4-carboxylic acid amide;
464) 5-(2-propyl-pentanoylamino)-3-pyridin-2-yl-isoxazole-4-carboxylic acid amide;
465) 5-(3-methyl-butyrylamino)-3-pyridin-4-yl-isoxazole-4-carboxylic acid amide;
466) 5-[(benzo[1,3]dioxole-5-carbonyl)-amino·-3-pyridin-4-yl-isoxazole-4-carboxylic acid amide;
467) 5-(2-ethyl-butyrylamino)-3-pyridin-4-yl-isoxazole-4-carboxylic acid amide; 468) 5-(2-propyl-pentanoylamino)-3-pyridin-4-yl-isoxazole-4-carboxylic acid amide;
469) 5-(3-methyl-butyrylamino)-3-thiophen-2-yl-isoxazole-4-carboxylic acid amide;
470) 5-[(benzo[1,3]dioxole-5-carbonyl)-amino]-3-thiophen-2-yl-isoxazole-4-carboxylic acid amide;
471) 5-(2-ethyl-butyrylamino)-3-thiophen-2-yl-isoxazole-4-carboxylic acid amide;
472) 5-(2-propyl-pentanoylamino)-3-thiophen-2-yl-isoxazole-4-carboxylic acid amide;
473) 5-[2-(3,4-dimethoxy-phenyl)-acetylamino]-3-thiophen-2-yl-isoxazole-4-carboxylic acid amide;
474) 5-(3-methyl-butyrylamino)-3-thiophen-3-yl-isoxazole-4-carboxylic acid amide;
475) 5-[(benzo[1,3]dioxole-5-carbonyl)-amino]-3-thiophen-3-yl-isoxazole-4-carboxylic acid amide;
476) 5-(2-ethyl-butyrylamino)-3-thiophen-3-yl-isoxazole-4-carboxylic acid amide;
477) 5-(2-propyl-pentanoylamino)-3-thiophen-3-yl-isoxazole-4-carboxylic acid amide;
478) 5-(3-methyl-butyrylamino)-3-thiazol-2-yl-isoxazole-4-carboxylic acid amide;
479) 5-[(benzo[1,3]dioxole-5-carbonyl)-amino]-3-thiazol-2-yl-isoxazole-4-carboxylic acid amide;
480) 5-(2-ethyl-butyrylamino)-3-thiazol-2-yl-isoxazole-4-carboxylic acid amide;
481) 5-(2-propyl-pentanoylamino)-3-thiazol-2-yl-isoxazole-4-carboxylic acid amide;
482) 5-(2-ethyl-butyrylamino)-3-pyridin-3-yl-isoxazole-4-carboxylic acid amide;
483) 5-(2-propyl-pentanoylamino)-3-pyridin-3-yl-isoxazole-4-carboxylic acid amide;
484) 3-(3,5-dimethyl-1-phenyl-1H-pyrazol-4-yl)-5-(3-methyl-butyrylamino)-isoxazole-4-carboxylic acid amide;
485) 5-[(benzo[1,3]dioxole-5-carbonyl)-amino]-3-(3,5-dimethyl-1-phenyl-1H-pyrazol-4-yl)-isoxazole-4-carboxylic acid amide;
486) 3-(3,5-dimethyl-1-phenyl-1H-pyrazol-4-yl)-5-(2-ethyl-butyrylamino)-isoxazole-4-carboxylic acid amide;
487) 3-(3,5-dimethyl-1-phenyl-1H-pyrazol-4-yl)-5-(2-propyl-pentanoylamino)-isoxazole-4-carboxylic acid amide;
488) 5-(3-methyl-butyrylamino)-3-quinolin-2-yl-isoxazole-4-carboxylic acid amide;
489) 5-(2-ethyl-butyrylamino)-3-quinolin-2-yl-isoxazole-4-carboxylic acid amide;
490) 5-Phenylacetylamino-3-thiophen-2-yl-isoxazole-4-carboxylic acid butylamide;
491) 5-Phenylacetylamino-3-thiophen-3-yl-isoxazole-4-carboxylic acid butylamide;
492) 5-Butyrylamino-3-thiophen-3-yl-isoxazole-4-carboxylic acid butylamide;
493) 5-[(Furan-2-carbonyl)-amino]-3-thiophen-3-yl-isoxazole-4-carboxylic acid butylamide;
494) 5-Benzoylamino-3-thiophen-3-yl-isoxazole-4-carboxylic acid butylamide;
495) 5-Phenylacetylamino-3-thiophen-3-yl-isoxazole-4-carboxylic acid (4-methoxy-phenyl)-amide;
496) 5-Butyrylamino-3-thiophen-3-yl-isoxazole-4-carboxylic acid (4-methoxy-phenyl)-amide;
497) 5-[(Furan-2-carbonyl)-amino]-3-thiophen-3-yl-isoxazole-4-carboxylic acid (4-methoxy-phenyl)-amide;
498) 5-Benzoylamino-3-thiophen-3-yl-isoxazole-4-carboxylic acid (4-methoxy-phenyl)-amide;
499) 5-Phenylacetylamino-3-pyridin-2-yl-isoxazole-4-carboxylic acid butylamide;
500) 5-Butyrylamino-3-pyridin-2-yl-isoxazole-4-carboxylic acid butylamide;
501) 5-[(Furan-2-carbonyl)-amino]-3-pyridin-2-yl-isoxazole-4-carboxylic acid butylamide;
502) 5-Benzoylamino-3-pyridin-2-yl-isoxazole-4-carboxylic acid butylamide;
503) 5-Phenylacetylamino-3-pyridin-2-yl-isoxazole-4-carboxylic acid (4-methoxy-phenyl)-amide;
504) 5-Butyrylamino-3-pyridin-2-yl-isoxazole-4-carboxylic acid (4-methoxy-phenyl)-amide;
505) 5-[(Furan-2-carbonyl)-amino]-3-pyridin-2-yl-isoxazole-4-carboxylic acid (4-methoxy-phenyl)-amide;
506) 5-Benzoylamino-3-pyridin-2-yl-isoxazole-4-carboxylic acid (4-methoxy-phenyl)-amide;
507) 5-(4-Acetylamino-benzenesulfonylamino)-3-pyridin-2-yl-isoxazole-4-carboxylic acid phenylamide;
508) 5-(4-Acetylamino-benzenesulfonylamino)-3-pyridin-2-yl-isoxazole-4-carboxylic acid amide;
509) 5-(Butane-1-sulfonylamino)-3-pyridin-2-yl-isoxazole-4-carboxylic acid amide;
510) 3-Pyridin-2-yl-5-(toluene-4-sulfonylamino)-isoxazole-4-carboxylic acid amide;
511) 5-(2,5-Dichloro-thiophene-3-sulfonylamino)-3-pyridin-2-yl-isoxazole-4-carboxylic acid amide;
512) 5-(Butane-1-sulfonylamino)-3-pyridin-2-yl-isoxazole-4-carboxylic acid acetylamide;
513) 3-Pyridin-2-yl-5-(toluene-4-sulfonylamino)-isoxazole-4-carboxylic acid benzylamide;
514) 5-(2,5-Dichloro-thiophene-3-sulfonylamino)-3-pyridin-2-yl-isoxazole-4-carboxylic acid phenylamide;
515) 5-Methanesulfonylamino-3-thiophen-3-yl-isoxazole-4-carboxylic acid amide;
516) 5-Benzenesulfonylamino-3-thiophen-3-yl-isoxazole-4-carboxylic acid amide;
517) 5-Phenylmethanesulfonylamino-3-thiophen-3-yl-isoxazole-4-carboxylic acid amide;
518) 5-(Thiophene-2-sulfonylamino)-3-thiophen-3-yl-isoxazole-4-carboxylic acid amide;
519) 5-Benzenesulfonylamino-3-pyridin-2-yl-isoxazole-4-carboxylic acid amide;
520) 5-Phenylmethanesulfonylamino-3-pyridin-2-yl-isoxazole-4-carboxylic acid amide;
521) 3-Pyridin-2-yl-5-(thiophene-2-sulfonylamino)-isoxazole-4-carboxylic acid amide;
522) 5-Methanesulfonylamino-3-pyridin-2-yl-isoxazole-4-carboxylic acid amide;
523) 5-Methanesulfonylamino-3-thiophen-3-yl-isoxazole-4-carboxylic acid butylamide;
524) 5-Benzenesulfonylamino-3-thiophen-3-yl-isoxazole-4-carboxylic acid butylamide;
525) 5-Phenylmethanesulfonylamino-3-thiophen-3-yl-isoxazole-4-carboxylic acid butylamide;
526) 5-(Thiophene-2-sulfonylamino)-3-thiophen-3-yl-isoxazole-4-carboxylic acid butylamide;
527) 5-Methanesulfonylamino-3-thiophen-3-yl-isoxazole-4-carboxylic acid (4-methoxy-phenyl)-amide;
528) 5-Benzenesulfonylamino-3-thiophen-3-yl-isoxazole-4-carboxylic acid (4-methoxy-phenyl)-amide;
529) 5-Phenylmethanesulfonylamino-3-thiophen-3-yl-isoxazole-4-carboxylic acid (4-methoxy-phenyl)-amide;
530) 5-(Thiophene-2-sulfonylamino)-3-thiophen-3-yl-isoxazole-4-carboxylic acid (4-methoxy-phenyl)-amide;
531) 5-Benzenesulfonylamino-3-pyridin-2-yl-isoxazole-4-carboxylic acid butylamide;
532) 5-Phenylmethanesulfonylamino-3-pyridin-2-yl-isoxazole-4-carboxylic acid butylamide;
533) 3-Pyridin-2-yl-5-(thiophene-2-sulfonylamino)-isoxazole-4-carboxylic acid butylamide;
534) 5-Methanesulfonylamino-3-pyridin-2-yl-isoxazole-4-carboxylic acid butylamide;
535) 5-Methanesulfonylamino-3-pyridin-2-yl-isoxazole-4-carboxylic acid (4-methoxy-phenyl)-amide;
536) 5-Benzenesulfonylamino-3-pyridin-2-yl-isoxazole-4-carboxylic acid (4-methoxy-phenyl)-amide;
537) 5-Phenylmethanesulfonylamino-3-pyridin-2-yl-isoxazole-4-carboxylic acid (4-methoxy-phenyl)-amide;
538) 3-Pyridin-2-yl-5-(thiophene-2-sulfonylamino)-isoxazole-4-carboxylic acid (4-methoxy-phenyl)-amide;
539) Phenyl 5-amino-3-(2-pyridinyl)-4-isoxazolecarboxylate
540) Cyclohexyl 5-amino-3-(2-pyridinyl)-4-isoxazolecarboxylate
541) 5-Benzothiophenyl 5-amino-3-(2-pyridinyl)-4-isoxazolecarboxylate;
542) Phenyl 5-acetylamino-3-(2-pyridinyl)-4-isoxazolecarboxylate;
543) Ciclohexyl 5-benzoylamino-3-(2-pyridinyl)-4-isoxazolecarboxylate;
544) 5-Benzothiophenyl 5-(2,4-dimethoxybenzoylamino)-3-(2-pyridinyl)-4-isoxazolecarboxylate;
545) 5-(3-Phenyl-ureido)-3-thiophen-2-yl-isoxazole-4-carboxylic acid amide;
546) 5-(3-Isopropyl-ureido)-3-thiophen-2-yl-isoxazole-4-carboxylic acid amide;
547) 5-(3-Phenyl-ureido)-3-thiophen-3-yl-isoxazole-4-carboxylic acid butylamide;
548) 5-(3-Benzyl-ureido)-3-thiophen-3-yl-isoxazole-4-carboxylic acid butylamide;
549) 5-(3-Isopropyl-ureido)-3-thiophen-3-yl-isoxazole-4-carboxylic acid butylamide;
550) 5-(3-Pyridin-3-yl-ureido)-3-thiophen-3-yl-isoxazole-4-carboxylic acid butylamide;
551) 5-(3-Phenyl-ureido)-3-thiophen-3-yl-isoxazole-4-carboxylic acid (4-methoxy-phenyl)-amide;
552) 5-(3-Benzyl-ureido)-3-thiophen-3-yl-isoxazole-4-carboxylic acid (4-methoxy-phenyl)-amide;
553) 5-(3-Isopropyl-ureido)-3-thiophen-3-yl-isoxazole-4-carboxylic acid (4-methoxy-phenyl)-amide;
554) 5-(3-Pyridin-3-yl-ureido)-3-thiophen-3-yl-isoxazole-4-carboxylic acid (4-methoxy-phenyl)-amide;
555) 5-(3-Phenyl-ureido)-3-pyridin-2-yl-isoxazole-4-carboxylic acid butylamide;
556) 5-(3-Benzyl-ureido)-3-pyridin-2-yl-isoxazole-4-carboxylic acid butylamide;
557) 5-(3-Isopropyl-ureido)-3-pyridin-2-yl-isoxazole-4-carboxylic acid butylamide;
558) 3-Pyridin-2-yl-5-(3-pyridin-3-yl-ureido)-isoxazole-4-carboxylic acid butylamide;
559) 5-(3-Phenyl-ureido)-3-pyridin-2-yl-isoxazole-4-carboxylic acid (4-methoxy-phenyl)-amide;
560) 5-(3-Benzyl-ureido)-3-pyridin-2-yl-isoxazole-4-carboxylic acid (4-methoxy-phenyl)-amide;
561) 5-(3-Isopropyl-ureido)-3-pyridin-2-yl-isoxazole-4-carboxylic acid (4-methoxy-phenyl)-amide;
562) 3-Pyridin-2-yl-5-(3-pyridin-3-yl-ureido)-isoxazole-4-carboxylic acid (4-methoxy-phenyl)-amide;
563) 5-amino-N-benzyl-3-{5-[3-(trifluoromethyl)phenyl]-2-furyl}isoxazole-4-carboxamide;
564) 5-amino-N-(2-furylmethyl)-3-{5-[3-(trifluoromethyl)phenyl]-2-furyl}isoxazole-4-carboxamide;
565) 5-amino-N-(2-phenylethyl)-3-{5-[3-(trifluoromethyl)phenyl]-2-furyl}isoxazole-4-carboxamide;
566) 5-amino-N-cyclohexyl-3-{5-[3-(trifluoromethyl)phenyl]-2-furyl}isoxazole-4-carboxamide;
567) 5-amino-N-(4-methoxybenzyl)-3-{5-[3-(trifluoromethyl)phenyl]-2-furyl}isoxazole-4-carboxamide;
568) 5-amino-N-hexyl-3-{5-[3-(trifluoromethyl)phenyl]-2-furyl}isoxazole-4-carboxamide;
569) 5-amino-3-{5-[3-(trifluoromethyl)phenyl]-2-furyl}-N-(tetrahydrofuran-2-ylmethyl)isoxazole-4-carboxamide;
570) 5-amino-3-{5-[3-(trifluoromethyl)phenyl]-2-furyl}-N-(thien-2-ylmethyl)isoxazole-4-carboxamide;
571) 5-amino-N-cyclopentyl-3-{5-[3-(trifluoromethyl)phenyl]-2-furyl}isoxazole-4-carboxamide;
572) 5-amino-N-(1,5-dimethylhexyl)-3-{5-[3-(trifluoromethyl)phenyl]-2-furyl}isoxazole-4-carboxamide;
573) 5-amino-N-isopentyl-3-{5-[3-(trifluoromethyl)phenyl]-2-furyl}isoxazole-4-carboxamide;
574) 5-amino-N-(2-ethylhexyl)-3-{5-[3-(trifluoromethyl)phenyl]-2-furyl}isoxazole-4-carboxamide;
575) 5-amino-3-{5-[3-(trifluoromethyl)phenyl]-2-furyl}-N-(4-phenylbutyl)isoxazole-4-carboxamide;
576) 5-amino-N-(2-methylcyclohexyl)-3-{5-[3-(trifluoromethyl)phenyl]-2-furyl}isoxazole-4-carboxamide;
577) 5-amino-N-[3,5-bis(trifluoromethyl)benzyl]-3-{5-[3-(trifluoromethyl)phenyl]-2-furyl}isoxazole-4-carboxamide;
578) 5-amino-3-{5-[3-(trifluoromethyl)phenyl]-2-furyl}-N-[3-(trifluoromethyl)benzyl]isoxazole-4-carboxamide;
579) 5-amino-3-{5-[3-(trifluoromethyl)phenyl]-2-furyl}-N-(3-phenylpropyl)isoxazole-4-carboxamide;
580) 5-amino-N-isobutyl-3-{5-[3-(trifluoromethyl)phenyl]-2-furyl}isoxazole-4-carboxamide;
581) 5-amino-3-{5-[3-(trifluoromethyl)phenyl]-2-furyl}-N-[2-(trifluoromethyl)benzyl]isoxazole-4-carboxamide;
582) 5-amino-N-(2-methoxybenzyl)-3-{5-[3-(trifluoromethyl)phenyl]-2-furyl}isoxazole-4-carboxamide;
583) 5-amino-N-(2-fluorobenzyl)-3-{5-[3-(trifluoromethyl)phenyl]-2-furyl}isoxazole-4-carboxamide;
584) 5-amino-3-{5-[3-(trifluoromethyl)phenyl]-2-furyl}-N-[4-(trifluoromethyl)benzyl]isoxazole-4-carboxamide;
585) 5-amino-N-(3,4-difluorobenzyl)-3-{5-[3-(trifluoromethyl)phenyl]-2-furyl}isoxazole-4-carboxamide;
586) 5-amino-N-(3-chlorobenzyl)-3-{5-[3-(trifluoromethyl)phenyl]-2-furyl}isoxazole-4-carboxamide;
587) 5-amino-N-(2-chlorobenzyl)-3-{5-[3-(trifluoromethyl)phenyl]-2-furyl}isoxazole-4-carboxamide;
588) 5-amino-N-(3-methylbenzyl)-3-{5-[3-(trifluoromethyl)phenyl]-2-furyl}isoxazole-4-carboxamide;
589) 5-amino-3-{5-[3-(trifluoromethyl)phenyl]-2-furyl}-N-(1-naphthylmethyl)isoxazole-4-carboxamide;
590) 5-amino-N-(cyclopropylmethyl)-3-{5-[3-(trifluoromethyl)phenyl]-2-furyl}isoxazole-4-carboxamide;
591) 5-amino-N-cycloheptyl-3-{5-[3-(trifluoromethyl)phenyl]-2-furyl}isoxazole-4-carboxamide;
592) 5-amino-N-[2-(4-methoxyphenyl)ethyl]-3-{5-[3-(trifluoromethyl)phenyl]-2-furyl}isoxazole-4-carboxamide;
593) 5-amino-N-(2-cyclohex-1-en-1-ylethyl)-3-{5-[3-(trifluoromethyl)phenyl]-2-furyl}isoxazole-4-carboxamide;
594) 5-amino-N-(1,2-dimethylpropyl)-3-{5-[3-(trifluoromethyl)phenyl]-2-furyl}isoxazole-4-carboxamide;
595) 5-amino-N-(3,3-diphenylpropyl)-3-{5-[3-(trifluoromethyl)phenyl]-2-furyl}isoxazole-4-carboxamide;
596) 5-amino-N-(1,2-diphenylethyl)-3-{5-[3-(trifluoromethyl)phenyl]-2-furyl}isoxazole-4-carboxamide;
597) 5-amino-N-(2,4-dimethoxybenzyl)-3-{5-[3-(trifluoromethyl)phenyl]-2-furyl}isoxazole-4-carboxamide;
598) 5-amino-N-[3-(dibutylamino)propyl]-3-{5-[3-(trifluoromethyl)phenyl]-2-furyl}isoxazole-4-carboxamide;
599) 5-amino-N-butyl-3-{5-[3-(trifluoromethyl)phenyl]-2-furyl}isoxazole-4-carboxamide;
600) 5-amino-3-{5-[3-(trifluoromethyl)phenyl]-2-furyl)-N-[4-(trifluoromethoxy)benzyl]isoxazole-4-carboxamide;
601) 5-amino-N-(1-methylhexyl)-3-{5-[3-(trifluoromethyl)phenyl]-2-furyl}isoxazole-4-carboxamide;
602) 5-amino-N-(2-methoxy-1-methylethyl)-3-{5-[3-(trifluoromethyl)phenyl]-2-furyl}isoxazole-4-carboxamide;
603) 5-amino-N-(1-methylbutyl)-3-{5-[3-(trifluoromethyl)phenyl]-2-furyl}isoxazole-4-carboxamide;
604) 5-amino-N-(3-fluorobenzyl)-3-{5-[3-(trifluoromethyl)phenyl]-2-furyl}isoxazole-4-carboxamide;
605) 5-amino-N-(4-methylbenzyl)-3-{5-[3-(trifluoromethyl)phenyl]-2-furyl}isoxazole-4-carboxamide;
606) 5-amino-N-(1,3-dimethylbutyl)-3-{5-[3-(trifluoromethyl)phenyl]-2-furyl}isoxazole-4-carboxamide;
607) 5-amino-3-{5-[3-(trifluoromethyl)phenyl]-2-furyl}-N-(1-methyl-3-phenylpropyl)isoxazole-4-carboxamide;
608) 5-amino-3-{5-[3-(trifluoromethyl)phenyl]-2-furyl}-N-[3-(methylthio)propyl]isoxazole-4-carboxamide;
609) 5-amino-N-(1,3-benzodioxol-5-ylmethyl)-3-{5-[3-(trifluoromethyl)phenyl]-2-furyl}isoxazole-4-carboxamide;
610) 5-amino-3-{5-[3-(trifluoromethyl)phenyl]-2-furyl}-N-pentylisoxazole-4-carboxamide;
611) 5-amino-N-(cyclohexylmethyl)-3-{5-[3-(trifluoromethyl)phenyl]-2-furyl}isoxazole-4-carboxamide;
612) 5-amino-N-[2-(3-chlorophenyl)ethyl]-3-{5-[3-(trifluoromethyl)phenyl]-2-furyl}isoxazole-4-carboxamide;
613) 5-amino-N-(1-ethylpropyl)-3-(5-[3-(trifluoromethyl)phenyl]-2-furyl}isoxazole-4-carboxamide;
614) 5-amino-3-{5-[3-(trifluoromethyl)phenyl]-2-furyl}-N-(1-phenylethyl)isoxazole-4-carboxamide;
615) 5-amino-N-(2-methylbenzyl)-3-{5-[3-(trifluoromethyl)phenyl]-2-furyl}isoxazole-4-carboxamide;
616) 5-amino-N-cyclobutyl-3-{5-[3-(trifluoromethyl)phenyl]-2-furyl}isoxazole-4-carboxamide;
617) 5-amino-3-{5-[3-(trifluoromethyl)phenyl]-2-furyl}-N-octylisoxazole-4-carboxamide;
618) 5-amino-N-(2-bromobenzyl)-3-{5-[3-(trifluoromethyl)phenyl]-2-furyl}isoxazole-4-carboxamide;
619) 5-amino-N-(1-methylheptyl)-3-{5-[3-(trifluoromethyl)phenyl]-2-furyl}isoxazole-4-carboxamide;
620) 5-amino-N-heptyl-3-{5-[3-(trifluoromethyl)phenyl]-2-furyl}isoxazole-4-carboxamide;
621) 5-amino-N-(tert-butyl)-3-{5-[3-(trifluoromethyl)phenyl]-2-furyl}isoxazole-4-carboxamide;
622) 5-amino-N-(3,4-dimethoxybenzyl)-3-{5-[3-(trifluoromethyl)phenyl]-2-furyl}isoxazole-4-carboxamide;
623) 5-amino-N-(2,3-dihydro-1H-inden-1-yl)-3-{5-[3-(trifluoromethyl)phenyl]-2-furyl}isoxazole-4-carboxamide;
624) 5-amino-N-(1,2,3,4-tetrahydronaphthalen-1-yl)-3-{5-[3-(trifluoromethyl)phenyl]-2-furyl}isoxazole-4-carboxamide;
625) 5-amino-N-benzyl-3-(5-bromothien-2-yl)isoxazole-4-carboxamide;
626) 5-amino-3-(5-bromothien-2-yl)-N-(2-furylmethyl)isoxazole-4-carboxamide;
627) 5-amino-3-(5-bromothien-2-yl)-N-(2-phenylethyl)isoxazole-4-carboxamide;
628) 5-amino-3-(5-bromothien-2-yl)-N-(sec-butyl)isoxazole-4-carboxamide;
629) 5-amino-3-(5-bromothien-2-yl).-N-cyclohexylisoxazole-4-carboxamide;
630) 5-amino-3-(5-bromothien-2-yl)-N-(4-methoxybenzyl)isoxazole-4-carboxamide;
631) 5-amino-3-(5-bromothien-2-yl)-N-isopropylisoxazole-4-carboxamide;
632) 5-amino-3-(5-bromothien-2-yl)-N-hexylisoxazole-4-carboxamide;
633) N-allyl-5-amino-3-(5-bromothien-2-yl)isoxazole-4-carboxamide;
634) 5-amino-3-(5-bromothien-2-yl)-N-(tetrahydrofuran-2-ylmethyl)isoxazole-4-carboxamide;
635) 5-amino-3-(5-bromothien-2-yl)-N-(thien-2-ylmethyl)isoxazole-4-carboxamide;
636) 5-amino-3-(5-bromothien-2-yl)-N-cyclopentylisoxazole-4-carboxamide;
637) 5-amino-3-(5-bromothien-2-yl)-N-(1,5-dimethylhexyl)isoxazole-4-carboxamide;
638) 5-amino-3-(5-bromothien-2-yl)-N-isopentylisoxazole-4-carboxamide;
639) 5-amino-3-(5-bromothien-2-yl)-N-(2-ethylhexyl)isoxazole-4-carboxamide;
640) 5-amino-3-(5-bromothien-2-yl)-N-(4-phenylbutyl)isoxazole-4-carboxamide;
641) 5-amino-3-(5-bromothien-2-yl)-N-(2-methylcyclohexyl)isoxazole-4-carboxamide;
642) 5-amino-N-[3,5-bis(trifluoromethyl)benzyl]-3-(5-bromothien-2-yl)isoxazole-4-carboxamide;
643) 5-amino-3-(5-bromothien-2-yl)-N-[3-(trifluoromethyl)benzyl]isoxazole-4-carboxamide;
644) 5-amino-3-(5-bromothien-2-yl)-N-(3-phenylpropyl)isoxazole-4-carboxamide;
645) 5-amino-3-(5-bromothien-2-yl)-N-isobutylisoxazole-4-carboxamide;
646) 5-amino-3-(5-bromothien-2-yl)-N-[2-(trifluoromethyl)benzyl]isoxazole-4-carboxamide;
647) 5-amino-3-(5-bromothien-2-yl)-N-(2-methoxybenzyl)isoxazole-4-carboxamide;
648) 5-amino-3-(5-bromothien-2-yl)-N-(2-fluorobenzyl)isoxazole-4-carboxamide;
649) 5-amino-3-(5-bromothien-2-yl)-N-[4-(trifluoromethyl)benzyl]isoxazole-4-carboxamide;
650) 5-amino-3-(5-bromothien-2-yl)-N-(3,4-difluorobenzyl)isoxazole-4-carboxamide;
651) 5-amino-3-(5-bromothien-2-yl)-N-(3-chlorobenzyl)isoxazole-4-carboxamide;
652) 5-amino-3-(5-bromothien-2-yl)-N-(2-chlorobenzyl)isoxazole-4-carboxamide;
653) 5-amino-3-(5-bromothien-2-yl)-N-(3-methylbenzyl)isoxazole-4-carboxamide;
654) 5-amino-3-(5-bromothien-2-yl)-N-(1-naphthylmethyl)isoxazole-4-carboxamide;
655) 5-amino-3-(5-bromothien-2-yl)-N-(cyclopropylmethyl)isoxazole-4-carboxamide;
656) 5-amino-3-(5-bromothien-2-yl)-N-cycloheptylisoxazole-4-carboxamide;
657) 5-amino-3-(5-bromothien-2-yl)-N-[2-(4-methoxyphenyl)ethyl]isoxazole-4-carboxamide;
658) 5-amino-3-(5-bromothien-2-yl)-N-(2-cyclohex-1-en-1-ylethyl)isoxazole-4-carboxamide;
659) 5-amino-3-(5-bromothien-2-yl)-N-prop-2-ynylisoxazole-4-carboxamide;
660) 5-amino-3-(5-bromothien-2-yl)-N-(1,2-dimethylpropyl)isoxazole-4-carboxamide;
661) 5-amino-3-(5-bromothien-2-yl)-N-(3,3-diphenylpropyl)isoxazole-4-carboxamide;
662) 5-amino-3-(5-bromothien-2-yl)-N-(1,2-diphenylethyl)isoxazole-4-carboxamide;
663) 5-amino-3-(5-bromothien-2-yl)-N-[3-(dibutylamino)propyl]isoxazole-4-carboxamide;
664) 5-amino-3-(5-bromothien-2-yl)-N-butylisoxazole-4-carboxamide;
665) 5-amino-3-(5-bromothien-2-yl)-N-[4-(trifluoromethoxy)benzyl]isoxazole-4-carboxamide;
666) 5-amino-3-(5-bromothien-2-yl)-N-[(5-methyl-2-furyl)methyl]isoxazole-4-carboxamide;
667) 5-amino-3-(5-bromothien-2-yl)-N-(1-methylhexyl)isoxazole-4-carboxamide;
668) 5-amino-3-(5-bromothien-2-yl)-N-(2-methoxy-1-methylethyl)isoxazole-4-carboxamide;
669) 5-amino-3-(5-bromothien-2-yl)-N-(4-tert-butylcyclohexyl)isoxazole-4-carboxamide;
670) 5-amino-3-(5-bromothien-2-yl)-N-(1-methylbutyl)isoxazole-4-carboxamide;
671) 5-amino-3-(5-bromothien-2-yl)-N-(3-fluorobenzyl)isoxazole-4-carboxamide;
672) 5-amino-3-(5-bromothien-2-yl)-N-(4-methylbenzyl)isoxazole-4-carboxamide;
673) 5-amino-3-(5-bromothien-2-yl)-N-(1,3-dimethylbutyl)isoxazole-4-carboxamide;
674) 5-amino-3-(5-bromothien-2-yl)-N-(1-methyl-3-phenylpropyl)isoxazole-4-carboxamide;
675) 5-amino-3-(5-bromothien-2-yl)-N-[3-(methylthio)propyl]isoxazole-4-carboxamide;
676) 5-amino-N-(1,3-benzodioxol-5-ylmethyl)-3-(5-bromothien-2-yl)isoxazole-4-carboxamide;
677) 5-amino-3-(5-bromothien-2-yl)-N-pentylisoxazole-4-carboxamide;
678) 5-amino-3-(5-bromothien-2-yl)-N-(cyclohexylmethyl)isoxazole-4-carboxamide;
679) ethyl 4-({[5-amino-3-(5-bromothien-2-yl)isoxazol-4-yl]carbonyl}amino)piperidine-1-carboxylate;
680) 5-amino-3-(5-bromothien-2-yl)-N-[2-(3-chlorophenyl)ethyl]isoxazole-4-carboxamide;
681) 5-amino-3-(5-bromothien-2-yl)-N-(1-ethylpropyl)isoxazole-4-carboxamide;
682) 5-amino-3-(5-bromothien-2-yl)-N-(1-phenylethyl)isoxazole-4-carboxamide;
683) 5-amino-3-(5-bromothien-2-yl)-N-propylisoxazole-4-carboxamide;
684) 5-amino-3-(5-bromothien-2-yl)-N-(2-methylbenzyl)isoxazole-4-carboxamide;
685) 5-amino-3-(5-bromothien-2-yl)-N-cyclobutylisoxazole-4-carboxamide;
686) 5-amino-3-(5-bromothien-2-yl)-N-(3-methoxypropyl)isoxazole-4-carboxamide;
687) 5-amino-3-(5-bromothien-2-yl)-N-octylisoxazole-4-carboxamide;
688) 5-amino-N-(2-bromobenzyl)-3-(5-bromothien-2-yl)isoxazole-4-carboxamide;
689) 5-amino-3-(5-bromothien-2-yl)-N-(1-methylheptyl)isoxazole-4-carboxamide;
690) 5-amino-3-(5-bromothien-2-yl)-N-heptylisoxazole-4-carboxamide;
691) 5-amino-3-(5-bromothien-2-yl)-N-(tert-butyl)isoxazole-4-carboxamide;
692) 5-amino-3-(5-bromothien-2-yl)-N-(2,3-dihydro-1H-inden-1-yl)isoxazole-4-carboxamide;
693) 5-amino-3-(5-bromothien-2-yl)-N-(1,2,3,4-tetrahydronaphthalen-1-yl)isoxazole-4-carboxamide;
694) 5-amino-N-benzyl-3-(5-chlorothien-2-yl)isoxazole-4-carboxamide;
695) 5-amino-3-(5-chlorothien-2-yl)-N-(2-furylmethyl)isoxazole-4-carboxamide;
696) 5-amino-3-(5-chlorothien-2-yl)-N-(2-phenylethyl)isoxazole-4-carboxamide;
697) 5-amino-N-(sec-butyl)-3-(5-chlorothien-2-yl)isoxazole-4-carboxamide;
698) 5-amino-3-(5-chlorothien-2-yl)-N-cyclohexylisoxazole-4-carboxamide;
699) 5-amino-3-(5-chlorothien-2-yl)-N-(4-methoxybenzyl)isoxazole-4-carboxamide;
700) 5-amino-3-(5-chlorothien-2-yl)-N-isopropylisoxazole-4-carboxamide;
701) 5-amino-3-(5-chlorothien-2-yl)-N-hexylisoxazole-4-carboxamide;
702) N-allyl-5-amino-3-(5-chlorothien-2-yl)isoxazole-4-carboxamide;
703) 5-amino-3-(5-chlorothien-2-yl)-N-(thien-2-ylmethyl)isoxazole-4-carboxamide;
704) 5-amino-3-(5-chlorothien-2-yl)-N-cyclopentylisoxazole-4-carboxamide;
705) 5-amino-3-(5-chlorothien-2-yl)-N-isopentylisoxazole-4-carboxamide;
706) 5-amino-3-(5-chlorothien-2-yl)-N-(2-ethylhexyl)isoxazole-4-carboxamide;
707) 5-amino-3-(5-chlorothien-2-yl)-N-(4-phenylbutyl)isoxazole-4-carboxamide;
708) 5-amino-3-(5-chlorothien-2-yl)-N-(2-methylcyclohexyl)isoxazole-4-carboxamide;
709) 5-amino-3-(5-chlorothien-2-yl)-N-[3-(trifluoromethyl)benzyl]isoxazole-4-carboxamide;
710) 5-amino-3-(5-chlorothien-2-yl)-N-(2-fluorobenzyl)isoxazole-4-carboxamide;
711) 5-amino-3-(5-chlorothien-2-yl)-N-(3,4-difluorobenzyl)isoxazole-4-carboxamide;
712) 5-amino-N-(3-chlorobenzyl)-3-(5-chlorothien-2-yl)isoxazole-4-carboxamide;
713) 5-amino-N-(2-chlorobenzyl)-3-(5-chlorothien-2-yl)isoxazole-4-carboxamide;
714) 5-amino-3-(5-chlorothien-2-yl)-N-(1-naphthylmethyl)isoxazole-4-carboxamide;
715) 5-amino-3-(5-chlorothien-2-yl)-N-(cyclopropylmethyl)isoxazole-4-carboxamide;
716) 5-amino-3-(5-chlorothien-2-yl)-N-cycloheptylisoxazole-4-carboxamide;
717) 5-amino-3-(5-chlorothien-2-yl)-N-[2-(4-methoxyphenyl)ethyl]isoxazole-4-carboxamide;
718) 5-amino-3-(5-chlorothien-2-yl)-N-(2-cyclohex-1-en-1-ylethyl)isoxazole-4-carboxamide;
719) 5-amino-3-(5-chlorothien-2-yl)-N-(3,3-diphenylpropyl)isoxazole-4-carboxamide;
720) 5-amino-3-(5-chlorothien-2-yl)-N-(1,2-diphenylethyl)isoxazole-4-carboxamide;
721) 5-amino-3-(5-chlorothien-2-yl)-N-[3-(dibutylamino)propyl]isoxazole-4-carboxamide;
722) 5-amino-N-butyl-3-(5-chlorothien-2-yl)isoxazole-4-carboxamide;
723) 5-amino-3-(5-chlorothien-2-yl)-N-[4-(trifluoromethoxy)benzyl]isoxazole-4-carboxamide;
724) 5-amino-3-(5-chlorothien-2-yl)-N-[(5-methyl-2-furyl)methyl]isoxazole-4-carboxamide;
725) 5-amino-3-(5-chlorothien-2-yl)-N-(1-methylhexyl)isoxazole-4-carboxamide;
726) 5-amino-N-(1-benzylpiperidin-4-yl)-3-(5-chlorothien-2-yl)isoxazole-4-carboxamide;
727) 5-amino-3-(5-chlorothien-2-yl)-N-(2-methoxy-1-methylethyl)isoxazole-4-carboxamide;
728) 5-amino-3-(5-chlorothien-2-yl)-N-(1-methylbutyl)isoxazole-4-carboxamide;
729) 5-amino-3-(5-chlorothien-2-yl)-N-(3-fluorobenzyl)isoxazole-4-carboxamide;
730) 5-amino-3-(5-chlorothien-2-yl)-N-(1,3-dimethylbutyl)isoxazole-4-carboxamide;
731) 5-amino-3-(5-chlorothien-2-yl)-N-(1-methyl-3-phenylpropyl)isoxazole-4-carboxamide;
732) 5-amino-3-(5-chlorothien-2-yl)-N-[3-(methylthio)propyl]isoxazole-4-carboxamide;
733) 5-amino-N-(1,3-benzodioxol-5-ylmethyl)-3-(5-chlorothien-2-yl)isoxazole-4-carboxamide;
734) 5-amino-3-(5-chlorothien-2-yl)-N-pentylisoxazole-4-carboxamide;
735) 5-amino-3-(5-chlorothien-2-yl)-N-(cyclohexylmethyl)isoxazole-4-carboxamide;
736) ethyl 4-({[5-amino-3-(5-chlorothien-2-yl)isoxazol-4-yl]carbonyl}amino)piperidine-1-carboxylate;
737) 5-amino-N-[2-(3-chlorophenyl)ethyl]-3-(5-chlorothien-2-yl)isoxazole-4-carboxamide;
738) 5-amino-3-(5-chlorothien-2-yl)-N-(1-ethylpropyl)isoxazole-4-carboxamide;
739) 5-amino-3-(5-chlorothien-2-yl)-N-(1-phenylethyl)isoxazole-4-carboxamide;
740) 5-amino-3-(5-chlorothien-2-yl)-N-propylisoxazole-4-carboxamide;
741) 5-amino-3-(5-chlorothien-2-yl)-N-(2-methylbenzyl)isoxazole-4-carboxamide;
742) 5-amino-3-(5-chlorothien-2-yl)-N-cyclobutylisoxazole-4-carboxamide;
743) 5-amino-3-(5-chlorothien-2-yl)-N-(3-methoxypropyl)isoxazole-4-carboxamide;
744) 5-amino-N-(2-bromobenzyl)-3-(5-chlorothien-2-yl)isoxazole-4-carboxamide;
745) 5-amino-3-(5-chlorothien-2-yl)-N-(1-methylheptyl)isoxazole-4-carboxamide;
746) 5-amino-3-(5-chlorothien-2-yl)-N-heptylisoxazole-4-carboxamide;
747) 5-amino-N-(tert-butyl)-3-(5-chlorothien-2-yl)isoxazole-4-carboxamide;
748) 5-amino-3-(5-chlorothien-2-yl)-N-(3,4-dimethoxybenzyl)isoxazole-4-carboxamide;
749) 5-amino-3-(5-chlorothien-2-yl)-N-(2,3-dihydro-1H-inden-1-yl)isoxazole-4-carboxamide;
750) 5-amino-3-(5-chlorothien-2-yl)-N-(1,2,3,4-tetrahydronaphthalen-1-yl)isoxazole-4-carboxamide;
751) 5-amino-N-benzyl-3-(5-methylthien-2-yl)isoxazole-4-carboxamide;
752) 5-amino-N-(2-furylmethyl)-3-(5-methylthien-2-yl)isoxazole-4-carboxamide;
753) 5-amino-3-(5-methylthien-2-yl)-N-(2-phenylethyl)isoxazole-4-carboxamide;
754) 5-amino-N-(sec-butyl)-3-(5-methylthien-2-yl)isoxazole-4-carboxamide;
755) 5-amino-N-cyclohexyl-3-(5-methylthien-2-yl)isoxazole-4-carboxamide;
756) 5-amino-N-(4-methoxybenzyl)-3-(5-methylthien-2-yl)isoxazole-4-carboxamide;
757) 5-amino-N-isopropyl-3-(5-methylthien-2-yl)isoxazole-4-carboxamide;
758) 5-amino-N-hexyl-3-(5-methylthien-2-yl)isoxazole-4-carboxamide;
759) N-allyl-5-amino-3-(5-methylthien-2-yl)isoxazole-4-carboxamide;
760) 5-amino-3-(5-methylthien-2-yl)-N-(thien-2-ylmethyl)isoxazole-4-carboxamide;
761) 5-amino-N-cyclopentyl-3-(5-methylthien-2-yl)isoxazole-4-carboxamide;
762) 5-amino-N-(1,5-dimethylhexyl)-3-(5-methylthien-2-yl)isoxazole-4-carboxamide;
763) 5-amino-N-isopentyl-3-(5-methylthien-2-yl)isoxazole-4-carboxamide;
764) 5-amino-N-(2-ethylhexyl)-3-(5-methylthien-2-yl)isoxazole-4-carboxamide;
765) 5-amino-3-(5-methylthien-2-yl)-N-(4-phenylbutyl)isoxazole-4-carboxamide;
766) 5-amino-N-(2-methylcyclohexyl)-3-(5-methylthien-2-yl)isoxazole-4-carboxamide;
767) 5-amino-N-[3,5-bis(trifluoromethyl)benzyl]-3-(5-methylthien-2-yl)isoxazole-4-carboxamide;
768) 5-amino-3-(5-methylthien-2-yl)-N-[3-(trifluoromethyl)benzyl]isoxazole-4-carboxamide;
769) 5-amino-3-(5-methylthien-2-yl)-N-(3-phenylpropyl)isoxazole-4-carboxamide;
770) 5-amino-N-isobutyl-3-(5-methylthien-2-yl)isoxazole-4-carboxamide;
771) 5-amino-3-(5-methylthien-2-yl)-N-[2-(trifluoromethyl)benzyl]isoxazole-4-carboxamide;
772) 5-amino-N-(2-methoxybenzyl)-3-(5-methylthien-2-yl)isoxazole-4-carboxamide;
773) 5-amino-N-(2-fluorobenzyl)-3-(5-methylthien-2-yl)isoxazole-4-carboxamide;
774) 5-amino-N-(3,4-difluorobenzyl)-3-(5-methylthien-2-yl)isoxazole-4-carboxamide;
775) 5-amino-N-(3-chlorobenzyl)-3-(5-methylthien-2-yl)isoxazole-4-carboxamide;
776) 5-amino-N-cycloheptyl-3-(5-methylthien-2-yl)isoxazole-4-carboxamide;
777) 5-amino-N-(1,2-dimethylpropyl)-3-(5-methylthien-2-yl)isoxazole-4-carboxamide;
778) 5-amino-N-(3,3-diphenylpropyl)-3-(5-methylthien-2-yl)isoxazole-4-carboxamide;
779) 5-amino-N-(1,2-diphenylethyl)-3-(5-methylthien-2-yl)isoxazole-4-carboxamide;
780) 5-amino-N-(2,4-dimethoxybenzyl)-3-(5-methylthien-2-yl)isoxazole-4-carboxamide;
781) 5-amino-N-[3-(dibutylamino)propyl]-3-(5-methylthien-2-yl)isoxazole-4-carboxamide;
782) 5-amino-N-butyl-3-(5-methylthien-2-yl)isoxazole-4-carboxamide;
783) 5-amino-3-(5-methylthien-2-yl)-N-[4-(trifluoromethoxy)benzyl]isoxazole-4-carboxamide;
784) 5-amino-N-[(5-methyl-2-furyl)methyl]-3-(5-methylthien-2-yl)isoxazole-4-carboxamide;
785) 5-amino-N-(1-methylhexyl)-3-(5-methylthien-2-yl)isoxazole-4-carboxamide;
786) 5-amino-N-(2-methoxy-1-methylethyl)-3-(5-methylthien-2-yl)isoxazole-4-carboxamide;
787) 5-amino-N-(4-tert-butylcyclohexyl)-3-(5-methylthien-2-yl)isoxazole-4-carboxamide;
788) 5-amino-N-(1-methylbutyl)-3-(5-methylthien-2-yl)isoxazole-4-carboxamide;
789) 5-amino-N-(3-fluorobenzyl)-3-(5-methylthien-2-yl)isoxazole-4-carboxamide;
790) 5-amino-N-(4-methylbenzyl)-3-(5-methylthien-2-yl)isoxazole-4-carboxamide;
791) 5-amino-N-(1-methyl-3-phenylpropyl)-3-(5-methylthien-2-yl)isoxazole-4-carboxamide;
792) 5-amino-N-(1,3-benzodioxol-5-ylmethyl)-3-(5-methylthien-2-yl)isoxazole-4-carboxamide;
793) 5-amino-3-(5-methylthien-2-yl)-N-pentylisoxazole-4-carboxamide;
794) 5-amino-N-(cyclohexylmethyl)-3-(5-methylthien-2-yl)isoxazole-4-carboxamide;
795) 5-amino-3-(5-methylthien-2-yl)-N-(1-phenylethyl)isoxazole-4-carboxamide;
796) 5-amino-3-(5-methylthien-2-yl)-N-propylisoxazole-4-carboxamide;
797) 5-amino-N-(2-methylbenzyl)-3-(5-methylthien-2-yl)isoxazole-4-carboxamide;
798) 5-amino-N-cyclobutyl-3-(5-methylthien-2-yl)isoxazole-4-carboxamide;
799) 5-amino-N-(3-methoxypropyl)-3-(5-methylthien-2-yl)isoxazole-4-carboxamide;
800) 5-amino-3-(5-methylthien-2-yl)-N-octylisoxazole-4-carboxamide;
801) 5-amino-N-(1-methylheptyl)-3-(5-methylthien-2-yl)isoxazole-4-carboxamide;
802) 5-amino-N-heptyl-3-(5-methylthien-2-yl)isoxazole-4-carboxamide;
803) 5-amino-N-(tert-butyl)-3-(5-methylthien-2-yl)isoxazole-4-carboxamide;
804) 5-amino-N-(3,4-dimethoxybenzyl)-3-(5-methylthien-2-yl)isoxazole-4-carboxamide;
805) 5-amino-N-(2,3-dihydro-1H-inden-1-yl)-3-(5-methylthien-2-yl)isoxazole-4-carboxamide;
806) 5-amino-3-(5-methylthien-2-yl)-N-(1,2,3,4-tetrahydronaphthalen-1-yl)isoxazole-4-carboxamide;
807) 5-amino-N-benzyl-3-(3-methylthien-2yl)isoxazole-4-carboxamide;
808) 5-amino-N-(2-furylmethyl)-3-(3-methylthien-2-yl)isoxazole-4-carboxamide;
809) 5-amino-3-(3-methylthien-2-yl)-N-(2-phenylethyl)isoxazole-4-carboxamide;
810) 5-amino-N-(sec-butyl)-3-(3-methylthien-2-yl)isoxazole-4-carboxamide;
811) 5-amino-N-cyclohexyl-3-(3-methylthien-2-yl)isoxazole-4-carboxamide;
812) 5-amino-N-(4-methoxybenzyl)-3-(3-methylthien-2-yl)isoxazole-4-carboxamide; 813) 5-amino-N-isopropyl-3-(3-methylthien-2-yl)isoxazole-4-carboxamide;
814) 5-amino-N-hexyl-3-(3-methylthien-2-yl)isoxazole-4-carboxamide;
815) N-allyl-5-amino-3-(3-methylthien-2-yl)isoxazole-4-carboxamide;
816) 5-amino-3-(3-methylthien-2-yl)-N-(tetrahydrofuran-2-ylmethyl)isoxazole-4-carboxamide;
817) 5-amino-N-cyclopentyl-3-(3-methylthien-2-yl)isoxazole-4-carboxamide;
818) 5-amino-N-(1,5-dimethylhexyl)-3-(3-methylthien-2-yl)isoxazole-4-carboxamide;
819) 5-amino-N-isopentyl-3-(3-methylthien-2-yl)isoxazole-4-carboxamide;
820) 5-amino-N-(2-ethylhexyl)-3-(3-methylthien-2-yl)isoxazole-4-carboxamide;
821) 5-amino-3-(3-methylthien-2-yl)-N-(4-phenylbutyl)isoxazole-4-carboxamide;
822) 5-amino-N-(2-methylcyclohexyl)-3-(3-methylthien-2-yl)isoxazole-4-carboxamide;
823) 5-amino-N-[3,5-bis(trifluoromethyl)benzyl]-3-(3-methylthien-2-yl)isoxazole-4-carboxamide;
824) 5-amino-3-(3-methylthien-2-yl)-N-[3-(trifluoromethyl)benzyl]isoxazole-4-carboxamide;
825) 5-amino-3-(3-methylthien-2-yl)-N-(3-phenylpropyl)isoxazole-4-carboxamide;
826) 5-amino-N-isobutyl-3-(3-methylthien-2-yl)isoxazole-4-carboxamide;
827) 5-amino-3-(3-methylthien-2-yl)-N-[2-(trifluoromethyl)benzyl]isoxazole-4-carboxamide;
828) 5-amino-N-(2-methoxybenzyl)-3-(3-methylthien-2-yl)isoxazole-4-carboxamide;
829) 5-amino-N-(2-fluorobenzyl)-3-(3-methylthien-2-yl)isoxazole-4-carboxamide;
830) 5-amino-3-(3-methylthien-2-yl)-N-[4-(trifluoromethyl)benzyl]isoxazole-4-carboxamide;
831) 5-amino-N-(3,4-difluorobenzyl)-3-(3-methylthien-2-yl)isoxazole-4-carboxamide;
832) 5-amino-N-(3-chlorobenzyl)-3-(3-methylthien-2-yl)isoxazole-4-carboxamide;
833) 5-amino-N-(2-chlorobenzyl)-3-(3-methylthien-2-yl)isoxazole-4-carboxamide;
834) 5-amino-N-(3-methylbenzyl)-3-(3-methylthien-2-yl)isoxazole-4-carboxamide;
835) 5-amino-3-(3-methylthien-2-yl)-N-(1-naphthylmethyl)isoxazole-4-carboxamide;
836) 5-amino-N-(cyclopropylmethyl)-3-(3-methylthien-2-yl)isoxazole-4-carboxamide;
837) 5-amino-N-cycloheptyl-3-(3-methylthien-2-yl)isoxazole-4-carboxamide;
838) 5-amino-N-[2-(4-methoxyphenyl)ethyl]-3-(3-methylthien-2-yl)isoxazole-4-carboxamide;
839) 5-amino-N-(2-cyclohex-1-en-1-ylethyl)-3-(3-methylthien-2-yl)isoxazole-4-carboxamide;
840) 5-amino-N-(1,2-dimethylpropyl)-3-(3-methylthien-2-yl)isoxazole-4-carboxamide;
841) 5-amino-N-(3,3-diphenylpropyl)-3-(3-methylthien-2-yl)isoxazole-4-carboxamide;
842) 5-amino-N-(2,4-dimethoxybenzyl)-3-(3-methylthien-2-yl)isoxazole-4-carboxamide;
843) 5-amino-N-[3-(dibutylamino)propyl]-3 (3-methylthien-2-yl)isoxazole-4-carboxamide;
844) 5-amino-N-butyl-3-(3-methylthien-2-yl)isoxazole-4-carboxamide;
845) 5-amino-3-(3-methylthien-2-yl)-N-[4-(trifluoromethoxy)benzyl]isoxazole-4-carboxamide;
846) 5-amino-N-[(5-methyl-2-furyl)methyl]-3-(3-methylthien-2-yl)isoxazole-4-carboxamide;
847) 5-amino-N-(1-benzylpiperidin-4-yl)-3-(3-methylthien-2-yl)isoxazole-4-carboxamide;
848) 5-amino-N-(2-methoxy-1-methylethyl)-3-(3-methylthien-2-yl)isoxazole-4-carboxamide;
849) 5-amino-N-(4-tert-butylcyclohexyl)-3-(3-methylthien-2-yl)isoxazole-4-carboxamide;
850) 5-amino-N-(1-methylbutyl)-3-(3-methylthien-2-yl)isoxazole-4-carboxamide;
851) 5-amino-N-(3-fluorobenzyl)-3-(3-methylthien-2-yl)isoxazole-4-carboxamide;
852) 5-amino-N-(4-methylbenzyl)-3-(3-methylthien-2-yl)isoxazole-4-carboxamide;
853) 5-amino-N-(1,3-dimethylbutyl)-3-(3-methylthien-2-yl)isoxazole-4-carboxamide;
854) 5-amino-N-(1-methyl-3-phenylpropyl)-3-(3-methylthien-2-yl)isoxazole-4-carboxamide;
855) 5-amino-3-(3-methylthien-2-yl)-N-[3-(methylthio)propyl]isoxazole-4-carboxamide;
856) 5-amino-N-(1,3-benzodioxol-5-ylmethyl)-3-(3-methylthien-2-yl)isoxazole-4-carboxamide;
857) 5-amino-3-(3-methylthien-2-yl)-N-pentylisoxazole-4-carboxamide;
858) 5-amino-N-(cyclohexylmethyl)-3-(3-methylthien-2-yl)isoxazole-4-carboxamide;
859) ethyl 4-({[5-amino-3-(3-methylthien-2-yl)isoxazol-4-yl]carbonyl}amino)piperidine-1-carboxylate;
860) 5-amino-N-[2-(3-chlorophenyl)ethyl]-3-(3-methylthien-2-yl)isoxazole-4-carboxamide;
861) 5-amino-N-(1-ethylpropyl)-3-(3-methylthien-2-yl)isoxazole-4-carboxamide;
862) 5-amino-3-(3-methylthien-2-yl)-N-(1-phenylethyl)isoxazole-4-carboxamide;
863) 5-amino-N-(2-methylbenzyl)-3-(3-methylthien-2-yl)isoxazole-4-carboxamide;
864) 5-amino-N-cyclobutyl-3-(3-methylthien-2-yl)isoxazole-4-carboxamide;
865) 5-amino-N-benzhydryl-3-(3-methylthien-2-yl)isoxazole-4-carboxamide;
866) 5-amino-3-(3-methylthien-2-yl)-N-octylisoxazole-4-carboxamide;
867) 5-amino-N-(2-bromobenzyl)-3-(3-methylthien-2-yl)isoxazole-4-carboxamide;
868) 5-amino-N-(1-methylheptyl)-3-(3-methylthien-2-yl)isoxazole-4-carboxamide;
869) 5-amino-N-heptyl-3-(3-methylthien-2-yl)isoxazole-4-carboxamide;
870) 5-amino-N-(tert-butyl)-3-(3-methylthien-2-yl)isoxazole-4-carboxamide;
871) 5-amino-N-(3,4-dimethoxybenzyl)-3-(3-methylthien-2-yl)isoxazole-4-carboxamide;
872) 5-amino-N-(2,3-dihydro-1H-inden-1-yl)-3-(3-methylthien-2-yl)isoxazole-4-carboxamide;
873) 5-amino-3-(3-methylthien-2-yl)-N-(1,2,3,4-tetrahydronaphthalen-1-yl)isoxazole-4-carboxamide;
874) 5-amino-3-[5-(3-chlorophenyl)-2-furyl]-N-(2-furylmethyl)isoxazole-4-carboxamide;
875) 5-amino-3-[5-(3-chlorophenyl)-2-furyl]-N-(2-phenylethyl)isoxazole-4-carboxamide;
876) 5-amino-N-(sec-butyl)-3-[5-(3-chlorophenyl)-2-furyl]isoxazole-4-carboxamide;
877) 5-amino-3-[5-(3-chlorophenyl)-2-furyl]-N-cyclohexylisoxazole-4-carboxamide;
878) 5-amino-3-[5-(3-chlorophenyl)-2-furyl]-N-(4-methoxybenzyl)isoxazole-4-carboxamide;
879) 5-amino-3-[5-(3-chlorophenyl)-2-furyl]-N-isopropylisoxazole-4-carboxamide;
880) 5-amino-3-[5-(3-chlorophenyl)-2-furyl]-N-hexylisoxazole-4-carboxamide;
881) 5-amino-3-[5-(3-chlorophenyl)-2-furyl]-N-(tetrahydrofuran-2-ylmethyl)isoxazole-4-carboxamide;
882) 5-amino-3-[5-(3-chlorophenyl)-2-furyl]-N-cyclopentylisoxazole-4-carboxamide;
883) 5-amino-3-[5-(3-chlorophenyl)-2-furyl]-N-(1,5-dimethylhexyl)isoxazole-4-carboxamide;
884) 5-amino-3-[5-(3-chlorophenyl)-2-furyl]-N-isopentylisoxazole-4-carboxamide;
885) 5-amino-3-[5-(3-chlorophenyl)-2-furyl]-N-(2-ethylhexyl)isoxazole-4-carboxamide;
886) 5-amino-3-[5-(3-chlorophenyl)-2-furyl]-N-(4-phenylbutyl)isoxazole-4-carboxamide;
887) 5-amino-3-[5-(3-chlorophenyl)-2-furyl]-N-(2-methylcyclohexyl)isoxazole-4-carboxamide;
888) 5-amino-N-[3,5-bis(trifluoromethyl)benzyl]-3-[5-(3-chlorophenyl)-2-furyl]isoxazole-4-carboxamide;
889) 5-amino-3-[5-(3-chlorophenyl)-2-furyl]-N-[3-(trifluoromethyl)benzyl]isoxazole-4-carboxamide;
890) 5-amino-3-[5-(3-chlorophenyl)-2-furyl]-N-(3-phenylpropyl)isoxazole-4-carboxamide;
891) 5-amino-3-[5-(3-chlorophenyl)-2-furyl]-N-isobutylisoxazole-4-carboxamide;
892) 5-amino-3-[5-(3-chlorophenyl)-2-furyl]-N-[2-(trifluoromethyl)benzyl]isoxazole-4-carboxamide;
893) 5-amino-3-[5-(3-chlorophenyl)-2-furyl]-N-(2-methoxybenzyl)isoxazole-4-carboxamide;
894) 5-amino-3-[5-(3-chlorophenyl)-2-furyl]-N-(2-fluorobenzyl)isoxazole-4-carboxamide;
895) 5-amino-3-[5-(3-chlorophenyl)-2-furyl]-N-[4-(trifluoromethyl)benzyl]isoxazole-4-carboxamide;
896) 5-amino-3-[5-(3-chlorophenyl)-2-furyl]-N-(3,4-difluorobenzyl)isoxazole-4-carboxamide;
897) 5-amino-N-(3-chlorobenzyl)-3-[5-(3-chlorophenyl)-2-furyl]isoxazole-4-carboxamide;
898) 5-amino-N-(2-chlorobenzyl)-3-[5-(3-chlorophenyl)-2-furyl]isoxazole-4-carboxamide;
899) 5-amino-3-[5-(3-chlorophenyl)-2-furyl]-N-(3-methylbenzyl)isoxazole-4-carboxamide;
900) 5-amino-3-[5-(3-chlorophenyl)-2-furyl]-N-(1-naphthylmethyl)isoxazole-4-carboxamide;
901) 5-amino-3-[5-(3-chlorophenyl)-2-furyl]-N-(cyclopropylmethyl)isoxazole-4-carboxamide;
902) 5-amino-3-[5-(3-chlorophenyl)-2-furyl]-N-cycloheptylisoxazole-4-carboxamide;
903) 5-amino-3-[5-(3-chlorophenyl)-2-furyl]-N-[2-(4-methoxyphenyl)ethyl]isoxazole-4-carboxamide;
904) 5-amino-3-[5-(3-chlorophenyl)-2-furyl]-N-(2-cyclohex-1-en-1-ylethyl)isoxazole-4-carboxamide;
905) 5-amino-3-[5-(3-chlorophenyl)-2-furyl]-N-(1,2-dimethylpropyl)isoxazole-4-carboxamide;
906) 5-amino-3-[5-(3-chlorophenyl)-2-furyl]-N-(3,3-diphenylpropyl)isoxazole-4-carboxamide;
907) 5-amino-3-[5-(3-chlorophenyl)-2-furyl]-N-(1,2-diphenylethyl)isoxazole-4-carboxamide;
908) 5-amino-3-[5-(3-chlorophenyl)-2-furyl]-N-(2,4-dimethoxybenzyl)isoxazole-4-carboxamide;
909) 5-amino-3-[5-(3-chlorophenyl)-2-furyl]-N-[3-(dibutylamino)propyl]isoxazole-4-carboxamide;
910) 5-amino-N-butyl-3-[5-(3-chlorophenyl)-2-furyl]isoxazole-4-carboxamide;
911) 5-amino-3-[5-(3-chlorophenyl)-2-furyl]-N-[4-(trifluoromethoxy)benzyl]isoxazole-4-carboxamide;
912) 5-amino-3-[5-(3-chlorophenyl)-2-furyl]-N-[(5-methyl-2-furyl)methyl]isoxazole-4-carboxamide;
913) 5-amino-3-[5-(3-chlorophenyl)-2-furyl]-N-(1-methylhexyl)isoxazole-4-carboxamide;
914) 5-amino-N-(1-benzylpiperidin-4-yl)-3-[5-(3-chlorophenyl)-2-furyl]isoxazole-4-carboxamide;
915) 5-amino-3-[5-(3-chlorophenyl)-2-furyl]-N-(1,3-dimethylbutyl)isoxazole-4-carboxamide;
916) 5-amino-3-[5-(3-chlorophenyl)-2-furyl]-N-(1-methyl-3-phenylpropyl)isoxazole-4-carboxamide;
917) 5-amino-3-[5-(3-chlorophenyl)-2-furyl]-N-[3-(methylthio)propyl]isoxazole-4-carboxamide;
918) 5-amino-3-[5-(3-chlorophenyl)-2-furyl]-N-pentylisoxazole-4-carboxamide;
919) ethyl 4-[({5-amino-3-[5-(3-chlorophenyl)-2-furyl]isoxazol-4-yl}carbonyl)amino]piperidine-1-carboxylate;
920) 5-amino-N-[2-(3-chlorophenyl)ethyl]-3-[5-(3-chlorophenyl)-2-furyl]isoxazole-4-carboxamide;
921) 5-amino-3-[5-(3-chlorophenyl)-2-furyl]-N-(1-ethylpropyl)isoxazole-4-carboxamide;
922) 5-amino-3-[5-(3-chlorophenyl)-2-furyl]-N-(1-phenylethyl)isoxazole-4-carboxamide;
923) 5-amino-3-[5-(3-chlorophenyl)-2-furyl]-N-(2-methylbenzyl)isoxazole-4-carboxamide;
924) 5-amino-3-[5-(3-chlorophenyl)-2-furyl]-N-cyclobutylisoxazole-4-carboxamide;
925) 5-amino-N-benzhydryl-3-[5-(3-chlorophenyl)-2-furyl]isoxazole-4-carboxamide;
926) 5-amino-3-[5-(3-chlorophenyl)-2-furyl]-N-octylisoxazole-4-carboxamide;
927) 5-amino-N-(2-bromobenzyl)-3-[5-(3-chlorophenyl)-2-furyl]isoxazole-4-carboxamide;
928) 5-amino-3-[5-(3-chlorophenyl)-2-furyl]-N-(1-methylheptyl)isoxazole-4-carboxamide;
929) 5-amino-3-[5-(3-chlorophenyl)-2-furyl]-N-heptylisoxazole-4-carboxamide;
930) 5-amino-N-(tert-butyl)-3-[5-(3-chlorophenyl)-2-furyl]isoxazole-4-carboxamide;
931) 5-amino-3-[5-(3-chlorophenyl)-2-furyl]-N-(3,4-dimethoxybenzyl)isoxazole-4-carboxamide;
932) 5-amino-3-[5-(3-chlorophenyl)-2-furyl]-N-(2,3-dihydro-1H-inden-1-yl)isoxazole-4-carboxamide;
933) 5-amino-3-[5-(3-chlorophenyl)-2-furyl]-N-(1,2,3,4-tetrahydronaphthalen-1-yl)isoxazole-4-carboxamide;
934) 5-amino-N-benzyl-3-(5-methyl-2-furyl)isoxazole-4-carboxamide;
935) 5-amino-N-(2-furylmethyl)-3-(5-methyl-2-furyl)isoxazole-4-carboxamide;
936) 5-amino-3-(5-methyl-2-furyl)-N-(2-phenylethyl)isoxazole-4-carboxamide;
937) 5-amino-N-(sec-butyl)-3-(5-methyl-2-furyl)isoxazole-4-carboxamide;
938) 5-amino-N-cyclohexyl-3-(5-methyl-2-furyl)isoxazole-4-carboxamide;
939) 5-amino-N-(4-methoxybenzyl)-3-(5-methyl-2-furyl)isoxazole-4-carboxamide;
940) 5-amino-N-hexyl-3-(5-methyl-2-furyl)isoxazole-4-carboxamide;
941) 5-amino-3-(5-methyl-2-furyl)-N-(tetrahydrofuran-2-ylmethyl)isoxazole-4-carboxamide;
942) 5-amino-3-(5-methyl-2-furyl)-N-(thien-2-ylmethyl)isoxazole-4-carboxamide;
943) 5-amino-N-cyclopentyl-3-(5-methyl-2-furyl)isoxazole-4-carboxamide;
944) 5-amino-N-(1,5-dimethylhexyl)-3-(5-methyl-2-furyl)isoxazole-4-carboxamide;
945) 5-amino-N-isopentyl-3-(5-methyl-2-furyl)isoxazole-4-carboxamide;
946) 5-amino-N-(2-ethylhexyl)-3-(5-methyl-2-furyl)isoxazole-4-carboxamide;
947) 5-amino-3-(5-methyl-2-furyl)-N-(4-phenylbutyl)isoxazole-4-carboxamide;
948) 5-amino-N-(2-methylcyclohexyl)-3-(5-methyl-2-furyl)isoxazole-4-carboxamide;
949) 5-amino-3-(5-methyl-2-furyl)-N-[3-(trifluoromethyl)benzyl]isoxazole-4-carboxamide;
950) 5-amino-3-(5-methyl-2-furyl)-N-(3-phenylpropyl)isoxazole-4-carboxamide;
951) 5-amino-N-isobutyl-3-(5-methyl-2-furyl)isoxazole-4-carboxamide;
952) 5-amino-3-(5-methyl-2-furyl)-N-[2-(trifluoromethyl)benzyl]isoxazole-4-carboxamide;
953) 5-amino-N-(2-methoxybenzyl)-3-(5-methyl-2-furyl)isoxazole-4-carboxamide;
954) 5-amino-N-(2-fluorobenzyl)-3-(5-methyl-2-furyl)isoxazole-4-carboxamide;
955) 5-amino-3-(5-methyl-2-furyl)-N-[4-(trifluoromethyl)benzyl]isoxazole-4-carboxamide;
956) 5-amino-N-(3,4-difluorobenzyl)-3-(5-methyl-2-furyl)isoxazole-4-carboxamide;
957) 5-amino-N-(3-chlorobenzyl)-3-(5-methyl-2-furyl)isoxazole-4-carboxamide;
958) 5-amino-N-(2-chlorobenzyl)-3-(5-methyl-2-furyl)isoxazole-4-carboxamide;
959) 5-amino-N-(3-methylbenzyl)-3-(5-methyl-2-furyl)isoxazole-4-carboxamide;
960) 5-amino-3-(5-methyl-2-furyl)-N-(1-naphthylmethyl)isoxazole-4-carboxamide;
961) 5-amino-N-(cyclopropylmethyl)-3-(5-methyl-2-furyl)isoxazole-4-carboxamide;
962) 5-amino-N-cycloheptyl-3-(5-methyl-2-furyl)isoxazole-4-carboxamide;
963) 5-amino-N-[2-(4-methoxyphenyl)ethyl]-3-(5-methyl-2-furyl)isoxazole-4-carboxamide;
964) 5-amino-N-(2-cyclohex-1-en-1-ylethyl)-3-(5-methyl-2-furyl)isoxazole-4-carboxamide;
965) 5-amino-N-(1,2-dimethylpropyl)-3-(5-methyl-2-furyl)isoxazole-4-carboxamide;
966) 5-amino-N-(3,3-diphenylpropyl)-3-(5-methyl-2-furyl)isoxazole-4-carboxamide;
967) 5-amino-N-(1,2-diphenylethyl)-3-(5-methyl-2-furyl)isoxazole-4-carboxamide;
968) 5-amino-N-(2,4-dimethoxybenzyl)-3-(5-methyl-2-furyl)isoxazole-4-carboxamide;
969) 5-amino-N-[3-(dibutylamino)propyl]-3-(5-methyl-2-furyl)isoxazole-4-carboxamide;
970) 5-amino-N-butyl-3-(5-methyl-2-furyl)isoxazole-4-carboxamide;
971) 5-amino-3-(5-methyl-2-furyl)-N-[4-(trifluoromethoxy)benzyl]isoxazole-4-carboxamide;
972) 5-amino-3-(5-methyl-2-furyl)-N-[(5-methyl-2-furyl)methyl]isoxazole-4-carboxamide;
973) 5-amino-3-(5-methyl-2-furyl)-N-(1-methylhexyl)isoxazole-4-carboxamide;
974) 5-amino-N-(1-benzylpiperidin-4-yl)-3-(5-methyl-2-furyl)isoxazole-4-carboxamide;
975) 5-amino-N-(2-methoxy-1-methylethyl)-3-(5-methyl-2-furyl)isoxazole-4-carboxamide;
976) 5-amino-N-(1-methylbutyl)-3-(5-methyl-2-furyl)isoxazole-4-carboxamide;
977) 5-amino-N-(3-fluorobenzyl)-3-(5-methyl-2-furyl)isoxazole-4-carboxamide;
978) 5-amino-N-(4-methylbenzyl)-3-(5-methyl-2-furyl)isoxazole-4-carboxamide;
979) 5-amino-N-(1,3-dimethylbutyl)-3-(5-methyl-2-furyl)isoxazole-4-carboxamide;
980) 5-amino-3-(5-methyl-2-furyl)-N-(1-methyl-3-phenylpropyl)isoxazole-4-carboxamide;
981) 5-amino-3-(5-methyl-2-furyl)-N-[3-(methylthio)propyl]isoxazole-4-carboxamide;
982) 5-amino-N-(1,3-benzodioxol-5-ylmethyl)-3-(5-methyl-2-furyl)isoxazole-4-carboxamide;
983) 5-amino-3-(5-methyl-2-furyl)-N-pentylisoxazole-4-carboxamide;
984) 5-amino-N-(cyclohexylmethyl)-3-(5-methyl-2-furyl)isoxazole-4-carboxamide;
985) ethyl 4-({[5-amino-3-(5-methyl-2-furyl)isoxazol-4-yl]carbonyl}amino)piperidine-1-carboxylate;
986) 5-amino-N-[2-(3-chlorophenyl)ethyl]-3-(5-methyl-2-furyl)isoxazole-4-carboxamide;
987) 5-amino-N-(1-ethylpropyl)-3-(5-methyl-2-furyl)isoxazole-4-carboxamide;
988) 5-amino-3-(5-methyl-2-furyl)-N-(1-phenylethyl)isoxazole-4-carboxamide;
989) 5-amino-N-(2-methylbenzyl)-3-(5-methyl-2-furyl)isoxazole-4-carboxamide;
990) 5-amino-N-cyclobutyl-3-(5-methyl-2-furyl)isoxazole-4-carboxamide;
991) 5-amino-N-benzhydryl-3-(5-methyl-2-furyl)isoxazole-4-carboxamide;
992) 5-amino-3-(5-methyl-2-furyl)-N-octylisoxazole-4-carboxamide;
993) 5-amino-N-(2-bromobenzyl)-3-(5-methyl-2-furyl)isoxazole-4-carboxamide;
994) 5-amino-3-(5-methyl-2-furyl)-N-(1-methylheptyl)isoxazole-4-carboxamide;
995) 5-amino-N-heptyl-3-(5-methyl-2-furyl)isoxazole-4-carboxamide;
996) 5-amino-N-(tert-butyl)-3-(5-methyl-2-furyl)isoxazole-4-carboxamide;
997) 5-amino-N-(3,4-dimethoxybenzyl)-3-(5-methyl-2-furyl)isoxazole-4-carboxamide;
998) 5-amino-N-(2,3-dihydro-1H-inden-1-yl)-3-(5-methyl-2-furyl)isoxazole-4-carboxamide;
999) 5-amino-3-(5-methyl-2-furyl)-N-(1,2,3,4-tetrahydronaphthalen-1-yl)isoxazole-4-carboxamide;
1000) 5-amino-N-benzyl-3-(2-furyl)isoxazole-4-carboxamide;
1001) 5-amino-3-(2-furyl)-N-(2-furylmethyl)isoxazole-4-carboxamide;
1002) 5-amino-3-(2-furyl)-N-(2-phenylethyl)isoxazole-4-carboxamide;
1003) 5-amino-N-(sec-butyl)-3-(2-furyl)isoxazole-4-carboxamide;
1004) 5-amino-3-(2-furyl)-N-(4-methoxybenzyl)isoxazole-4-carboxamide;
1005) 5-amino-3-(2-furyl)-N-hexylisoxazole-4-carboxamide;
1006) 5-amino-3-(2-furyl)-N-(tetrahydrofuran-2-ylmethyl)isoxazole-4-carboxamide;
1007) 5-amino-3-(2-furyl)-N-(thien-2-ylmethyl)isoxazole-4-carboxamide;
1008) 5-amino-N-cyclopentyl-3-(2-furyl)isoxazole-4-carboxamide;
1009) 5-amino-N-(1,5-dimethylhexyl)-3-(2-furyl)isoxazole-4-carboxamide;
1010) 5-amino-3-(2-furyl)-N-isopentylisoxazole-4-carboxamide;
1011) 5-amino-N-(2-ethylhexyl)-3-(2-furyl)isoxazole-4-carboxamide;
1012) 5-amino-3-(2-furyl)-N-(4-phenylbutyl)isoxazole-4-carboxamide;
1013) 5-amino-3-(2-furyl)-N-(2-methylcyclohexyl)isoxazole-4-carboxamide;
1014) 5-amino-N-[3,5-bis(trifluoromethyl)benzyl]-3-(2-furyl)isoxazole-4-carboxamide;
1015) 5-amino-3-(2-furyl)-N-[3-(trifluoromethyl)benzyl]isoxazole-4-carboxamide;
1016) 5-amino-3-(2-furyl)-N-(3-phenylpropyl)isoxazole-4-carboxamide;
1017) 5-amino-3-(2-furyl)-N-isobutylisoxazole-4-carboxamide;
1018) 5-amino-3-(2-furyl)-N-[2-(trifluoromethyl)benzyl]isoxazole-4-carboxamide;
1019) 5-amino-3-(2-furyl)-N-(2-methoxybenzyl)isoxazole-4-carboxamide;
1020) 5-amino-N-(2-fluorobenzyl)-3-(2-furyl)isoxazole-4-carboxamide;
1021) 5-amino-3-(2-furyl)-N-[4-(trifluoromethyl)benzyl]isoxazole-4-carboxamide;
1022) 5-amino-N-(3,4-difluorobenzyl)-3-(2-furyl)isoxazole-4-carboxamide;
1023) 5-amino-N-(3-chlorobenzyl)-3-(2-furyl)isoxazole-4-carboxamide;
1024) 5-amino-N-(2-chlorobenzyl)-3-(2-furyl)isoxazole-4-carboxamide;
1025) 5-amino-3-(2-furyl)-N-(3-methylbenzyl)isoxazole-4-carboxamide;
1026) 5-amino-3-(2-furyl)-N-(1-naphthylmethyl)isoxazole-4-carboxamide;
1027) 5-amino-N-(cyclopropylmethyl)-3-(2-furyl)isoxazole-4-carboxamide;
1028) 5-amino-N-cycloheptyl-3-(2-furyl)isoxazole-4-carboxamide;
1029) 5-amino-3-(2-furyl)-N-[2-(4-methoxyphenyl)ethyl]isoxazole-4-carboxamide;
1030) 5-amino-N-(2-cyclohex-1-en-1-ylethyl)-3-(2-furyl)isoxazole-4-carboxamide;
1031) 5-amino-N-(1,2-dimethylpropyl)-3-(2-furyl)isoxazole-4-carboxamide;
1032) 5-amino-N-(3,3-diphenylpropyl)-3-(2-furyl)isoxazole-4-carboxamide;
1033) 5-amino-N-(1,2-diphenylethyl)-3-(2-furyl)isoxazole-4-carboxamide;
1034) 5-amino-N-(2,4-dimethoxybenzyl)-3-(2-furyl)isoxazole-4-carboxamide;
1035) 5-amino-N-[3-(dibutylamino)propyl]-3-(2-furyl)isoxazole-4-carboxamide;
1036) 5-amino-N-butyl-3-(2-furyl)isoxazole-4-carboxamide;
1037) 5-amino-3-(2-furyl)-N-[4-(trifluoromethoxy)benzyl]isoxazole-4-carboxamide;
1038) 5-amino-3-(2-furyl)-N-[(5-methyl-2-furyl)methyl]isoxazole-4-carboxamide;
1039) 5-amino-3-(2-furyl)-N-(1-methylhexyl)isoxazole-4-carboxamide;
1040) 5-amino-N-(1-benzylpiperidin-4-yl)-3-(2-furyl)isoxazole-4-carboxamide;
1041) 5-amino-3-(2-furyl)-N-(2-methoxy-1-methylethyl)isoxazole-4-carboxamide;
1042) 5-amino-N-(4-tert-butylcyclohexyl)-3-(2-furyl)isoxazole-4-carboxamide;
1043) 5-amino-3-(2-furyl)-N-(1-methylbutyl)isoxazole-4-carboxamide;
1044) 5-amino-N-(3-fluorobenzyl)-3-(2-furyl)isoxazole-4-carboxamide;
1045) 5-amino-3-(2-furyl)-N-(4-methylbenzyl)isoxazole-4-carboxamide;
1046) 5-amino-N-(1,3-dimethylbutyl)-3-(2-furyl)isoxazole-4-carboxamide;
1047) 5-amino-3-(2-furyl)-N-(1-methyl-3-phenylpropyl)isoxazole-4-carboxamide;
1048) 5-amino-3-(2-furyl)-N-[3-(methylthio)propyl]isoxazole-4-carboxamide;
1049) 5-amino-N-(1,3-benzodioxol-5-ylmethyl)-3-(2-furyl)isoxazole-4-carboxamide;
1050) 5-amino-3-(2-furyl)-N-pentylisoxazole-4-carboxamide;
1051) 5-amino-N-(cyclohexylmethyl)-3-(2-furyl)isoxazole-4-carboxamide;
1052) ethyl 4-({[5-amino-3-(2-furyl)isoxazol-4-yl]carbonyl}amino)piperidine-1-carboxylate;
1053) 5-amino-N-[2-(3-chlorophenyl)ethyl]-3-(2-furyl)isoxazole-4-carboxamide;
1054) 5-amino-N-(1-ethylpropyl)-3-(2-furyl)isoxazole-4-carboxamide;
1055) 5-amino-3-(2-furyl)-N-(1-phenylethyl)isoxazole-4-carboxamide;
1056) 5-amino-3-(2-furyl)-N-(2-methylbenzyl)isoxazole-4-carboxamide;
1057) 5-amino-N-cyclobutyl-3-(2-furyl)isoxazole-4-carboxamide;
1058) 5-amino-N-benzhydryl-3-(2-furyl)isoxazole-4-carboxamide;
1059) 5-amino-3-(2-furyl)-N-octylisoxazole-4-carboxamide;
1060) 5-amino-N-(2-bromobenzyl)-3-(2-furyl)isoxazole-4-carboxamide;
1061) 5-amino-3-(2-furyl)-N-(1-methylheptyl)isoxazole-4-carboxamide;
1062) 5-amino-3-(2-furyl)-N-heptylisoxazole-4-carboxamide;
1063) 5-amino-N-(tert-butyl)-3-(2-furyl)isoxazole-4-carboxamide;
1064) 5-amino-N-(3,4-dimethoxybenzyl)-3-(2-furyl)isoxazole-4-carboxamide;
1065) 5-amino-N-(2,3-dihydro-1H-inden-1-yl)-3-(2-furyl)isoxazole-4-carboxamide;
1066) 5-amino-3-(2-furyl)-N-(1,2,3,4-tetrahydronaphthalen-1-yl)isoxazole-4-carboxamide;
1067) 5-amino-3-(1-benzofuran-3-yl)-N-benzylisoxazole-4-carboxamide;
1068) 5-amino-3-(1-benzofuran-3-yl)-N-(2-furylmethyl)isoxazole-4-carboxamide;
1069) 5-amino-3-(1-benzofuran-3-yl)-N-(2-phenylethyl)isoxazole-4-carboxamide;
1070) 5-amino-3-(1-benzofuran-3-yl)-N-(sec-butyl)isoxazole-4-carboxamide;
1071) 5-amino-3-(1-benzofuran-3-yl)-N-cyclohexylisoxazole-4-carboxamide;
1072) 5-amino-3-(1-benzofuran-3-yl)-N-(4-methoxybenzyl)isoxazole-4-carboxamide;
1073) 5-amino-3-(1-benzofuran-3-yl)-N-hexylisoxazole-4-carboxamide;
1074) 5-amino-3-(1-benzofuran-3-yl)-N-(tetrahydrofuran-2-ylmethyl)isoxazole-4-carboxamide;
1075) 5-amino-3-(1-benzofuran-3-yl)-N-(thien-2-ylmethyl)isoxazole-4-carboxamide;
1076) 5-amino-3-(1-benzofuran-3-yl)-N-cyclopentylisoxazole-4-carboxamide;
1077) 5-amino-3-(1-benzofuran-3-yl)-N-(1,5-dimethylhexyl)isoxazole-4-carboxamide;
1078) 5-amino-3-(1-benzofuran-3-yl)-N-isopentylisoxazole-4-carboxamide;
1079) 5-amino-3-(1-benzofuran-3-yl)-N-(2-ethylhexyl)isoxazole-4-carboxamide;
1080) 5-amino-3-(1-benzofuran-3-yl)-N-(4-phenylbutyl)isoxazole-4-carboxamide;
1081) 5-amino-3-(1-benzofuran-3-yl)-N-(2-methylcyclohexyl)isoxazole-4-carboxamide;
1082) 5-amino-3-(1-benzofuran-3-yl)-N-[3,5-bis(trifluoromethyl)benzyl]isoxazole-4-carboxamide;
1083) 5-amino-3-(1-benzofuran-3-yl)-N-[3-(trifluoromethyl)benzyl]isoxazole-4-carboxamide;
1084) 5-amino-3-(1-benzofuran-3-yl)-N-(3-phenylpropyl)isoxazole-4-carboxamide;
1085) 5-amino-3-(1-benzofuran-3-yl)-N-isobutylisoxazole-4-carboxamide;
1086) 5-amino-3-(1-benzofuran-3-yl)-N-[2-(trifluoromethyl)benzyl]isoxazole-4-carboxamide;
1087) 5-amino-3-(1-benzofuran-3-yl)-N-(2-methoxybenzyl)isoxazole-4-carboxamide;
1088) 5-amino-3-(1-benzofuran-3-yl)-N-(2-fluorobenzyl)isoxazole-4-carboxamide;
1089) 5-amino-3-(1-benzofuran-3-yl)-N-[4-(trifluoromethyl)benzyl]isoxazole-4-carboxamide;
1090) 5-amino-3-(1-benzofuran-3-yl)-N-(3,4-difluorobenzyl)isoxazole-4-carboxamide;
1091) 5-amino-3-(1-benzofuran-3-yl)-N-(3-chlorobenzyl)isoxazole-4-carboxamide;
1092) 5-amino-3-(1-benzofuran-3-yl)-N-(2-chlorobenzyl)isoxazole-4-carboxamide;
1093) 5-amino-3-(1-benzofuran-3-yl)-N-(3-methylbenzyl)isoxazole-4-carboxamide;
1094) 5-amino-3-(1-benzofuran-3-yl)-N-(1-naphthylmethyl)isoxazole-4-carboxamide;
1095) 5-amino-3-(1-benzofuran-3-yl)-N-(cyclopropylmethyl)isoxazole-4-carboxamide;
1096) 5-amino-3-(1-benzofuran-3-yl)-N-cycloheptylisoxazole-4-carboxamide;
1097) 5-amino-3-(1-benzofuran-3-yl)-N-[2-(4-methoxyphenyl)ethyl]isoxazole-4-carboxamide;
1098) 5-amino-3-(1-benzofuran-3-yl)-N-(2-cyclohex-1-en-1-ylethyl)isoxazole-4-carboxamide;
1099) 5-amino-3-(1-benzofuran-3-yl)-N-(1,2-dimethylpropyl)isoxazole-4-carboxamide;
1100) 5-amino-3-(1-benzofuran-3-yl)-N-(3,3-diphenylpropyl)isoxazole-4-carboxamide;
1101) 5-amino-3-(1-benzofuran-3-yl)-N-(1,2-diphenylethyl)isoxazole-4-carboxamide;
1102) 5-amino-3-(1-benzofuran-3-yl)-N-(2,4-dimethoxybenzyl)isoxazole-4-carboxamide;
1103) 5-amino-3-(1-benzofuran-3-yl)-N-[3-(dibutylamino)propyl]isoxazole-4-carboxamide;
1104) 5-amino-3-(1-benzofuran-3-yl)-N-butylisoxazole-4-carboxamide;
1105) 5-amino-3-(1-benzofuran-3-yl)-N-[4-(trifluoromethoxy)benzyl]isoxazole-4-carboxamide;
1106) 5-amino-3-(1-benzofuran-3-yl)-N-[(5-methyl-2-furyl)methyl]isoxazole-4-carboxamide;
1107) 5-amino-3-(1-benzofuran-3-yl)-N-(1-methylhexyl)isoxazole-4-carboxamide;
1108) 5-amino-3-(1-benzofuran-3-yl)-N-(1-benzylpiperidin-4-yl)isoxazole-4-carboxamide;
1109) 5-amino-3-(1-benzofuran-3-yl)-N-(2-methoxy-1-methylethyl)isoxazole-4-carboxamide;
1110) 5-amino-3-(1-benzofuran-3-yl)-N-(1-methylbutyl)isoxazole-4-carboxamide;
1111) 5-amino-3-(1-benzofuran-3-yl)-N-(3-fluorobenzyl)isoxazole-4-carboxamide;
1112) 5-amino-3-(1-benzofuran-3-yl)-N-(1,3-dimethylbutyl)isoxazole-4-carboxamide;
1113) 5-amino-3-(1-benzofuran-3-yl)-N-(1-methyl-3-phenylpropyl)isoxazole-4-carboxamide;
1114) 5-amino-3-(1-benzofuran-3-yl)-N-[3-(methylthio)propyl]isoxazole-4-carboxamide;
1115) 5-amino-N-(1,3-benzodioxol-5-ylmethyl)-3-(1-benzofuran-3-yl)isoxazole-4-carboxamide;
1116) 5-amino-3-(1-benzofuran-3-yl)-N-pentylisoxazole-4-carboxamide;
1117) 5-amino-3-(1-benzofuran-3-yl)-N-(cyclohexylmethyl)isoxazole-4-carboxamide;
1118) ethyl 4-({[5-amino-3-(1-benzofuran-3-yl)isoxazol-4-yl]carbonyl}amino)piperidine-1-carboxylate;
1119) 5-amino-3-(1-benzofuran-3-yl)-N-[2-(3-chlorophenyl)ethyl]isoxazole-4-carboxamide;
1120) 5-amino-3-(1-benzofuran-3-yl)-N-(1-ethylpropyl)isoxazole-4-carboxamide;
1121) 5-amino-3-(1-benzofuran-3-yl)-N-(1-phenylethyl)isoxazole-4-carboxamide;
1122) 5-amino-3-(1-benzofuran-3-yl)-N-(2-methylbenzyl)isoxazole-4-carboxamide;
1123) 5-amino-3-(1-benzofuran-3-yl)-N-cyclobutylisoxazole-4-carboxamide;
1124) 5-amino-N-benzhydryl-3-(1-benzofuran-3-yl)isoxazole-4-carboxamide;
1125) 5-amino-3-(1-benzofuran-3-yl)-N-octylisoxazole-4-carboxamide;
1126) 5-amino-3-(1-benzofuran-3-yl)-N-(2-bromobenzyl)isoxazole-4-carboxamide;
1127) 5-amino-3-(1-benzofuran-3-yl)-N-(1-methylheptyl)isoxazole-4-carboxamide;
1128) 5-amino-3-(1-benzofuran-3-yl)-N-heptylisoxazole-4-carboxamide;
1129) 5-amino-3-(1-benzofuran-3-yl)-N-(tert-butyl)isoxazole-4-carboxamide;
1130) 5-amino-3-(1-benzofuran-3-yl)-N-(3,4-dimethoxybenzyl)isoxazole-4-carboxamide;
1131) 5-amino-3-(1-benzofuran-3-yl)-N-(2,3-dihydro-1H-inden-1-yl)isoxazole-4-carboxamide;
1132) 5-amino-3-(1-benzofuran-3-yl)-N-(1,2,3,4-tetrahydronaphthalen-1-yl)isoxazole-4-carboxamide;
1133) 5-amino-N-benzyl-3-(5-ethylthien-2-yl)isoxazole-4-carboxamide;
1134) 5-amino-3-(5-ethylthien-2-yl)-N-(2-furylmethyl)isoxazole-4-carboxamide;
1135) 5-amino-3-(5-ethylthien-2-yl)-N-(2-phenylethyl)isoxazole-4-carboxamide;
1136) 5-amino-N-(sec-butyl)-3-(5-ethylthien-2-yl)isoxazole-4-carboxamide;
1137) 5-amino-N-cyclohexyl-3-(5-ethylthien-2-yl)isoxazole-4-carboxamide;
1138) 5-amino-3-(5-ethylthien-2-yl)-N-(4-methoxybenzyl)isoxazole-4-carboxamide;
1139) 5-amino-3-(5-ethylthien-2-yl)-N-hexylisoxazole-4-carboxamide;
1140) 5-amino-3-(5-ethylthien-2-yl)-N-(thien-2-ylmethyl)isoxazole-4-carboxamide;
1141) 5-amino-N-cyclopentyl-3-(5-ethylthien-2-yl)isoxazole-4-carboxamide;
1142) 5-amino-N-(1,5-dimethylhexyl)-3-(5-ethylthien-2-yl)isoxazole-4-carboxamide;
1143) 5-amino-3-(5-ethylthien-2-yl)-N-isopentylisoxazole-4-carboxamide;
1144) 5-amino-N-(2-ethylhexyl)-3-(5-ethylthien-2-yl)isoxazole-4-carboxamide;
1145) 5-amino-3-(5-ethylthien-2-yl)-N-(4-phenylbutyl)isoxazole-4-carboxamide;
1146) 5-amino-3-(5-ethylthien-2-yl)-N-(2-methylcyclohexyl)isoxazole-4-carboxamide;
1147) 5-amino-N-[3,5-bis(trifluoromethyl)benzyl]-3-(5-ethylthien-2-yl)isoxazole-4-carboxamide;
1148) 5-amino-3-(5-ethylthien-2-yl)-N-[3-(trifluoromethyl)benzyl]isoxazole-4-carboxamide;
1149) 5-amino-3-(5-ethylthien-2-yl)-N-(3-phenylpropyl)isoxazole-4-carboxamide;
1150) 5-amino-3-(5-ethylthien-2-yl)-N-isobutylisoxazole-4-carboxamide;
1151) 5-amino-3-(5-ethylthien-2-yl)-N-[2-(trifluoromethyl)benzyl]isoxazole-4-carboxamide;
1152) 5-amino-3-(5-ethylthien-2-yl)-N-(2-methoxybenzyl)isoxazole-4-carboxamide;
1153) 5-amino-3-(5-ethylthien-2-yl)-N-(2-fluorobenzyl)isoxazole-4-carboxamide;
1154) 5-amino-N-(3,4-difluorobenzyl)-3-(5-ethylthien-2-yl)isoxazole-4-carboxamide;
1155) 5-amino-N-(3-chlorobenzyl)-3-(5-ethylthien-2-yl)isoxazole-4-carboxamide;
1156) 5-amino-N-(2-chlorobenzyl)-3-(5-ethylthien-2-yl)isoxazole-4-carboxamide;
1157) 5-amino-3-(5-ethylthien-2-yl)-N-(3-methylbenzyl)isoxazole-4-carboxamide;
1158) 5-amino-3-(5-ethylthien-2-yl)-N-(1-naphthylmethyl)isoxazole-4-carboxamide;
1159) 5-amino-N-(cyclopropylmethyl)-3-(5-ethylthien-2-yl)isoxazole-4-carboxamide;
1160) 5-amino-N-cycloheptyl-3-(5-ethylthien-2-yl)isoxazole-4-carboxamide;
1161) 5-amino-3-(5-ethylthien-2-yl)-N-[2-(4-methoxyphenyl)ethyl]isoxazole-4-carboxamide;
1162) 5-amino-N-(2-cyclohex-1-en-1-ylethyl)-3-(5-ethylthien-2-yl)isoxazole-4-carboxamide;
1163) 5-amino-N-(1,2-dimethylpropyl)-3-(5-ethylthien-2-yl)isoxazole-4-carboxamide;
1164) 5-amino-N-(3,3-diphenylpropyl)-3-(5-ethylthien-2-yl)isoxazole-4-carboxamide;
1165) 5-amino-N-(1,2-diphenylethyl)-3-(5-ethylthien-2-yl)isoxazole-4-carboxamide;
1166) 5-amino-N-(2,4-dimethoxybenzyl)-3-(5-ethylthien-2-yl)isoxazole-4-carboxamide;
1167) 5-amino-N-[3-(dibutylamino)propyl]-3-(5-ethylthien-2-yl)isoxazole-4-carboxamide;
1168) 5-amino-N-butyl-3-(5-ethylthien-2-yl)isoxazole-4-carboxamide;
1169) 5-amino-3-(5-ethylthien-2-yl)-N-[4-(trifluoromethoxy)benzyl]isoxazole-4-carboxamide;
1170) 5-amino-3-(5-ethylthien-2-yl)-N-[(5-methyl-2-furyl)methyl]isoxazole-4-carboxamide;
1171) 5-amino-3-(5-ethylthien-2-yl)-N-(1-methylhexyl)isoxazole-4-carboxamide;
1172) 5-amino-3-(5-ethylthien-2-yl)-N-(2-methoxy-1-methylethyl)isoxazole-4-carboxamide;
1173) 5-amino-N-(4-tert-butylcyclohexyl)-3-(5-ethylthien-2-yl)isoxazole-4-carboxamide;
1174) 5-amino-3-(5-ethylthien-2-yl)-N-(1-methylbutyl)isoxazole-4-carboxamide;
1175) 5-amino-3-(5-ethylthien-2-yl)-N-(3-fluorobenzyl)isoxazole-4-carboxamide;
1176) 5-amino-3-(5-ethylthien-2-yl)-N-(4-methylbenzyl)isoxazole-4-carboxamide;
1177) 5-amino-N-(1,3-dimethylbutyl)-3-(5-ethylthien-2-yl)isoxazole-4-carboxamide;
1178) 5-amino-3-(5-ethylthien-2-yl)-N-(1-methyl-3-phenylpropyl)isoxazole-4-carboxamide;
1179) 5-amino-3-(5-ethylthien-2-yl)-N-[3-(methylthio)propyl]isoxazole-4-carboxamide;
1180) 5-amino-N-(1,3-benzodioxol-5-ylmethyl)-3-(5-ethylthien-2-yl)isoxazole-4-carboxamide;
1181) 5-amino-3-(5-ethylthien-2-yl)-N-pentylisoxazole-4-carboxamide;
1182) 5-amino-N-(cyclohexylmethyl)-3-(5-ethylthien-2-yl)isoxazole-4-carboxamide;
1183) ethyl 4-({[5-amino-3-(5-ethylthien-2-yl)isoxazol-4-yl]carbonyl}amino)piperidine-1-carboxylate;
1184) 5-amino-N-[2-(3-chlorophenyl)ethyl]-3-(5-ethylthien-2-yl)isoxazole-4-carboxamide;
1185) 5-amino-N-(1-ethylpropyl)-3-(5-ethylthien-2-yl)isoxazole-4-carboxamide;
1186) 5-amino-3-(5-ethylthien-2-yl)-N-(1-phenylethyl)isoxazole-4-carboxamide;
1187) 5-amino-3-(5-ethylthien-2-yl)-N-(2-methylbenzyl)isoxazole-4-carboxamide;
1188) 5-amino-N-cyclobutyl-3-(5-ethylthien-2-yl)isoxazole-4-carboxamide;
1189) 5-amino-N-benzhydryl-3-(5-ethylthien-2-yl)isoxazole-4-carboxamide;
1190) 5-amino-3-(5-ethylthien-2-yl)-N-octylisoxazole-4-carboxamide;
1191) 5-amino-N-(2-bromobenzyl)-3-(5-ethylthien-2-yl)isoxazole-4-carboxamide;
1192) 5-amino-3-(5-ethylthien-2-yl)-N-(1-methylheptyl)isoxazole-4-carboxamide;
1193) 5-amino-3-(5-ethylthien-2-yl)-N-heptylisoxazole-4-carboxamide;
1194) 5-amino-N-(tert-butyl)-3-(5-ethylthien-2-yl)isoxazole-4-carboxamide;
1195) 5-amino-N-(3,4-dimethoxybenzyl)-3-(5-ethylthien-2-yl)isoxazole-4-carboxamide;
1196) 5-amino-N-(2,3-dihydro-1H-inden-1-yl)-3-(5-ethylthien-2-yl)isoxazole-4-carboxamide;
1197) 5-amino-3-(5-ethylthien-2-yl)-N-(1,2,3,4-tetrahydronaphthalen-1-yl)isoxazole-4-carboxamide;
1198) 5-amino-N-benzyl-3-(5-chloro-2-furyl)isoxazole-4-carboxamide;
1199) 5-amino-3-(5-chloro-2-furyl)-N-(2-furylmethyl)isoxazole-4-carboxamide;
1200) 5-amino-3-(5-chloro-2-furyl)-N-(2-phenylethyl)isoxazole-4-carboxamide;
1201) 5-amino-N-(sec-butyl)-3-(5-chloro-2-furyl)isoxazole-4-carboxamide;
1202) 5-amino-3-(5-chloro-2-furyl)-N-cyclohexylisoxazole-4-carboxamide;
1203) 5-amino-3-(5-chloro-2-furyl)-N-(4-methoxybenzyl)isoxazole-4-carboxamide;
1204) 5-amino-3-(5-chloro-2-furyl)-N-hexylisoxazole-4-carboxamide;
1205) 5-amino-3-(5-chloro-2-furyl)-N-cyclopentylisoxazole-4-carboxamide;
1206) 5-amino-3-(5-chloro-2-furyl)-N-(1,5-dimethylhexyl)isoxazole-4-carboxamide;
1207) 5-amino-3-(5-chloro-2-furyl)-N-isopentylisoxazole-4-carboxamide;
1208) 5-amino-3-(5-chloro-2-furyl)-N-(2-ethylhexyl)isoxazole-4-carboxamide;
1209) 5-amino-3-(5-chloro-2-furyl)-N-(4-phenylbutyl)isoxazole-4-carboxamide;
1210) 5-amino-3-(5-chloro-2-furyl)-N-(2-methylcyclohexyl)isoxazole-4-carboxamide;
1211) 5-amino-N-[3,5-bis(trifluoromethyl)benzyl]-3-(5-chloro-2-furyl)isoxazole-4-carboxamide;
1212) 5-amino-3-(5-chloro-2-furyl)-N-[3-(trifluoromethyl)benzyl]isoxazole-4-carboxamide;
1213) 5-amino-3-(5-chloro-2-furyl)-N-(3-phenylpropyl)isoxazole-4-carboxamide;
1214) 5-amino-3-(5-chloro-2-furyl)-N-isobutylisoxazole-4-carboxamide;
1215) 5-amino-3-(5-chloro-2-furyl)-N-[2-(trifluoromethyl)benzyl]isoxazole-4-carboxamide;
1216) 5-amino-3-(5-chloro-2-furyl)-N-(2-methoxybenzyl)isoxazole-4-carboxamide
1217) 5-amino-3-(5-chloro-2-furyl)-N-(2-fluorobenzyl)isoxazole-4-carboxamide;
1218) 5-amino-3-(5-chloro-2-furyl)-N-[4-(trifluoromethyl)benzyl]isoxazole-4-carboxamide;
1219) 5-amino-3-(5-chloro-2-furyl)-N-(3,4-difluorobenzyl)isoxazole-4-carboxamide;
1220) 5-amino-N-(3-chlorobenzyl)-3-(5-chloro-2-furyl)isoxazole-4-carboxamide;
1221) 5-amino-N-(2-chlorobenzyl)-3-(5-chloro-2-furyl)isoxazole-4-carboxamide;
1222) 5-amino-3-(5-chloro-2-furyl)-N-(3-methylbenzyl)isoxazole-4-carboxamide;
1223) 5-amino-3-(5-chloro-2-furyl)-N-(1-naphthylmethyl)isoxazole-4-carboxamide;
1224) 5-amino-3-(5-chloro-2-furyl)-N-(cyclopropylmethyl)isoxazole-4-carboxamide;
1225) 5-amino-3-(5-chloro-2-furyl)-N-cycloheptylisoxazole-4-carboxamide;
1226) 5-amino-3-(5-chloro-2-furyl)-N-[2-(4-methoxyphenyl)ethyl]isoxazole-4-carboxamide;
1227) 5-amino-3-(5-chloro-2-furyl)-N-(2-cyclohex-1-en-1-ylethyl)isoxazole-4-carboxamide;
1228) 5-amino-3-(5-chloro-2-furyl)-N-(1,2-dimethylpropyl)isoxazole-4-carboxamide;
1229) 5-amino-3-(5-chloro-2-furyl)-N-(3,3-diphenylpropyl)isoxazole-4-carboxamide;
1230) 5-amino-3-(5-chloro-2-furyl)-N-(1,2-diphenylethyl)isoxazole-4-carboxamide;
1231) 5-amino-3-(5-chloro-2-furyl)-N-(2,4-dimethoxybenzyl)isoxazole-4-carboxamide;
1232) 5-amino-3-(5-chloro-2-furyl)-N-[3-(dibutylamino)propyl]isoxazole-4-carboxamide;
1233) 5-amino-N-butyl-3-(5-chloro-2-furyl)isoxazole-4-carboxamide;
1234) 5-amino-3-(5-chloro-2-furyl)-N-[4-(trifluoromethoxy)benzyl]isoxazole-4-carboxamide;
1235) 5-amino-3-(5-chloro-2-furyl)-N-[(5-methyl-2-furyl)methyl]isoxazole-4-carboxamide;
1236) 5-amino-3-(5-chloro-2-furyl)-N-(1-methylhexyl)isoxazole-4-carboxamide;
1237) 5-amino-N-(1-benzylpiperidin-4-yl)-3-(5-chloro-2-furyl)isoxazole-4-carboxamide;
1238) 5-amino-3-(5-chloro-2-furyl)-N-(2-methoxy-1-methylethyl)isoxazole-4-carboxamide;
1239) 5-amino-N-(4-tert-butylcyclohexyl)-3-(5-chloro-2-furyl)isoxazole-4-carboxamide;
1240) 5-amino-3-(5-chloro-2-furyl)-N-(1-methylbutyl)isoxazole-4-carboxamide;
1241) 5-amino-3-(5-chloro-2-furyl)-N-(3-fluorobenzyl)isoxazole-4-carboxamide;
1242) 5-amino-3-(5-chloro-2-furyl)-N-(4-methylbenzyl)isoxazole-4-carboxamide;
1243) 5-amino-3-(5-chloro-2-furyl)-N-(1,3-dimethylbutyl)isoxazole-4-carboxamide;
1244) 5-amino-3-(5-chloro-2-furyl)-N-(1-methyl-3-phenylpropyl)isoxazole-4-carboxamide;
1245) 5-amino-3-(5-chloro-2-furyl)-N-[3-(methylthio)propyl]isoxazole-4-carboxamide;
1246) 5-amino-N-(1,3-benzodioxol-5-ylmethyl)-3-(5-chloro-2-furyl)isoxazole-4-carboxamide;
1247) 5-amino-3-(5-chloro-2-furyl)-N-pentylisoxazole-4-carboxamide;
1248) 5-amino-3-(5-chloro-2-furyl)-N-(cyclohexylmethyl)isoxazole-4-carboxamide;
1249) ethyl 4-({[5-amino-3-(5-chloro-2-furyl)isoxazol-4-yl]carbonyl}amino)piperidine-1-carboxylate;
1250) 5-amino-3-(5-chloro-2-furyl)-N-[2-(3-chlorophenyl)ethyl]isoxazole-4-carboxamide;
1251) 5-amino-3-(5-chloro-2-furyl)-N-(1-ethylpropyl)isoxazole-4-carboxamide;
1252) 5-amino-3-(5-chloro-2-furyl)-N-(1-phenylethyl)isoxazole-4-carboxamide;
1253) 5-amino-3-(5-chloro-2-furyl)-N-(2-methylbenzyl)isoxazole-4-carboxamide;
1254) 5-amino-3-(5-chloro-2-furyl)-N-cyclobutylisoxazole-4-carboxamide;
1255) 5-amino-N-benzhydryl-3-(5-chloro-2-furyl)isoxazole-4-carboxamide;
1256) 5-amino-3-(5-chloro-2-furyl)-N-octylisoxazole-4-carboxamide;
1257) 5-amino-N-(2-bromobenzyl)-3-(5-chloro-2-furyl)isoxazole-4-carboxamide;
1258) 5-amino-3-(5-chloro-2-furyl)-N-(1-methylheptyl)isoxazole-4-carboxamide;
1259) 5-amino-3-(5-chloro-2-furyl)-N-heptylisoxazole-4-carboxamide;
1260) 5-amino-N-(tert-butyl)-3-(5-chloro-2-furyl)isoxazole-4-carboxamide;
1261) 5-amino-3-(5-chloro-2-furyl)-N-(3,4-dimethoxybenzyl)isoxazole-4-carboxamide; 1262) 5-amino-3-(5-chloro-2-furyl)-N-(2,3-dihydro-1H-inden-1-yl)isoxazole-4-carboxamide;
1263) 5-amino-3-(5-chloro-2-furyl)-N-(1,2,3,4-tetrahydronaphthalen-1-yl)isoxazole-4-carboxamide;
1264) 5-amino-N-benzyl-3-(6-methoxypyridin-3-yl)isoxazole-4-carboxamide;
1265) 5-amino-N-(2-furylmethyl)-3-(6-methoxypyridin-3-yl)isoxazole-4-carboxamide;
1266) 5-amino-3-(6-methoxypyridin-3-yl)-N-(2-phenylethyl)isoxazole-4-carboxamide;
1267) 5-amino-N-(sec-butyl)-3-(6-methoxypyridin-3-yl)isoxazole-4-carboxamide;
1268) 5-amino-N-cyclohexyl-3-(6-methoxypyridin-3-yl)isoxazole-4-carboxamide;
1269) 5-amino-N-(4-methoxybenzyl)-3-(6-methoxypyridin-3-yl)isoxazole-4-carboxamide;
1270) 5-amino-N-hexyl-3-(6-methoxypyridin-3-yl)isoxazole-4-carboxamide;
1271) 5-amino-3-(6-methoxypyridin-3-yl)-N-(thien-2-ylmethyl)isoxazole-4-carboxamide;
1272) 5-amino-N-cyclopentyl-3-(6-methoxypyridin-3-yl)isoxazole-4-carboxamide;
1273) 5-amino-N-(1,5-dimethylhexyl)-3-(6-methoxypyridin-3-yl)isoxazole-4-carboxamide;
1274) 5-amino-N-isopentyl-3-(6-methoxypyridin-3-yl)isoxazole-4-carboxamide;
1275) 5-amino-N-(2-ethylhexyl)-3-(6-methoxypyridin-3-yl)isoxazole-4-carboxamide;
1276) 5-amino-3-(6-methoxypyridin-3-yl)-N-(4-phenylbutyl)isoxazole-4-carboxamide;
1277) 5-amino-3-(6-methoxypyridin-3-yl)-N-(2-methylcyclohexyl)isoxazole-4-carboxamide;
1278) 5-amino-N-[3,5-bis(trifluoromethyl)benzyl]-3-(6-methoxypyridin-3-yl)isoxazole-4-carboxamide;
1279) 5-amino-3-(6-methoxypyridin-3-yl)-N-[3-(trifluoromethyl)benzyl]isoxazole-4-carboxamide;
1280) 5-amino-3-(6-methoxypyridin-3-yl)-N-(3-phenylpropyl)isoxazole-4-carboxamide;
1281) 5-amino-N-isobutyl-3-(6-methoxypyridin-3-yl)isoxazole-4-carboxamide;
1282) 5-amino-3-(6-methoxypyridin-3-yl)-N-[2-(trifluoromethyl)benzyl]isoxazole-4-carboxamide;
1283) 5-amino-N-(2-methoxybenzyl)-3-(6-methoxypyridin-3-yl)isoxazole-4-carboxamide;
1284) 5-amino-N-(2-fluorobenzyl)-3-(6-methoxypyridin-3-yl)isoxazole-4-carboxamide;
1285) 5-amino-3-(6-methoxypyridin-3-yl)-N-[4-(trifluoromethyl)benzyl]isoxazole-4-carboxamide;
1286) 5-amino-N-(3,4-difluorobenzyl)-3-(6-methoxypyridin-3-yl)isoxazole-4-carboxamide;
1287) 5-amino-N-(3-chlorobenzyl)-3-(6-methoxypyridin-3-yl)isoxazole-4-carboxamide;
1288) 5-amino-N-(2-chlorobenzyl)-3-(6-methoxypyridin-3-yl)isoxazole-4-carboxamide;
1289) 5-amino-3-(6-methoxypyridin-3-yl)-N-(3-methylbenzyl)isoxazole-4-carboxamide;
1290) 5-amino-3-(6-methoxypyridin-3-yl)-N-(1-naphthylmethyl)isoxazole-4-carboxamide;
1291) 5-amino-N-(cyclopropylmethyl)-3-(6-methoxypyridin-3-yl)isoxazole-4-carboxamide;
1292) 5-amino-N-cycloheptyl-3-(6-methoxypyridin-3-yl)isoxazole-4-carboxamide;
1293) 5-amino-N-[2-(4-methoxyphenyl)ethyl]-3-(6-methoxypyridin-3-yl)isoxazole-4-carboxamide;
1294) 5-amino-N-(2-cyclohex-1-en-1-ylethyl)-3-(6-methoxypyridin-3-yl)isoxazole-4-carboxamide;
1295) 5-amino-N-(1,2-dimethylpropyl)-3-(6-methoxypyridin-3-yl)isoxazole-4-carboxamide;
1296) 5-amino-N-(3,3-diphenylpropyl)-3-(6-methoxypyridin-3-yl)isoxazole-4-carboxamide;
1297) 5-amino-N-(1,2-diphenylethyl)-3-(6-methoxypyridin-3-yl)isoxazole-4-carboxamide;
1298) 5-amino-N-(2,4-dimethoxybenzyl)-3-(6-methoxypyridin-3-yl)isoxazole-4-carboxamide;
1299) 5-amino-N-[3-(dibutylamino)propyl]-3-(6-methoxypyridin-3-yl)isoxazole-4-carboxamide;
1300) 5-amino-N-butyl-3-(6-methoxypyridin-3-yl)isoxazole-4-carboxamide;
1301) 5-amino-3-(6-methoxypyridin-3-yl)-N-[4-(trifluoromethoxy)benzyl]isoxazole-4-carboxamide;
1302) 5-amino-3-(6-methoxypyridin-3-yl)-N-[(5-methyl-2-furyl)methyl]isoxazole-4-carboxamide;
1303) 5-amino-3-(6-methoxypyridin-3-yl)-N-(1-methylhexyl)isoxazole-4-carboxamide;
1304) 5-amino-N-(1-benzylpiperidin-4-yl)-3-(6-methoxypyridin-3-yl)isoxazole-4-carboxamide;
1305) 5-amino-N-(2-methoxy-1-methylethyl)-3-(6-methoxypyridin-3-yl)isoxazole-4-carboxamide;
1306) 5-amino-N-(4-tert-butylcyclohexyl)-3-(6-methoxypyridin-3-yl)isoxazole-4-carboxamide;
1307) 5-amino-3-(6-methoxypyridin-3-yl)-N-(1-methylbutyl)isoxazole-4-carboxamide;
1308) 5-amino-N-(3-fluorobenzyl)-3-(6-methoxypyridin-3-yl)isoxazole-4-carboxamide;
1309) 5-amino-3-(6-methoxypyridin-3-yl)-N-(4-methylbenzyl)isoxazole-4-carboxamide;
1310) 5-amino-N-(1,3-dimethylbutyl)-3-(6-methoxypyridin-3-yl)isoxazole-4-carboxamide;
1311) 5-amino-3-(6-methoxypyridin-3-yl)-N-(1-methyl-3-phenylpropyl)isoxazole-4-carboxamide;
1312) 5-amino-3-(6-methoxypyridin-3-yl)-N-[3-(methylthio)propyl]isoxazole-4-carboxamide;
1313) 5-amino-N-(1,3-benzodioxol-5-ylmethyl)-3-(6-methoxypyridin-3-yl)isoxazole-4-carboxamide;
1314) 5-amino-3-(6-methoxypyridin-3-yl)-N-pentylisoxazole-4-carboxamide;
1315) 5-amino-N-(cyclohexylmethyl)-3-(6-methoxypyridin-3-yl)isoxazole-4-carboxamide;
1316) ethyl 4-({[5-amino-3-(6-methoxypyridin-3-yl)isoxazol-4-yl]carbonyl}amino)piperidine-1-carboxylate;
1317) 5-amino-N-[2-(3-chlorophenyl)ethyl]-3-(6-methoxypyridin-3-yl)isoxazole-4-carboxamide;
1318) 5-amino-N-(1-ethylpropyl)-3-(6-methoxypyridin-3-yl)isoxazole-4-carboxamide;
1319) 5-amino-3-(6-methoxypyridin-3-yl)-N-(1-phenylethyl)isoxazole-4-carboxamide;
1320) 5-amino-3-(6-methoxypyridin-3-yl)-N-(2-methylbenzyl)isoxazole-4-carboxamide;
1321) 5-amino-N-cyclobutyl-3-(6-methoxypyridin-3-yl)isoxazole-4-carboxamide;
1322) 5-amino-N-benzhydryl-3-(6-methoxypyridin-3-yl)isoxazole-4-carboxamide;
1323) 5-amino-3-(6-methoxypyridin-3-yl)-N-octylisoxazole-4-carboxamide;
1324) 5-amino-N-(2-bromobenzyl)-3-(6-methoxypyridin-3-yl)isoxazole-4-carboxamide;
1325) 5-amino-3-(6-methoxypyridin-3-yl)-N-(1-methylheptyl)isoxazole-4-carboxamide;
1326) 5-amino-N-heptyl-3-(6-methoxypyridin-3-yl)isoxazole-4-carboxamide;
1327) 5-amino-N-(tert-butyl)-3-(6-methoxypyridin-3-yl)isoxazole-4-carboxamide;
1328) 5-amino-N-(3,4-dimethoxybenzyl)-3-(6-methoxypyridin-3-yl)isoxazole-4-carboxamide;
1329) 5-amino-N-(2,3-dihydro-1H-inden-1-yl)-3-(6-methoxypyridin-3-yl)isoxazole-4-carboxamide;
1330) 5-amino-3-(6-methoxypyridin-3-yl)-N-(1,2,3,4-tetrahydronaphthalen-1-yl)isoxazole-4-carboxamide;
1331) 5-amino-N-benzyl-3-pyridin-3-ylisoxazole-4-carboxamide;
1332) 5-amino-N-(2-furylmethyl)-3-pyridin-3-ylisoxazole-4-carboxamide;
1333) 5-amino-N-(2-phenylethyl)-3-pyridin-3-ylisoxazole-4-carboxamide;
1334) 5-amino-N-cyclohexyl-3-pyridin-3-ylisoxazole-4-carboxamide;
1335) 5-amino-N-(4-methoxybenzyl)-3-pyridin-3-ylisoxazole-4-carboxamide;
1336) 5-amino-N-hexyl-3-pyridin-3-ylisoxazole-4-carboxamide;
1337) 5-amino-3-pyridin-3-yl-N-(thien-2-ylmethyl)isoxazole-4-carboxamide;
1338) 5-amino-N-cyclopentyl-3-pyridin-3-ylisoxazole-4-carboxamide;
1339) 5-amino-N-(1,5-dimethylhexyl)-3-pyridin-3-ylisoxazole-4-carboxamide;
1340) 5-amino-N-isopentyl-3-pyridin-3-ylisoxazole-4-carboxamide;
1341) 5-amino-N-(2-ethylhexyl)-3-pyridin-3-ylisoxazole-4-carboxamide;
1342) 5-amino-N-(4-phenylbutyl)-3-pyridin-3-ylisoxazole-4-carboxamide;
1343) 5-amino-N-(2-methylcyclohexyl)-3-pyridin-3-ylisoxazole-4-carboxamide;
1344) 5-amino-N-[3,5-bis(trifluoromethyl)benzyl]-3-pyridin-3-ylisoxazole-4-carboxamide;
1345) 5-amino-3-pyridin-3-yl-N-[3-(trifluoromethyl)benzyl]isoxazole-4-carboxamide;
1346) 5-amino-N-(3-phenylpropyl)-3-pyridin-3-ylisoxazole-4-carboxamide;
1347) 5-amino-N-isobutyl-3-pyridin-3-ylisoxazole-4-carboxamide;
1348) 5-amino-3-pyridin-3-yl-N-[2-(trifluoromethyl)benzyl]isoxazole-4-carboxamide;
1349) 5-amino-N-(2-methoxybenzyl)-3-pyridin-3-ylisoxazole-4-carboxamide;
1350) 5-amino-N-(2-fluorobenzyl)-3-pyridin3-ylisoxazole-4-carboxamide;
1351) 5-amino-3-pyridin-3-yl-N-[4-(trifluoromethyl)benzyl]isoxazole-4-carboxamide;
1352) 5-amino-N-(3,4-difluorobenzyl)-3-pyridin-3-ylisoxazole-4-carboxamide;
1353) 5-amino-N-(3-chlorobenzyl)-3-pyridin-3-ylisoxazole-4-carboxamide;
1354) 5-amino-N-(2-chlorobenzyl)-3-pyridin-3-ylisoxazole-4-carboxamide;
1355) 5-amino-N-(3-methylbenzyl)-3-pyridin-3-ylisoxazole-4-carboxamide;
1356) 5-amino-N-(1-naphthylmethyl)-3-pyridin-3-ylisoxazole-4-carboxamide;
1357) 5-amino-N-(cyclopropylmethyl)-3-pyridin-3-ylisoxazole-4-carboxamide;
1358) 5-amino-N-cycloheptyl-3-pyridin-3-ylisoxazole-4-carboxamide;
1359) 5-amino-N-[2-(4-methoxyphenyl)ethyl]-3-pyridin-3-ylisoxazole-4-carboxamide;
1360) 5-amino-N-(2-cyclohex-1-en-1-ylethyl)-3-pyridin-3-ylisoxazole-4-carboxamide;
1361) 5-amino-N-(1,2-dimethylpropyl)-3-pyridin-3-ylisoxazole-4-carboxamide;
1362) 5-amino-N-(3,3-diphenylpropyl)-3-pyridin-3-ylisoxazole-4-carboxamide;
1363) 5-amino-N-(1,2-diphenylethyl)-3-pyridin-3-ylisoxazole-4-carboxamide;
1364) 5-amino-N-(2,4-dimethoxybenzyl)-3-pyridin-3-ylisoxazole-4-carboxamide;
1365) 5-amino-N-[3-(dibutylamino)propyl]-3-pyridin-3-ylisoxazole-4-carboxamide;
1366) 5-amino-N-butyl-3-pyridin-3-ylisoxazole-4-carboxamide;
1367) 5-amino-3-pyridin-3-yl-N-[4-(trifluoromethoxy)benzyl]isoxazole-4-carboxamide;
1368) 5-amino-N-[(5-methyl-2-furyl)methyl]-3-pyridin-3-ylisoxazole-4-carboxamide;
1369) 5-amino-N-(1-methylhexyl)-3-pyridin-3-ylisoxazole-4-carboxamide;
1370) 5-amino-N-(1-methylbutyl)-3-pyridin-3-ylisoxazole-4-carboxamide;
1371) 5-amino-N-(3-fluorobenzyl)-3-pyridin-3-ylisoxazole-4-carboxamide;
1372) 5-amino-N-(4-methylbenzyl)-3-pyridin-3-ylisoxazole-4-carboxamide;
1373) 5-amino-N-(1,3-dimethylbutyl)-3-pyridin-3-ylisoxazole-4-carboxamide;
1374) 5-amino-N-(1-methyl-3-phenylpropyl)-3-pyridin-3-ylisoxazole-4-carboxamide;
1375) 5-amino-N-[3-(methylthio)propyl]-3-pyridin-3-ylisoxazole-4-carboxamide;
1376) 5-amino-N-(1,3-benzodioxol-5-ylmethyl)-3-pyridin-3-ylisoxazole-4-carboxamide;
1377) 5-amino-N-pentyl-3-pyridin-3-ylisoxazole-4-carboxamide;
1378) 5-amino-N-(cyclohexylmethyl)-3-pyridin-3-ylisoxazole-4-carboxamide;
1379) ethyl 4-{[(5-amino-3-pyridin-3-ylisoxazol-4-yl)carbonyl]amino}piperidine-1-carboxylate;
1380) 5-amino-N-[2-(3-chlorophenyl)ethyl]-3-pyridin-3-ylisoxazole-4-carboxamide;
1381) 5-amino-N-(1-ethylpropyl)-3-pyridin-3-ylisoxazole-4-carboxamide;
1382) 5-amino-N-(1-phenylethyl)-3-pyridin-3-ylisoxazole-4-carboxamide;
1383) 5-amino-N-(2-methylbenzyl)-3-pyridin-3-ylisoxazole-4-carboxamide;
1384) 5-amino-N-cyclobutyl-3-pyridin-3-ylisoxazole-4-carboxamide;
1385) 5-amino-N-benzhydryl-3-pyridin-3-ylisoxazole-4-carboxamide;
1386) 5-amino-N-octyl-3-pyridin-3-ylisoxazole-4-carboxamide;
1387) 5-amino-N-(2-bromobenzyl)-3-pyridin-3-ylisoxazole-4-carboxamide;
1388) 5-amino-N-(1-methylheptyl)-3-pyridin-3-ylisoxazole-4-carboxamide;
1389) 5-amino-N-heptyl-3-pyridin-3-ylisoxazole-4-carboxamide;
1390) 5-amino-N-(tert-butyl)-3-pyridin-3-ylisoxazole-4-carboxamide;
1391) 5-amino-N-(3,4-dimethoxybenzyl)-3-pyridin-3-ylisoxazole-4-carboxamide;
1392) 5-amino-N-(2,3-dihydro-1H-inden-1-yl)-3-pyridin-3-ylisoxazole-4-carboxamide;
1393) 5-amino-3-pyridin-3-yl-N-(1,2,3,4-tetrahydronaphthalen-1-yl)isoxazole-4-carboxamide.

24. A process for preparing the compounds of formula (I) and the pharmaceutically acceptable salts, as defined in claim 13, which process comprises:
a) reacting a compound of formula (II) with a compound of formula (III) 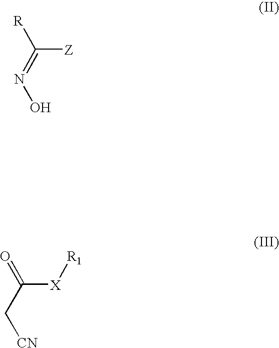
wherein R, R₁ and X are as above defined and Z represents a halogen atom or a suitable leaving group, so as to obtain a compound of formula (I) 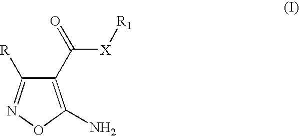
and, optionally, reacting the compound of formula (I) according to any one of the alternative steps b) below
b.1) with a compound of formula (IV), (V) or (VI)
R₂—COW (IV),
R₂—SO₂W′ (V),
R₂—NCO (VI)
wherein R₂ is as above defined, W is hydroxy or a suitable leaving group and W′ is a suitable leaving group, so as to obtain the compounds of formula (I) wherein Y is —CO—, —SO₂— or —CONH—, respectively, and R, R₁, R₂ and X are as above defined;
b.2) with a suitable aldehyde or ketone derivative of formula (VII)
R₂—CO—R₃ (VII)
wherein R₂ and R₃ are as above defined, under reductive conditions, so as to obtain a compound of formula (I) wherein Y is a group —CH(R₃)— and R, R₁, R₂, R₃ and X are as above defined;
b.3) with a suitable acylating agent in the presence of ammonia, so as to obtain a compound of formula (I) wherein Y is —CONH—, R₂ is hydrogen and R, R₁ and X are as above defined; and, optionally
c) converting the thus obtained compound of formula (I) into another compound of formula (I) and/or into a pharmaceutically acceptable salt thereof.

25. The process of claim 24 wherein, within the compound of formula (IV) or (V) in step b.1), W is hydroxy or a chlorine or bromine atom and W′ is a chlorine or bromine atom.

26. The process of claim 24 wherein, within step b.2), reductive conditions occur in the presence of sodium borohydride, sodium cyanoborohydride or sodium triacetoxyborohydride.

27. The process of claim 24 wherein, in step b.3), the acylating agent is selected from triphosgene or trichloromethyl chloroformate.

28. A process under solid phase synthesis (SPS) conditions for preparing the compounds of formula (I) and the pharmaceutically acceptable salts, as defined in claim 13, which process comprises:
a′) reacting cyanoacetic acid with a suitable polystyrenic resin of formula (X) or (XI)
Resin-W (X),
Resin-NHR₁ (XI)
wherein R₁ is as above defined and W is hydroxy or a suitable leaving group, so as to obtain the compounds of formula (XII) or (XIII), respectively 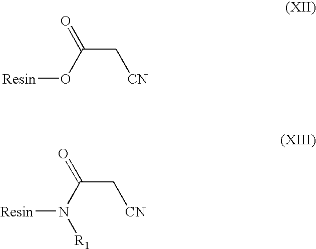
wherein R₁ is as above reported;
b′) reacting any one of the compounds of formula (XII) or (XIII) with a compound of formula (II) 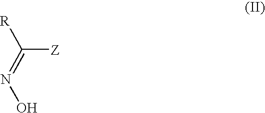
wherein R and Z are as above defined, so as to obtain a compound of formula (XIV) or (XV) 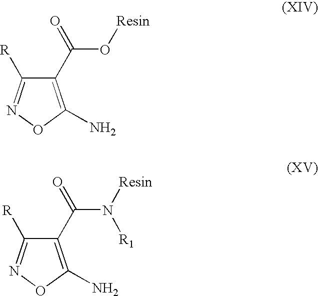
c′) cleaving the resin from the compounds of formula (XIV) or (XV) under acidic or basic conditions, so as to obtain a compound of formula (I) wherein X is a group —O— and R₁ is hydrogen, or X is a group —N(R₃)— wherein R₃ is hydrogen and R₁ is as above defined, respectively; or, alternatively, reacting the above compounds of formula (XIV) or (XV) according to any one of the alternative steps d′) below d.1′) with a compound of formula (IV), (V) or (VI)
R₂—COW (IV),
R₂—SO₂W′ (V),
R₂—NCO (VI)
wherein R₂ is as above defined, W is hydroxy or a suitable leaving group and W′ is a suitable leaving group, so as to obtain the compounds of formula (XVI) or (XVII) 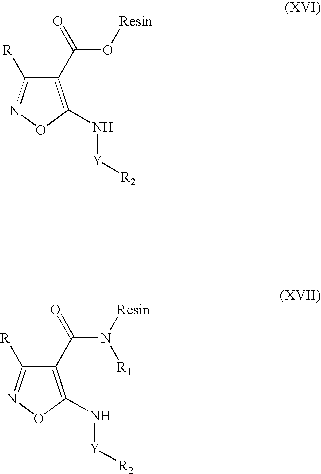
wherein Y is —CO—, —SO₂— or —CONH—, respectively, and R, R₁ and R₂ are as above defined;
d.2′) with a suitable aldehyde or ketone derivative of formula (VII)
R₂—CO—R₃ (VII)
wherein R₂ and R₃ are as above defined, under reductive conditions, so as to obtain the compounds of the above formula (XVI) or (XVII) wherein Y is a group —CH(R₃)— and R, R₁, R₂ and R₃ are as above defined;
d.3′) with a suitable acylating agent in the presence of ammonia, so as to obtain a compound of the above formula (XVI) or (XVII) wherein Y is —CONH—, R₂ is hydrogen and R and R₁ are as above defined;
e′) cleaving the resin from the compounds of formula (XVI) or (XVII) under acidic or basic conditions, so as to obtain a compound of formula (I) wherein X is a group —O— and R₁ is hydrogen, or X is a group —N(R₃)— wherein R₃ is hydrogen and R₁ is as above defined, respectively; and, optionally,
f′) converting the thus obtained compound of formula (I) into another compound of formula (I) and/or into a pharmaceutically acceptable salt thereof.

29. A library of two or more compounds of formula (I) 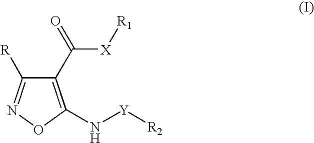
wherein
R is an optionally substituted 5 or 6 membered heteroaryl group with from 1 to 3 heteroatoms selected among nitrogen, oxygen or sulfur, optionally further condensed with a 5 to 7 membered aromatic or non-aromatic carbocycle or heterocycle with from 1 to 3 heteroatoms selected among nitrogen, oxygen or sulfur;
X is a divalent group selected from —N(R₃)— or —O—;
Y is a divalent group selected from —CH(R₃)—, —CO—, —CONH— or —SO₂—, or Y may also be a single bond when R₂ is a hydrogen atom or a C₃-C₆ cycloalkyl group;
R₁ is a hydrogen atom or a group, optionally further substituted, selected from straight or branched C₁-C₆ alkyl, C₃-C₆ cycloalkyl, aryl or aryl C₁-C₆ alkyl, 5 or 6 membered heterocyclyl or heterocyclyl C₁-C₆ alkyl having from 1 to 3 heteroatoms selected among nitrogen, oxygen or sulfur; the said cycloalkyl, aryl or heterocyclyl groups being optionally further condensed with a 5 to 7 membered aromatic or non-aromatic carbocycle or heterocycle with from 1 to 3 heteroatoms selected among nitrogen, oxygen or sulfur;
R₂ and R₃ have, each independently, the meanings above reported for R₁ or represent an optionally substituted straight or branched C₂-C₆ alkenyl or alkynyl group; and the pharmaceutically acceptable salts thereof.

30. Any specific compound of formula (I), as defined in claim 13, which is obtainable, for instance through a combinatorial chemistry technique as per the process of claim 28, by reacting each of the derivatives of formula (II), as set forth in table I, with any one of the derivatives of formula (XIII) which are obtainable from the amines of formula (IX), as set forth in table II, and by subsequently reacting any one of the obtained derivatives of formula (XV) with any one of the derivatives of formula (IV), as set forth in table III.

31. Any specific compound of formula (I), as defined in claim 13, which is obtainable, for instance through a combinatorial chemistry technique as per the process of claim 28, by reacting each of the derivatives of formula (II), as set forth in table I, with any one of the derivatives of formula (XIII) which are obtainable from the amines of formula (IX), as set forth in table II, and by subsequently reacting any one of the obtained derivatives of formula (XV) with any one of the derivatives of formula (V), as set forth in table IV.

32. Any specific compound of formula (I), as defined in claim 13, which is obtainable, for instance through a combinatorial chemistry technique as per the process of claim 28, by reacting each of the derivatives of formula (II), as set forth in table I, with any one of the derivatives of formula (XIII) which are obtainable from the amines of formula (IX), as set forth in table II, and by subsequently reacting any one of the obtained derivatives of formula (XV) with any one of the derivatives of formula (VI), as set forth in table V.

33. A pharmaceutical composition comprising an effective amount of an aminoisoxazole of formula (I) as defined in claim 13 and, at least, one pharmaceutically acceptable excipient, carrier or diluent.

34. A pharmaceutical composition according to claim 33 further comprising one or more chemotherapeutic agents, as a combined preparation for simultaneous, separate or sequential use in anticancer therapy.

35. A product or kit comprising a compound of claim 13 or a pharmaceutical composition thereof as defined in claim 34, and one or more chemotherapeutic agents, as a combined preparation for simultaneous, separate or sequential use in anticancer therapy.

36. A compound of formula (I) or a pharmaceutically acceptable salt thereof, as defined in claim 13, for use as a medicament.

37. Use of a compound of formula (I) or a pharmaceutically acceptable salt thereof, as defined in claim 13, in the manufacture of a medicament for treating diseases caused by and/or associated with an altered protein kinase activity.

38. Use according to claim 37 for treating tumors.
